(19) United States
(12) Patent Application Publication
(10) Pub. No.: US 2025/0122162 A1
(43) Pub. Date: Apr. 17, 2025

(54) ORGANIC COMPOUND, ORGANIC SEMICONDUCTOR DEVICE, LIGHT-EMITTING DEVICE, AND ELECTRONIC DEVICE

(71) Applicant: SEMICONDUCTOR ENERGY LABORATORY CO., LTD., Atsugi-shi (JP)

(72) Inventors: Masanori TAKEDA, Atsugi (JP); Kazuki KAJIYAMA, Hadano (JP); Sachiko KAWAKAMI, Atsugi (JP); Naoaki HASHIMOTO, Sagamihara (JP); Tsunenori SUZUKI, Yokohama (JP)

(21) Appl. No.: 18/897,912

(22) Filed: Sep. 26, 2024

(30) Foreign Application Priority Data
Sep. 29, 2023 (JP) 2023-170482

(51) Int. Cl. C07D 307/91; C07C 211/61; C07D 333/76; C07D 335/02; H10K 50/11; H10K 59/35

(52) U.S. Cl. CPC C07D 307/91; C07C 211/61; C07D 333/76; C07D 335/02; C07C 2601/14; C07C 2603/18; H10K 50/11; H10K 59/353

(57) ABSTRACT

An organic compound that can provide a light-emitting device having a high hole-transport property and high reliability. An organic compound represented by General Formula (G1) shown below is provided. In General Formula (G1), X represents a sulfur atom or an oxygen atom, each of R¹ to R²² independently represents any one of hydrogen, halogen, a nitrile group, an alkenyl group, a vinyl group, an alkynyl group, a straight-chain alkyl group having 1 to 6 carbon atoms, a cycloalkyl group having 3 to 10 carbon atoms, an alkoxy group having 1 to 6 carbon atoms, an alkylsilyl group having 3 to 12 carbon atoms, an aryl group having 6 to 30 carbon atoms, and a heteroaryl group having 2 to 30 carbon atoms. At least one of R¹⁶ to R²² represents a naphthyl group, n represents an integer of 0 to 4, and Ar¹ represents a fluorenyl group or a spirofluorenyl group.

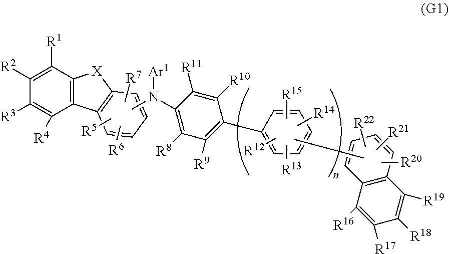

### BACKGROUND OF THE INVENTION

### 1. Field of the Invention

One embodiment of the present invention relates to an organic compound, an organic electronic device, a light-emitting device, and an electronic device.

Note that one embodiment of the present invention is not limited to the above technical field. Examples of the technical field of one embodiment of the present invention include a compound, a light-emitting device, a semiconductor device, a display device, a light-emitting apparatus, a power storage device, a memory device, an electronic device, a lighting device, an input device (e.g., a touch sensor), an input/output device (e.g., a touch panel), a method for driving any of them, and a method for manufacturing any of them.

### 2. Description of the Related Art

Recent display devices have been expected to be applied to a variety of uses. Usage examples of large-sized display devices include a television device for home use (also referred to as TV or television receiver), digital signage, and a public information display (PID). In addition, a smartphone, a tablet terminal, and the like each including a touch panel are being developed as portable information terminals.

An increase in the resolution of display devices is also required. For example, devices for virtual reality (VR), augmented reality (AR), substitutional reality (SR), or mixed reality (MR) are given as devices requiring high-resolution display devices and have been actively developed.

Light-emitting apparatuses including light-emitting devices (also referred to as light-emitting elements) using organic compounds have been developed as display devices. Light-emitting devices utilizing electroluminescence (hereinafter referred to as EL; such devices are also referred to as organic EL devices or light-emitting devices) have features such as ease of reduction in thickness and weight, high-speed response to input signals, and driving with a constant DC voltage power source, and have been used in display devices.

Displays or lighting devices including light-emitting devices are suitable for a variety of electronic devices, and research and development of materials and devices have progressed to obtain light-emitting devices with more favorable characteristics (see Patent Document 1, for example).

### REFERENCE

### Patent Document

[Patent Document 1] PCT International Publication No. WO2021/132995

### SUMMARY OF THE INVENTION

An object of one embodiment of the present invention is to provide a novel organic compound. Another object of one embodiment of the present invention is to provide a novel carrier-transport material. Another object of one embodiment of the present invention is to provide a novel hole-transport material. Another object of one embodiment of the present invention is to provide a highly heat-resistant carrier-transport material or hole-transport material.

An object of another embodiment of the present invention is to provide a light-emitting device having a low driving voltage. Another object of one embodiment of the present invention is to provide a light-emitting device, a light-emitting apparatus, an electronic device, and a display device each having low power consumption.

Note that the description of these objects does not preclude the existence of other objects. One embodiment of the present invention does not necessarily achieve all of these objects. Other objects can be derived from the description of the specification, the drawings, the claims, and the like.

One embodiment of the present invention is an organic compound represented by General Formula (GI) below.

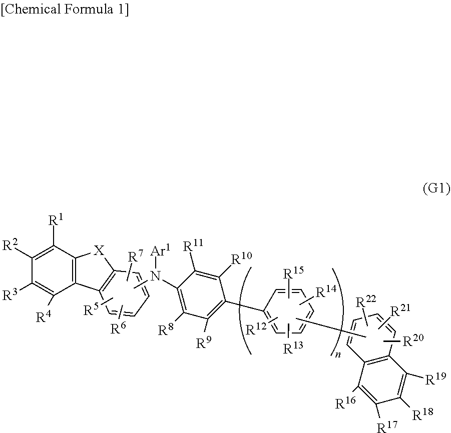

In General Formula (GI), X represents a sulfur atom or an oxygen atom; each of R¹ to R²² independently represents any one of hydrogen (including deuterium), halogen, a nitrile group, a substituted or unsubstituted alkenyl group, a substituted or unsubstituted vinyl group, a substituted or unsubstituted alkynyl group, a substituted or unsubstituted straight-chain alkyl group having 1 to 6 carbon atoms, a substituted or unsubstituted cycloalkyl group having 3 to 10 carbon atoms, a substituted or unsubstituted alkoxy group having 1 to 6 carbon atoms, a substituted or unsubstituted alkylsilyl group having 3 to 12 carbon atoms, a substituted or unsubstituted aryl group having 6 to 30 carbon atoms, and a substituted or unsubstituted heteroaryl group having 2 to 30 carbon atoms; at least one of R¹⁶ to R²² represents a substituted or unsubstituted naphthyl group; n represents an integer of 0 to 4; and when n is greater than or equal to 2, R¹²s may be the same as or different from each other, R¹³s may be the same as or different from each other, R¹⁴s may be the same as or different from each other, and R¹⁵s may be the same as or different from each other. Ar¹ is represented by General Formula (g1-1) or General Formula (g1-2).

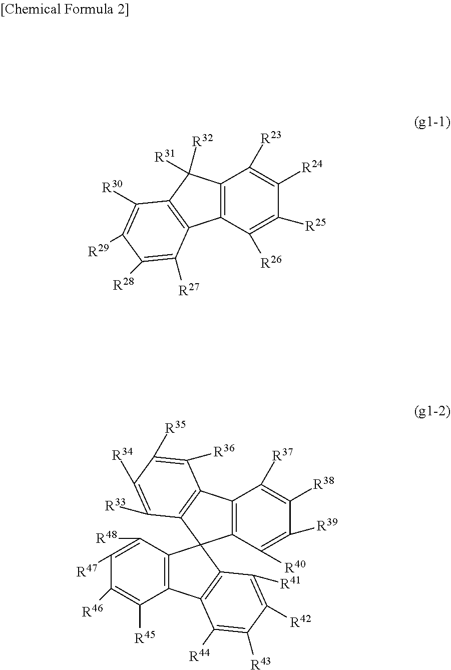

In General Formula (g1-1) and General Formula (g1-2), any one of R²³ to R³⁰ or any one of R³³ to R⁴⁸ is bonded to nitrogen of amine in General Formula (Gi); and each of the others, R³¹, and R³² independently represents any one of hydrogen (including deuterium), halogen, a nitrile group, a substituted or unsubstituted alkenyl group, a substituted or unsubstituted vinyl group, a substituted or unsubstituted alkynyl group, a substituted or unsubstituted straight-chain alkyl group having 1 to 6 carbon atoms, a substituted or unsubstituted cycloalkyl group having 3 to 10 carbon atoms, a substituted or unsubstituted alkoxy group having 1 to 6 carbon atoms, a substituted or unsubstituted alkylsilyl group having 3 to 12 carbon atoms, a substituted or unsubstituted aryl group having 6 to 30 carbon atoms, and a substituted or unsubstituted heteroaryl group having 2 to 30 carbon atoms.

Another embodiment of the present invention is the organic compound with the above structure, in which n is 0 or 1.

Another embodiment of the present invention is an organic compound represented by General Formula (G2).

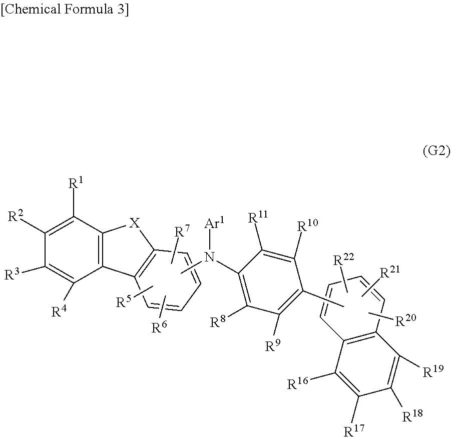

In General Formula (G2), X represents a sulfur atom or an oxygen atom; each of R¹ to R¹¹ and R¹⁶ to R²² independently represents any one of hydrogen (including deuterium), halogen, a nitrile group, a substituted or unsubstituted alkenyl group, a substituted or unsubstituted vinyl group, a substituted or unsubstituted alkynyl group, a substituted or unsubstituted straight-chain alkyl group having 1 to 6 carbon atoms, a substituted or unsubstituted cycloalkyl group having 3 to 10 carbon atoms, a substituted or unsubstituted alkoxy group having 1 to 6 carbon atoms, a substituted or unsubstituted alkylsilyl group having 3 to 12 carbon atoms, a substituted or unsubstituted aryl group having 6 to 30 carbon atoms, and a substituted or unsubstituted heteroaryl group having 2 to 30 carbon atoms; and at least one of R¹⁶ to R¹⁹ represents a substituted or unsubstituted naphthyl group. Ar¹ is represented by General Formula (g1-1) or General Formula (g1-2).

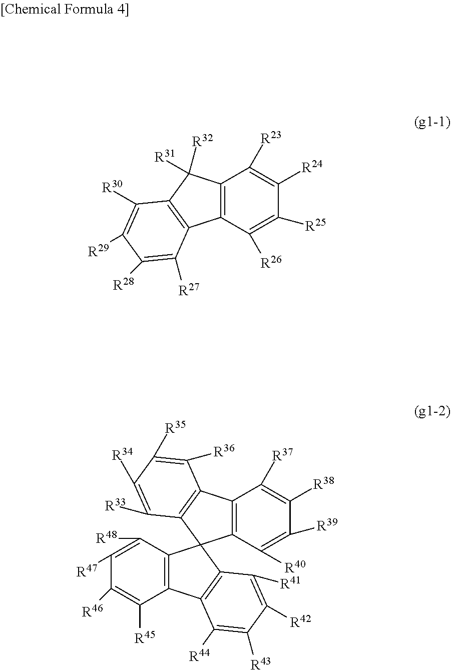

In General Formula (g1-1) and General Formula (g1-2), any one of R²³ to R³¹ or any one of R³³ to R⁴⁸ is bonded to nitrogen of amine in General Formula (G2); and each of the others, R³¹, and R³² independently represents any one of hydrogen (including deuterium), halogen, a nitrile group, a substituted or unsubstituted alkenyl group, a substituted or unsubstituted vinyl group, a substituted or unsubstituted alkynyl group, a substituted or unsubstituted straight-chain alkyl group having 1 to 6 carbon atoms, a substituted or unsubstituted cycloalkyl group having 3 to 10 carbon atoms, a substituted or unsubstituted alkoxy group having 1 to 6 carbon atoms, a substituted or unsubstituted alkylsilyl group having 3 to 12 carbon atoms, a substituted or unsubstituted aryl group having 6 to 30 carbon atoms, and a substituted or unsubstituted heteroaryl group having 2 to 30 carbon atoms.

Another embodiment of the present invention is an organic compound represented by General Formula (G3) below.

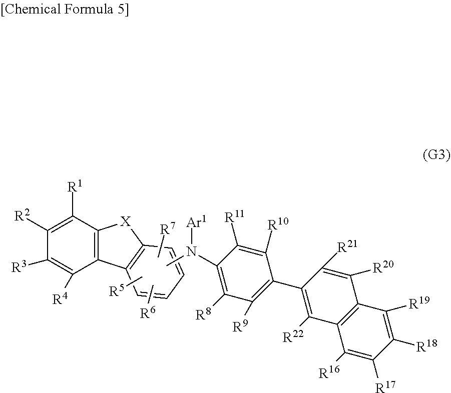

In General Formula (G3), X represents a sulfur atom or an oxygen atom; each of R¹ to R¹¹ and R¹⁶ to R²² independently represents any one of hydrogen (including deuterium), halogen, a nitrile group, a substituted or unsubstituted alkenyl group, a substituted or unsubstituted vinyl group, a substituted or unsubstituted alkynyl group, a substituted or unsubstituted straight-chain alkyl group having 1 to 6 carbon atoms, a substituted or unsubstituted cycloalkyl group having 3 to 10 carbon atoms, a substituted or unsubstituted alkoxy group having 1 to 6 carbon atoms, a substituted or unsubstituted alkylsilyl group having 3 to 12 carbon atoms, a substituted or unsubstituted aryl group having 6 to 30 carbon atoms, and a substituted or unsubstituted heteroaryl group having 2 to 30 carbon atoms; and at least one of R¹⁶ to R²² represents a substituted or unsubstituted naphthyl group. Ar¹ is represented by General Formula (g1-1) or General Formula (g1-2).

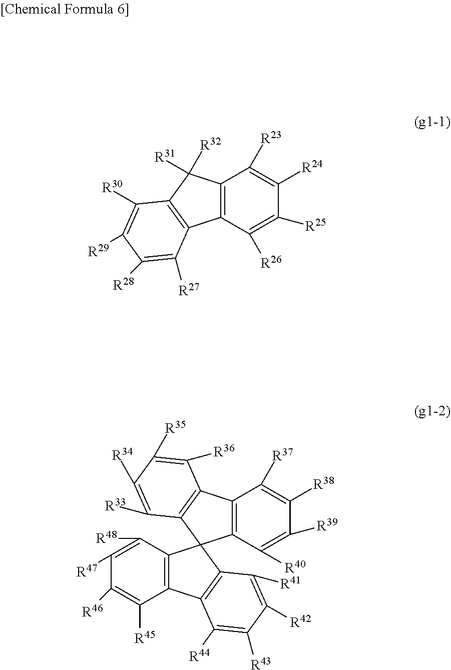

In General Formula (g1-1) and General Formula (g1-2), any one of R²³ to R³¹ or any one of R³³ to R⁴⁸ is bonded to nitrogen of amine in General Formula (G3); and each of the others, R³¹, and R³² independently represents any one of hydrogen (including deuterium), halogen, a nitrile group, a substituted or unsubstituted alkenyl group, a substituted or unsubstituted vinyl group, a substituted or unsubstituted alkynyl group, a substituted or unsubstituted straight-chain alkyl group having 1 to 6 carbon atoms, a substituted or unsubstituted cycloalkyl group having 3 to 10 carbon atoms, a substituted or unsubstituted alkoxy group having 1 to 6 carbon atoms, a substituted or unsubstituted alkylsilyl group having 3 to 12 carbon atoms, a substituted or unsubstituted aryl group having 6 to 30 carbon atoms, and a substituted or unsubstituted heteroaryl group having 2 to 30 carbon atoms.

Another embodiment of the present invention is the organic compound with the above structure, in which any one of R¹⁶ to R²² is a group represented by General Formula (g2) below.

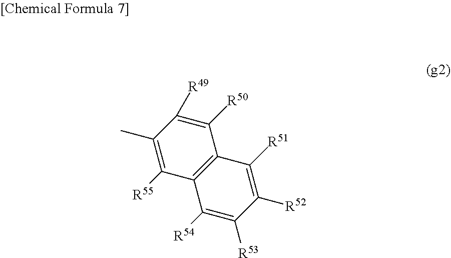

In General Formula (g2), each of R⁴⁹ to R⁵⁵ independently represents any one of hydrogen (including deuterium), halogen, a nitrile group, a substituted or unsubstituted alkenyl group, a substituted or unsubstituted vinyl group, a substituted or unsubstituted alkynyl group, a substituted or unsubstituted straight-chain alkyl group having 1 to 6 carbon atoms, a substituted or unsubstituted cycloalkyl group having 3 to 10 carbon atoms, a substituted or unsubstituted alkoxy group having 1 to 6 carbon atoms, a substituted or unsubstituted alkylsilyl group having 3 to 12 carbon atoms, a substituted or unsubstituted aryl group having 6 to 30 carbon atoms, and a substituted or unsubstituted heteroaryl group having 2 to 30 carbon atoms.

Another embodiment of the present invention is an organic compound represented by General Formula (G4).

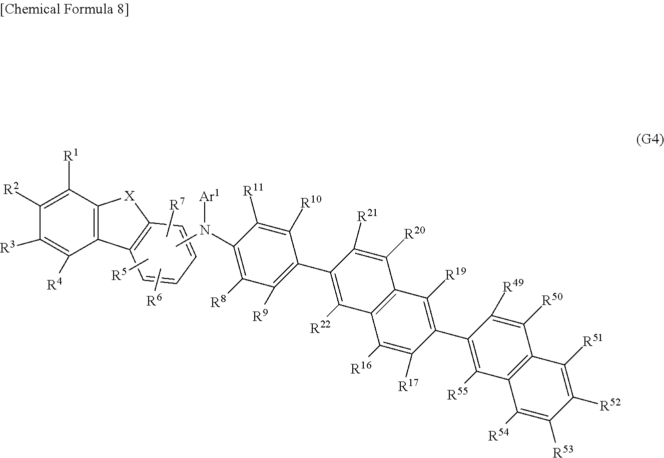

In General Formula (G4), X represents a sulfur atom or an oxygen atom; and each of R¹ to R¹¹, R¹⁶, R¹⁷, R¹⁹ to R²², and R⁴⁹ to R⁵⁵ independently represents any one of hydrogen (including deuterium), halogen, a nitrile group, a substituted or unsubstituted alkenyl group, a substituted or unsubstituted vinyl group, a substituted or unsubstituted alkynyl group, a substituted or unsubstituted straight-chain alkyl group having 1 to 6 carbon atoms, a substituted or unsubstituted cycloalkyl group having 3 to 10 carbon atoms, a substituted or unsubstituted alkoxy group having I to 6 carbon atoms, a substituted or unsubstituted alkylsilyl group having 3 to 12 carbon atoms, a substituted or unsubstituted aryl group having 6 to 30 carbon atoms, and a substituted or unsubstituted heteroaryl group having 2 to 30 carbon atoms. Ar¹ is represented by General Formula (g1-1) or General Formula (g1-2).

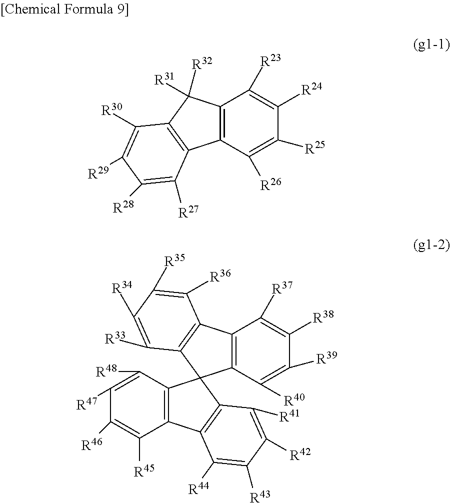

In General Formula (g1-1) and General Formula (g1-2), any one of R²³ to R³¹ or any one of R³³ to R⁴⁸ is bonded to nitrogen of amine in General Formula (G4); and each of the others, R³¹, and R³² independently represents any one of hydrogen (including deuterium), halogen, a nitrile group, a substituted or unsubstituted alkenyl group, a substituted or unsubstituted vinyl group, a substituted or unsubstituted alkynyl group, a substituted or unsubstituted straight-chain alkyl group having 1 to 6 carbon atoms, a substituted or unsubstituted cycloalkyl group having 3 to 10 carbon atoms, a substituted or unsubstituted alkoxy group having I to 6 carbon atoms, a substituted or unsubstituted alkylsilyl group having 3 to 12 carbon atoms, a substituted or unsubstituted aryl group having 6 to 30 carbon atoms, and a substituted or unsubstituted heteroaryl group having 2 to 30 carbon atoms.

Another embodiment of the present invention is an organic compound represented by General Formula (G5) below.

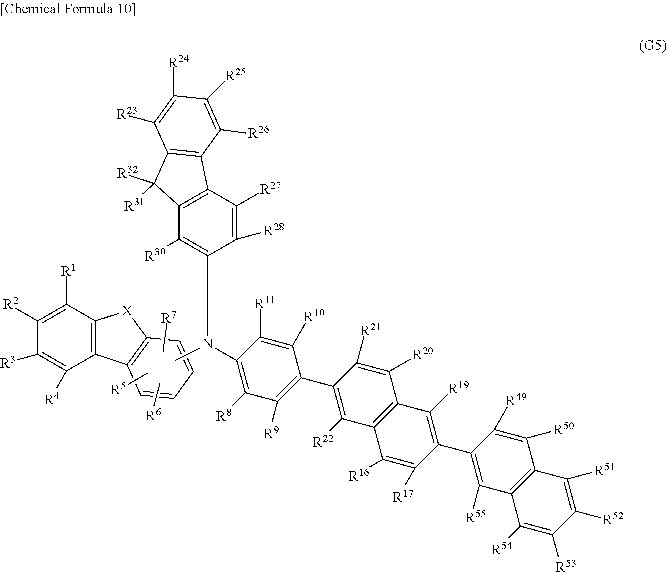

In General Formula (G5), X represents a sulfur atom or an oxygen atom; and each of R¹ to R¹¹, R¹⁶, R¹⁷, R¹⁹ to R²⁸, R³⁰ to R³², and R⁴⁹ to R⁵⁵ independently represents any one of hydrogen (including deuterium), halogen, a nitrile group, a substituted or unsubstituted alkenyl group, a substituted or unsubstituted vinyl group, a substituted or unsubstituted alkynyl group, a substituted or unsubstituted straight-chain alkyl group having I to 6 carbon atoms, a substituted or unsubstituted cycloalkyl group having 3 to 10 carbon atoms, a substituted or unsubstituted alkoxy group having 1 to 6 carbon atoms, a substituted or unsubstituted alkylsilyl group having 3 to 12 carbon atoms, a substituted or unsubstituted aryl group having 6 to 30 carbon atoms, and a substituted or unsubstituted heteroaryl group having 2 to 30 carbon atoms.

Another embodiment of the present invention is an organic compound represented by General Formula (G6) below.

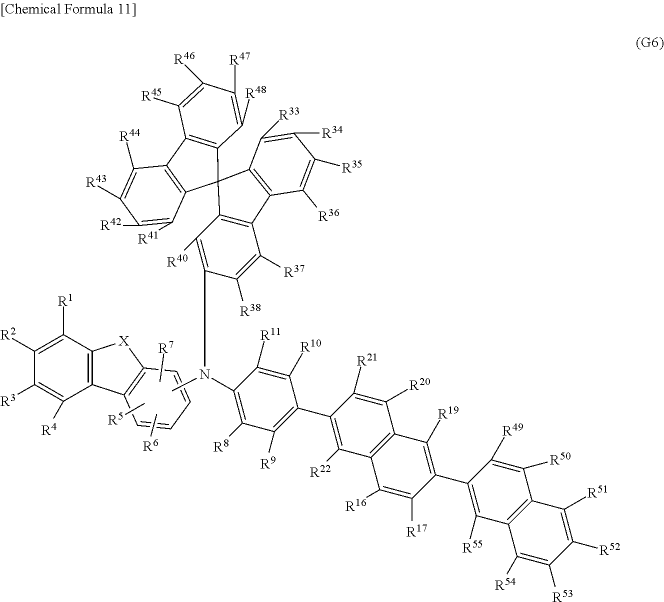

In General Formula (G6), X represents a sulfur atom or an oxygen atom; and each of R¹ to R¹¹, R¹⁶, R¹⁷, R¹⁹ to R²², R³³ to R³⁸, and R⁴⁰ to R⁵⁵ independently represents any one of hydrogen (including deuterium), halogen, a nitrile group, a substituted or unsubstituted alkenyl group, a substituted or unsubstituted vinyl group, a substituted or unsubstituted alkynyl group, a substituted or unsubstituted straight-chain alkyl group having 1 to 6 carbon atoms, a substituted or unsubstituted cycloalkyl group having 3 to 10 carbon atoms, a substituted or unsubstituted alkoxy group having 1 to 6 carbon atoms, a substituted or unsubstituted alkylsilyl group having 3 to 12 carbon atoms, a substituted or unsubstituted aryl group having 6 to 30 carbon atoms, and a substituted or unsubstituted heteroaryl group having 2 to 30 carbon atoms.

Another embodiment of the present invention is an organic semiconductor device including any of the organic compounds described above.

Another embodiment of the present invention is a light-emitting device including any of the organic compounds described above.

Another embodiment of the present invention is a light-receiving device including any of the organic compounds described above.

Another embodiment of the present invention is an organic electronic device including a light-emitting device containing any of the above organic compounds and a light-receiving device containing any of the above organic compounds on the same plane.

Another embodiment of the present invention is an organic electronic device using any of the organic compounds described above for a cap layer.

Another embodiment of the present invention is an electronic device including the above organic electronic device.

According to one embodiment of the present invention, a novel organic compound can be provided. According to another embodiment of the present invention, a novel carrier-transport material can be provided. According to another embodiment of the present invention, a novel hole-transport material can be provided. According to another embodiment of the present invention, a highly heat-resistant carrier-transport material or hole-transport material can be provided.

Another embodiment of the present invention can provide a light-emitting device having a low driving voltage. Another embodiment of the present invention can provide a light-emitting device, a light-emitting apparatus, an electronic device, and a display device each having low power consumption.

Another embodiment of the present invention can provide a novel light-emitting device, a novel display device, a novel display module, and a novel electronic device.

Note that the description of these effects does not preclude the existence of other effects. One embodiment of the present invention does not necessarily have all of these effects. Other effects can be derived from the description of the specification, the drawings, the claims, and the like.

### BRIEF DESCRIPTION OF THE DRAWINGS

FIGS. 1A to 1C are diagrams each illustrating a light-emitting device.

FIGS. 2A and 2B are a top view and a cross-sectional view illustrating a light-emitting apparatus.

FIGS. 3A to 3E are cross-sectional views illustrating an example of a method for manufacturing a display device.

FIGS. 4A to 4D are cross-sectional views illustrating an example of the method for manufacturing a display device.

FIGS. 5A to 5D are cross-sectional views illustrating an example of the method for manufacturing a display device.

FIGS. 6A to 6C are cross-sectional views illustrating an example of the method for manufacturing a display device.

FIGS. 7A to 7C are cross-sectional views illustrating an example of the method for manufacturing a display device.

FIGS. 8A to 8C are cross-sectional views illustrating an example of the method for manufacturing a display device.

FIGS. 9A and 9B are perspective views illustrating a structure example of a display module.

FIGS. 10A and 10B are cross-sectional views each illustrating a structure example of a display device.

FIG. 11 is a perspective view illustrating a structure example of a display device.

FIG. 12 is a cross-sectional view illustrating a structure example of a display device.

FIG. 13 is a cross-sectional view illustrating a structure example of a display device.

FIG. 14 is a cross-sectional view illustrating a structure example of a display device.

FIGS. 15A to 15D illustrate examples of electronic devices.

FIGS. 16A to 16F illustrate examples of electronic devices.

FIGS. 17A to 17G illustrate examples of electronic devices.

FIG. 18 illustrates a photosensor.

FIGS. 19A and 19B show a ¹H-NMR spectrum of Fr(2)F(4)A(βN2)B.

FIGS. 20A and 20B show an absorption spectrum and a PL spectrum of Fr(2)F(4)A(βN2)B.

FIGS. 21A and 21B show a ¹H-NMR spectrum of FrFA(βN2)B.

FIGS. 22A and 22B show an absorption spectrum and a PL spectrum of FrFA(βN2)B.

FIG. 23 shows luminance-current density characteristics of a light-emitting device of one embodiment of the present invention.

FIG. 24 shows current efficiency-luminance characteristics of the light-emitting device of one embodiment of the present invention.

FIG. 25 shows luminance-voltage characteristics of the light-emitting device of one embodiment of the present invention.

FIG. 26 shows current density-voltage characteristics of the light-emitting device of one embodiment of the present invention.

FIG. 27 shows external quantum efficiency-luminance characteristics of the light-emitting device of one embodiment of the present invention.

FIG. 28 shows an electroluminescence spectrum of the light-emitting device of one embodiment of the present invention.

FIGS. 29A and 29B show a ¹H-NMR spectrum of N-(chlorophenyl-4-yl)dibenzofuran-2-amine.

FIGS. 30A and 30B show a ¹H-NMR spectrum of N-[4-(2,2′-binaphthyl-6-yl)phenyl]-N-(9,9-dimethyl-9H-fluoren-4-yl)dibenzofuran-2-amine.

FIGS. 31A and 31B show a ¹H-NMR spectrum of N-[4-(2,2′-binaphthyl-6-yl)phenyl]-9,9-dimethyl-9H-fluoren-2-amine.

FIGS. 32A and 32B show a ¹H-NMR spectrum of Fr(2)FA(βN2)B.

FIGS. 33A and 33B show a ¹H-NMR spectrum of N-(chlorophenyl-4-yl)-N-(9,9-dimethyl-9H-fluoren-4-yl)dibenzofuran-4-amine.

FIGS. 34A and 34B show a ¹H-NMR spectrum of FrF(4)A(βN2)B.

FIGS. 35A and 35B show a ¹H-NMR spectrum of Fr(1)FA(βN2)B.

FIGS. 36A and 36B show a ¹H-NMR spectrum of N-(chlorophenyl-4-yl)-N-(9,9-diphenyl-9H-fluoren-2-yl)dibenzofuran-2-amine.

FIGS. 37A and 37B show a ¹H-NMR spectrum of Fr(2)FLP(2)A(βN2)B.

FIGS. 38A and 38B show a ¹H-NMR spectrum of N-(chlorophenyl-4-yl)-N-(9,9-diphenyl-9H-fluoren-2-yl)dibenzofuran-4-amine.

FIGS. 39A and 39B show a ¹H-NMR spectrum of FrFLP(2)A(βN2)B.

FIGS. 40A and 40B show a ¹H-NMR spectrum of FrSFA(βN2)B.

### DETAILED DESCRIPTION OF THE INVENTION

Embodiments will be described in detail with reference to the drawings. Note that the present invention is not limited to the following description, and it will be readily appreciated by those skilled in the art that modes and details of the present invention can be modified in various ways without departing from the spirit and scope of the present invention. Therefore, the present invention should not be construed as being limited to the description in the following embodiments.

### Embodiment 1

In this embodiment, an organic compound of one embodiment of the present invention will be described.

The organic compound of one embodiment of the present invention is represented by General Formula (G1).

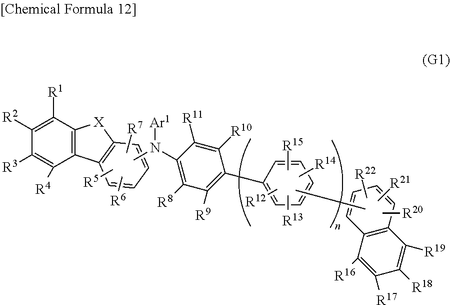

In the organic compound represented by General Formula (G1) above, at least one of R¹⁶ to R²² represents a substituted or unsubstituted naphthyl group, each of the others and R¹ to R¹⁵ independently represents any one of hydrogen (including deuterium), halogen, a nitrile group, a substituted or unsubstituted alkenyl group, a substituted or unsubstituted vinyl group, a substituted or unsubstituted alkynyl group, a substituted or unsubstituted straight-chain alkyl group having 1 to 6 carbon atoms, a substituted or unsubstituted cycloalkyl group having 3 to 10 carbon atoms, a substituted or unsubstituted alkoxy group having 1 to 6 carbon atoms, a substituted or unsubstituted alkylsilyl group having 3 to 12 carbon atoms, a substituted or unsubstituted aryl group having 6 to 30 carbon atoms, and a substituted or unsubstituted heteroaryl group having 2 to 30 carbon atoms. Specifically, it is preferable that each of the others and R¹ to R¹¹ independently represent hydrogen (including deuterium), a substituted or unsubstituted straight-chain alkyl group having 1 to 6 carbon atoms, or a substituted or unsubstituted cycloalkyl group having 3 to 10 carbon atoms, in which case a high heat-resistant material whose sublimation temperature is low can be provided. When a high heat-resistant material is used, an organic semiconductor device (e.g., a light-emitting device and a light-receiving device) having high heat resistance can be provided and thus can be suitably used for vehicles requiring high temperature resistance. In the case where a material with a low sublimation temperature is used, generation of thermal decomposition in a heating step necessary for evaporation in formation of an organic semiconductor device can be prevented or reduced, which is preferable because a highly purified film can be provided and accordingly a highly reliable organic semiconductor device can be provided. A material including an alkyl group or a cycloalkyl group is preferably used, in which case a film with a low refractive index can be formed; thus, with use of the material of the present invention for a transport layer of a light-emitting device, a device with high light extraction efficiency can be provided. Moreover, R⁶ to R²² other than groups representing a naphthyl group and R¹ to R are preferably hydrogen, in which case synthesis becomes simple.

The organic compound with such a structure has high heat resistance and a high hole-transport property owing to the substitution of a phenylene group bonded to nitrogen of amine at the para-position; thus, the material of the present invention is preferably used for an organic semiconductor device because the driving voltage of the device can be lowered, providing an organic semiconductor device with low power consumption.

At least one of R¹⁶ to R²² represents a substituted or unsubstituted naphthyl group. The case where one or two of R¹⁶ to R²² represent a substituted or unsubstituted naphthyl group is preferable because a change in voltage at the time of driving the device successively can be small and thus an organic semiconductor device with stable voltage at the time of driving can be provided. The case where only one of R¹⁶ to R²² represents a substituted or unsubstituted naphthyl group is preferable because the evaporation temperature can be prevented from being too high and the hole-transport property can be increased.

In the organic compound represented by General Formula (GI) above, n represents an integer of 0 to 4. When n is greater than or equal to 2, R¹²s may be the same as or different from each other. The same applies to R¹³s, R¹⁴s, and R¹⁵s. Note that n is preferably 0 or 1 in order to prevent the evaporation temperature from being too high, to increase the hole-transport property, and to provide a highly reliable organic semiconductor device with a small change in voltage at the time of driving. The phenylene group bonded to the amine is preferably a para-phenylene group in terms of increasing the hole-transport property; however, when three or more phenylene groups are bonded at para-positions, the effective conjugation length is extended and thus the absorption wavelength might extend to visible light; in such a case, the light extraction efficiency of the light-emitting device might be lowered. Accordingly, n is preferably 0 or 1, and when n is greater than or equal to 2, bonding at an ortho position, in which case the effective conjugation length is less likely to be extended, or bonding at an ortho position, in which case a three-dimensional twist occurs, are preferable to bonding at a para position.

In the organic compound represented by General Formula (G1) above, X represents a sulfur atom or an oxygen atom. X is preferably an oxygen atom because a compound containing an oxygen atom has a lower refractive index than a compound containing a sulfur atom and a device using the compound with a lower refractive index has an effect of increasing light extraction efficiency, offering a highly efficient light-emitting device. Alternatively, X is preferably a sulfur atom because a compound containing a sulfur atom has higher heat resistance than a compound containing an oxygen atom, offering a device resistant to high-temperature driving. Furthermore, since a compound containing a sulfur atom has a high refractive index, when the compound containing a sulfur atom is deposited over a cathode as a film having a high refractive index, a light-emitting device with high light extraction efficiency can be provided.

In the organic compound represented by General Formula (G1) above, Ar¹ represents a group represented by General Formula (g1-1) or (g1-2) shown below.

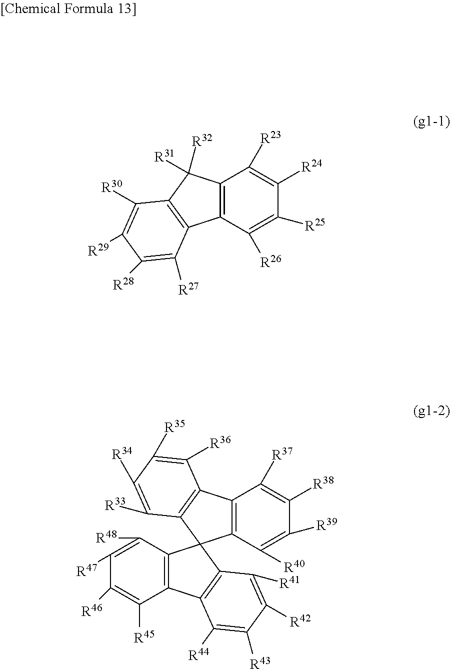

In the case where Ar¹ is a group represented by General Formula (g1-1), it is preferable that any one of R²³ to R³⁰ be bonded to nitrogen of amine in General Formula (G1) and each of the others, R³¹, and R³² independently represents any one of hydrogen (including deuterium), halogen, a nitrile group, a substituted or unsubstituted alkenyl group, a substituted or unsubstituted vinyl group, a substituted or unsubstituted alkynyl group, a substituted or unsubstituted straight-chain alkyl group having I to 6 carbon atoms, a substituted or unsubstituted cycloalkyl group having 3 to 10 carbon atoms, a substituted or unsubstituted alkoxy group having I to 6 carbon atoms, a substituted or unsubstituted alkylsilyl group having 3 to 12 carbon atoms, a substituted or unsubstituted aryl group having 6 to 30 carbon atoms, and a substituted or unsubstituted heteroaryl group having 2 to 30 carbon atoms. Specifically, it is preferable that each of the others, R³¹, and R³² independently represent hydrogen (including deuterium), a substituted or unsubstituted straight-chain alkyl group having 1 to 6 carbon atoms, or a substituted or unsubstituted cycloalkyl group having 3 to 10 carbon atoms.

In the case where Ar¹ is a group represented by General Formula (g1-2), it is preferable that any one of R³³ to R⁴⁸ be bonded to nitrogen of amine in General Formula (G1) and each of the others independently represents any one of hydrogen (including deuterium), halogen, a nitrile group, a substituted or unsubstituted alkenyl group, a substituted or unsubstituted vinyl group, a substituted or unsubstituted alkynyl group, a substituted or unsubstituted straight-chain alkyl group having I to 6 carbon atoms, a substituted or unsubstituted cycloalkyl group having 3 to 10 carbon atoms, a substituted or unsubstituted alkoxy group having 1 to 6 carbon atoms, a substituted or unsubstituted alkylsilyl group having 3 to 12 carbon atoms, a substituted or unsubstituted aryl group having 6 to 30 carbon atoms, and a substituted or unsubstituted heteroaryl group having 2 to 30 carbon atoms. Specifically, it is preferable that each of the others independently represent hydrogen (including deuterium), a substituted or unsubstituted straight-chain alkyl group having I to 6 carbon atoms, or a substituted or unsubstituted cycloalkyl group having 3 to 10 carbon atoms.

The organic compound with such a structure can be a highly heat-resistant material having a favorable hole-transport property. A thin film containing the organic compound with such a structure is preferable because it undergoes a small change in quality and can provide a device stable to heat or driving. A device using the organic compound with such a structure has a low driving voltage and a small variation in driving voltage; thus, the device can be highly reliable in voltage and high-temperature driving. The device is preferable also in terms of low power consumption. In addition, the organic compound with such a structure is preferable because it has a high sublimation property, is not decomposed in an evaporation process, and enables formation of a highly purified film, providing a highly reliable organic semiconductor device. The organic compound is preferable also in terms of a fabrication cost because it can be produced stably.

In the organic compound represented by General Formula (G1) above, n is preferably 0 in order to prevent the evaporation temperature from being too high, to increase the hole-transport property, and to provide a highly reliable organic semiconductor device with a small change in voltage at the time of driving. That is, the organic compound of one embodiment of the present invention is preferably an organic compound represented by General Formula (G2).

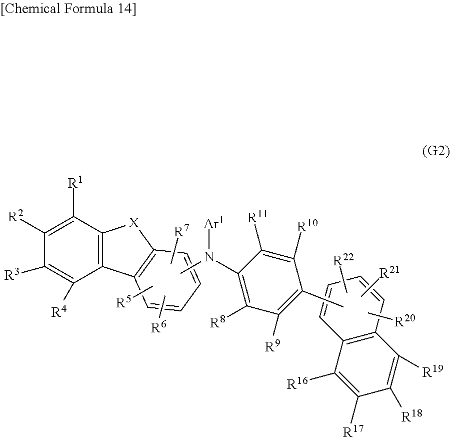

Since R¹ to R¹¹, R¹⁶ to R²², and Ar¹ in General Formula (G2) above are the same as those in General Formula (G1) above, repeated description thereof is omitted.

Note that in the organic compound represented by General Formula (G2) above, the naphthyl group is preferably bonded to the paraphenylene group at the 2-position in terms of achieving a high hole-transport property and high reliability at the time of driving. That is, the organic compound of one embodiment of the present invention is preferably an organic compound represented by General Formula (G3).

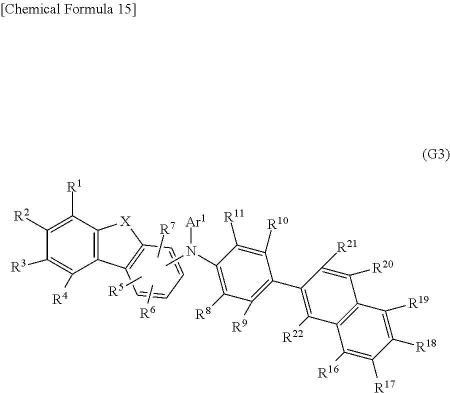

Since R¹ to R¹¹, R¹⁶ to R²², and Ar¹ in General Formula (G3) above are the same as those in General Formula (G1) above, repeated description thereof is omitted.

In the organic compounds represented by General Formulae (Gi) to (G3) above, at least one of R¹⁶ to R²² represents a substituted or unsubstituted naphthyl group, and the substituted or unsubstituted naphthyl group is preferably a substituted or unsubstituted 2-naphthyl group represented by General Formula (g2) below in terms of achieving a high hole-transport property and high reliability at the time of driving.

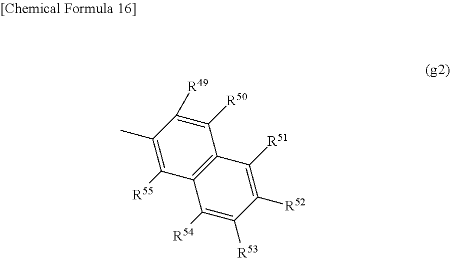

In General Formula (g2), each of R⁴⁹ to R⁵⁵ independently represents any one of hydrogen (including deuterium), halogen, a nitrile group, a substituted or unsubstituted alkenyl group, a substituted or unsubstituted vinyl group, a substituted or unsubstituted alkynyl group, a substituted or unsubstituted straight-chain alkyl group having 1 to 6 carbon atoms, a substituted or unsubstituted cycloalkyl group having 3 to 10 carbon atoms, a substituted or unsubstituted alkoxy group having 1 to 6 carbon atoms, a substituted or unsubstituted alkylsilyl group having 3 to 12 carbon atoms, a substituted or unsubstituted aryl group having 6 to 30 carbon atoms, and a substituted or unsubstituted heteroaryl group having 2 to 30 carbon atoms. It is preferable that each of R⁴⁹ to R⁵⁵ independently represent any one of hydrogen (including deuterium), halogen, a nitrile group, a substituted or unsubstituted alkenyl group, a substituted or unsubstituted vinyl group, a substituted or unsubstituted alkynyl group, a substituted or unsubstituted straight-chain alkyl group having I to 6 carbon atoms, a substituted or unsubstituted cycloalkyl group having 3 to 10 carbon atoms, a substituted or unsubstituted alkoxy group having I to 6 carbon atoms, and a substituted or unsubstituted alkylsilyl group having 3 to 12 carbon atoms, in which case a high hole-transport property, high heat resistance, or high stability of a thin film can be obtained; it is further preferable that each of R⁴⁹ to R⁵⁵ independently represent any one of hydrogen (including deuterium) and a substituted or unsubstituted straight-chain alkyl group having 1 to 6 carbon atoms, in which case the sublimation temperature and the evaporation temperature can be reduced and thermal decomposition at the time of evaporation can be inhibited; it is further preferable that each of R⁴⁹ to R⁵⁵ independently represent hydrogen (including deuterium), in which case the synthesis cost can be reduced.

The organic compound of one embodiment of the present invention has a structure in which a substituted or unsubstituted binaphthyl group is bonded to at least nitrogen of amine through a substituted or unsubstituted paraphenylene group, and the binaphthyl group is preferably a substituted or unsubstituted 2,2′-binaphthyl-6-yl group, in which case an organic semiconductor device including the organic compound of one embodiment of the present invention has reduced driving voltage, a small change in voltage caused by driving, small power consumption, high heat resistance, or high reliability with respect to driving. That is, the organic compound of one embodiment of the present invention is preferably an organic compound represented by General Formula (G4).

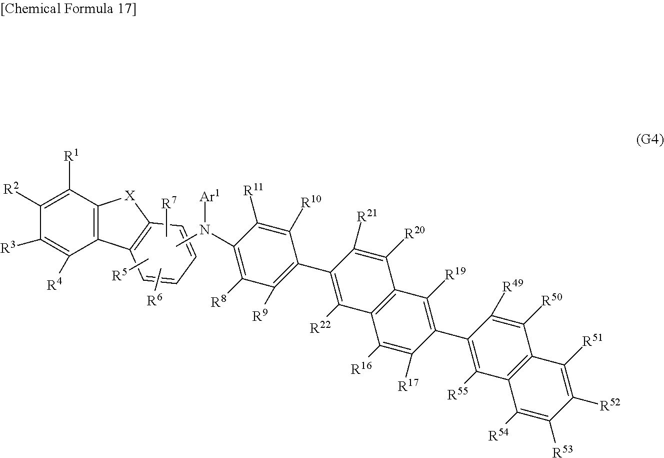

Since R¹ to R¹¹, R¹⁶, R¹⁷, and R¹⁹ to R²² in General Formula (G4) above are the same as those in General Formula (G1) above and R⁴⁹ to R⁵⁵ in General Formula (G4) above are the same as those in General Formula (g2), repeated description thereof is omitted.

In another embodiment of the present invention with the above structure, Ar¹ is preferably a substituted or unsubstituted fluoren-2-yl group, in which case the highest occupied molecular orbital (HOMO) level becomes relatively high and the organic compound has a high hole-transport property; thus, when the organic compound is used for a light-emitting device, the device can have low driving voltage, high emission efficiency, and high reliability. That is, the organic compound of one embodiment of the present invention is preferably an organic compound represented by General Formula (G5).

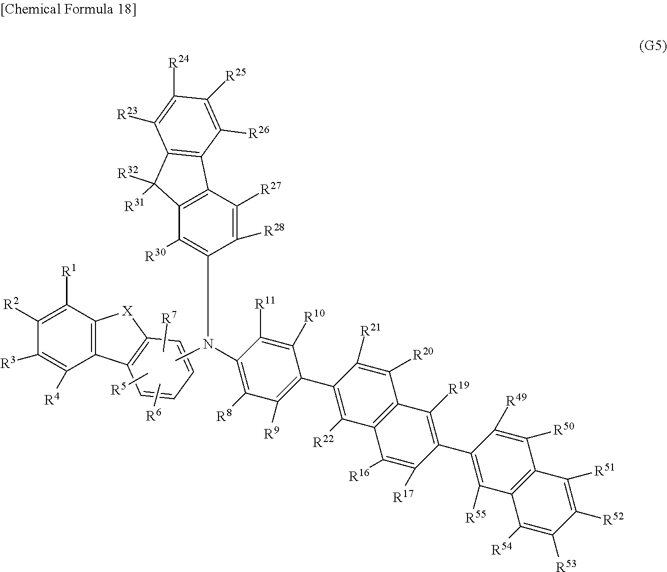

Since R¹ to R¹¹, R¹⁶, R¹⁷, and R¹⁹ to R²² in General Formula (G5) above are the same as those in General Formula (G1) above, R²³ to R²⁸ and R³⁰ to R³² in General Formula (G5) above are the same as those in General Formula (g1-1) above, and R⁴⁹ to R⁵⁵ in General Formula (G5) above are the same as those in General Formula (g2), repeated description thereof is omitted.

In another embodiment of the present invention with the above structure, when Ar¹ is preferably a substituted or unsubstituted 9,9′-spirobi[9H-fluoren]-2-yl group, the HOMO level becomes relatively high, providing an organic compound with a high hole-transport property. In addition, the organic compound has a relatively high HOMO level, and thus has an excellent property of hole injection to a light-emitting layer containing an organic compound with a high HOMO level, in particular, a phosphorescent device including a bipolar host or a mixed host of a hole-transporthost and an electron-transport host. Hence, when the organic compound is used for a phosphorescent light-emitting device, particularly for a green phosphorescent light-emitting device or a red phosphorescence light-emitting device as a material for a hole-transport layer in contact with the light-emitting layer, a phosphorescent light-emitting device reliable in high-temperature driving and having a low driving voltage can be provided. A light-emitting device with high emission efficiency can be provided. Furthermore, a highly reliable device can be provided.

That is, the organic compound of one embodiment of the present invention is preferably an organic compound represented by General Formula (G6).

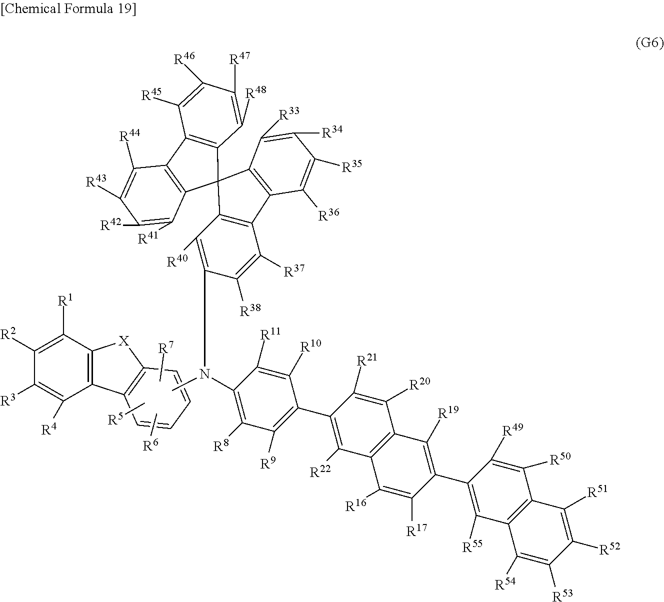

Since R¹ to R¹¹, R¹⁶, R¹⁷, and R¹⁹ to R²² in General Formula (G6) above are the same as those in General Formula (G1) above, R³³ to R³⁸ and R⁴⁰ to R⁴⁸ in General Formula (G6) above are the same as those in General Formula (g1-2) above, and R⁴⁹ to R⁵⁵ in General Formula (G6) above are the same as those in General Formula (g2), repeated description thereof is omitted.

In General Formulae (G1) to (G6), (g1-1) to (g1-2), and (g2), specific examples of halogen include fluorine, chlorine, bromine, and iodine.

In General Formulae (G1) to (G6), (g1-1), (g1-2), and (g2), examples of the alkenyl group include an ethenyl group, a 1-propenyl group, an allyl group, a 1-butenyl group, a 2-butenyl group, a 3-butenyl group, a sec-butenyl group, an isobutenyl group, a 1-pentenyl group, a 2-pentenyl group, a 3-pentenyl group, a 4-pentenyl group, an isopentenyl group, a 1-hexenyl group, a 2-hexenyl group, a 3-hexenyl group, a 4-hexenyl group, a 5-hexenyl group, a 1-heptenyl group, a 2-heptenyl group, a 3-heptenyl group, a 4-heptenyl group, a 5-heptenyl group, a 6-heptenyl group, a 1-octenyl group, a 2-octenyl group, a 3-octenyl group, a 4-octenyl group, a 5-octenyl group, a 6-octenyl group, and a 7-octenyl group.

In General Formulae (G1) to (G6), (g1-1), (g1-2), and (g2), examples of the alkynyl group include an ethynyl group, a 1-propynyl group, a 2-propynyl group, a 1-butynyl group, a 2-butynyl group, a 3-butynyl group, a sec-butynyl group, an isobutynyl group, a 1-pentynyl group, a 2-pentynyl group, a 3-pentynyl group, a 4-pentynyl group, an isopentynyl group, a 1-hexynyl group, a 2-hexynyl group, a 3-hexynyl group, a 4-hexynyl group, a 5-hexynyl group, a 1-heptynyl group, a 2-heptynyl group, a 3-heptynyl group, a 4-heptynyl group, a 5-heptynyl group, a 6-heptynyl group, a I-octynyl group, a 2-octynyl group, a 3-octynyl group, a 4-octynyl group, a 5-octynyl group, a 6-octynyl group, and a 7-octynyl group.

In General Formulae (G1) to (G6), (g1-1), (g1-2), and (g2), specific examples of the straight-chain alkyl group having I to 6 carbon atoms include a methyl group, an ethyl group, an n-propyl group, an n-butyl group, an n-pentyl group, and an n-hexyl group. In the case where the straight-chain alkyl group having I to 6 carbon atoms includes a substituent, the substituent is an alkyl group having 1 to 4 carbon atoms, a cycloalkyl group having 3 to 6 carbon atoms, or an aryl group having 6 to 13 carbon atoms.

In General Formulae (G1) to (G6), (g1-1), (g1-2), and (g2), specific examples of the cycloalkyl group having 3 to 10 carbon atoms include a cyclopropyl group, a cyclobutyl group, a cyclopentyl group, a cyclohexyl group, a cycloheptyl group, a cyclooctyl group, a cyclononanyl group, a cyclodecanyl group, an adamantyl group, a bicyclo[2.2.1]heptyl group, a tricyclo[5.2.1.0(2,6)]decanyl group, and a noradamantyl group. In the case where the cycloalkyl group having 3 to 10 carbon atoms includes a substituent, the substituent is an alkyl group having 1 to 4 carbon atoms, a cycloalkyl group having 3 to 6 carbon atoms, or an aryl group having 6 to 13 carbon atoms.

In General Formulae (G1) to (G6), (g1-1), (g1-2), and (g2), specific examples of the alkoxy group having 1 to 6 carbon atoms include a methoxy group, an ethoxy group, a propoxy group, an isopropoxy group, a tert-butoxy group, a sec-butoxy group, an isobutoxy group, a pentyloxy group, an octyloxy group, an allyloxy group, a cyclohexyloxy group, a phenoxy group, a benzyloxy group, a vinyloxy group, a propenyloxy group, a butenyloxy group, a pentenyloxy group, and a hexenyloxy group. In the case where the alkoxy group having I to 6 carbon atoms includes a substituent, the substituent is an alkyl group having 1 to 4 carbon atoms, a cycloalkyl group having 3 to 6 carbon atoms, or an aryl group having 6 to 13 carbon atoms.

In General Formulae (G1) to (G6), (g1-1), (g1-2), and (g2), examples of the alkylsilyl group having 3 to 12 carbon atoms include a trimethylsilyl group, a triethylsilyl group, a tert-butyl dimethylsilyl group, a triisopropylsilyl group, a tri-tert-butylsilyl group, and a tributylsilyl group. In the case where the alkylsilyl group having 3 to 12 carbon atoms includes a substituent, the substituent is an alkyl group having 1 to 4 carbon atoms, a cycloalkyl group having 3 to 6 carbon atoms, or an aryl group having 6 to 13 carbon atoms.

In General Formulae (G1) to (G6), (g1-1), (g1-2), and (g2), examples of the aryl group having 6 to 30 carbon atoms include a phenyl group, a tolyl group, a xylyl group, a biphenyl group, an indenyl group, a naphthyl group, a fluorenyl group, a spirofluorenyl group, a phenanthrenyl group, a triphenylenyl group, an anthracenyl group, and a fluoranthenyl group. In the case where the aryl group having 6 to 30 carbon atoms includes a substituent, the substituent is an alkyl group having 1 to 4 carbon atoms, a cycloalkyl group having 3 to 6 carbon atoms, or an aryl group having 6 to 13 carbon atoms.

In General Formulae (G1) to (G6), (g1-1), (g1-2), and (g2), examples of the heteroaryl group having 2 to 30 carbon atoms include a pyridin-yl group, a pyrimidin-yl group, a triazin-yl group, a phenanthrolin-yl group, a carbazol-yl group, a pyrrol-yl group, a thiophen-yl group, a furan-yl group, an imidazol-yl group, a bipyridin-yl group, a bipyrimidin-yl group, a pyrazin-yl group, a bipyrazin-yl group, a quinolin-yl group, an isoquinolin-yl group, a benzoquinolin-yl group, a quinoxalin-yl group, a benzoquinoxalin-yl group, a dibenzoquinoxalin-yl group, an azofluoren-yl group, a diazofluoren-yl group, a benzocarbazol-yl group, a dibenzocarbazol-yl group, a dibenzofuran-yl group, a benzonaphthofuran-yl group, a dinaphthofuran-yl group, a dibenzothiophen-yl group, a benzonaphthothiophen-yl group, a dinaphthothiophen-yl group, a benzofuropyridin-yl group, a benzofuropyrimidin-yl group, a benzothiopyridin-yl group, a benzothiopyrimidin-yl group, a naphthofuropyridin-yl group, a naphthofuropyrimidin-yl group, a naphthothiopyridin-yl group, a naphthothiopyrimidin-yl group, a dibenzoquinoxalin-yl group, an acridin-yl group, a xanthen-yl group, a phenothiazin-yl group, a phenoxazin-yl group, a phenazin-yl group, a triazol-yl group, an oxazol-yl group, an oxadiazol-yl group, a thiazol-yl group, a thiadiazol-yl group, a benzimidazol-yl group, and a pyrazol-yl group. In the case where the heteroaryl group having 2 to 30 carbon atoms includes a substituent, the substituent is an alkyl group having 1 to 4 carbon atoms, a cycloalkyl group having 3 to 6 carbon atoms, or an aryl group having 6 to 13 carbon atoms.

Note that hydrogen in General Formulae (G1) to (G6), (g1-1), (g1-2), and (g2) includes deuterium.

In the case where the above-described alkenyl group, vinyl group, alkynyl group, cycloalkyl group, alkoxy group, alkylsilyl group, aryl group, or heteroaryl group has a substituent, an alkyl group having 1 to 6 carbon atoms, a cycloalkyl group having 3 to 6 carbon atoms, or a substituted or unsubstituted aryl group having 6 to 12 carbon atoms can also be selected as the substituent. Specific examples of the alkyl group having I to 6 carbon atoms include a methyl group, an ethyl group, a propyl group, an isopropyl group, a butyl group, an isobutyl group, a tert-butyl group, and an n-hexyl group. Specific examples of the cycloalkyl group having 3 to 6 carbon atoms include a cyclopropyl group, a cyclobutyl group, a cyclopentyl group, a cyclohexyl group, an adamantyl group, and a norbornanyl group. Specific examples of the aryl group having 6 to 12 carbon atoms include a substituted or unsubstituted phenyl group, a substituted or unsubstituted naphthyl group, and a substituted or unsubstituted biphenyl group.

In the case where the straight-chain alkyl group has a substituent, a cycloalkyl group having 3 to 6 carbon atoms or a substituted or unsubstituted aryl group having 6 to 12 carbon atoms can be selected as the substituent. Specific examples of the cycloalkyl group having 3 to 6 carbon atoms include a cyclopropyl group, a cyclobutyl group, a cyclopentyl group, and a cyclohexyl group. Specific examples of the aryl group having 6 to 12 carbon atoms include a substituted or unsubstituted phenyl group, a substituted or unsubstituted naphthyl group, and a substituted or unsubstituted biphenyl group.

In the organic compounds represented by General Formulae (G1) to (G6), (g1-1), (g1-2), and (g2), specific examples of elements or groups that can be used as R¹ to R^{J} are groups represented by Structural Formulae (1-1) to (1-35) and (2-1) to (2-60) shown below. As for a group whose bond is not specified, a monovalent group from which any one of hydrogen atoms included in the group is released can be used.

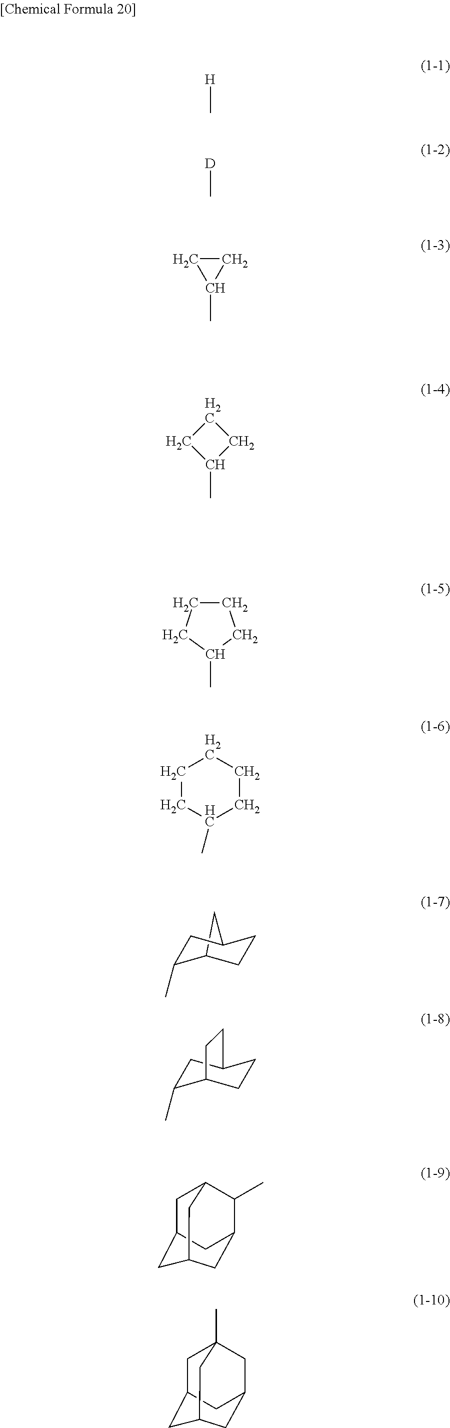

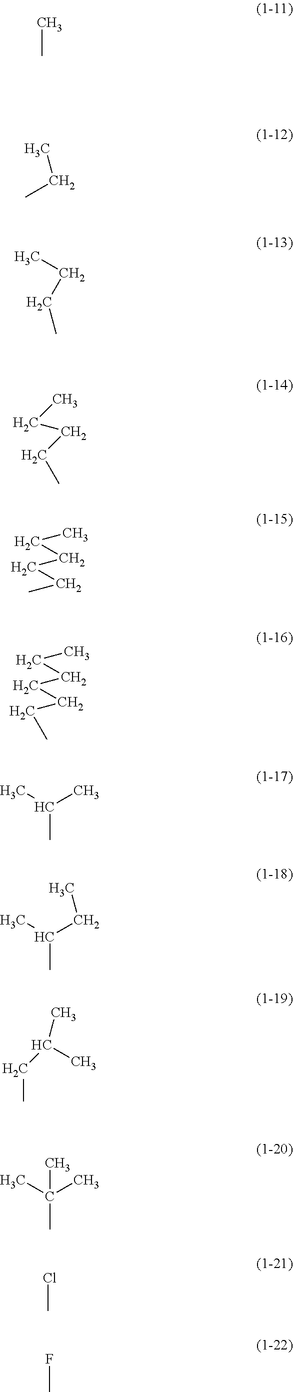

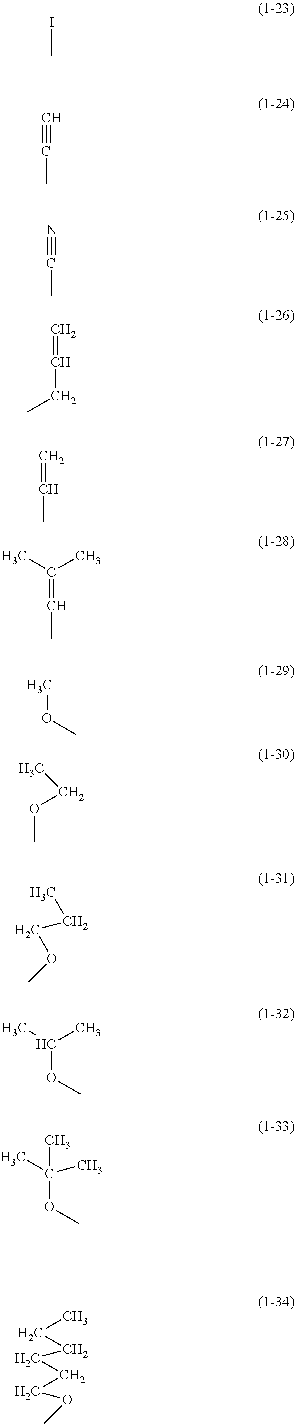

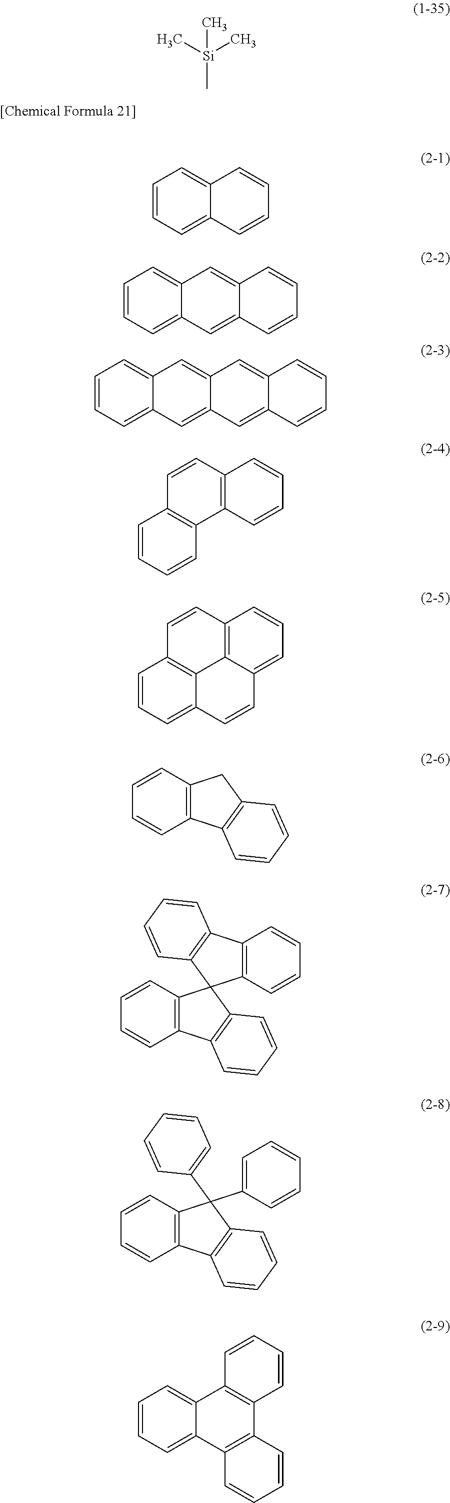

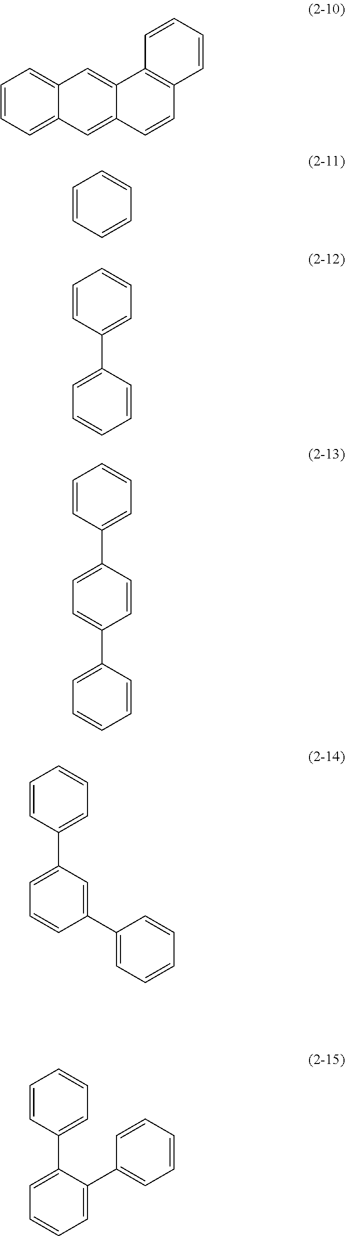

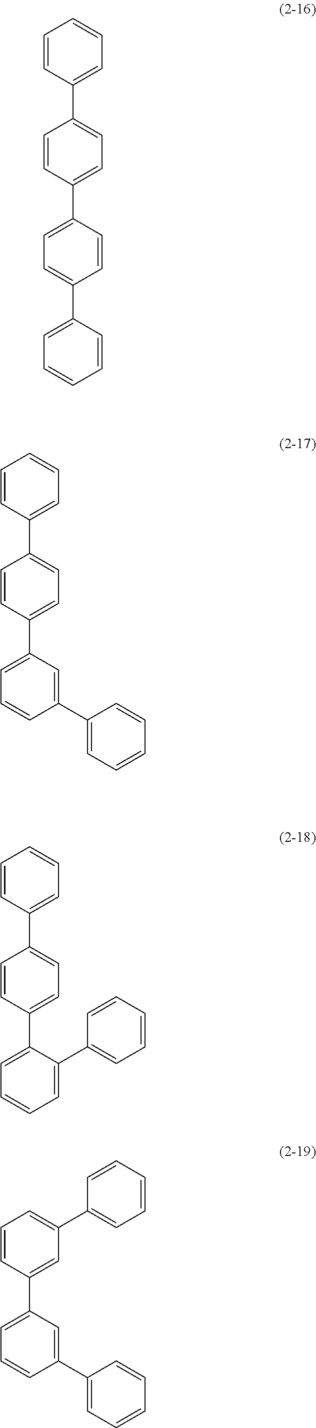

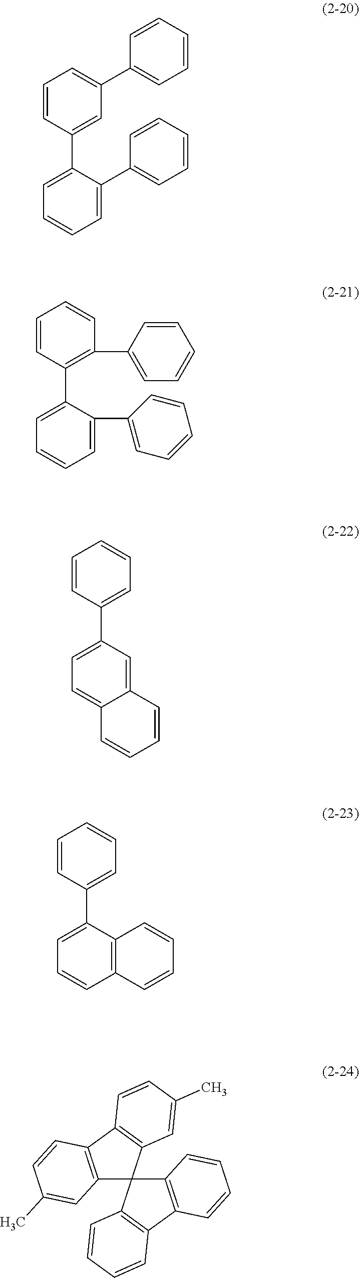

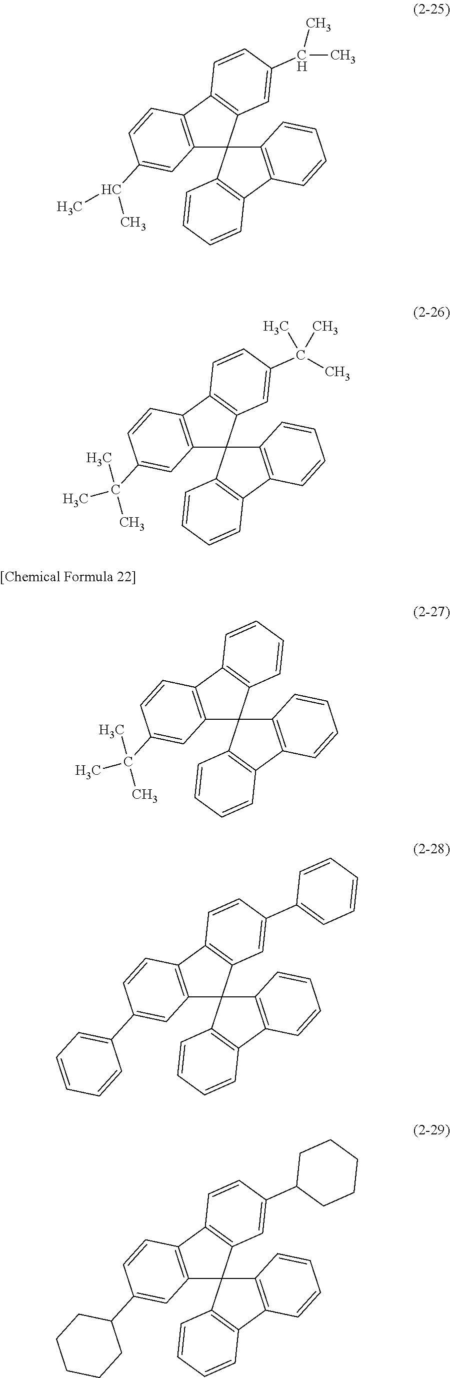

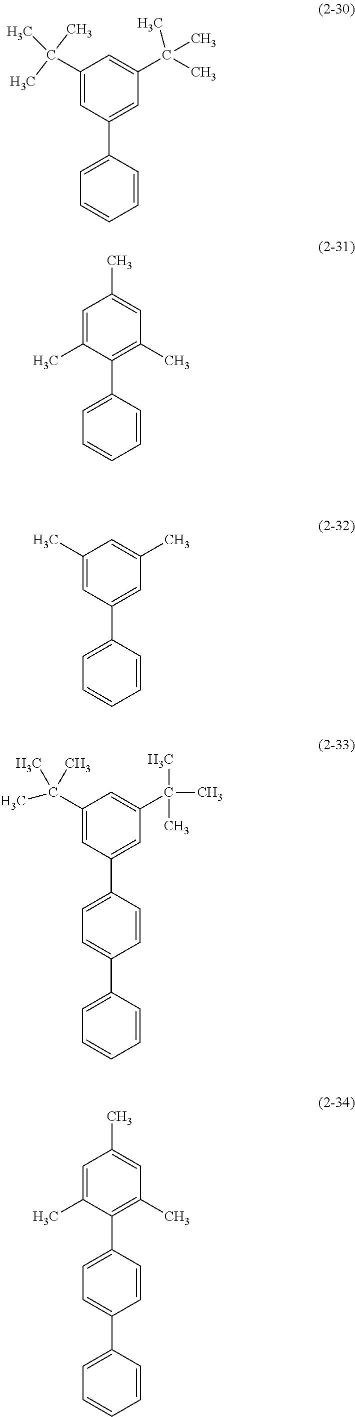

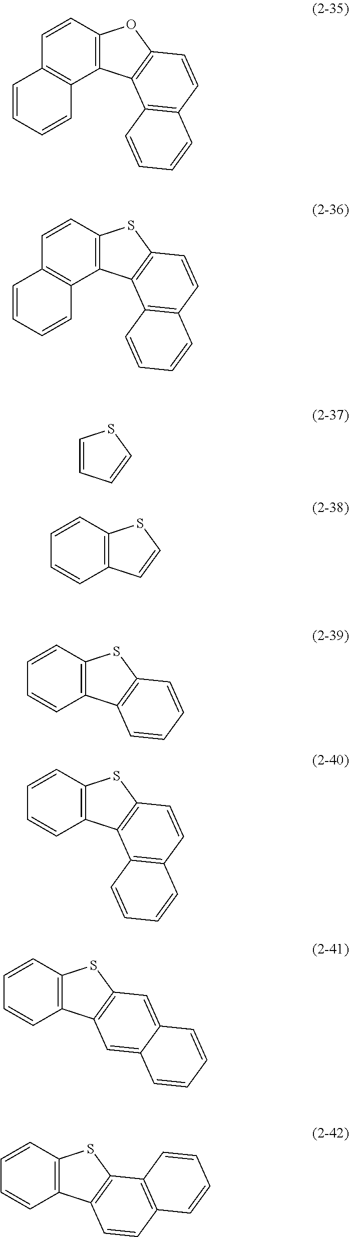

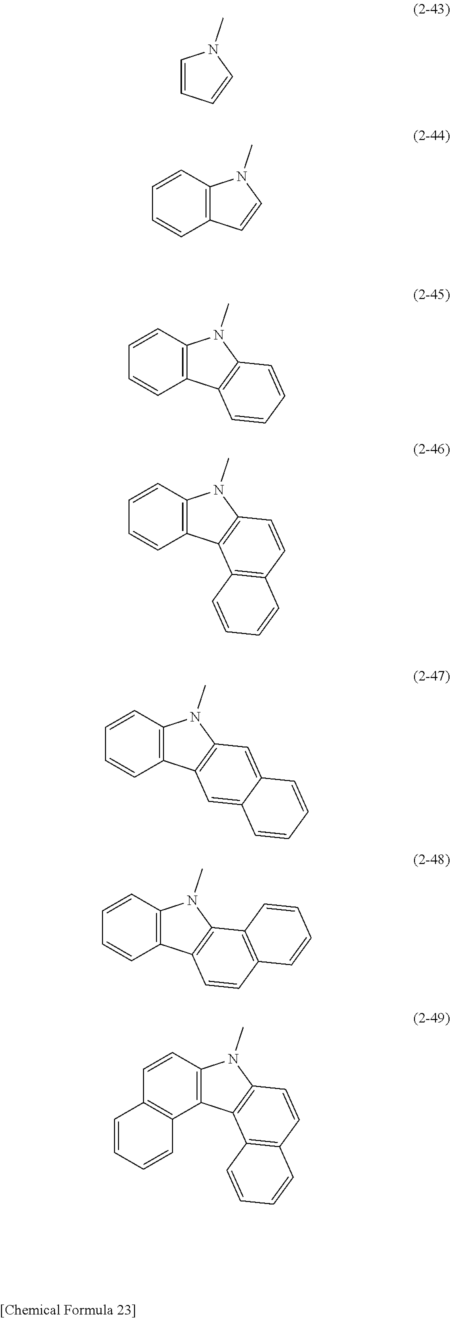

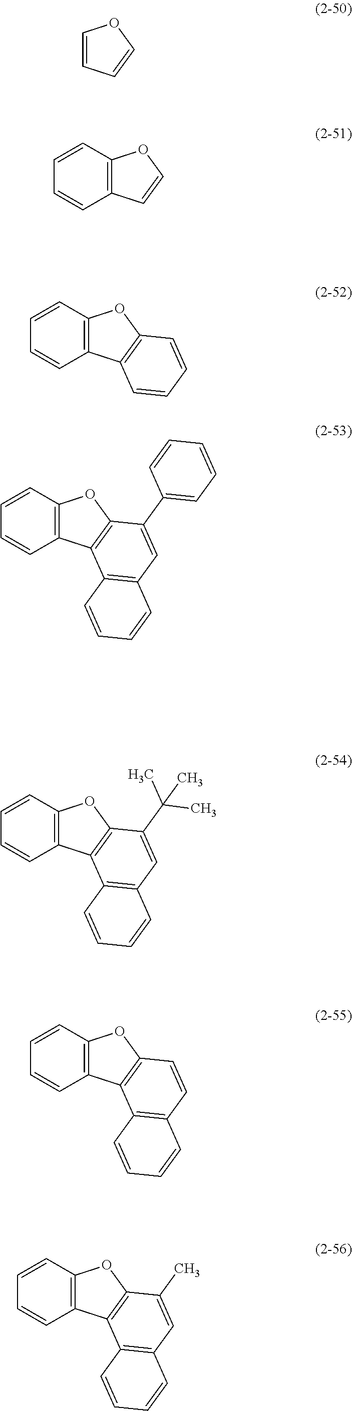

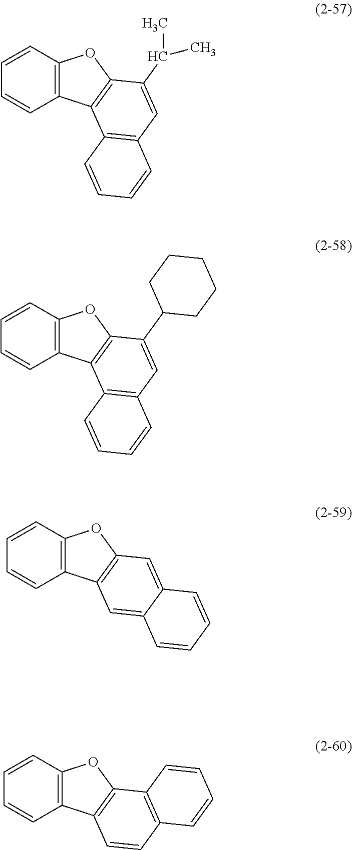 [SMILES: CC(C)c1cc2ccccc2c2c1oc1ccccc12 ; c1ccc2c(c1)cc(C1CCCCC1)c1oc3ccccc3c12 ; c1ccc2cc3c(cc2c1)oc1ccccc13 ; c1ccc2c(c1)ccc1c3ccccc3oc21]

The organic compound of one embodiment of the present invention having the above-described structure can be a highly heat-resistant material having a favorable hole-transport property. A thin film containing the compound with such a structure is preferable because it undergoes a small change in quality and can provide a device stable to heat or over driving time. A device using the compound with such a structure has a low driving voltage and a small variation in driving voltage; thus, the device can be highly reliable in voltage and high-temperature driving. The device can also have low power consumption. In addition, the organic compound with such a structure is preferable in terms of fabrication costs because it has a high sublimation property, is not decomposed in an evaporation process, and can be produced stably.

Next, a synthesis method of the organic compound represented by General Formula (G1) shown below is described.

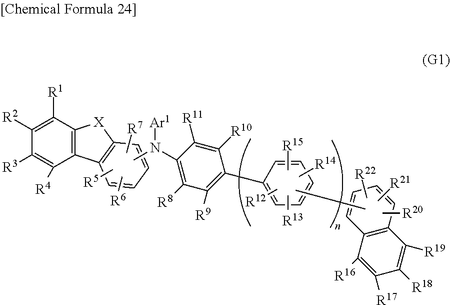

In General Formula (G1) above, R¹ to R²², X, n, and Ar¹ are the same as those described in this embodiment.

The organic compound represented by General Formula (G1) can be synthesized in a manner shown in Synthesis Schemes (a-1) and (a-2) shown below.

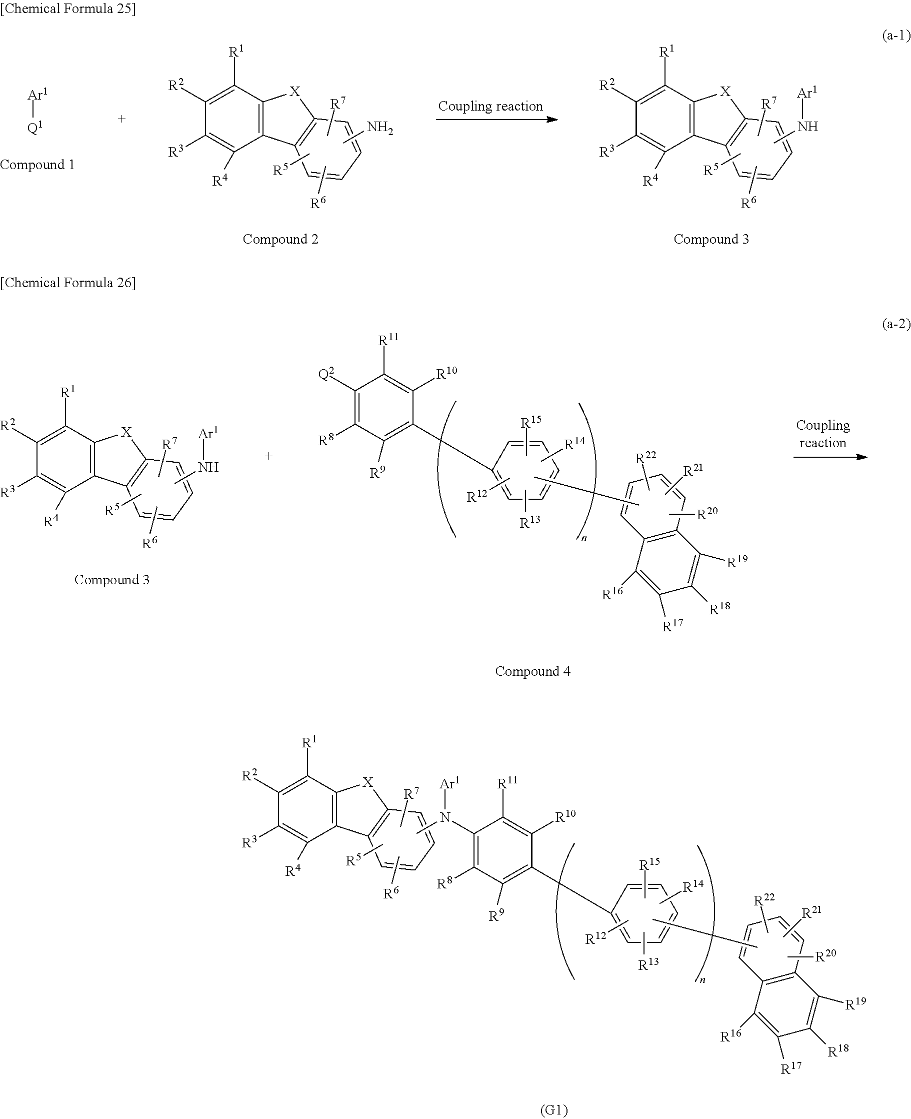

In Synthesis Schemes (a-1) and (a-2), each of Q¹ and Q² independently represents chlorine, bromine, iodine, or a triflate group. Furthermore, R¹ to R²², X, n, and Ar¹ in Compounds 1 to 4 are the same as those in the organic compound represented by General Formula (G1).

As for the organic compound represented by General Formula (G1), first, a compound (Compound 1) including a fluorene skeleton represented by Ar¹ ((g1-1) or (g1-2)) is cross-coupled with an amine compound (Compound 2) including a dibenzofuran skeleton or a dibenzothiophene skeleton, whereby a dibenzofuranylamine or dibenzothiophenylamine compound (Compound 3) including a fluorene skeleton represented by Ar¹ is obtained, as shown in Synthesis Scheme (a-1) above.

Next, in accordance with Synthesis Scheme (a-2), the dibenzofuranylamine or dibenzothiophenylamine compound (Compound 3) including a fluorene skeleton represented by Ar¹ is cross-coupled with a compound (Compound 4) including a binaphthalene skeleton, whereby the organic compound of one embodiment of the present invention represented by General Formula (G1) including a binaphthalene skeleton, a fluorene skeleton represented by Ar¹, and a dibenzofuran skeleton or a dibenzothiophene skeleton can be obtained.

For Synthesis Schemes (a-1) and (a-2) above, the Buchwald-Hartwig reaction using a palladium catalyst can be used. In that case, a palladium compound such as bis(dibenzylideneacetone)palladium(0), palladium(II) acetate, [1,1-bis(diphenylphosphino)ferrocene]palladium(II) dichloride, tetrakis(triphenylphosphine)palladium(0), or allylpalladium(II) chloride (dimer) can be used as the palladium catalyst, and tri(tert-butyl)phosphine, tri(n-hexyl)phosphine, tricyclohexylphosphine, di(1-adamantyl)-n-butylphosphine, 2-dicyclohexylphosphino-2′,6′-dimethoxybiphenyl, tri(ortho-tolyl)phosphine, or (S)-(6,6′-dimethoxybiphenyl-2,2′-diyl)bis(diisopropylphosphine) (abbreviation: cBRIDP) can be used as the ligand.

In the reaction, an organic base such as sodium tert-butoxide, an inorganic base such as potassium carbonate, cesium carbonate, or sodium carbonate, or the like can be used. As a solvent in the reaction, toluene, xylene, benzene, tetrahydrofuran, dioxane, or the like can be used. Reagents that can be used in the reaction are not limited to the above-described reagents.

Note that in Synthesis Schemes (a-1) and (a-2) above, a compound in which an organotin group is bonded to an amino group of Compound 2 or Compound 3 can be used instead of Compound 2 or Compound 3.

Compound 3 used in Synthesis Schemes (a-1) and (a-2) can be obtained also in a manner shown in Synthesis Scheme (a-1-1) below.

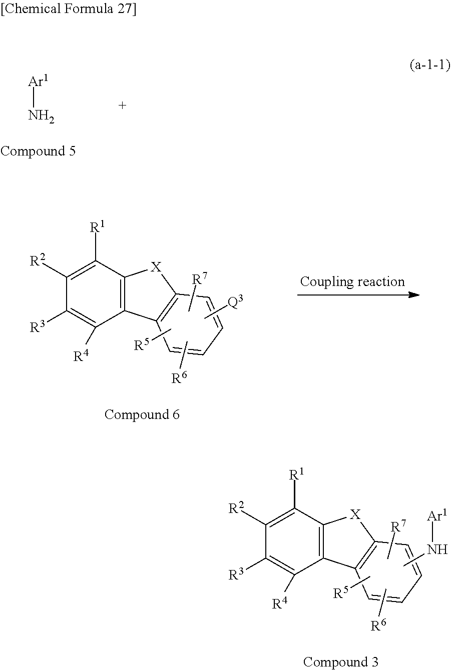

In Synthesis Scheme (a-1-1), Q³ represents chlorine, bromine, iodine, or a triflate group. In addition, R¹ to R⁷, X, and Ar¹ in Compound 3, Compound 5, and Compound 6 are the same as those in the organic compound represented by General Formula (G1). For Synthesis Scheme (a-1-1) above, the Buchwald-Hartwig reaction using a palladium catalyst can be performed, as in Synthesis Schemes (a-1) and (a-2).

The organic compound represented by General Formula (G1) can be synthesized in a manner shown in Synthesis Schemes (a-3) and (a-4) shown below.

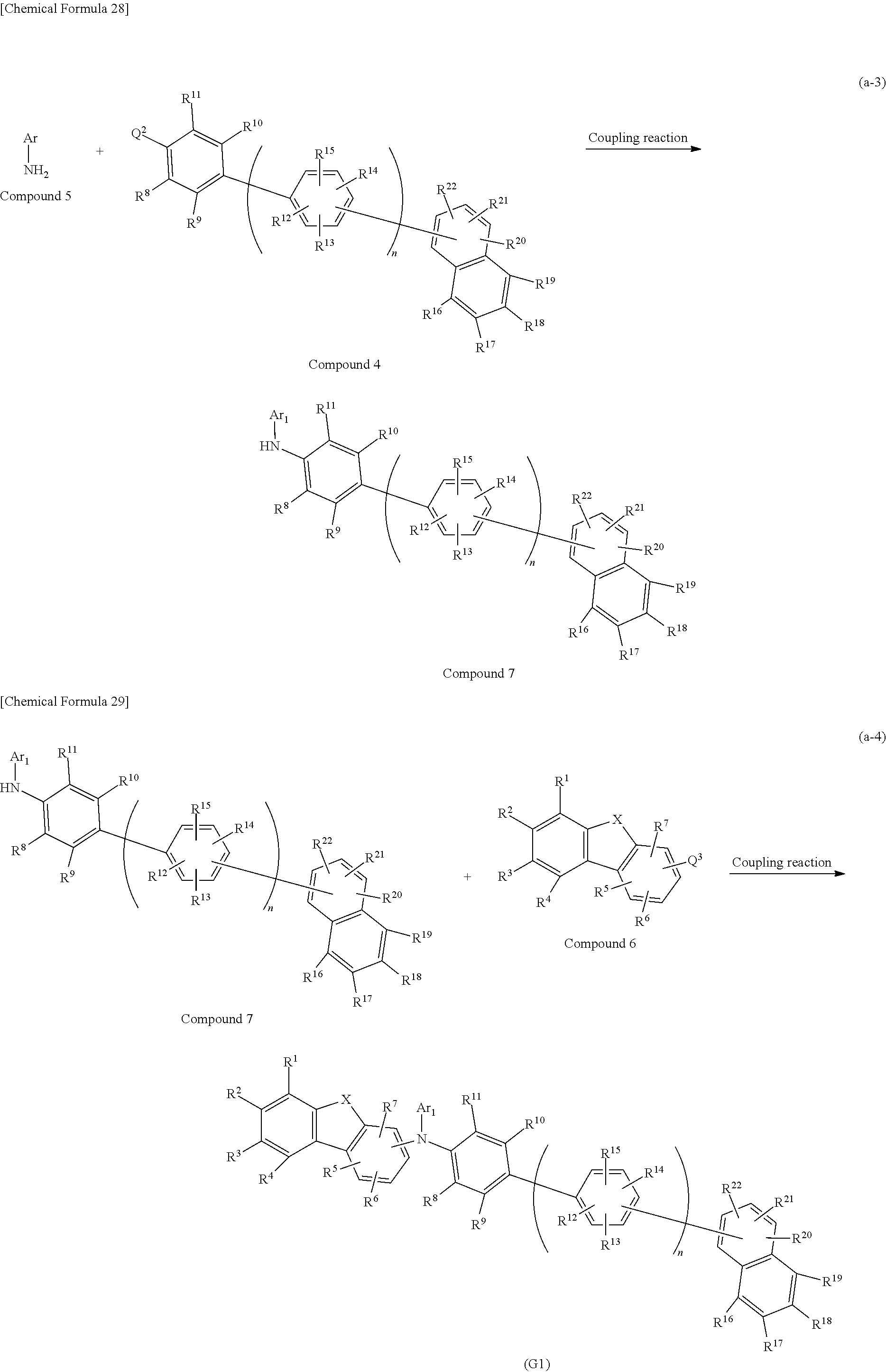

In Synthesis Schemes (a-3) and (a-4), each of Q² and Q³ independently represents chlorine, bromine, iodine, or a triflate group. In addition, R¹ to R²², X, n, and Ar¹ in Compounds 4 to 7 are the same as the organic compound represented by General Formula (G1). For Synthesis Schemes (a-3) and (a-4) above, the Buchwald-Hartwig reaction using a palladium catalyst can be performed, as in Synthesis Schemes (a-1) anti (a-2).

For the organic compound represented by General Formula (G1), first, an amine compound (Compound 5) including a fluorene skeleton represented by Ar¹ is cross-coupled with the compound (Compound 4) including a binaphthalene skeleton, whereby an amine compound (Compound 7) including a fluorene skeleton represented by Ar¹ and a binaphthalene skeleton can be obtained, as shown in Synthesis Scheme (a-3) above.

Next, in accordance with Synthesis Scheme (a-4), the amine compound (Compound 7) including a fluorene skeleton represented by Ar¹ and a binaphthalene skeleton is cross-bonded with a compound (Compound 6) including a dibenzofuran skeleton or a dibenzothiophene skeleton, whereby an organic compound of one embodiment of the present invention represented by General Formula (G1) including a binaphthalene skeleton, a fluorene skeleton represented by Ar¹, and a dibenzofuran skeleton or the dibenzothiophene skeleton can be obtained.

Compound 7 used in Synthesis Schemes (a-3) and (a-4) can be obtained also in a manner shown in Synthesis Schemes (a-3-1) and (a-3-2) below.

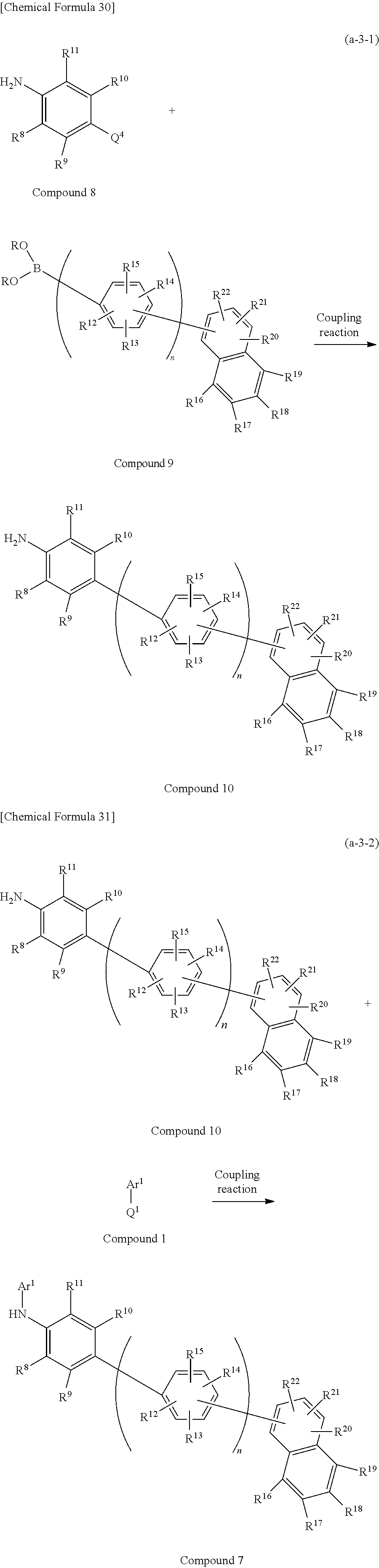

In Synthesis Schemes (a-3-1) and (a-3-2), each of Q¹ and Q⁴ independently represents chlorine, bromine, iodine, or a triflate group. Each of R⁸ to R²² independently represents hydrogen or an alkyl group having 1 to 6 carbon atoms, R⁸ to R²² may be bonded to each other to form a ring, and an example of a boron compound in that case is pinacol borane. Furthermore, R⁸ to R², X, n, and Ar¹ in Compounds 1 and 8 to 10 are the same as those in the organic compound represented by General Formula (G1).

First, as shown in Synthesis Scheme (a-3-1), an aniline compound (Compound 8) is cross-coupled with a boron compound (Compound 9) including a binaphthalene skeleton, whereby an amine compound (Compound 10) including a binaphthalene skeleton can be obtained.

Next, as shown in Synthesis Scheme (a-3-2), the amine compound (Compound 10) including a binaphthalene skeleton is cross-coupled with the compound (Compound 1) including a fluorene skeleton represented by Ar¹, whereby Compound 7 can be obtained.

In Synthesis Scheme (a-3-1) above, the Suzuki-Miyaura reaction using a palladium catalyst can be performed. In that case, examples of the palladium catalyst include a palladium compound, such as palladium(II) acetate, tetrakis(triphenylphosphine)palladium(0), or bis(triphenylphosphine)palladium(II) dichloride and examples of a ligand include tri(ortho-tolyl)phosphine, triphenylphosphine, and tricyclohexylphosphine.

As a base in the reaction, an organic base such as sodium tert-butoxide, an inorganic base such as potassium carbonate or sodium carbonate, or the like can be used. As a solvent in the reaction, a mixed solvent of toluene and water, a mixed solvent of xylene and water, a mixed solvent of benzene and water, a mixed solvent of water and an ether such as ethylene glycol dimethyl ether or 1,4-dioxane, or the like can be used. A boronic acid or a boron compound reacts at a higher rate when having higher solubility in an aqueous phase and thus potentially brings about an effect of increasing the yield; accordingly, it is preferable to add water. However, in the case where an ether is used as the solvent, a similar effect can be potentially brought about even when water is not added. In addition, as a reaction solvent, a mixed solvent of toluene, water, and alcohol such as ethanol, a mixed solvent of xylene, water, and alcohol such as ethanol, a mixed solvent of benzene, water, and alcohol such as ethanol, or the like can be used. In particular, a mixed solvent of toluene and water, a mixed solvent of toluene, water, and ethanol, or a mixed solvent of water and an ether such as ethylene glycol dimethyl ether is preferable.

Furthermore, instead of the organoboron compound or the boronic acid, an organoaluminum compound, an organozirconium compound, an organozinc compound, an organotin compound, or the like may be cross-coupled with a halide or a compound including a triflate group.

For Synthesis Scheme (a-3-2) above, the Buchwald-Hartwig reaction using a palladium catalyst can be performed, as in Synthesis Schemes (a-1) and (a-2).

Note that Compound 10 synthesized in Synthesis Scheme (a-3-1) above can be synthesized also in a manner shown in Synthesis Scheme (a-3-1-1) below.

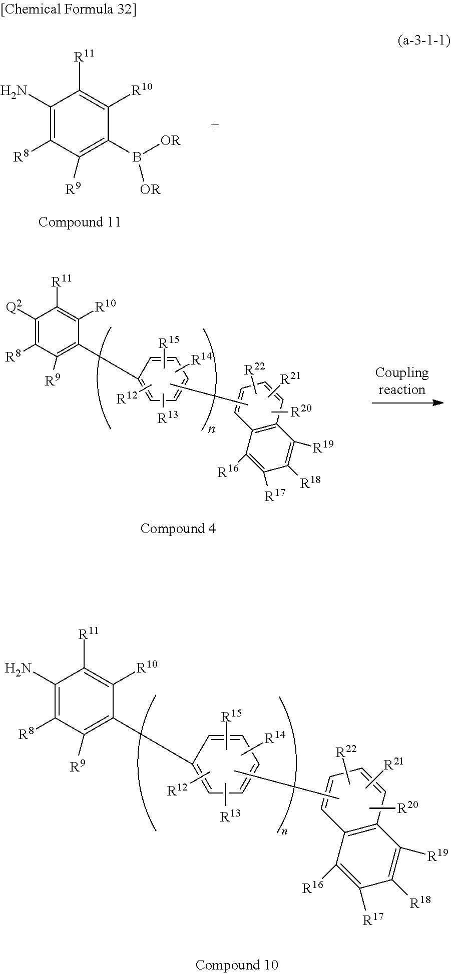

In Synthesis Scheme (a-3-1-1), Q² represents chlorine, bromine, iodine, or a triflate group. Each of R⁸ to R²² independently represents hydrogen or an alkyl group having 1 to 6 carbon atoms, R⁸ to R²² may be bonded to each other to form a ring, and an example of a boron compound in that case is pinacol borane. Furthermore, R⁸ to R¹¹, X, n, and Ar¹ in Compound 4, Compound 10, and Compound 11 are the same as those in the organic compound represented by General Formula (G1). For Synthesis Scheme (a-3-1-1) above, the Suzuki-Miyaura reaction using a palladium catalyst can be performed, as in Synthesis Scheme (a-3-1).

In Synthesis Scheme (a-3-1-1) above, a boron compound (Compound 11) including an aniline skeleton is cross-coupled with the compound (Compound 4) including a binaphthalene skeleton, whereby an aniline compound (Compound 10) including a binaphthalene skeleton can be obtained.

Compound 7 used in Synthesis Schemes (a-3) and (a-4) can be obtained also in a manner shown in Synthesis Schemes (a-3-2-1) and (a-3-2-2) below.

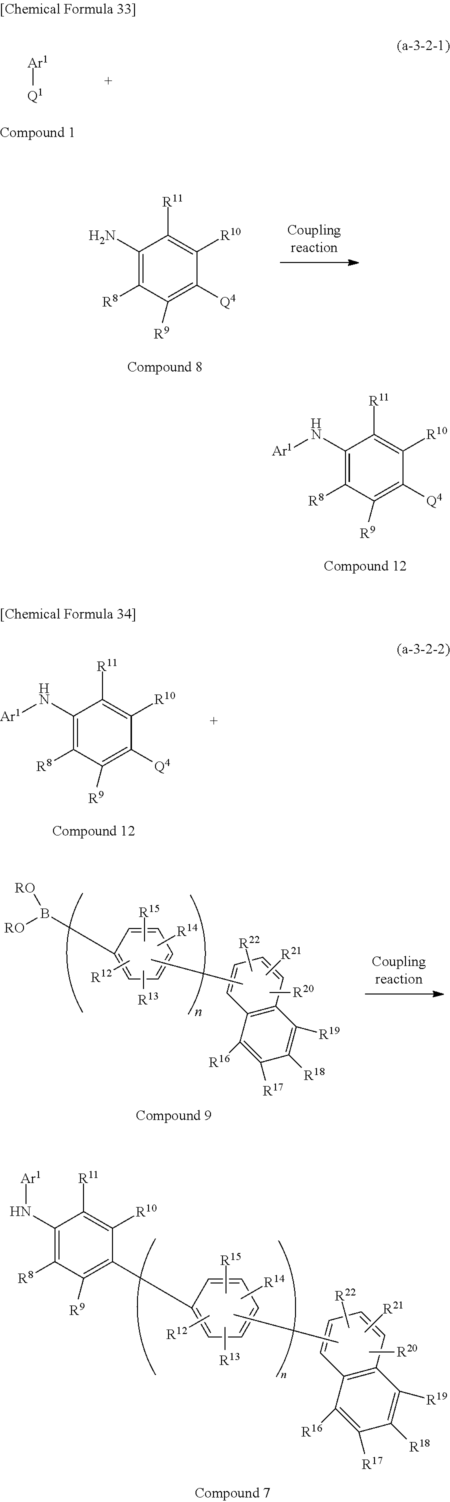

In Synthesis Schemes (a-3-2-1) and (a-3-2-2), each of Q¹ and Q⁴ independently represents chlorine, bromine, iodine, or a triflate group. Each of R⁸ to R²² independently represents hydrogen or an alkyl group having 1 to 6 carbon atoms, R⁸ to R²² may be bonded to each other to form a ring, and an example of a boron compound in that case is pinacol borane. Furthermore, R⁸ to R²², X, n, and Ar¹ in Compounds 1, 7 to 9, and 12 are the same as those in the organic compound represented by General Formula (G1).

First, as shown in Synthesis Scheme (a-3-2-1) above, the compound (Compound 1) including a fluorene skeleton represented by Ar¹ is cross-coupled with the aniline compound (Compound 8), whereby an amine compound (Compound 12) including a phenyl group and a fluorene skeleton represented by Ar¹ can be obtained.

Next, as shown in Synthesis Scheme (a-3-2-2), the amine compound (Compound 12) including a phenyl group and a fluorene skeleton represented by Ar¹ is cross-coupled with the boron compound (Compound 9) including a binaphthalene skeleton, whereby the amine compound (Compound 7) including a binaphthalene skeleton and a fluorene skeleton represented by Ar¹ can be obtained.

For Synthesis Scheme (a-3-2-1) above, the Buchwald-Hartwig reaction using a palladium catalyst can be performed, as in Synthesis Schemes (a-1) and (a-2). For Synthesis Scheme (a-3-2-2) above, the Suzuki-Miyaura reaction using a palladium catalyst can be performed, as in Synthesis Scheme (a-3-3-1).

The organic compound represented by General Formula (G1) can be synthesized in a manner shown in Synthesis Schemes (a-5) and (a-6) shown below.

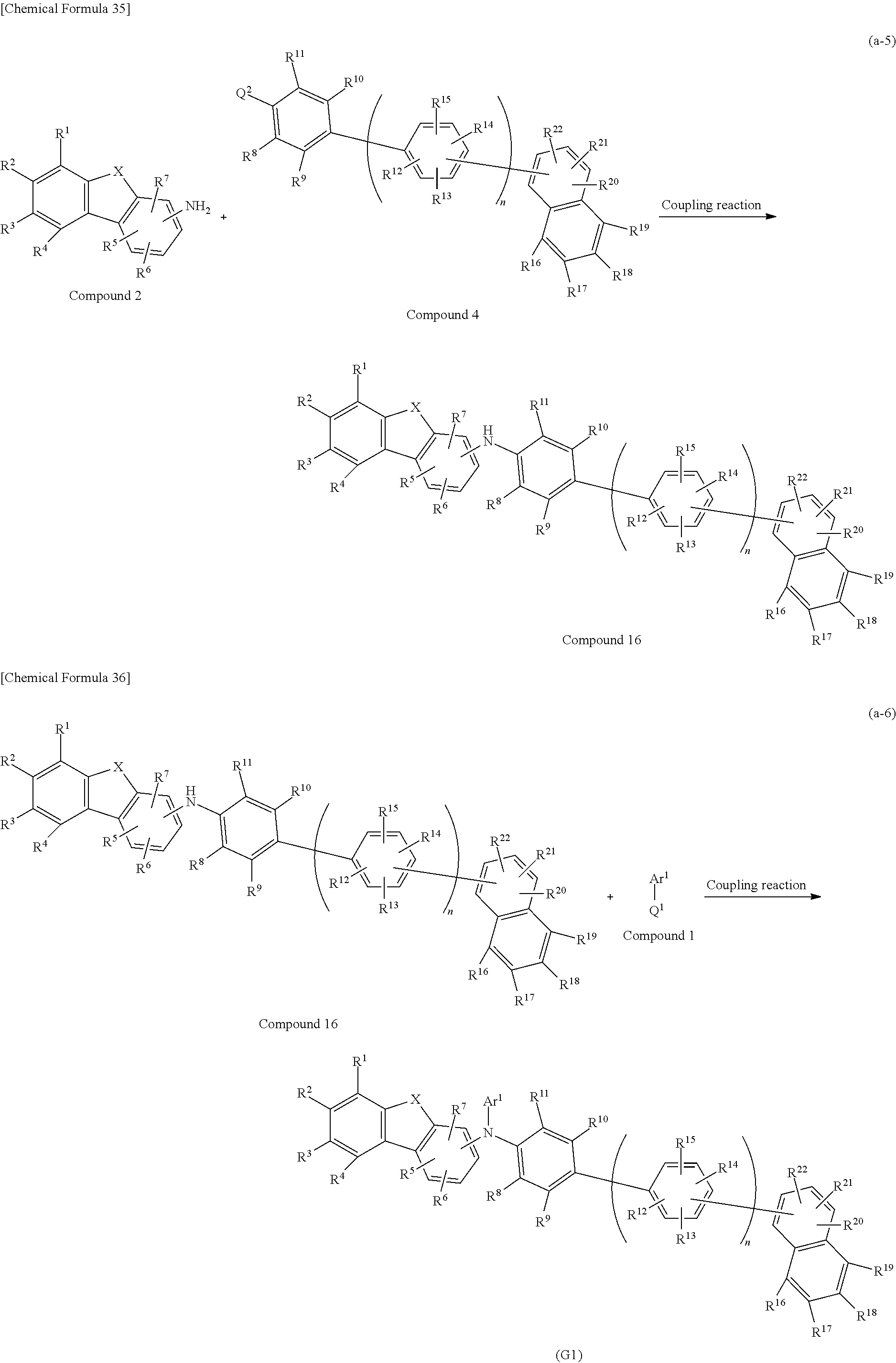

In Synthesis Schemes (a-3) and (a-4), each of Q¹ and Q² independently represents chlorine, bromine, iodine, or a triflate group. In addition, R¹ to R²², X, n, and Ar¹ in Compounds 1, 2, 4, and 16 are the same as the organic compound represented by General Formula (G1). For Synthesis Schemes (a-5) and (a-6) above, the Buchwald-Hartwig reaction using a palladium catalyst can be performed, as in Synthesis Schemes (a-1) and (a-2).

In this synthesis scheme, first, as shown in Synthesis Scheme (a-5) above, the amine compound (Compound 2) including a dibenzofuran skeleton or a dibenzothiophene skeleton is cross-coupled with the compound (Compound 4) including a binaphthalene skeleton, whereby an amine compound (Compound 16) including a binaphthalene skeleton and a dibenzofuranylamine skeleton or a dibenzothiophenyl skeleton can be obtained.

Next, in accordance with Synthesis Scheme (a-6), the amine compound (Compound 16) including a binaphthalene skeleton and a dibenzofuranylamine skeleton or a dibenzothiophenyl skeleton is cross-coupled with the compound (Compound 1) including a fluorene skeleton represented by Ar¹, whereby the organic compound of one embodiment of the present invention represented by General Formula (G1) including a binaphthalene skeleton, a fluorene skeleton represented by Art, and a dibenzofuran skeleton or a dibenzothiophene skeleton can be obtained.

Alternatively, Compound 16 can be synthesized in a manner shown in Synthesis Scheme (a-5-1) below, using the amine compound (Compound 10) including a binaphthalene skeleton, which is synthesized in Synthesis Scheme (a-3-1) or (a-3-1-1).

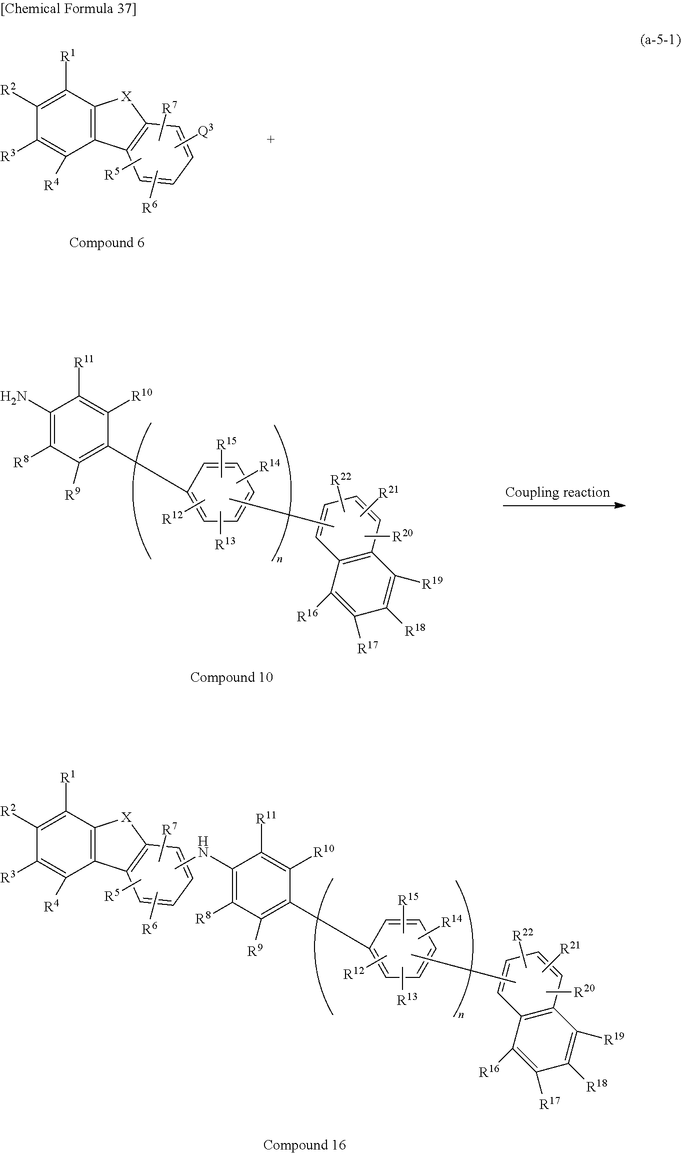

In Synthesis Scheme (a-5-1), Q³ represents chlorine, bromine, iodine, or a triflate group. In addition, R¹ to R²², X, and n in Compound 6, Compound 10, and Compound 16 are the same as those in the organic compound represented by General Formula (G1). For Synthesis Scheme (a-5-1) above, the Buchwald-Hartwig reaction using a palladium catalyst can be performed, as in Synthesis Schemes (a-1) and (a-2).

In Synthesis Scheme (a-5-1), the compound (Compound 6) including a dibenzofuran skeleton or a dibenzothiophene skeleton is cross-coupled with the amine compound (Compound 10) including a binaphthalene skeleton, whereby the amine compound (Compound 16) including a binaphthalene skeleton and a dibenzofuranylamine skeleton or a dibenzothiophenyl skeleton can be obtained.

Alternatively, Compound 16 can be synthesized in a manner shown in Synthesis Schemes (a-5-2) and (a-5-3) below.

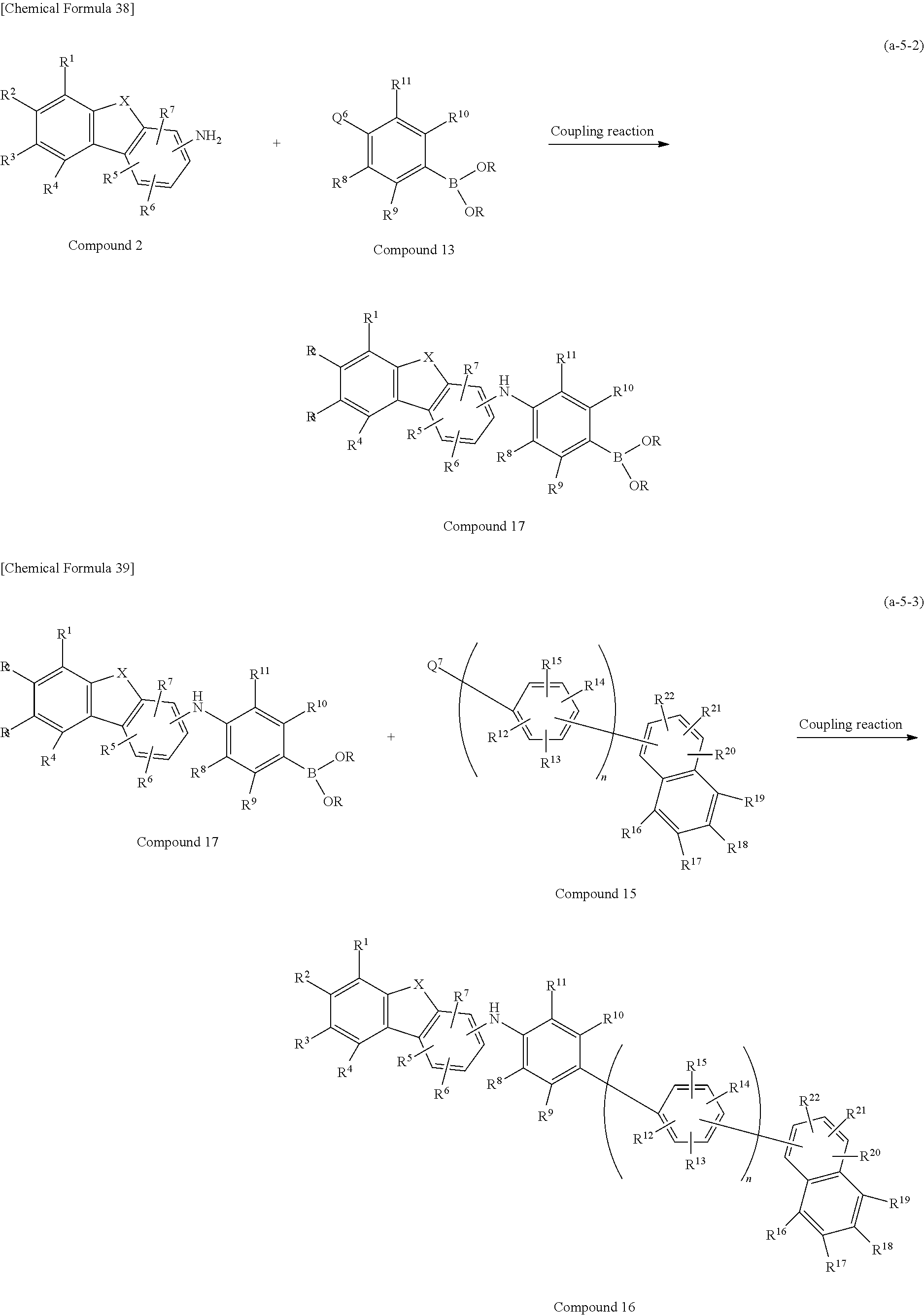

In Synthesis Schemes (a-5-2) and (a-5-3), each of Q⁶ and Q⁷ independently represents chlorine, bromine, iodine, or a triflate group. Each of R¹ to R²² independently represents hydrogen or an alkyl group having 1 to 6 carbon atoms, R¹ to R²² may be bonded to each other to form a ring, and an example of a boron compound in that case is pinacol borane. Furthermore, R¹ to R²², X, and n in Compounds 2, 13, 15, 16, and 17 are the same as those in the organic compound represented by General Formula (G1).

First, as in Synthesis Scheme (a-5-2) above, the amine compound (Compound 2) including a dibenzofuran skeleton or a dibenzothiophene skeleton is cross-coupled with a boron compound (Compound 13) including a phenyl group, whereby a dibenzofuranyl or dibenzothiophenylamine compound (Compound 17) including a phenyl group to which boron is added can be obtained.

Next, as in Synthesis Scheme (a-5-3) above, Compound 17 is cross-coupled with a boron compound (Compound 15) including a binaphthalene skeleton, whereby the amine compound (Compound 16) including a binaphthalene skeleton and a dibenzofuranylamine skeleton or a dibenzothiophenyl skeleton can be obtained.

For Synthesis Scheme (a-5-2) above, the Buchwald-Hartwig reaction using a palladium catalyst can be performed, as in Synthesis Schemes (a-1) and (a-2). For Synthesis Scheme (a-5-3) above, the Suzuki-Miyaura reaction using a palladium catalyst can be performed, as in Synthesis Scheme (a-3-1).

Compound 17 can be synthesized in a manner shown in Synthesis Scheme (a-5-2-1) below.

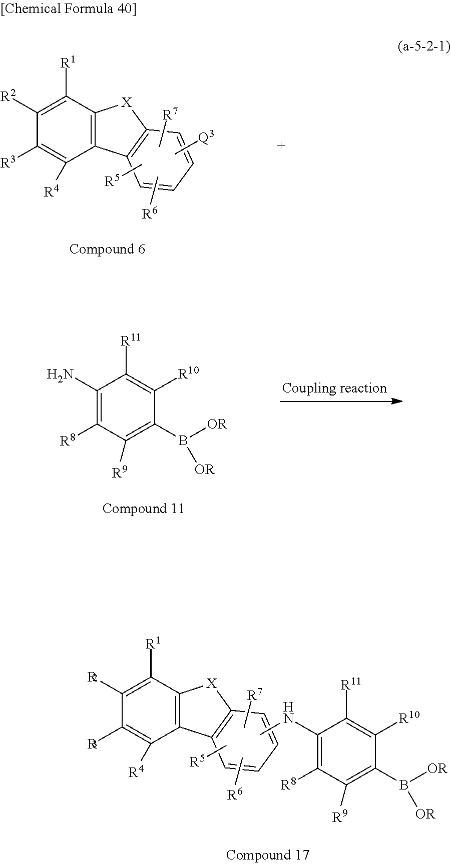

In Synthesis Scheme (a-5-2-1), Q³ represents chlorine, bromine, iodine, or a triflate group. Each of R¹ to R¹¹ independently represents hydrogen or an alkyl group having 1 to 6 carbon atoms, R¹ to R¹¹ may be bonded to each other to form a ring, and an example of a boron compound in that case is pinacol borane. Furthermore, R¹ to R¹¹ and X in Compounds 6, 11, and 17 are the same as those in the organic compound represented by General Formula (G1). For Synthesis Scheme (a-5-2-1) above, the Buchwald-Hartwig reaction using a palladium catalyst can be performed, as in Synthesis Schemes (a-1) and (a-2).

In Synthesis Scheme (a-5-2-1), the compound (Compound 6) including a dibenzofuran skeleton or a dibenzothiophene skeleton is cross-coupled with the boron compound (Compound 11) including an aniline skeleton, whereby the dibenzofuranyl or dibenzothiophenylamine compound (Compound 17) including a phenyl group to which boron is added can be obtained.

The organic compound represented by General Formula (G1) can be synthesized in a manner shown in Synthesis Schemes (a-7) and (a-8), using the dibenzofuranylamine or dibenzothiophenylamine compound (Compound 3) including a fluorene skeleton represented by Ar¹ synthesized in Synthesis Scheme (a-1) or (a-1-1) above.

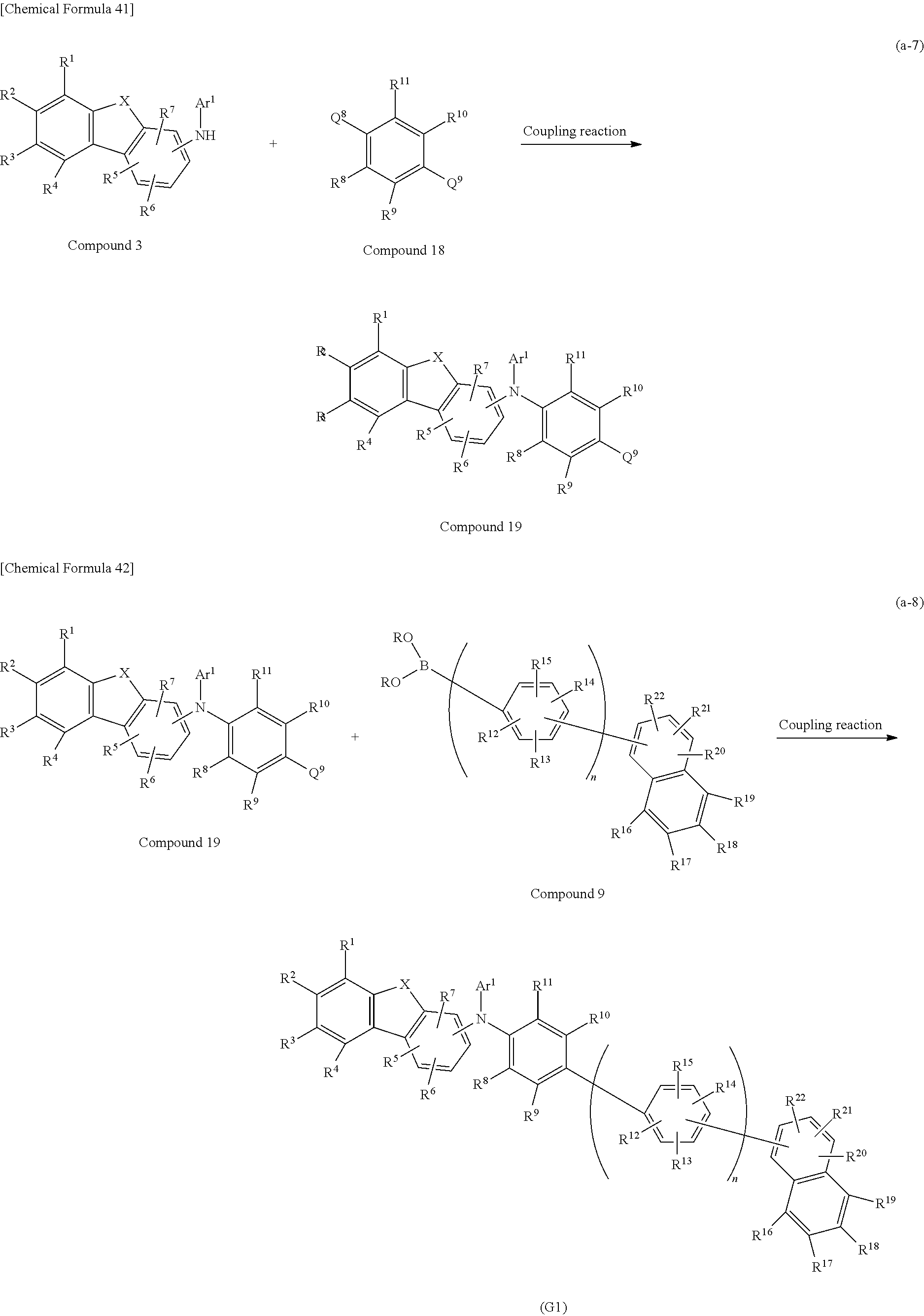

In Synthesis Schemes (a-7) and (a-8), each of Q⁸ and Q⁹ independently represents chlorine, bromine, iodine, or a triflate group. Each of R¹ to R²² independently represents hydrogen or an alkyl group having 1 to 6 carbon atoms, R¹ to R²² may be bonded to each other to form a ring, and an example of a boron compound in that case is pinacol borane. Furthermore, R¹ to R²², X, n, and Ar¹ in Compounds 3, 9, 18, and 19 are the same as those in the organic compound represented by General Formula (G1).

First, as shown in Synthesis Scheme (a-7) above, the dibenzofuranylamine or dibenzothiophenylamine compound (Compound 3) including a fluorene skeleton represented by Ar¹ is cross-coupled with the benzene compound (Compound 18), whereby an amine compound (Compound 19) including a dibenzofuran skeleton or a dibenzothiophene skeleton and a fluorene skeleton represented by Ar¹ can be obtained.

Next, in accordance with Synthesis Scheme (a-8), Compound 19 is cross-coupled with the boron compound (Compound 9) including a binaphthalene skeleton, whereby the organic compound of one embodiment of the present invention represented by General Formula (G1) including a binaphthalene skeleton, a fluorene skeleton represented by Ar¹, and a dibenzofuran skeleton or a dibenzothiophene skeleton can be obtained.

For Synthesis Scheme (a-7) above, the Buchwald-Hartwig reaction using a palladium catalyst can be performed, as in Synthesis Schemes (a-1) and (a-2). For Synthesis Scheme (a-8) above, the Suzuki-Miyaura reaction using a palladium catalyst can be performed, as in Synthesis Scheme (a-3-1).

The organic compound represented by General Formula (G1) can be synthesized in a manner shown in Synthesis Schemes (a-9) and (a-10), using the dibenzofuranylamine or dibenzothiophenylamine compound (Compound 3) including a fluorene skeleton represented by Ar¹ synthesized in Synthesis Scheme (a-1) or (a-1-1) above.

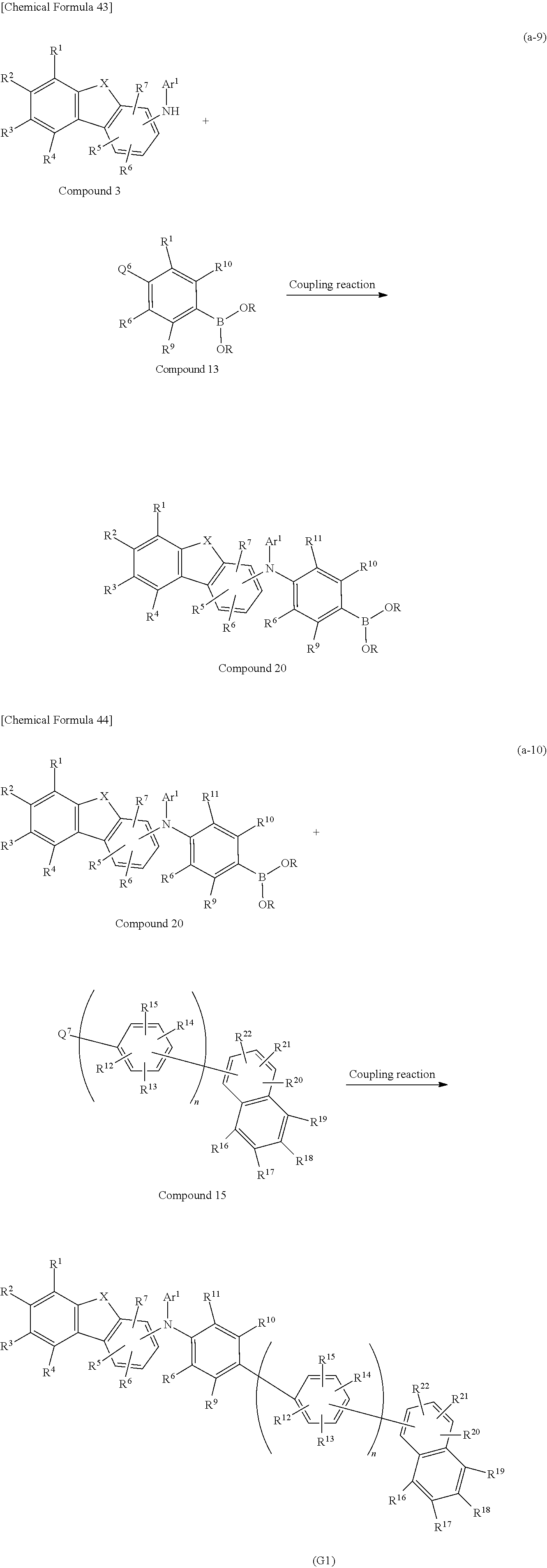

In Synthesis Schemes (a-9) and (a-10), each of Q⁶ and Q⁷ independently represents chlorine, bromine, iodine, or a triflate group. Each of R¹ to R²² independently represents hydrogen or an alkyl group having 1 to 6 carbon atoms, R¹ to R²² may be bonded to each other to form a ring, and an example of a boron compound in that case is pinacol borane. Furthermore, R¹ to R²², X, n, and Ar¹ in Compounds 3, 13, 15, and 20 are the same as those in the organic compound represented by General Formula (G1).

First, as shown in Synthesis Scheme (a-9) above, the dibenzofuranylamine or dibenzothiophenylamine compound (Compound 3) including a fluorene skeleton represented by Ar¹ is cross-coupled with the boron compound (Compound 13) including a phenyl group, a dibenzofuranylamine or dibenzothiophenylamine compound (Compound 20) including a phenyl group to which boron is added can be obtained.

Next, in accordance with Synthesis Scheme (a-8), Compound 20 is cross-coupled with the boron compound (Compound 15) including a binaphthalene skeleton, whereby the organic compound of one embodiment of the present invention represented by General Formula (G1) including a binaphthalene skeleton, a fluorene skeleton represented by Ar¹, and a dibenzofuran skeleton or a dibenzothiophene skeleton can be obtained.

For Synthesis Scheme (a-9) above, the Buchwald-Hartwig reaction using a palladium catalyst can be performed, as in Synthesis Schemes (a-1) and (a-2). For Synthesis Scheme (a-10) above, the Suzuki-Miyaura reaction using a palladium catalyst can be performed, as in Synthesis Scheme (a-3-1).

In the above manner, the organic compound of one embodiment of the present invention represented by General Formula (G1) can be obtained.

Note that specific examples of organic compounds that can be used as the secondary amine of Compound 3 in the above synthesis scheme are shown.

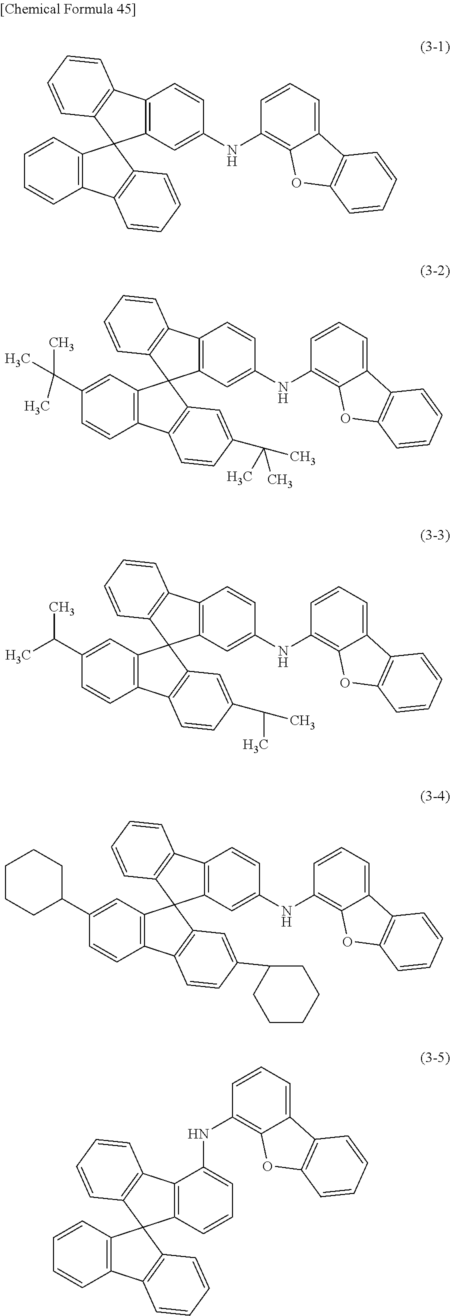

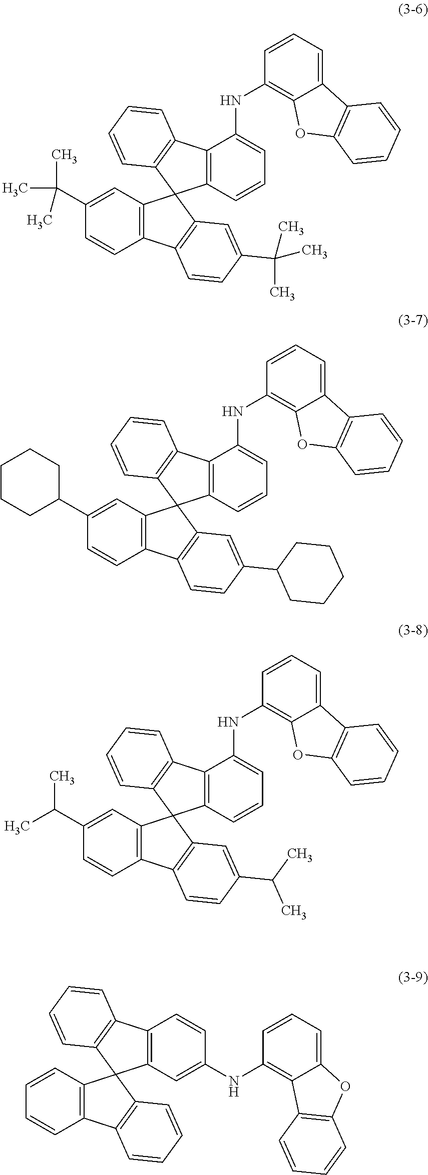 [SMILES: CC(C)(C)c1ccc2c(c1)C1(c3cc(C(C)(C)C)ccc3-2)c2ccccc2-c2c(Nc3cccc4c3oc3ccccc34)cccc21 ; c1ccc2c(c1)-c1c(Nc3cccc4c3oc3ccccc34)cccc1C21c2cc(C3CCCCC3)ccc2-c2ccc(C3CCCCC3)cc21 ; CC(C)c1ccc2c(c1)C1(c3cc(C(C)C)ccc3-2)c2ccccc2-c2c(Nc3cccc4c3oc3ccccc34)cccc21 ; c1ccc2c(c1)-c1ccccc1C21c2ccccc2-c2ccc(Nc3cccc4oc5ccccc5c34)cc21]

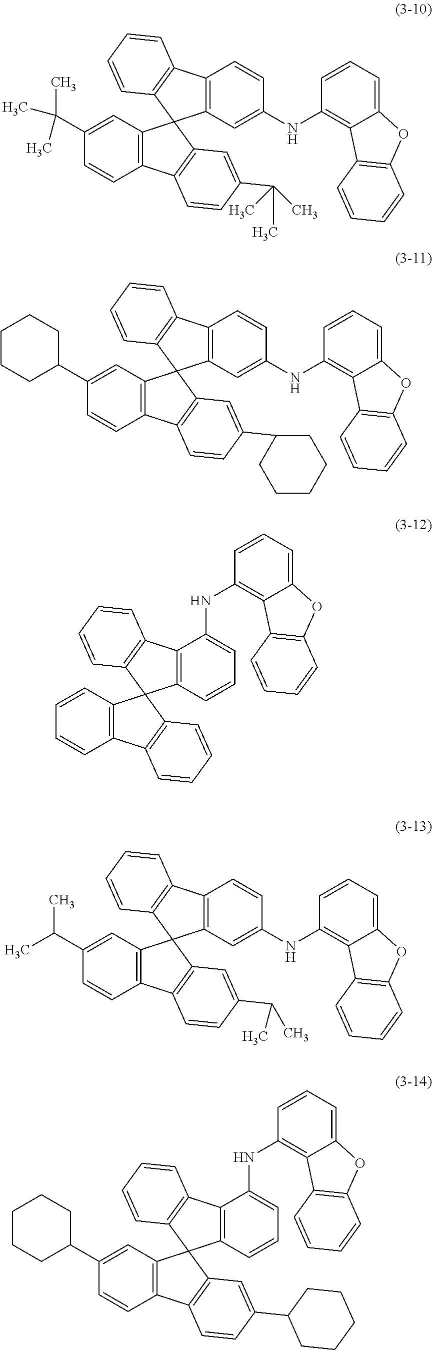

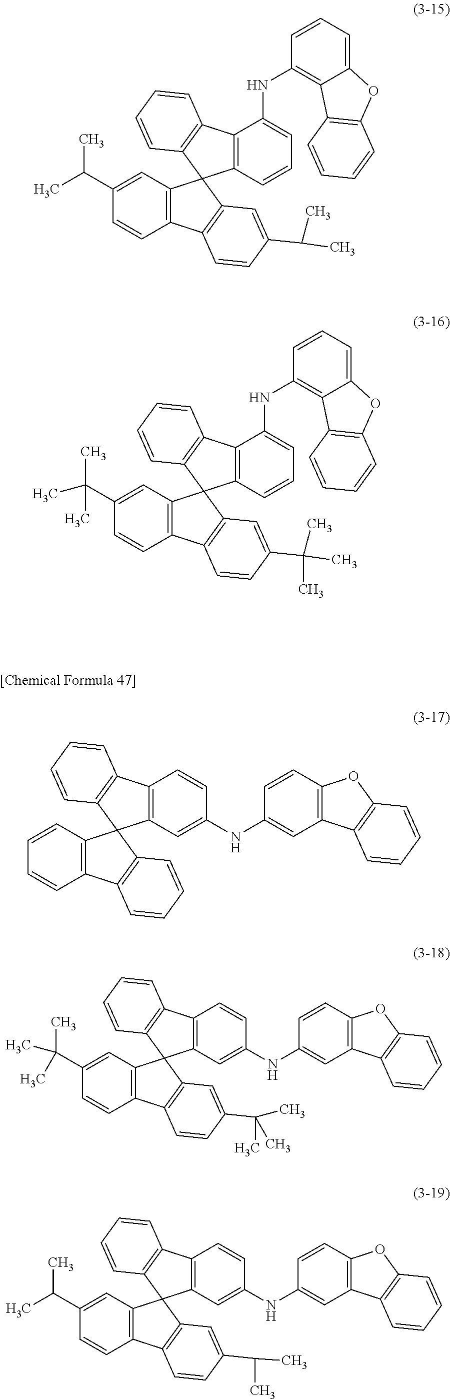 [SMILES: CC(C)c1ccc2c(c1)C1(c3cc(C(C)C)ccc3-2)c2ccccc2-c2c(Nc3cccc4oc5ccccc5c34)cccc21 ; CC(C)(C)c1ccc2c(c1)C1(c3cc(C(C)(C)C)ccc3-2)c2ccccc2-c2c(Nc3cccc4oc5ccccc5c34)cccc21 ; c1ccc2c(c1)-c1ccccc1C21c2ccccc2-c2ccc(Nc3ccc4oc5ccccc5c4c3)cc21 ; CC(C)(C)c1ccc2c(c1)C1(c3ccccc3-c3ccc(Nc4ccc5oc6ccccc6c5c4)cc31)c1cc(C(C)(C)C)ccc1-2 ; CC(C)c1ccc2c(c1)C1(c3ccccc3-c3ccc(Nc4ccc5oc6ccccc6c5c4)cc31)c1cc(C(C)C)ccc1-2]

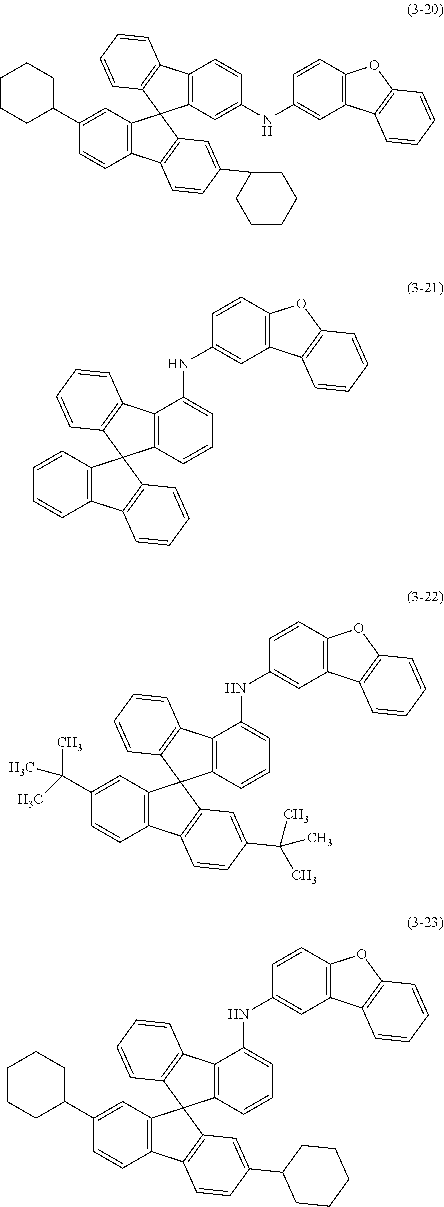 [SMILES: c1ccc2c(c1)-c1ccc(Nc3ccc4oc5ccccc5c4c3)cc1C21c2cc(C3CCCCC3)ccc2-c2ccc(C3CCCCC3)cc21 ; c1ccc2c(c1)-c1ccccc1C21c2ccccc2-c2c(Nc3ccc4oc5ccccc5c4c3)cccc21 ; CC(C)(C)c1ccc2c(c1)C1(c3cc(C(C)(C)C)ccc3-2)c2ccccc2-c2c(Nc3ccc4oc5ccccc5c4c3)cccc21 ; c1ccc2c(c1)-c1c(Nc3ccc4oc5ccccc5c4c3)cccc1C21c2cc(C3CCCCC3)ccc2-c2ccc(C3CCCCC3)cc21]

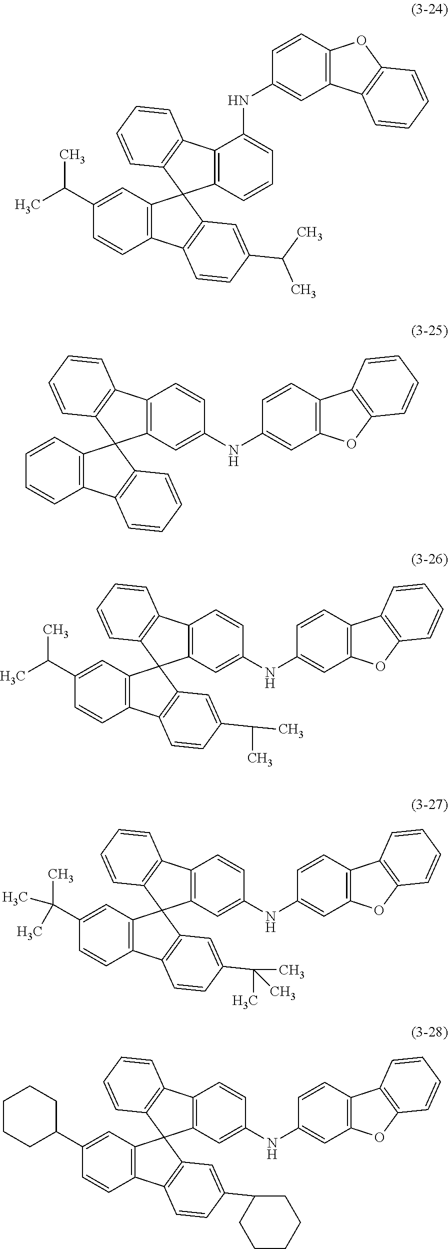 [SMILES: CC(C)c1ccc2c(c1)C1(c3cc(C(C)C)ccc3-2)c2ccccc2-c2c(Nc3ccc4oc5ccccc5c4c3)cccc21 ; c1ccc2c(c1)-c1ccccc1C21c2ccccc2-c2ccc(Nc3ccc4c(c3)oc3ccccc34)cc21 ; CC(C)c1ccc2c(c1)C1(c3ccccc3-c3ccc(Nc4ccc5c(c4)oc4ccccc45)cc31)c1cc(C(C)C)ccc1-2 ; CC(C)(C)c1ccc2c(c1)C1(c3ccccc3-c3ccc(Nc4ccc5c(c4)oc4ccccc45)cc31)c1cc(C(C)(C)C)ccc1-2 ; c1ccc2c(c1)-c1ccc(Nc3ccc4c(c3)oc3ccccc34)cc1C21c2cc(C3CCCCC3)ccc2-c2ccc(C3CCCCC3)cc21]

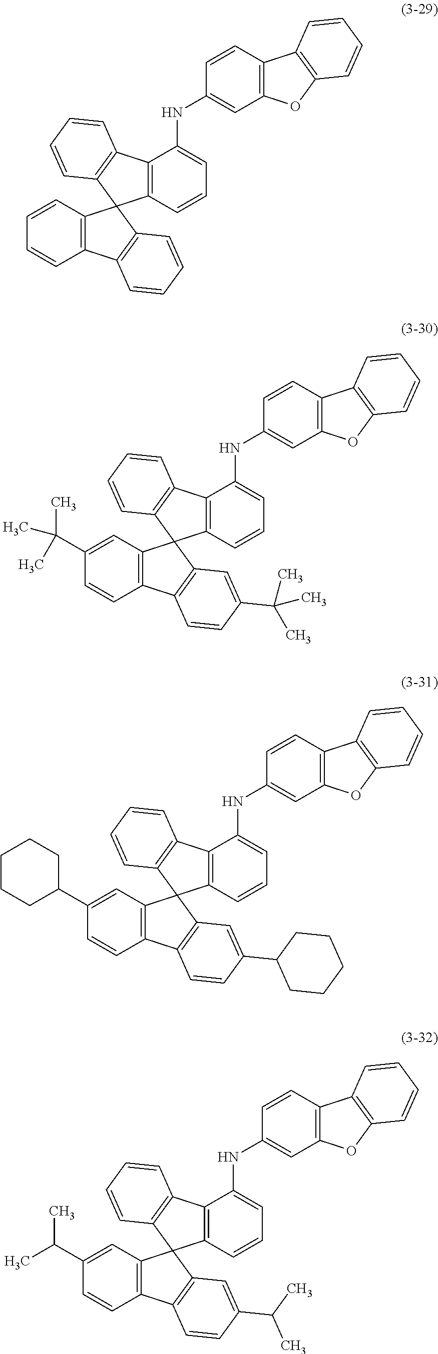 [SMILES: c1ccc2c(c1)-c1ccccc1C21c2ccccc2-c2c(Nc3ccc4c(c3)oc3ccccc34)cccc21 ; CC(C)(C)c1ccc2c(c1)C1(c3cc(C(C)(C)C)ccc3-2)c2ccccc2-c2c(Nc3ccc4c(c3)oc3ccccc34)cccc21 ; c1ccc2c(c1)-c1c(Nc3ccc4c(c3)oc3ccccc34)cccc1C21c2cc(C3CCCCC3)ccc2-c2ccc(C3CCCCC3)cc21 ; CC(C)c1ccc2c(c1)C1(c3cc(C(C)C)ccc3-2)c2ccccc2-c2c(Nc3ccc4c(c3)oc3ccccc34)cccc21]

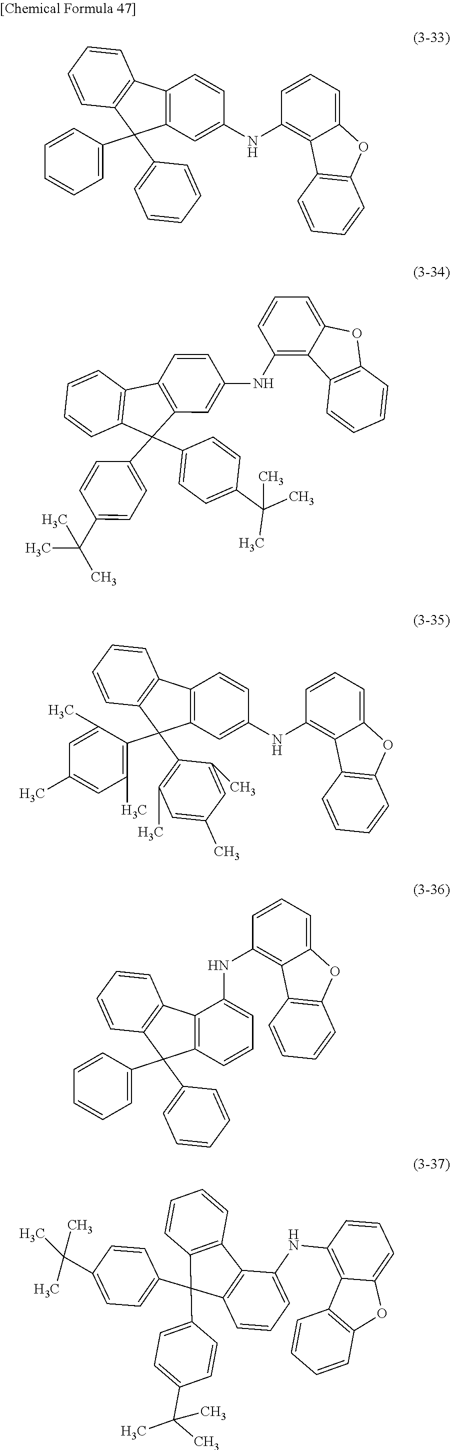

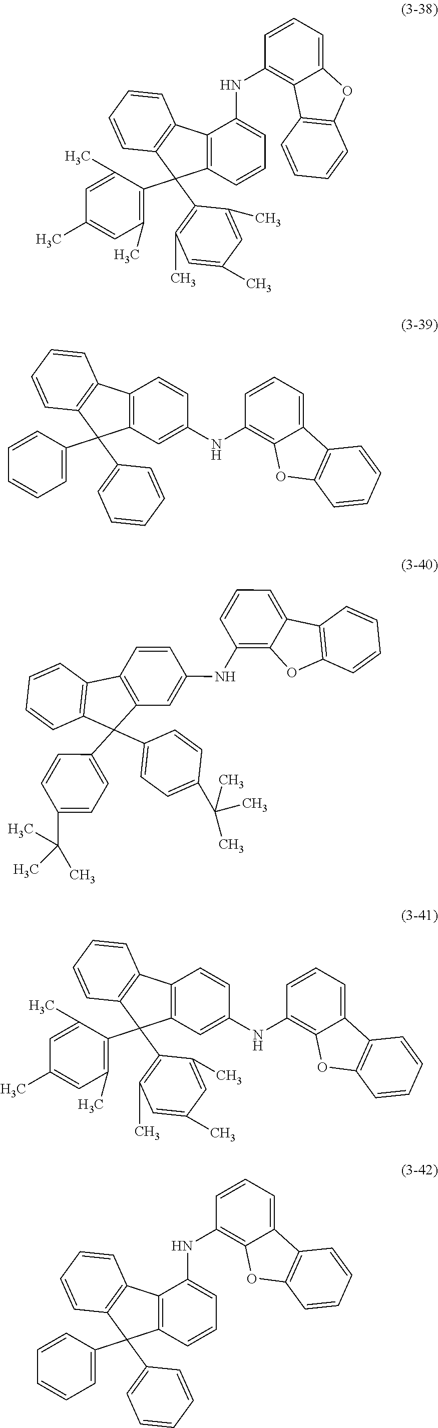 [SMILES: Cc1cc(C)c(C2(c3c(C)cc(C)cc3C)c3ccccc3-c3c(Nc4cccc5oc6ccccc6c45)cccc32)c(C)c1 ; c1ccc(C2(c3ccccc3)c3ccccc3-c3ccc(Nc4cccc5c4oc4ccccc45)cc32)cc1 ; CC(C)(C)c1ccc(C2(c3ccc(C(C)(C)C)cc3)c3ccccc3-c3ccc(Nc4cccc5c4oc4ccccc45)cc32)cc1 ; Cc1cc(C)c(C2(c3c(C)cc(C)cc3C)c3ccccc3-c3ccc(Nc4cccc5c4oc4ccccc45)cc32)c(C)c1 ; c1ccc(C2(c3ccccc3)c3ccccc3-c3c(Nc4cccc5c4oc4ccccc45)cccc32)cc1]

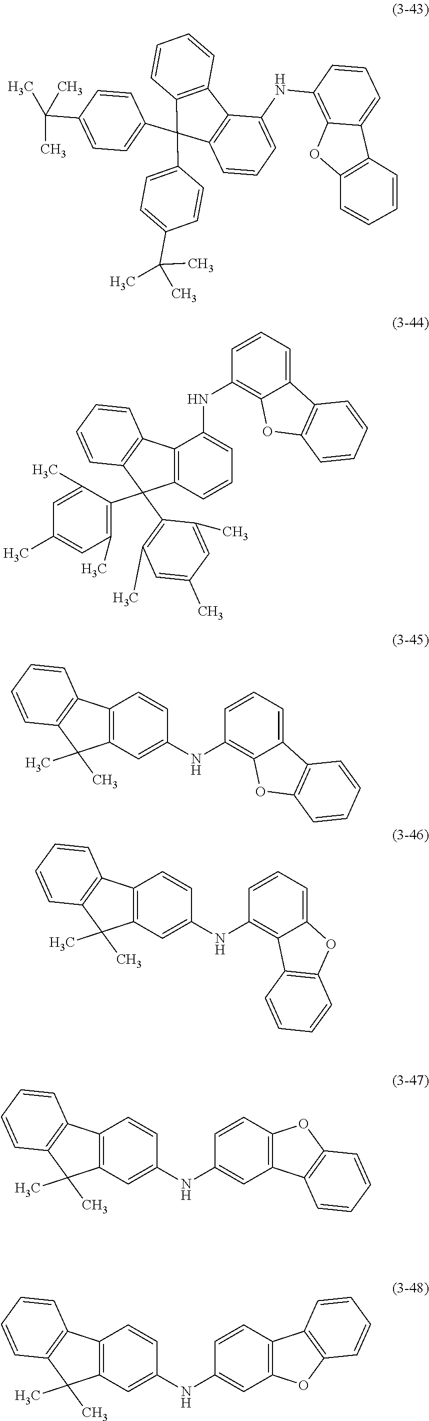 [SMILES: CC(C)(C)c1ccc(C2(c3ccc(C(C)(C)C)cc3)c3ccccc3-c3c(Nc4cccc5c4oc4ccccc45)cccc32)cc1 ; Cc1cc(C)c(C2(c3c(C)cc(C)cc3C)c3ccccc3-c3c(Nc4cccc5c4oc4ccccc45)cccc32)c(C)c1 ; CC1(C)c2ccccc2-c2ccc(Nc3cccc4c3oc3ccccc34)cc21 ; CC1(C)c2ccccc2-c2ccc(Nc3cccc4oc5ccccc5c34)cc21 ; CC1(C)c2ccccc2-c2ccc(Nc3ccc4oc5ccccc5c4c3)cc21 ; CC1(C)c2ccccc2-c2ccc(Nc3ccc4c(c3)oc3ccccc34)cc21]

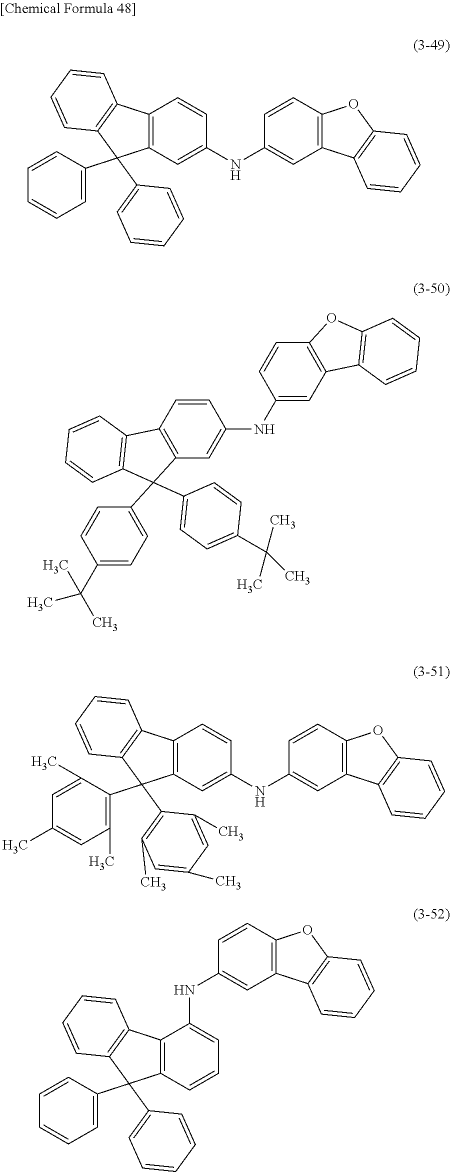 [SMILES: c1ccc(C2(c3ccccc3)c3ccccc3-c3ccc(Nc4ccc5oc6ccccc6c5c4)cc32)cc1 ; CC(C)(C)c1ccc(C2(c3ccc(C(C)(C)C)cc3)c3ccccc3-c3ccc(Nc4ccc5oc6ccccc6c5c4)cc32)cc1 ; Cc1cc(C)c(C2(c3c(C)cc(C)cc3C)c3ccccc3-c3ccc(Nc4ccc5oc6ccccc6c5c4)cc32)c(C)c1 ; c1ccc(C2(c3ccccc3)c3ccccc3-c3c(Nc4ccc5oc6ccccc6c5c4)cccc32)cc1]

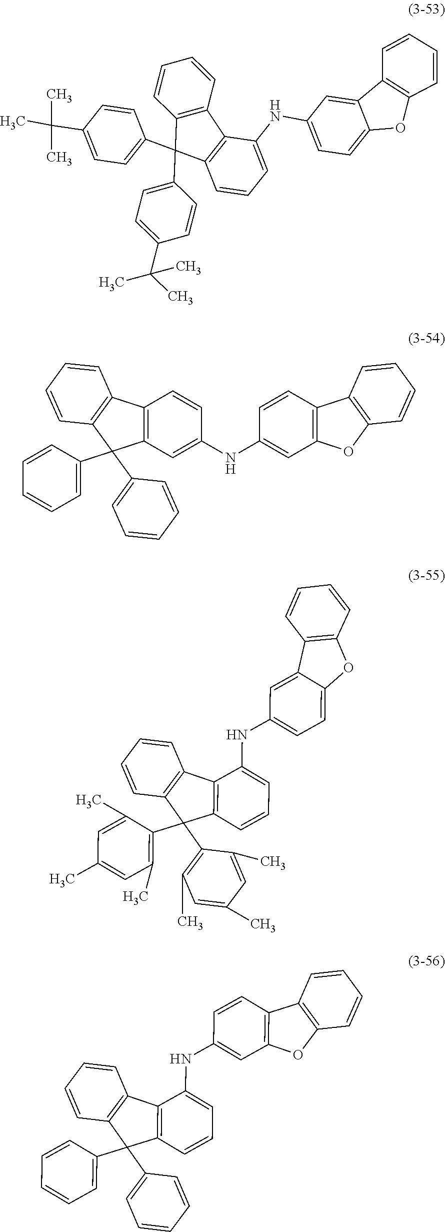 [SMILES: CC(C)(C)c1ccc(C2(c3ccc(C(C)(C)C)cc3)c3ccccc3-c3c(Nc4ccc5oc6ccccc6c5c4)cccc32)cc1 ; c1ccc(C2(c3ccccc3)c3ccccc3-c3ccc(Nc4ccc5c(c4)oc4ccccc45)cc32)cc1 ; Cc1cc(C)c(C2(c3c(C)cc(C)cc3C)c3ccccc3-c3c(Nc4ccc5oc6ccccc6c5c4)cccc32)c(C)c1 ; c1ccc(C2(c3ccccc3)c3ccccc3-c3c(Nc4ccc5c(c4)oc4ccccc45)cccc32)cc1]

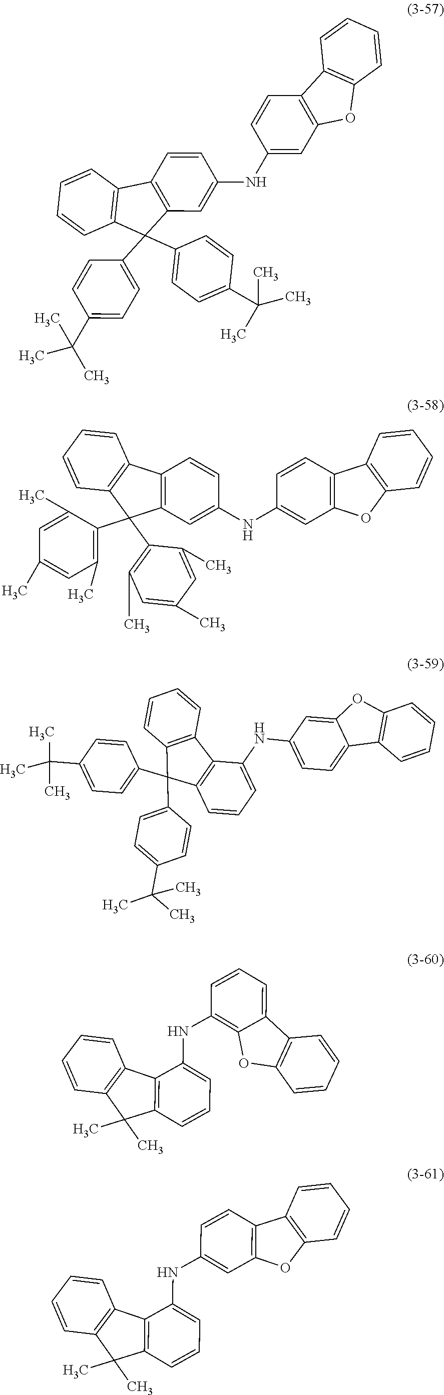 [SMILES: CC(C)(C)c1ccc(C2(c3ccc(C(C)(C)C)cc3)c3ccccc3-c3ccc(Nc4ccc5c(c4)oc4ccccc45)cc32)cc1 ; Cc1cc(C)c(C2(c3c(C)cc(C)cc3C)c3ccccc3-c3ccc(Nc4ccc5c(c4)oc4ccccc45)cc32)c(C)c1 ; CC(C)(C)c1ccc(C2(c3ccc(C(C)(C)C)cc3)c3ccccc3-c3c(Nc4ccc5c(c4)oc4ccccc45)cccc32)cc1 ; CC1(C)c2ccccc2-c2c(Nc3cccc4c3oc3ccccc34)cccc21 ; CC1(C)c2ccccc2-c2c(Nc3ccc4c(c3)oc3ccccc34)cccc21]

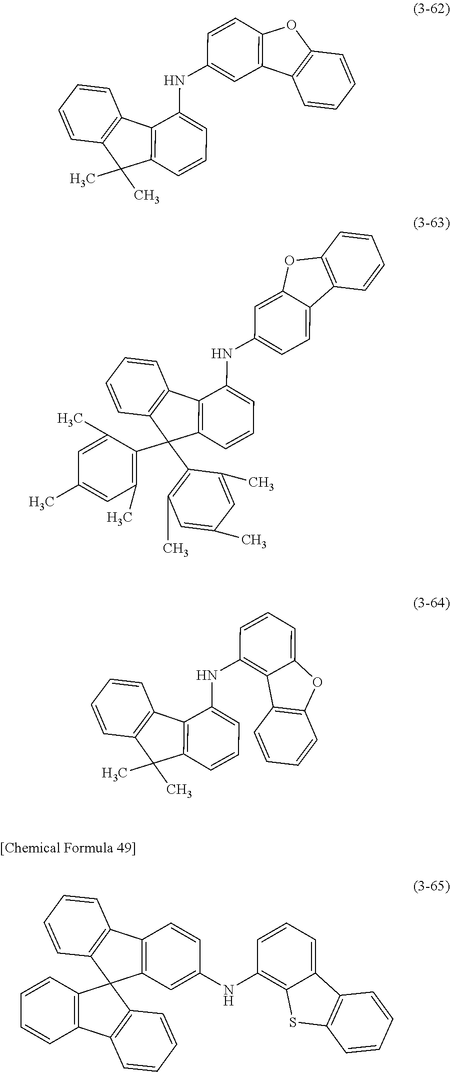 [SMILES: CC1(C)c2ccccc2-c2c(Nc3ccc4oc5ccccc5c4c3)cccc21 ; Cc1cc(C)c(C2(c3c(C)cc(C)cc3C)c3ccccc3-c3c(Nc4ccc5c(c4)oc4ccccc45)cccc32)c(C)c1 ; CC1(C)c2ccccc2-c2c(Nc3cccc4oc5ccccc5c34)cccc21 ; c1ccc2c(c1)-c1ccccc1C21c2ccccc2-c2ccc(Nc3cccc4c3sc3ccccc34)cc21]

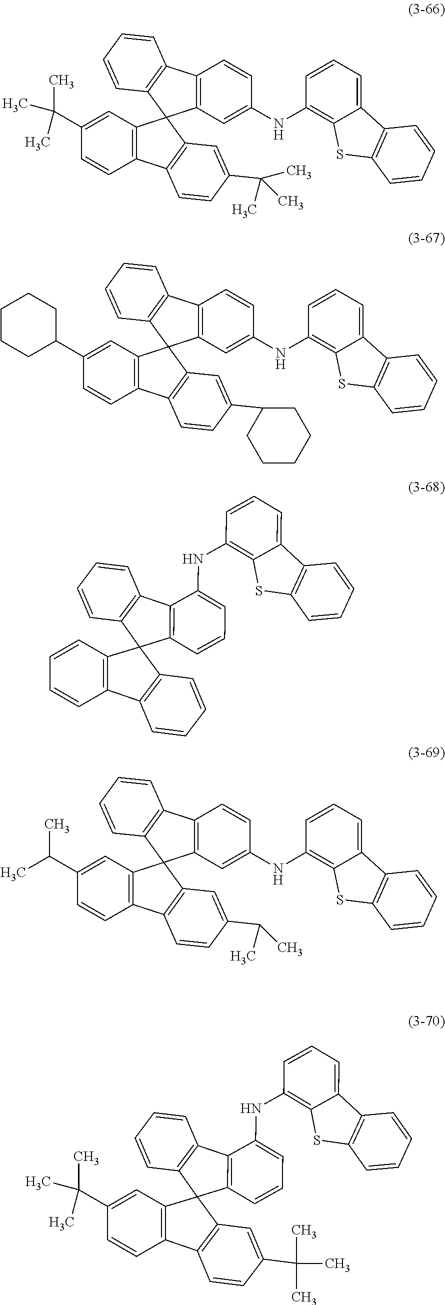

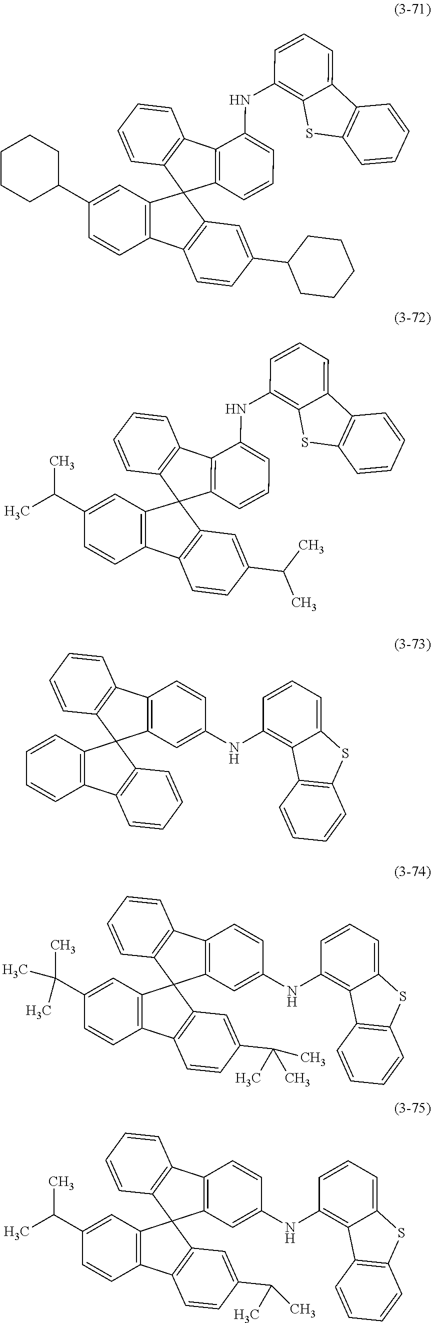 [SMILES: c1ccc2c(c1)-c1c(Nc3cccc4c3sc3ccccc34)cccc1C21c2cc(C3CCCCC3)ccc2-c2ccc(C3CCCCC3)cc21 ; CC(C)c1ccc2c(c1)C1(c3cc(C(C)C)ccc3-2)c2ccccc2-c2c(Nc3cccc4c3sc3ccccc34)cccc21 ; c1ccc2c(c1)-c1ccccc1C21c2ccccc2-c2ccc(Nc3cccc4sc5ccccc5c34)cc21 ; CC(C)(C)c1ccc2c(c1)C1(c3ccccc3-c3ccc(Nc4cccc5sc6ccccc6c45)cc31)c1cc(C(C)(C)C)ccc1-2 ; CC(C)c1ccc2c(c1)C1(c3ccccc3-c3ccc(Nc4cccc5sc6ccccc6c45)cc31)c1cc(C(C)C)ccc1-2]

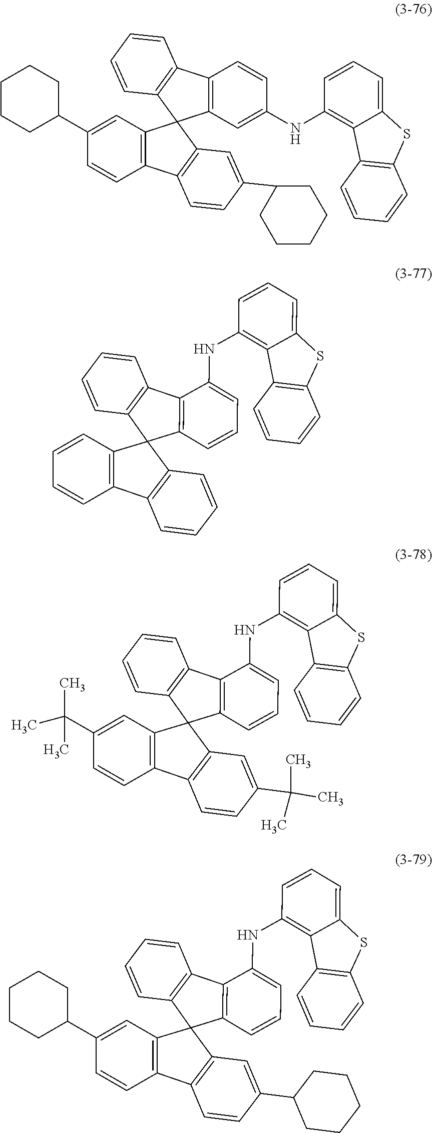

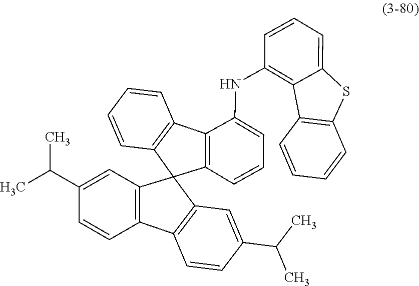 [SMILES: CC(C)c1ccc2c(c1)C1(c3cc(C(C)C)ccc3-2)c2ccccc2-c2c(Nc3cccc4sc5ccccc5c34)cccc21]

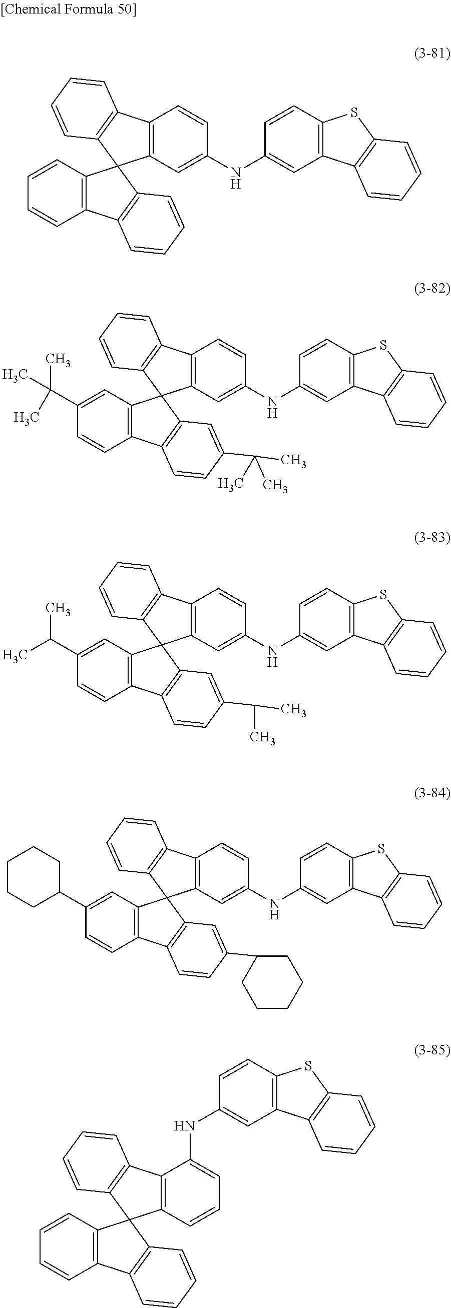 [SMILES: c1ccc2c(c1)-c1ccccc1C21c2ccccc2-c2ccc(Nc3ccc4sc5ccccc5c4c3)cc21 ; CC(C)(C)c1ccc2c(c1)C1(c3ccccc3-c3ccc(Nc4ccc5sc6ccccc6c5c4)cc31)c1cc(C(C)(C)C)ccc1-2 ; CC(C)c1ccc2c(c1)C1(c3ccccc3-c3ccc(Nc4ccc5sc6ccccc6c5c4)cc31)c1cc(C(C)C)ccc1-2 ; c1ccc2c(c1)-c1ccc(Nc3ccc4sc5ccccc5c4c3)cc1C21c2cc(C3CCCCC3)ccc2-c2ccc(C3CCCCC3)cc21 ; c1ccc2c(c1)-c1ccccc1C21c2ccccc2-c2c(Nc3ccc4sc5ccccc5c4c3)cccc21]

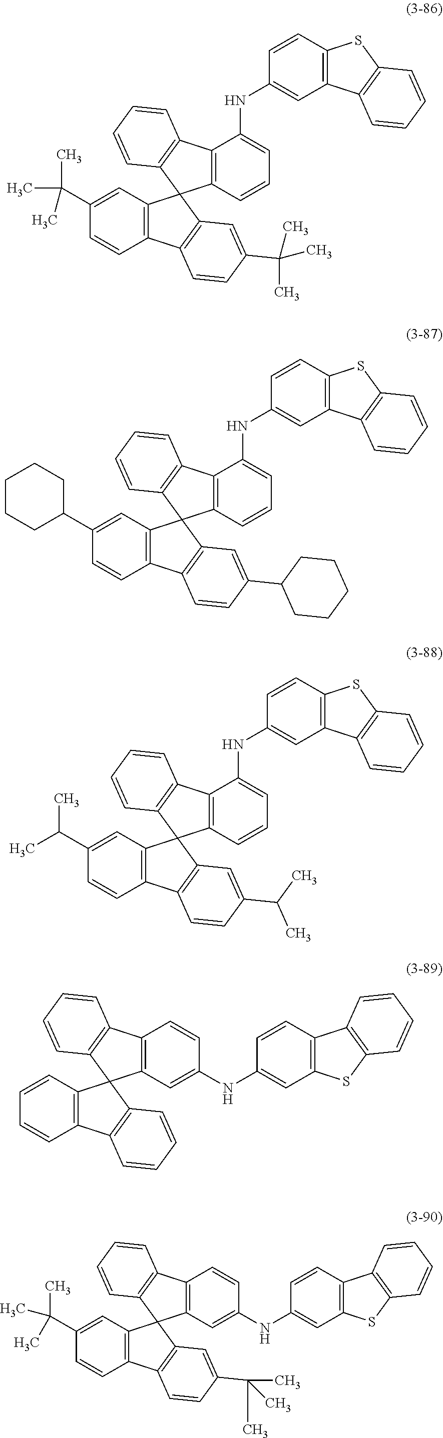 [SMILES: CC(C)(C)c1ccc2c(c1)C1(c3cc(C(C)(C)C)ccc3-2)c2ccccc2-c2c(Nc3ccc4sc5ccccc5c4c3)cccc21 ; c1ccc2c(c1)-c1c(Nc3ccc4sc5ccccc5c4c3)cccc1C21c2cc(C3CCCCC3)ccc2-c2ccc(C3CCCCC3)cc21 ; CC(C)c1ccc2c(c1)C1(c3cc(C(C)C)ccc3-2)c2ccccc2-c2c(Nc3ccc4sc5ccccc5c4c3)cccc21 ; c1ccc2c(c1)-c1ccccc1C21c2ccccc2-c2ccc(Nc3ccc4c(c3)sc3ccccc34)cc21 ; CC(C)(C)c1ccc2c(c1)C1(c3ccccc3-c3ccc(Nc4ccc5c(c4)sc4ccccc45)cc31)c1cc(C(C)(C)C)ccc1-2]

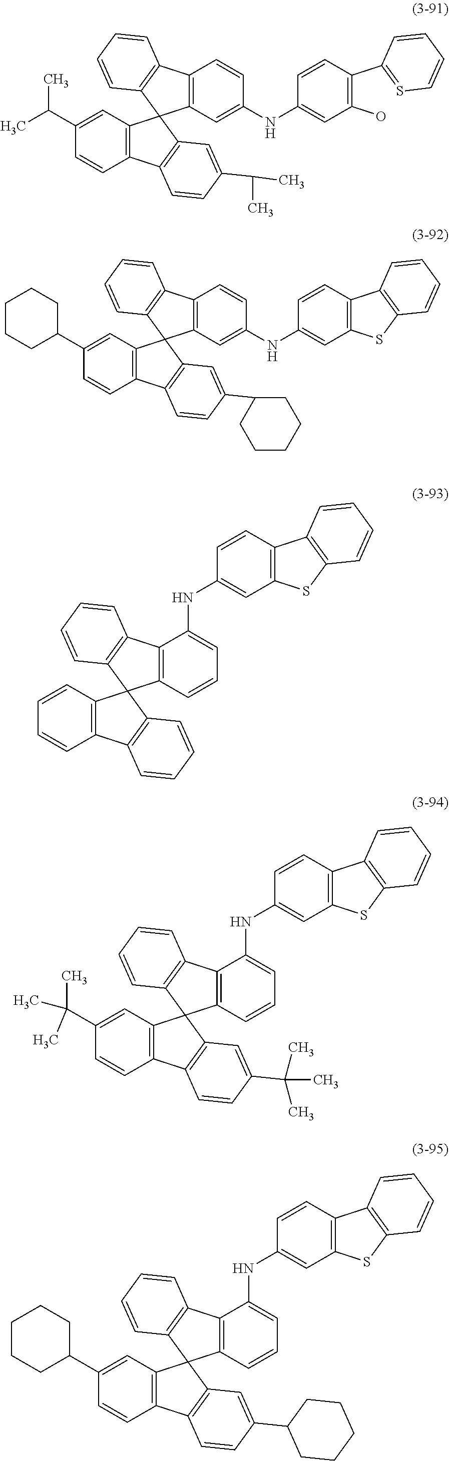

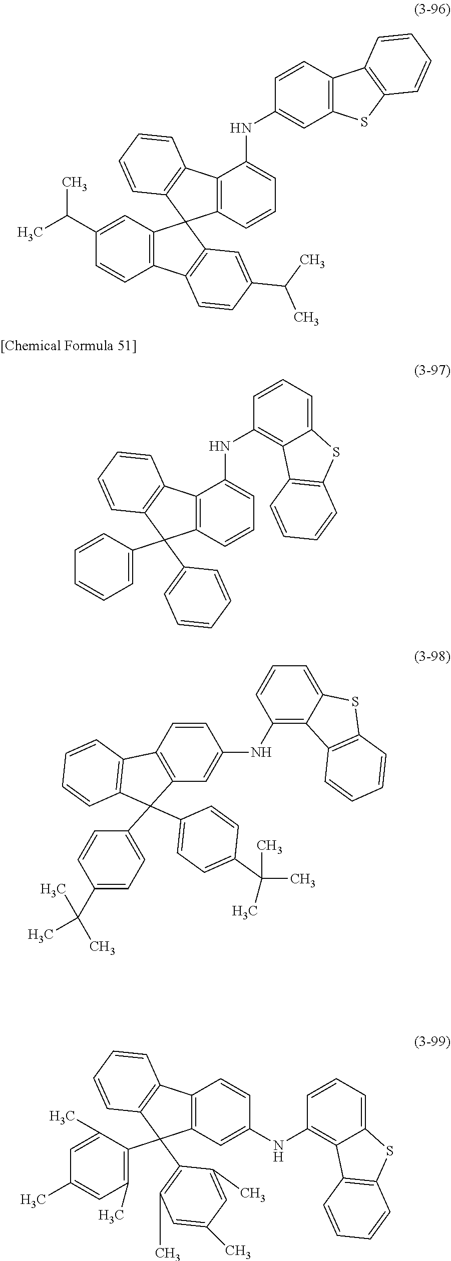 [SMILES: CC(C)c1ccc2c(c1)C1(c3cc(C(C)C)ccc3-2)c2ccccc2-c2c(Nc3ccc4c(c3)sc3ccccc34)cccc21 ; c1ccc(C2(c3ccccc3)c3ccccc3-c3c(Nc4cccc5sc6ccccc6c45)cccc32)cc1 ; CC(C)(C)c1ccc(C2(c3ccc(C(C)(C)C)cc3)c3ccccc3-c3ccc(Nc4cccc5sc6ccccc6c45)cc32)cc1 ; Cc1cc(C)c(C2(c3c(C)cc(C)cc3C)c3ccccc3-c3ccc(Nc4cccc5sc6ccccc6c45)cc32)c(C)c1]

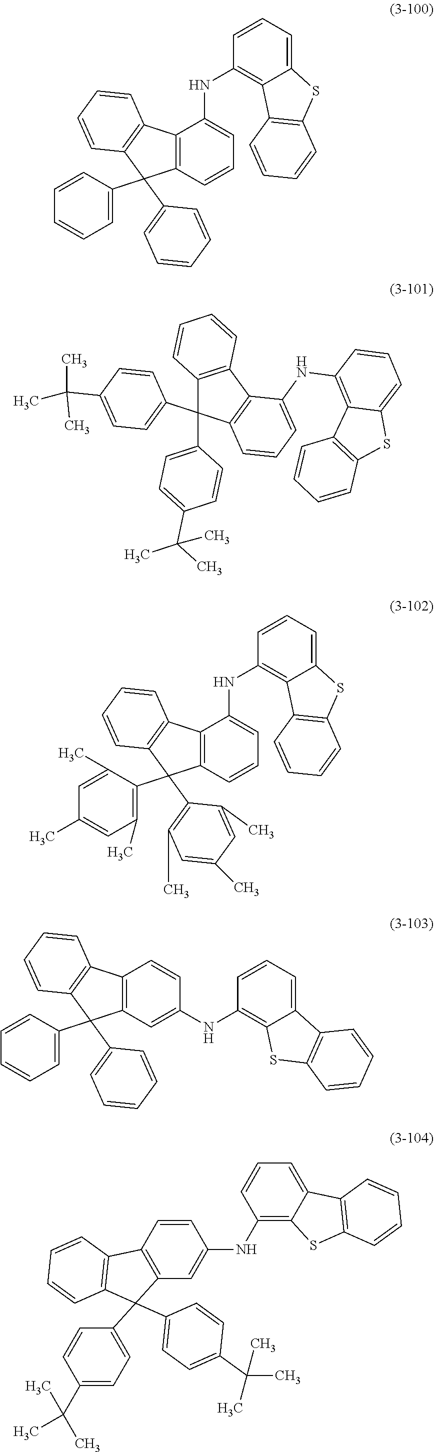 [SMILES: c1ccc(C2(c3ccccc3)c3ccccc3-c3c(Nc4cccc5sc6ccccc6c45)cccc32)cc1 ; CC(C)(C)c1ccc(C2(c3ccc(C(C)(C)C)cc3)c3ccccc3-c3c(Nc4cccc5sc6ccccc6c45)cccc32)cc1 ; Cc1cc(C)c(C2(c3c(C)cc(C)cc3C)c3ccccc3-c3c(Nc4cccc5sc6ccccc6c45)cccc32)c(C)c1 ; c1ccc(C2(c3ccccc3)c3ccccc3-c3ccc(Nc4cccc5c4sc4ccccc45)cc32)cc1 ; CC(C)(C)c1ccc(C2(c3ccc(C(C)(C)C)cc3)c3ccccc3-c3ccc(Nc4cccc5c4sc4ccccc45)cc32)cc1]

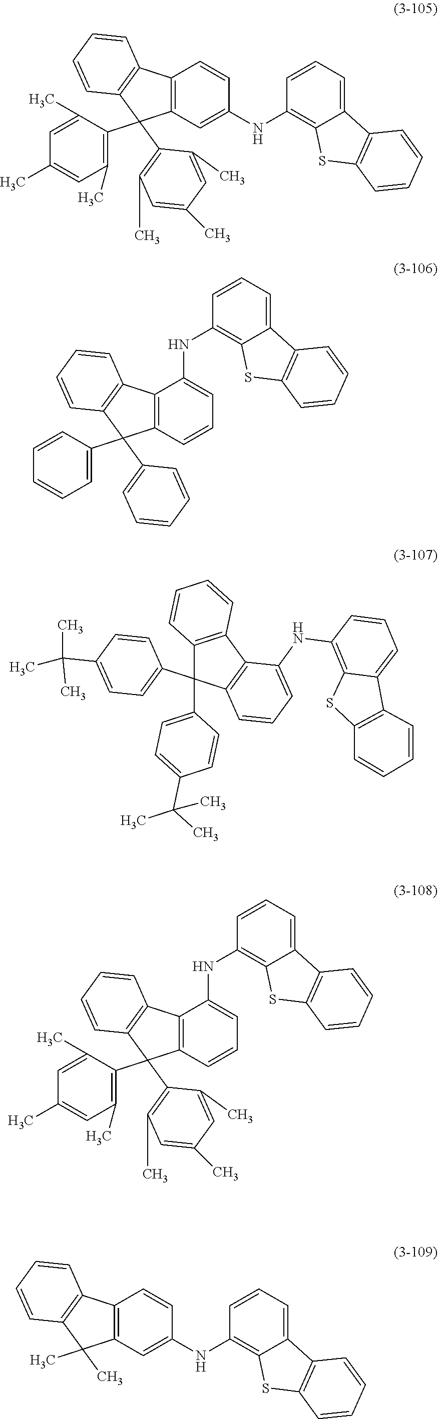 [SMILES: Cc1cc(C)c(C2(c3c(C)cc(C)cc3C)c3ccccc3-c3ccc(Nc4cccc5c4sc4ccccc45)cc32)c(C)c1 ; c1ccc(C2(c3ccccc3)c3ccccc3-c3c(Nc4cccc5c4sc4ccccc45)cccc32)cc1 ; CC(C)(C)c1ccc(C2(c3ccc(C(C)(C)C)cc3)c3ccccc3-c3c(Nc4cccc5c4sc4ccccc45)cccc32)cc1 ; Cc1cc(C)c(C2(c3c(C)cc(C)cc3C)c3ccccc3-c3c(Nc4cccc5c4sc4ccccc45)cccc32)c(C)c1 ; CC1(C)c2ccccc2-c2ccc(Nc3cccc4c3sc3ccccc34)cc21]

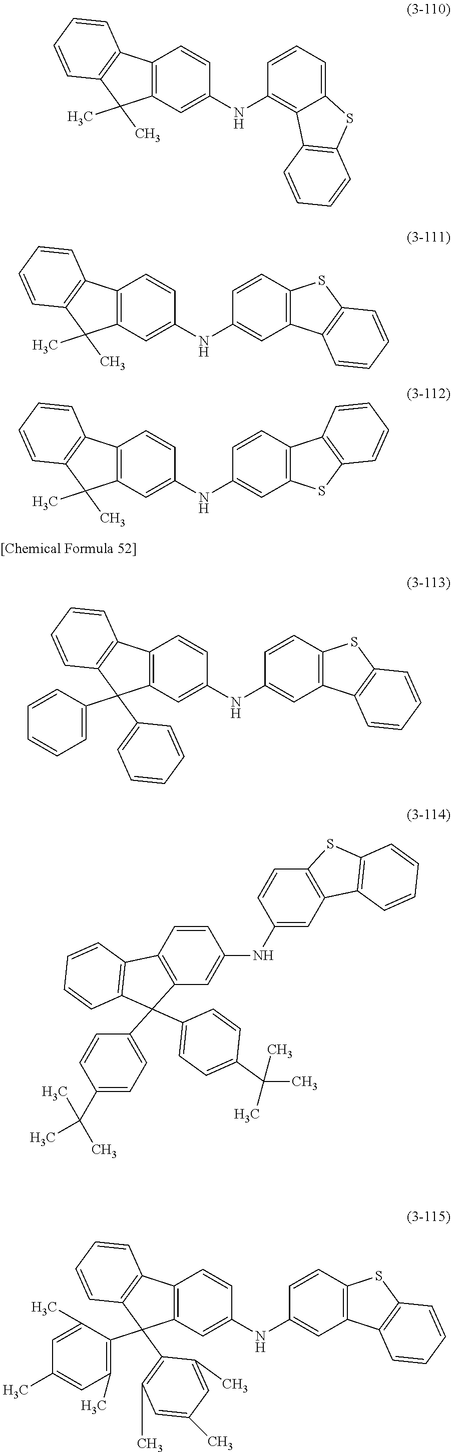

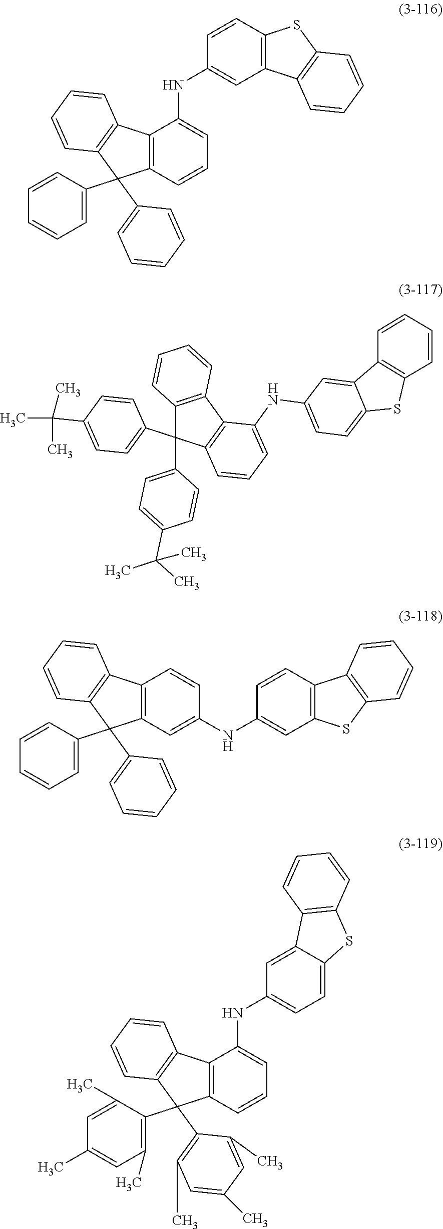 [SMILES: c1ccc(C2(c3ccccc3)c3ccccc3-c3c(Nc4ccc5sc6ccccc6c5c4)cccc32)cc1 ; CC(C)(C)c1ccc(C2(c3ccc(C(C)(C)C)cc3)c3ccccc3-c3c(Nc4ccc5sc6ccccc6c5c4)cccc32)cc1 ; c1ccc(C2(c3ccccc3)c3ccccc3-c3ccc(Nc4ccc5c(c4)sc4ccccc45)cc32)cc1 ; Cc1cc(C)c(C2(c3c(C)cc(C)cc3C)c3ccccc3-c3c(Nc4ccc5sc6ccccc6c5c4)cccc32)c(C)c1]

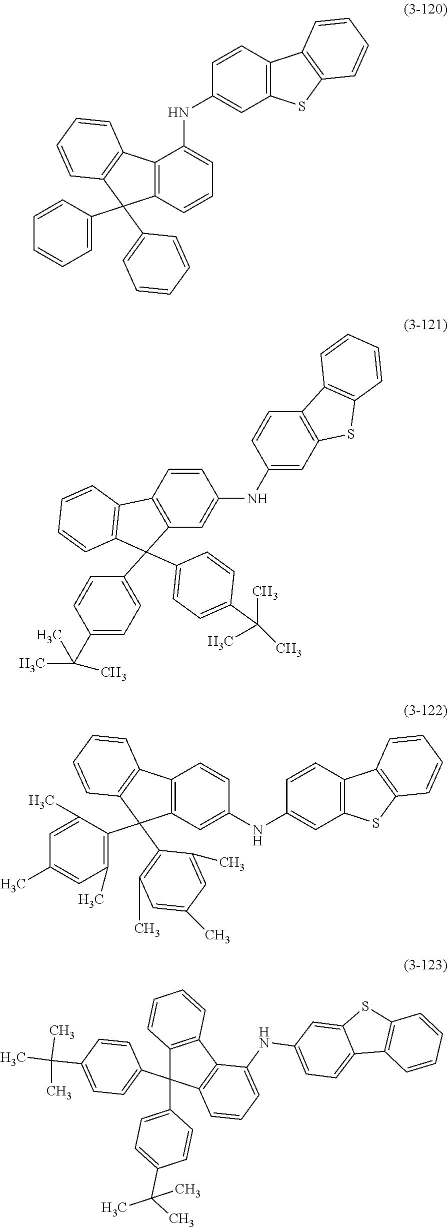 [SMILES: c1ccc(C2(c3ccccc3)c3ccccc3-c3c(Nc4ccc5c(c4)sc4ccccc45)cccc32)cc1 ; CC(C)(C)c1ccc(C2(c3ccc(C(C)(C)C)cc3)c3ccccc3-c3ccc(Nc4ccc5c(c4)sc4ccccc45)cc32)cc1 ; Cc1cc(C)c(C2(c3c(C)cc(C)cc3C)c3ccccc3-c3ccc(Nc4ccc5c(c4)sc4ccccc45)cc32)c(C)c1 ; CC(C)(C)c1ccc(C2(c3ccc(C(C)(C)C)cc3)c3ccccc3-c3c(Nc4ccc5c(c4)sc4ccccc45)cccc32)cc1]

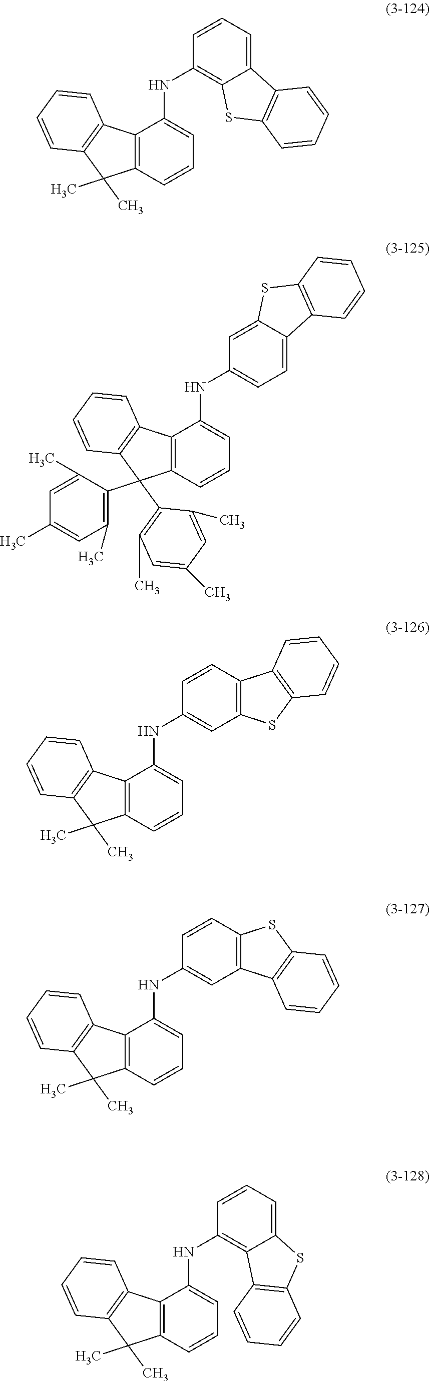 [SMILES: CC1(C)c2ccccc2-c2c(Nc3cccc4c3sc3ccccc34)cccc21 ; Cc1cc(C)c(C2(c3c(C)cc(C)cc3C)c3ccccc3-c3c(Nc4ccc5c(c4)sc4ccccc45)cccc32)c(C)c1 ; CC1(C)c2ccccc2-c2c(Nc3ccc4c(c3)sc3ccccc34)cccc21 ; CC1(C)c2ccccc2-c2c(Nc3ccc4sc5ccccc5c4c3)cccc21 ; CC1(C)c2ccccc2-c2c(Nc3cccc4sc5ccccc5c34)cccc21]

Note that specific examples of organic compounds that can be used as the secondary amine of Compound 7 in the above synthesis scheme are shown.

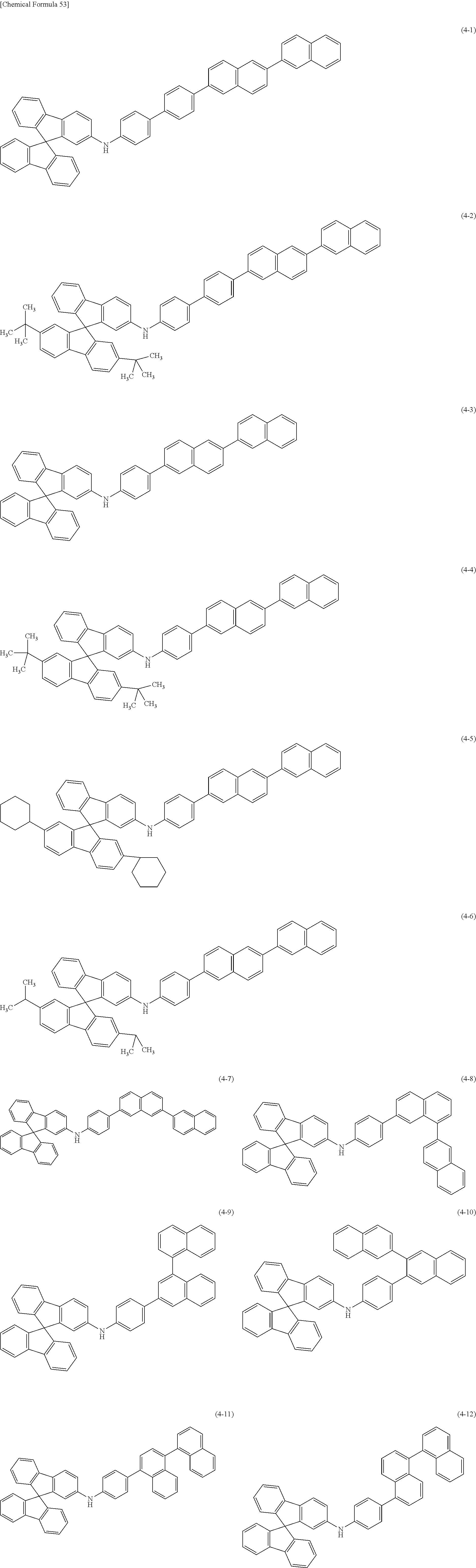

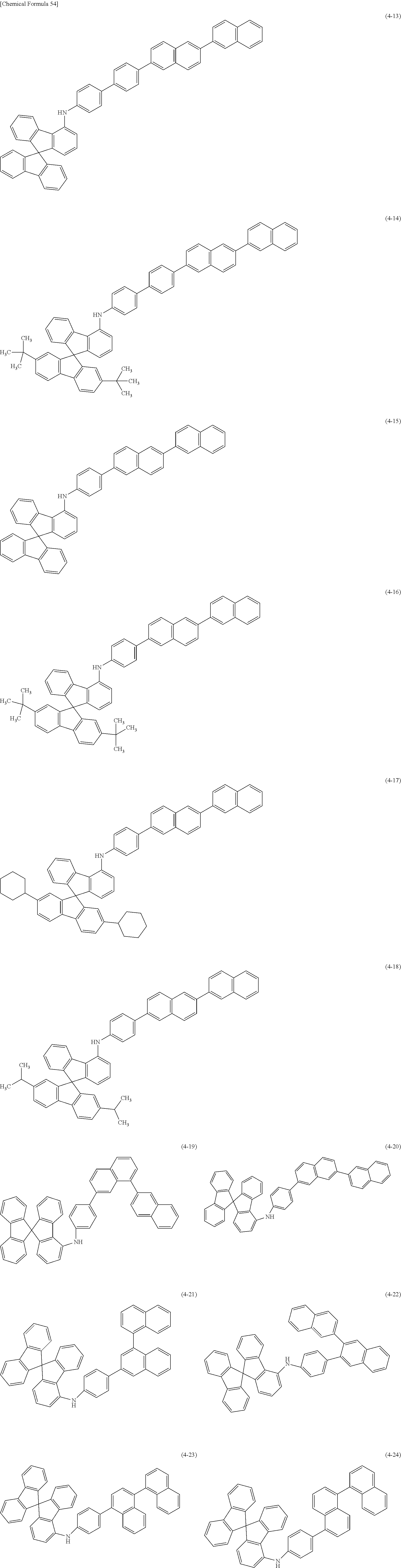

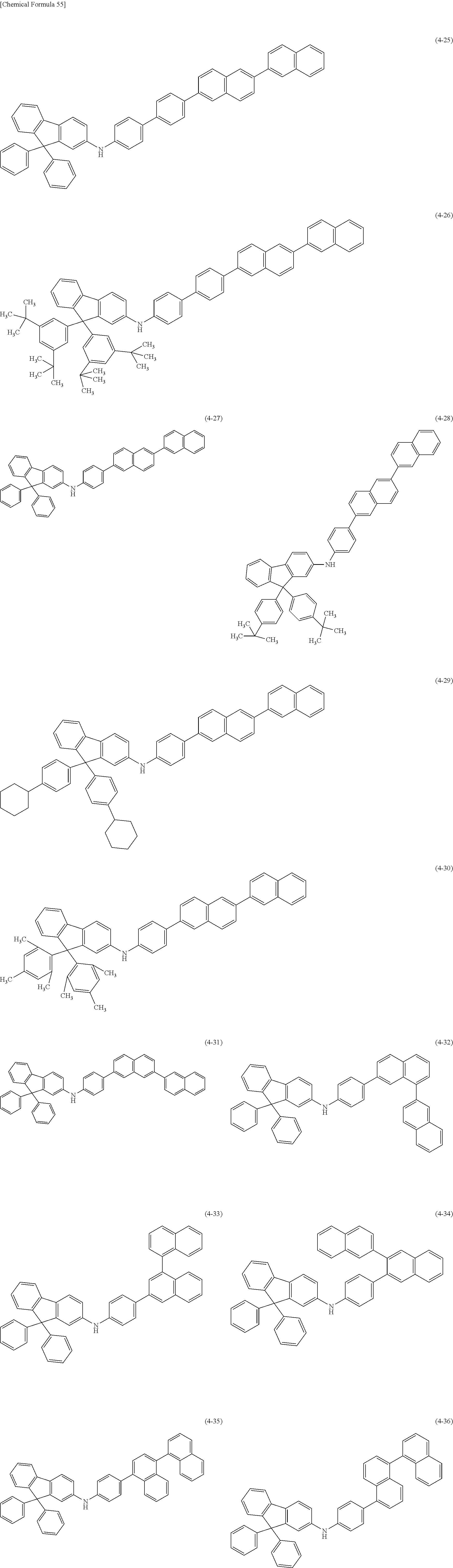

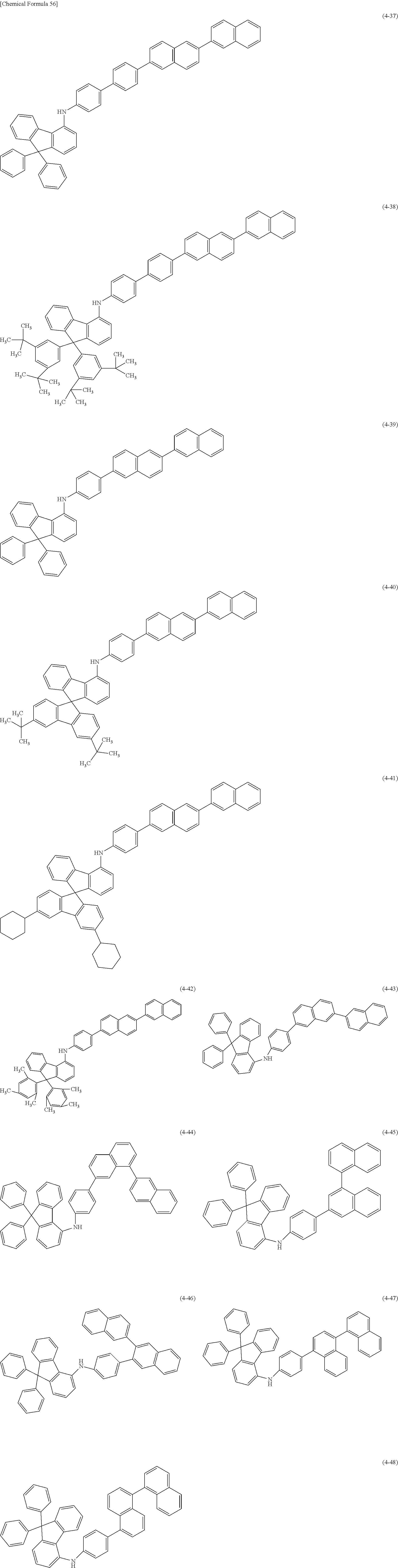

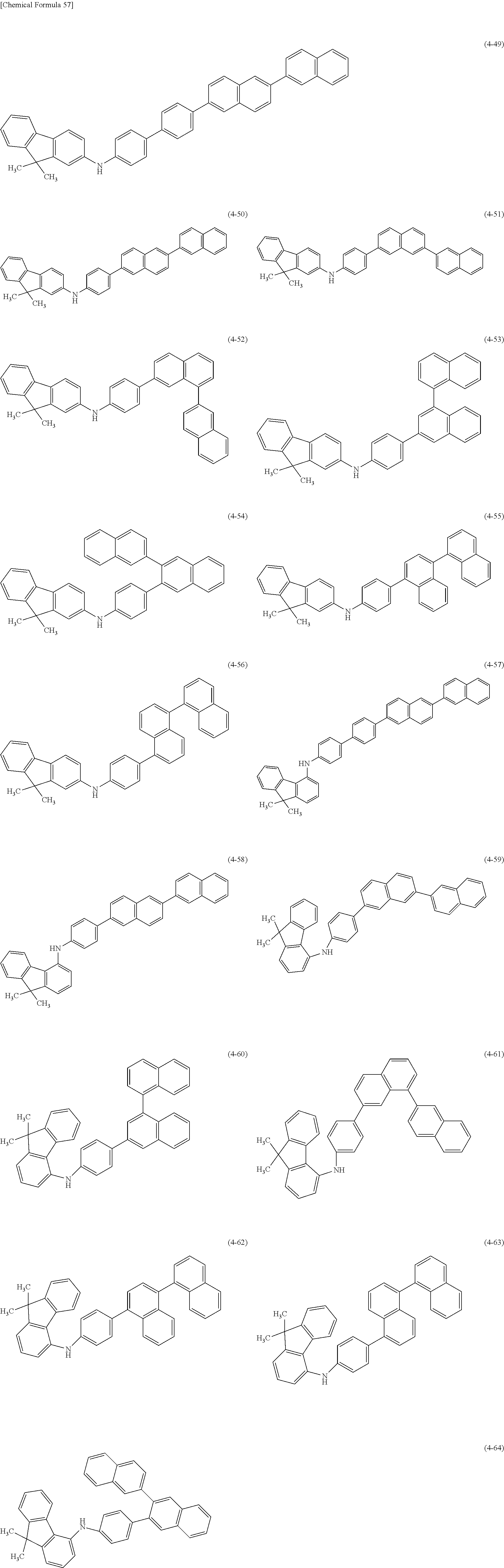

Note that specific examples of organic compounds that can be used as the secondary amine of Compound 16 in the above synthesis scheme are shown.

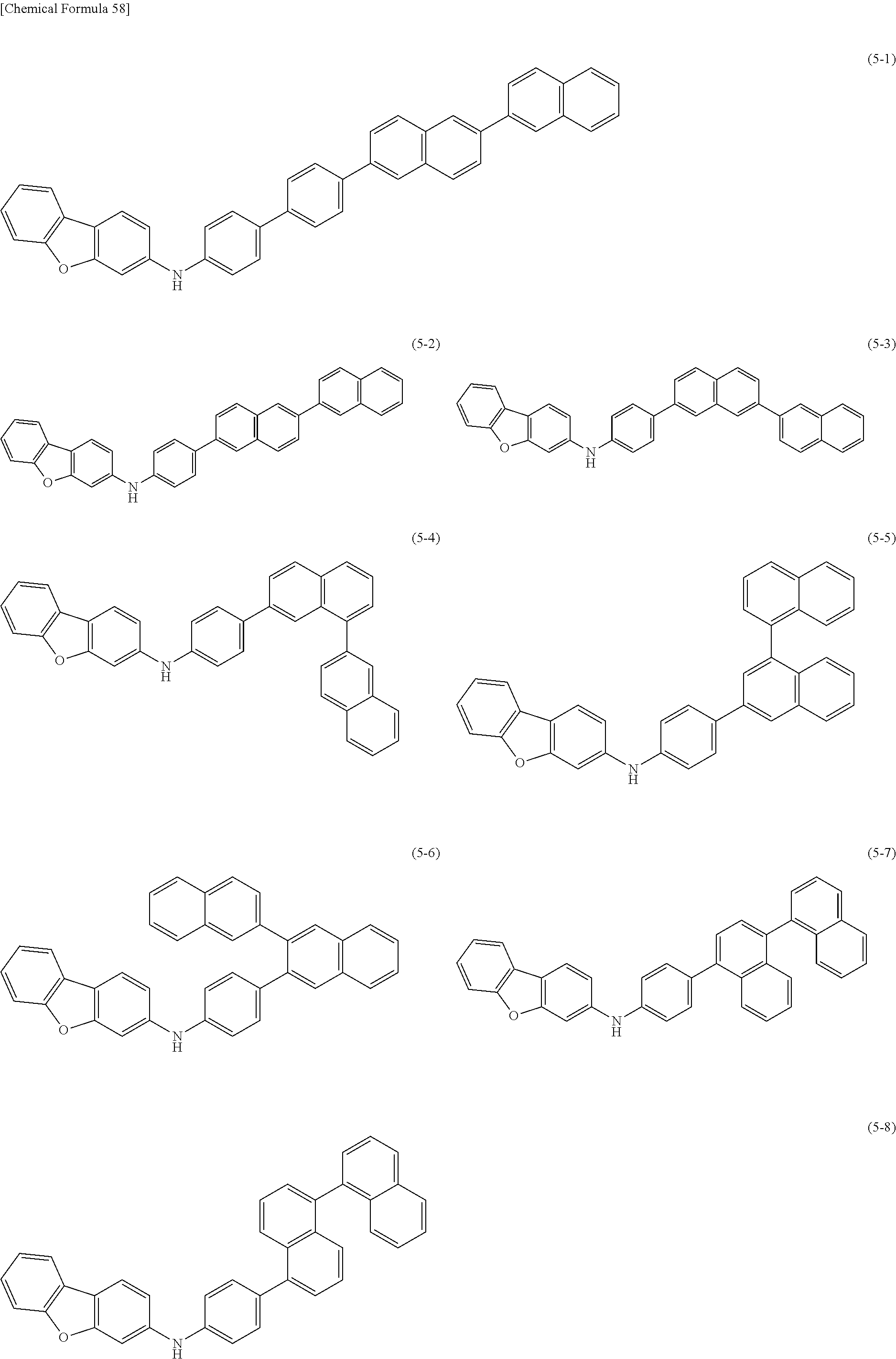

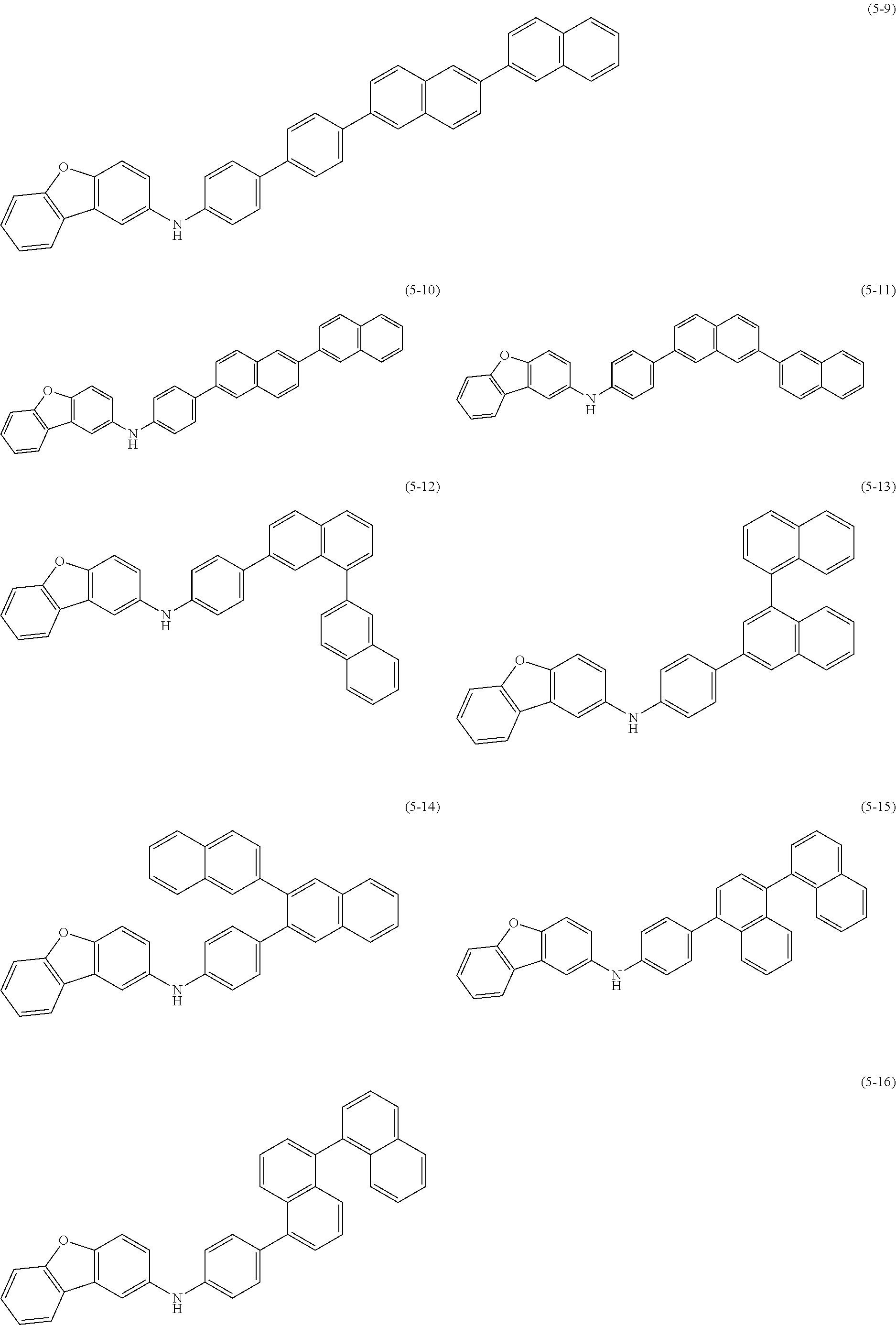

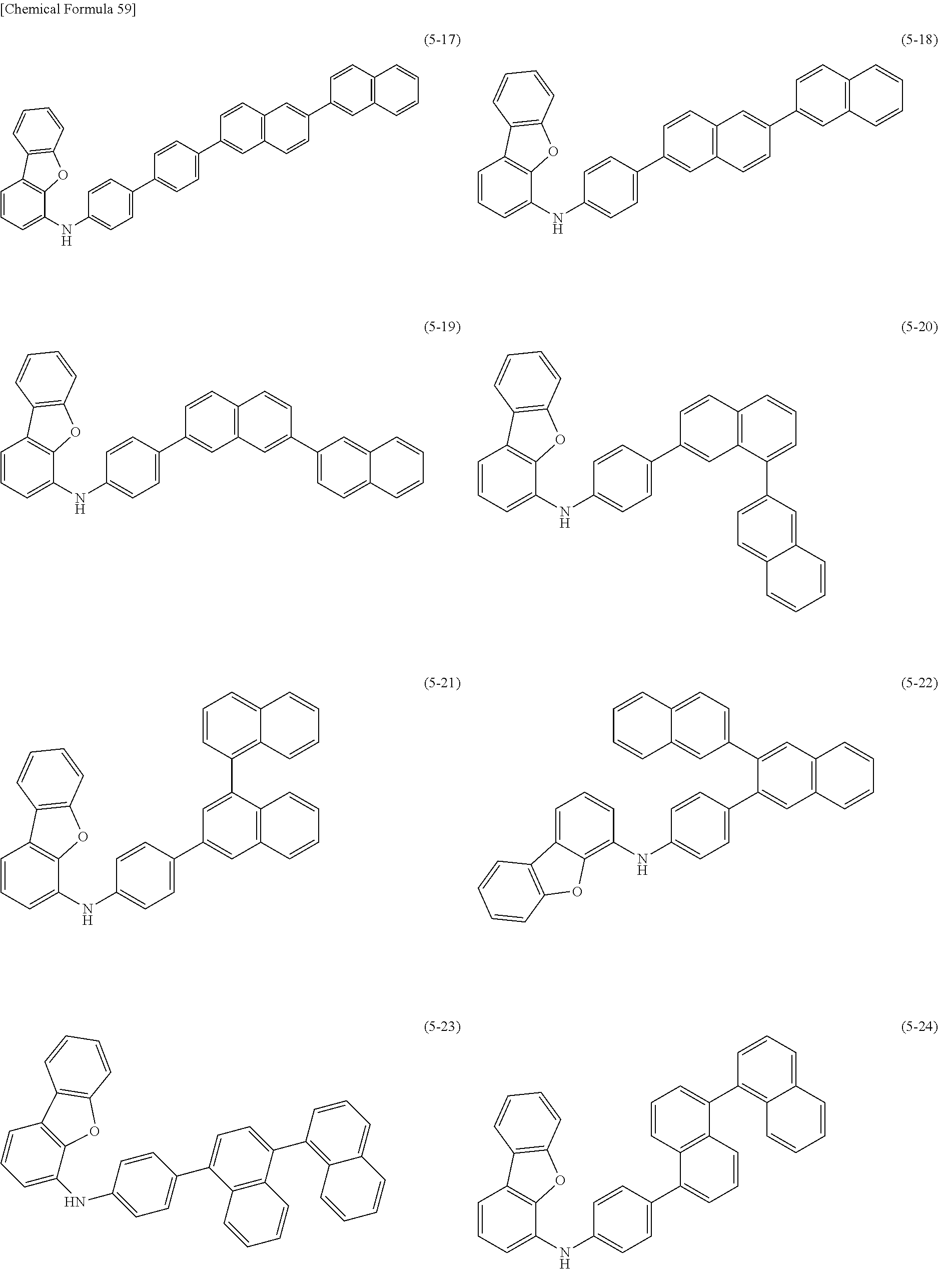

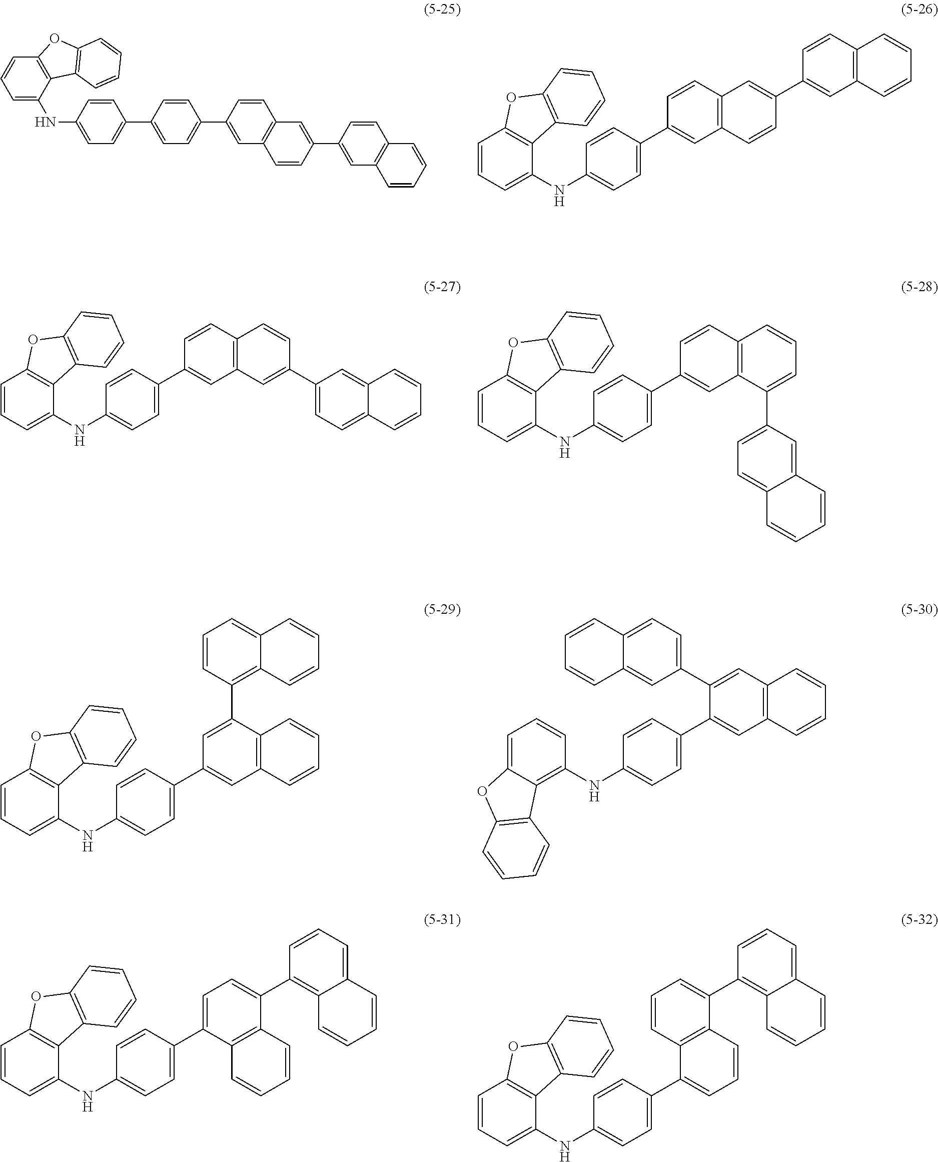

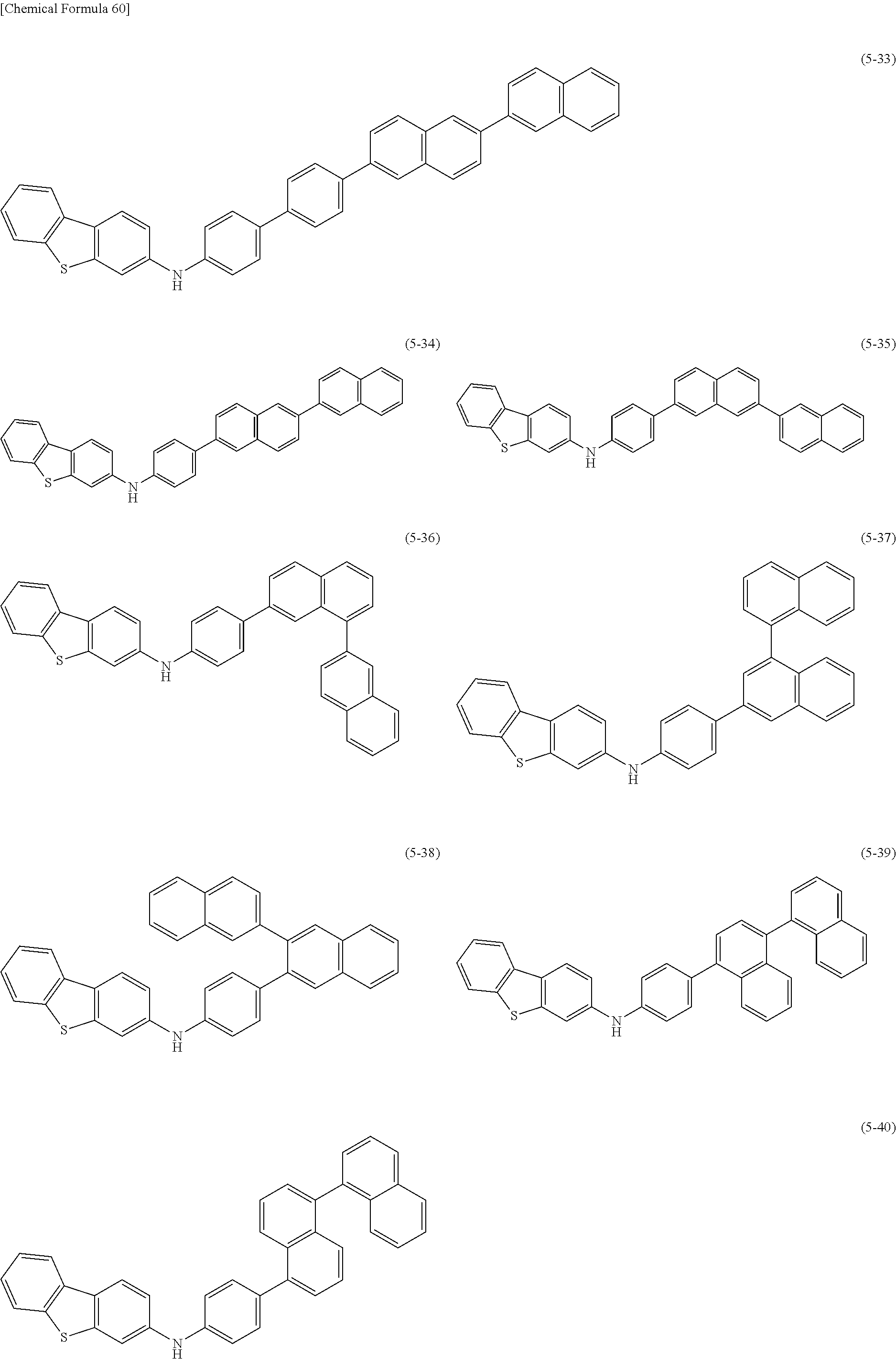

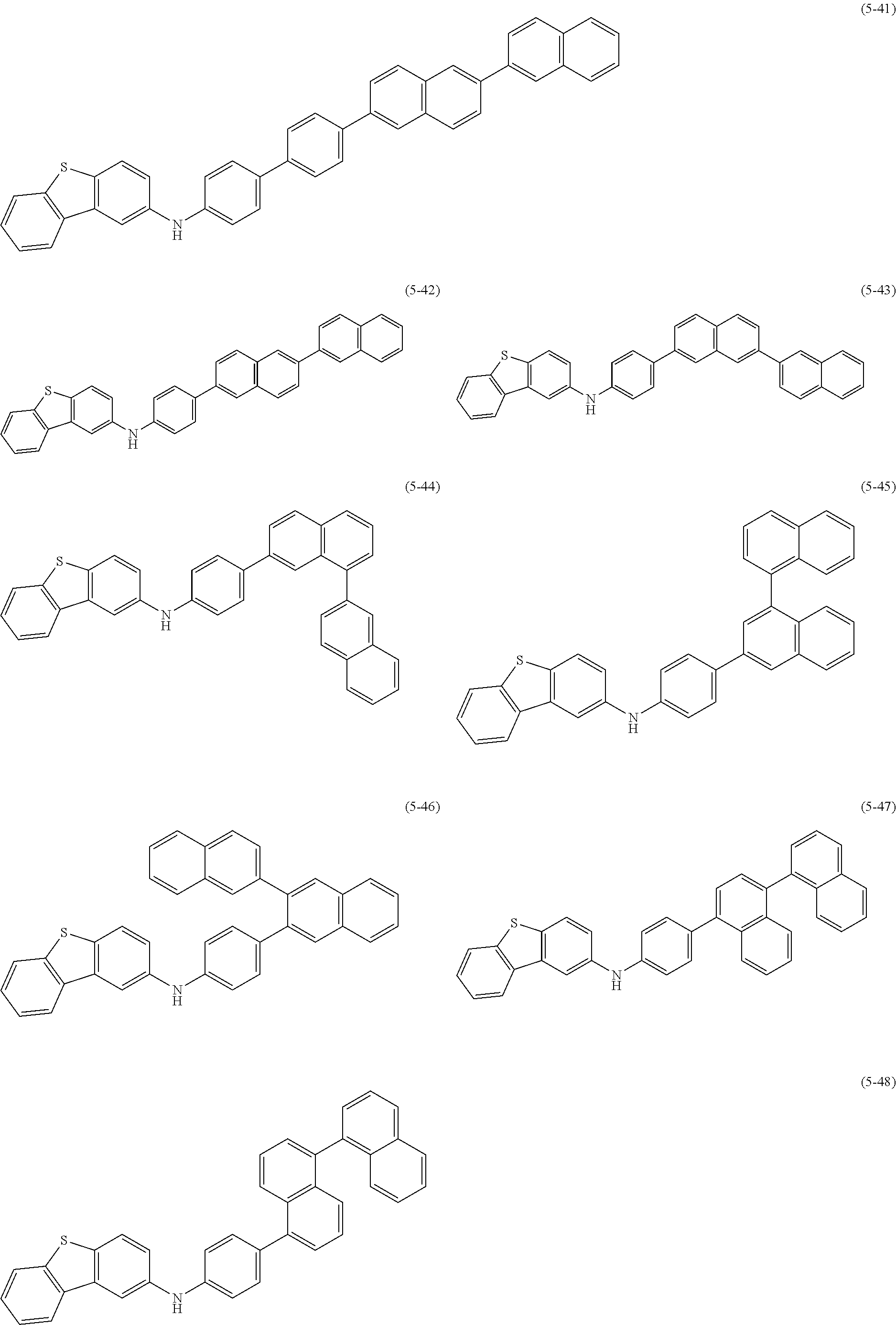

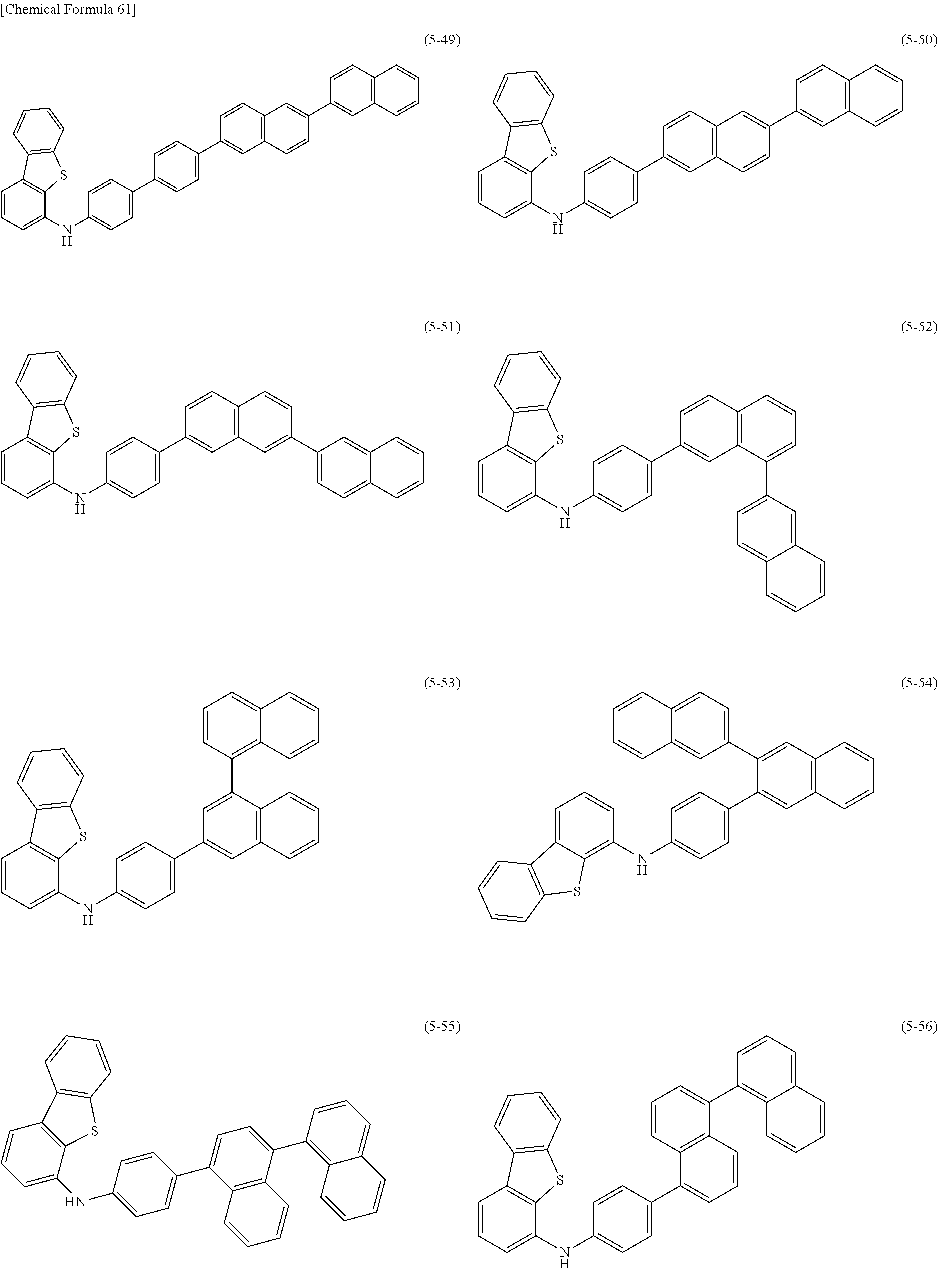

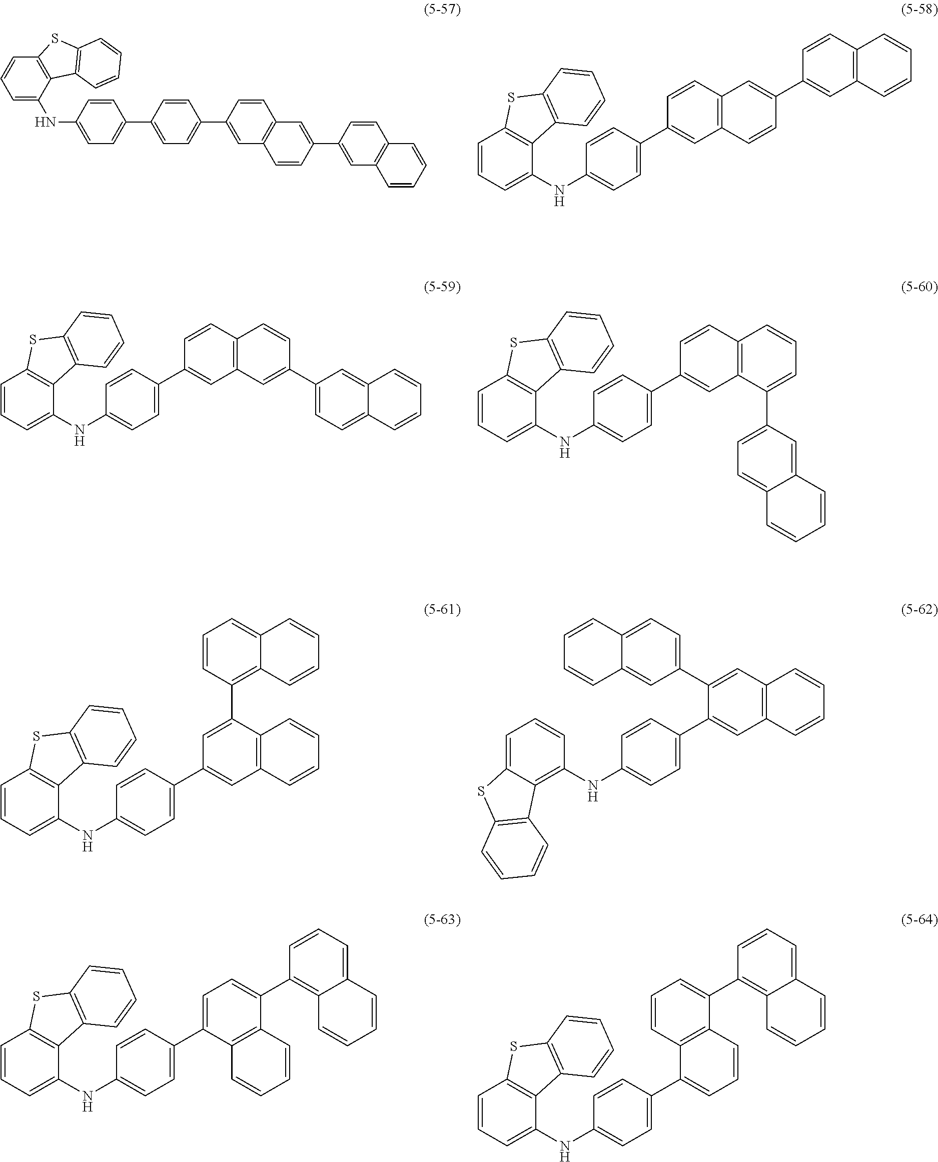

Specific examples of the organic compound of one embodiment of the present invention described in this embodiment include organic compounds represented by Structural Formulae (100) to (247) and (300) to (447).

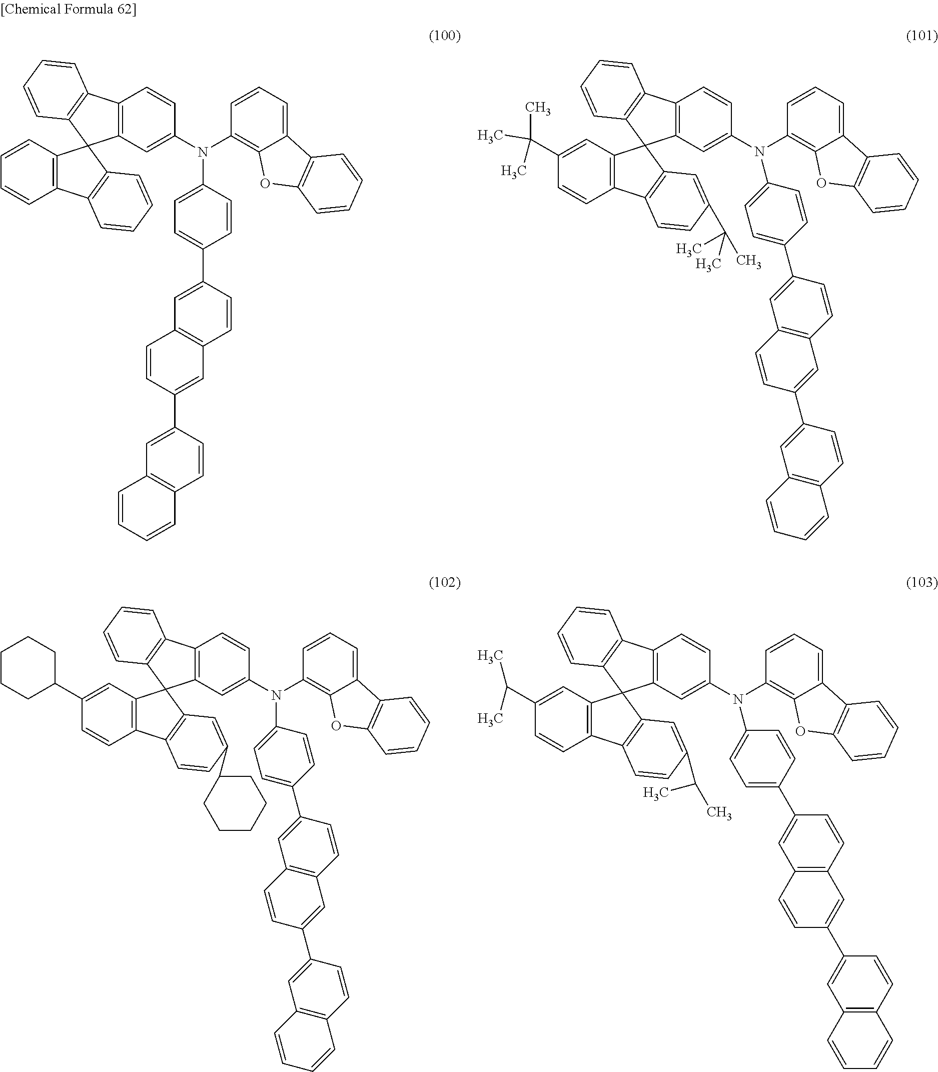 [SMILES: c1ccc2c(c1)-c1ccccc1C21c2ccccc2-c2ccc(N(c3ccc(-c4ccc5cc(-c6ccc7ccccc7c6)ccc5c4)cc3)c3cccc4c3oc3ccccc34)cc21 ; CC(C)(C)c1ccc2c(c1)C1(c3ccccc3-c3ccc(N(c4ccc(-c5ccc6cc(-c7ccc8ccccc8c7)ccc6c5)cc4)c4cccc5c4oc4ccccc45)cc31)c1cc(C(C)(C)C)ccc1-2 ; c1ccc2c(c1)-c1ccc(N(c3ccc(-c4ccc5cc(-c6ccc7ccccc7c6)ccc5c4)cc3)c3cccc4c3oc3ccccc34)cc1C21c2cc(C3CCCCC3)ccc2-c2ccc(C3CCCCC3)cc21 ; CC(C)c1ccc2c(c1)C1(c3ccccc3-c3ccc(N(c4ccc(-c5ccc6cc(-c7ccc8ccccc8c7)ccc6c5)cc4)c4cccc5c4oc4ccccc45)cc31)c1cc(C(C)C)ccc1-2]

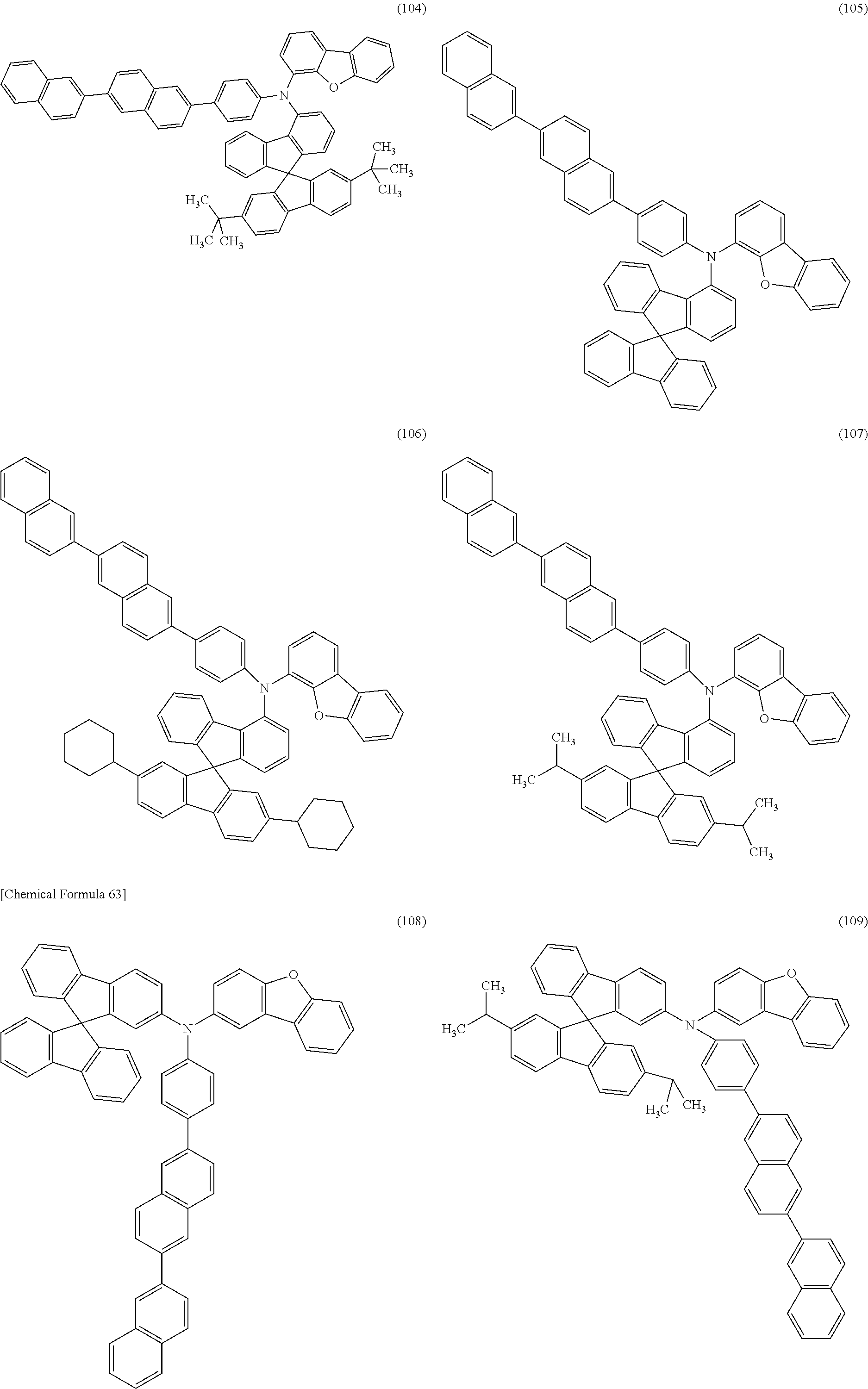

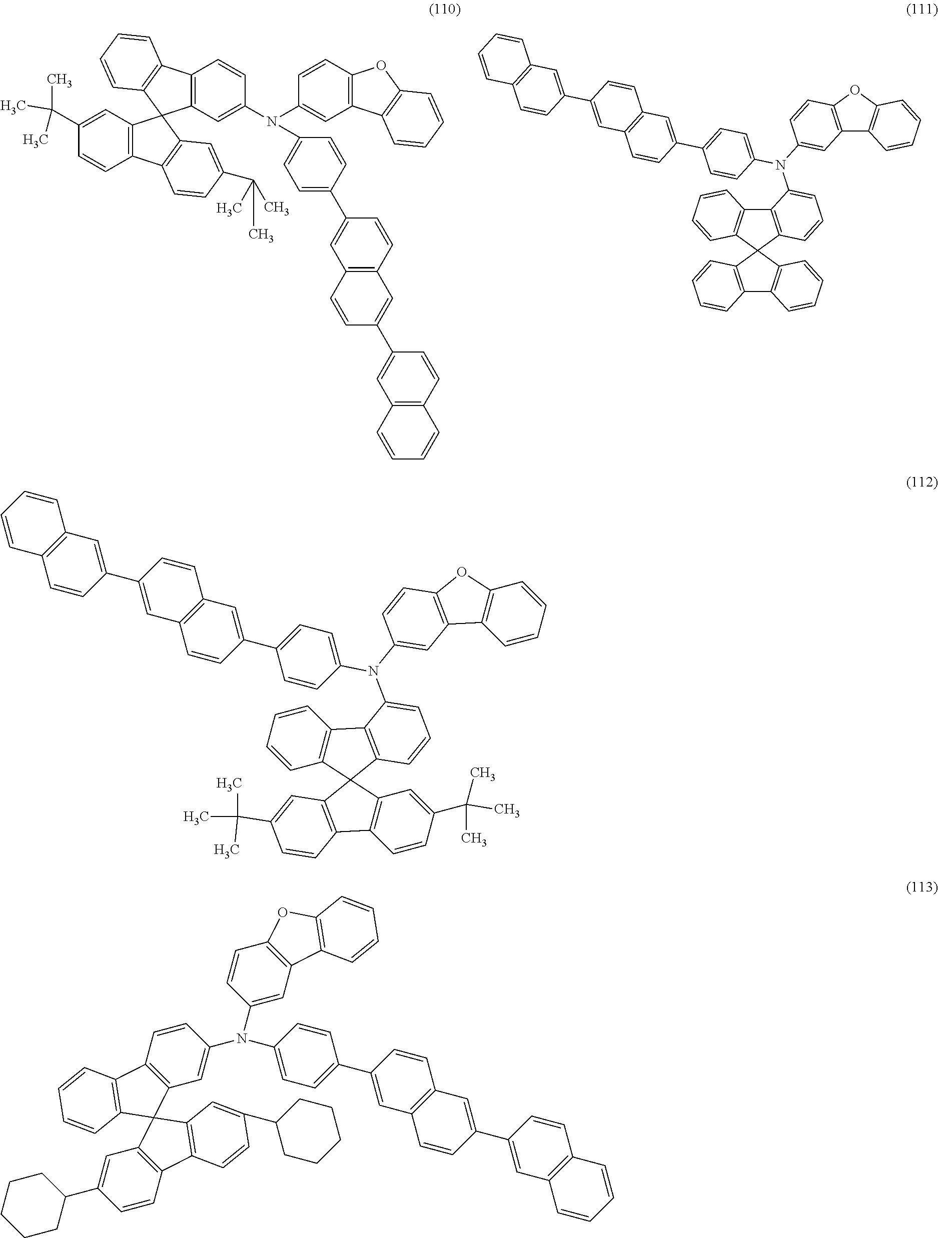 [SMILES: CC(C)(C)c1ccc2c(c1)C1(c3ccccc3-c3ccc(N(c4ccc(-c5ccc6cc(-c7ccc8ccccc8c7)ccc6c5)cc4)c4ccc5oc6ccccc6c5c4)cc31)c1cc(C(C)(C)C)ccc1-2 ; c1ccc2c(c1)-c1ccccc1C21c2ccccc2-c2c(N(c3ccc(-c4ccc5cc(-c6ccc7ccccc7c6)ccc5c4)cc3)c3ccc4oc5ccccc5c4c3)cccc21 ; CC(C)(C)c1ccc2c(c1)C1(c3cc(C(C)(C)C)ccc3-2)c2ccccc2-c2c(N(c3ccc(-c4ccc5cc(-c6ccc7ccccc7c6)ccc5c4)cc3)c3ccc4oc5ccccc5c4c3)cccc21 ; c1ccc2c(c1)-c1ccc(N(c3ccc(-c4ccc5cc(-c6ccc7ccccc7c6)ccc5c4)cc3)c3ccc4oc5ccccc5c4c3)cc1C21c2cc(C3CCCCC3)ccc2-c2ccc(C3CCCCC3)cc21]

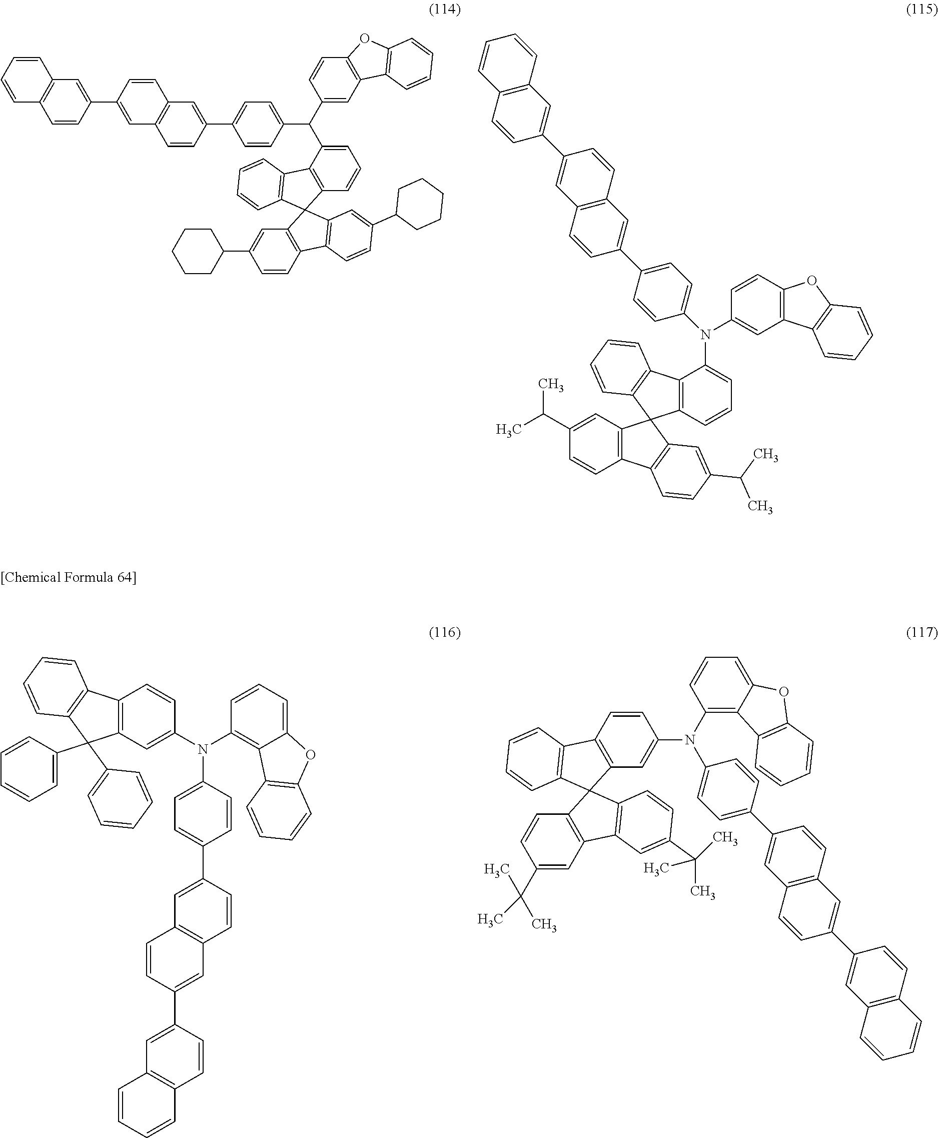 [SMILES: c1ccc2c(c1)-c1c(C(c3ccc(-c4ccc5cc(-c6ccc7ccccc7c6)ccc5c4)cc3)c3ccc4oc5ccccc5c4c3)cccc1C21c2cc(C3CCCCC3)ccc2-c2ccc(C3CCCCC3)cc21 ; CC(C)c1ccc2c(c1)C1(c3cc(C(C)C)ccc3-2)c2ccccc2-c2c(N(c3ccc(-c4ccc5cc(-c6ccc7ccccc7c6)ccc5c4)cc3)c3ccc4oc5ccccc5c4c3)cccc21 ; c1ccc(C2(c3ccccc3)c3ccccc3-c3ccc(N(c4ccc(-c5ccc6cc(-c7ccc8ccccc8c7)ccc6c5)cc4)c4cccc5oc6ccccc6c45)cc32)cc1 ; CC(C)(C)c1ccc2c(c1)-c1cc(C(C)(C)C)ccc1C21c2ccccc2-c2ccc(N(c3ccc(-c4ccc5cc(-c6ccc7ccccc7c6)ccc5c4)cc3)c3cccc4oc5ccccc5c34)cc21]

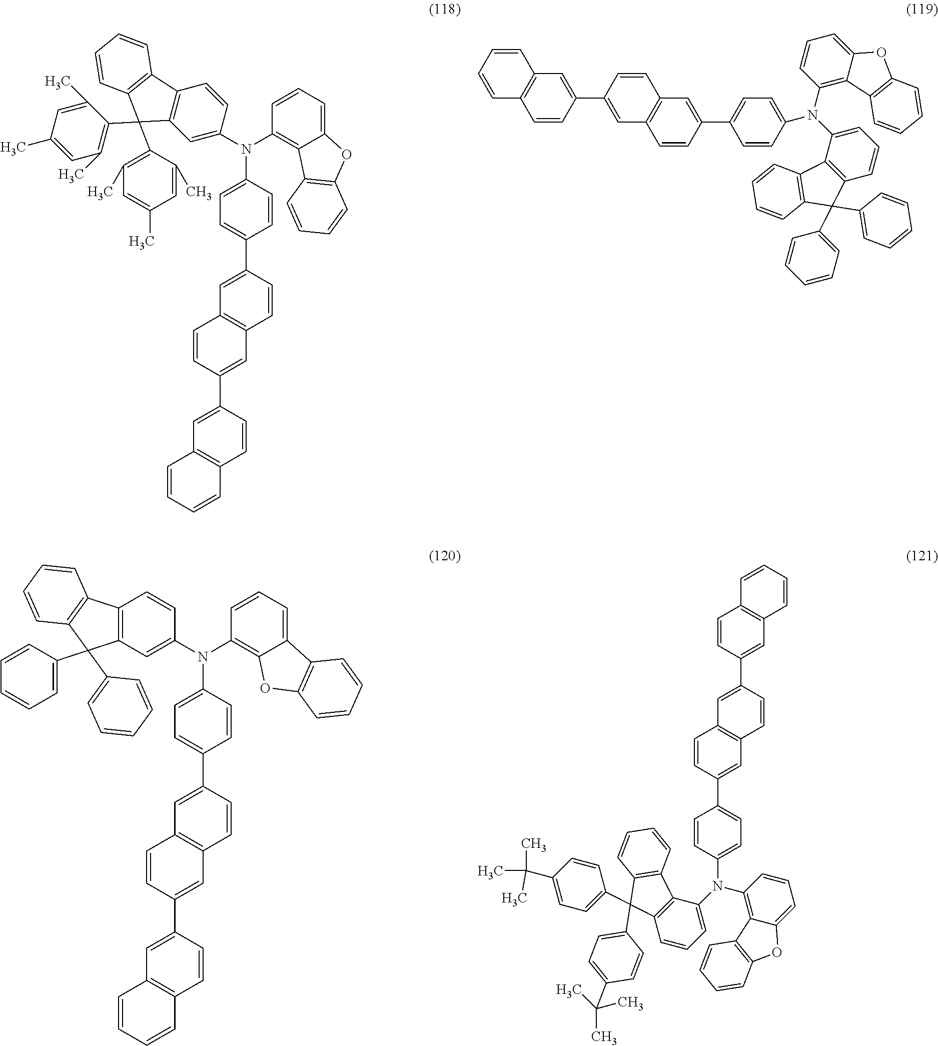 [SMILES: Cc1cc(C)c(C2(c3c(C)cc(C)cc3C)c3ccccc3-c3ccc(N(c4ccc(-c5ccc6cc(-c7ccc8ccccc8c7)ccc6c5)cc4)c4cccc5oc6ccccc6c45)cc32)c(C)c1 ; c1ccc(C2(c3ccccc3)c3ccccc3-c3c(N(c4ccc(-c5ccc6cc(-c7ccc8ccccc8c7)ccc6c5)cc4)c4cccc5oc6ccccc6c45)cccc32)cc1 ; c1ccc(C2(c3ccccc3)c3ccccc3-c3ccc(N(c4ccc(-c5ccc6cc(-c7ccc8ccccc8c7)ccc6c5)cc4)c4cccc5c4oc4ccccc45)cc32)cc1 ; CC(C)(C)c1ccc(C2(c3ccc(C(C)(C)C)cc3)c3ccccc3-c3c(N(c4ccc(-c5ccc6cc(-c7ccc8ccccc8c7)ccc6c5)cc4)c4cccc5oc6ccccc6c45)cccc32)cc1]

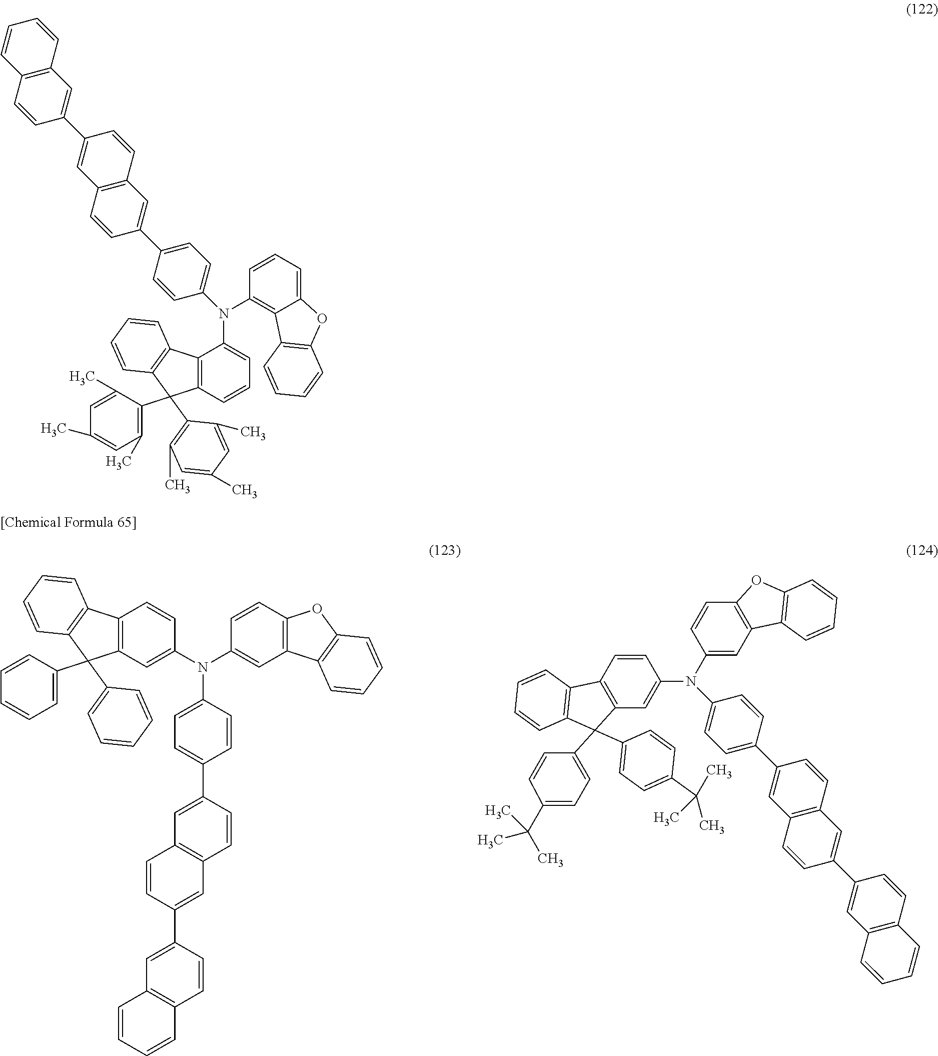 [SMILES: Cc1cc(C)c(C2(c3c(C)cc(C)cc3C)c3ccccc3-c3c(N(c4ccc(-c5ccc6cc(-c7ccc8ccccc8c7)ccc6c5)cc4)c4cccc5oc6ccccc6c45)cccc32)c(C)c1 ; c1ccc(C2(c3ccccc3)c3ccccc3-c3ccc(N(c4ccc(-c5ccc6cc(-c7ccc8ccccc8c7)ccc6c5)cc4)c4ccc5oc6ccccc6c5c4)cc32)cc1 ; CC(C)(C)c1ccc(C2(c3ccc(C(C)(C)C)cc3)c3ccccc3-c3ccc(N(c4ccc(-c5ccc6cc(-c7ccc8ccccc8c7)ccc6c5)cc4)c4ccc5oc6ccccc6c5c4)cc32)cc1]

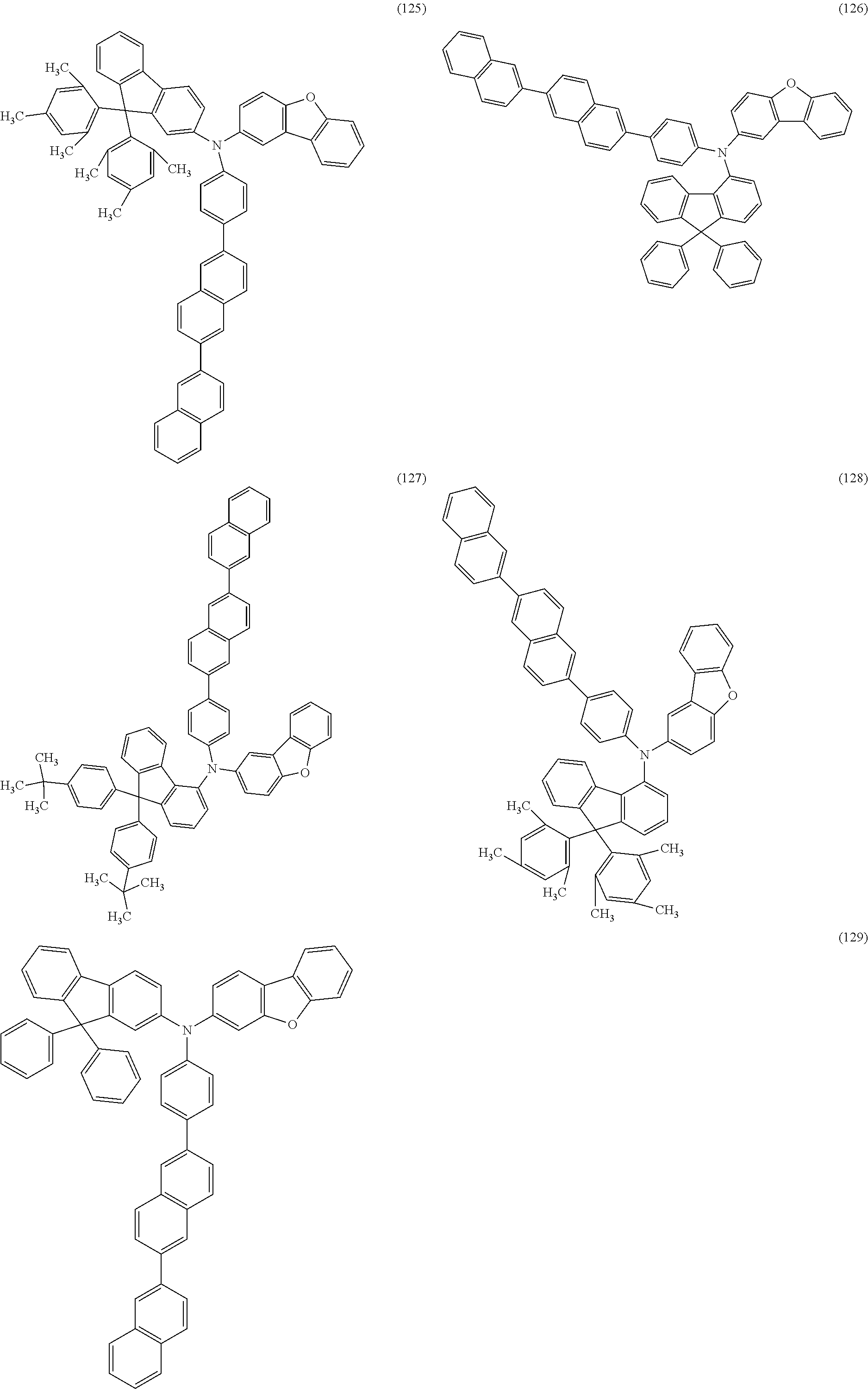

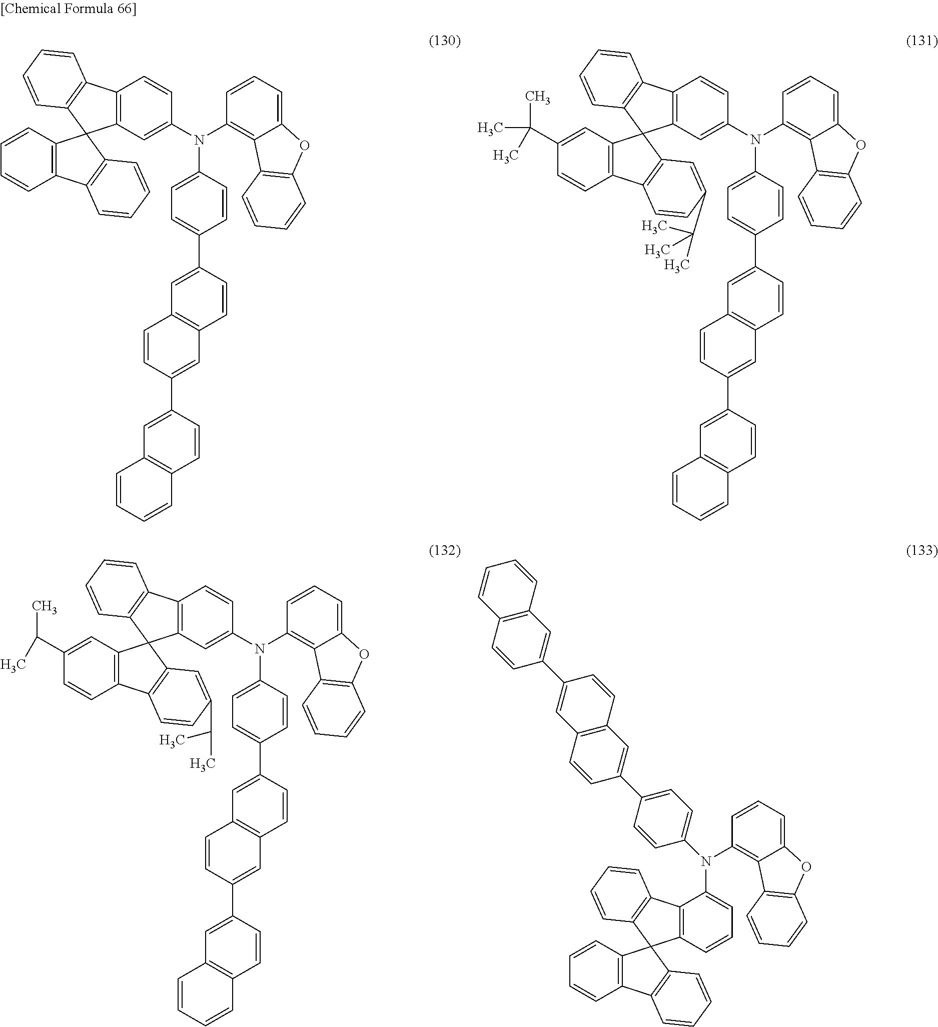

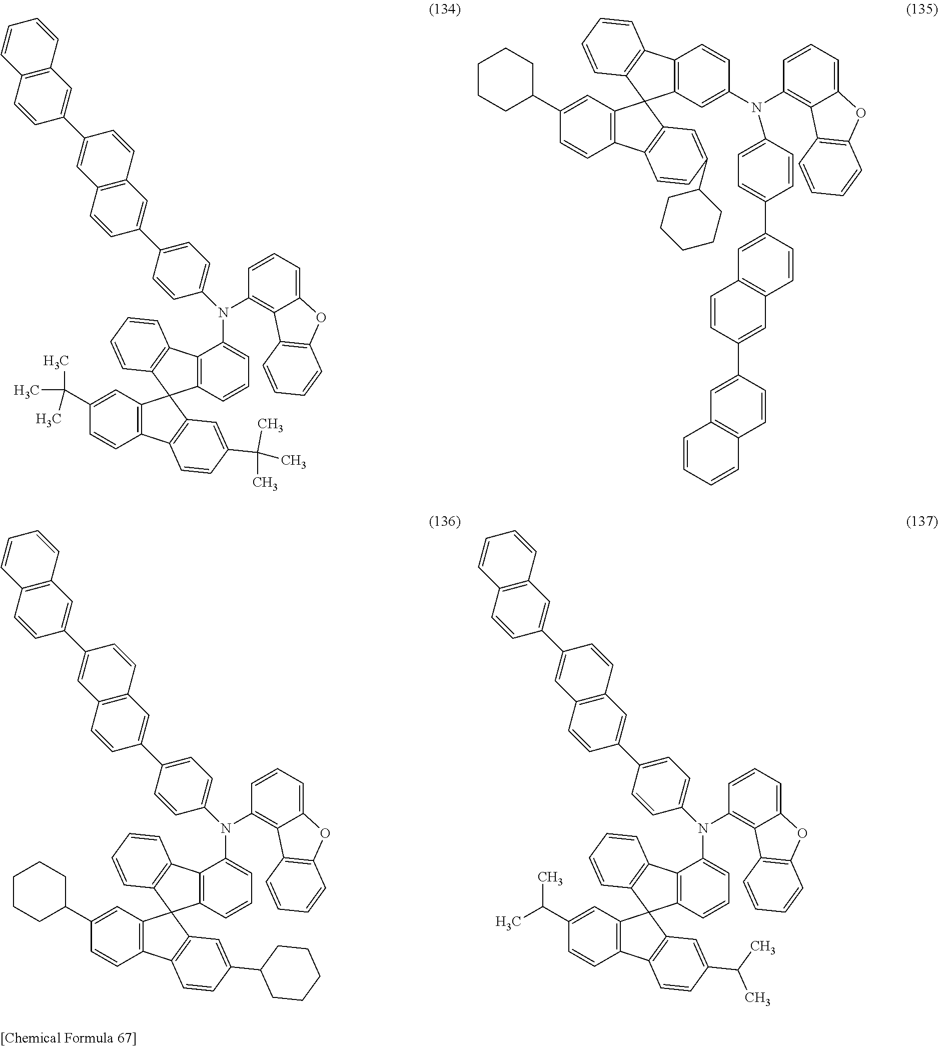

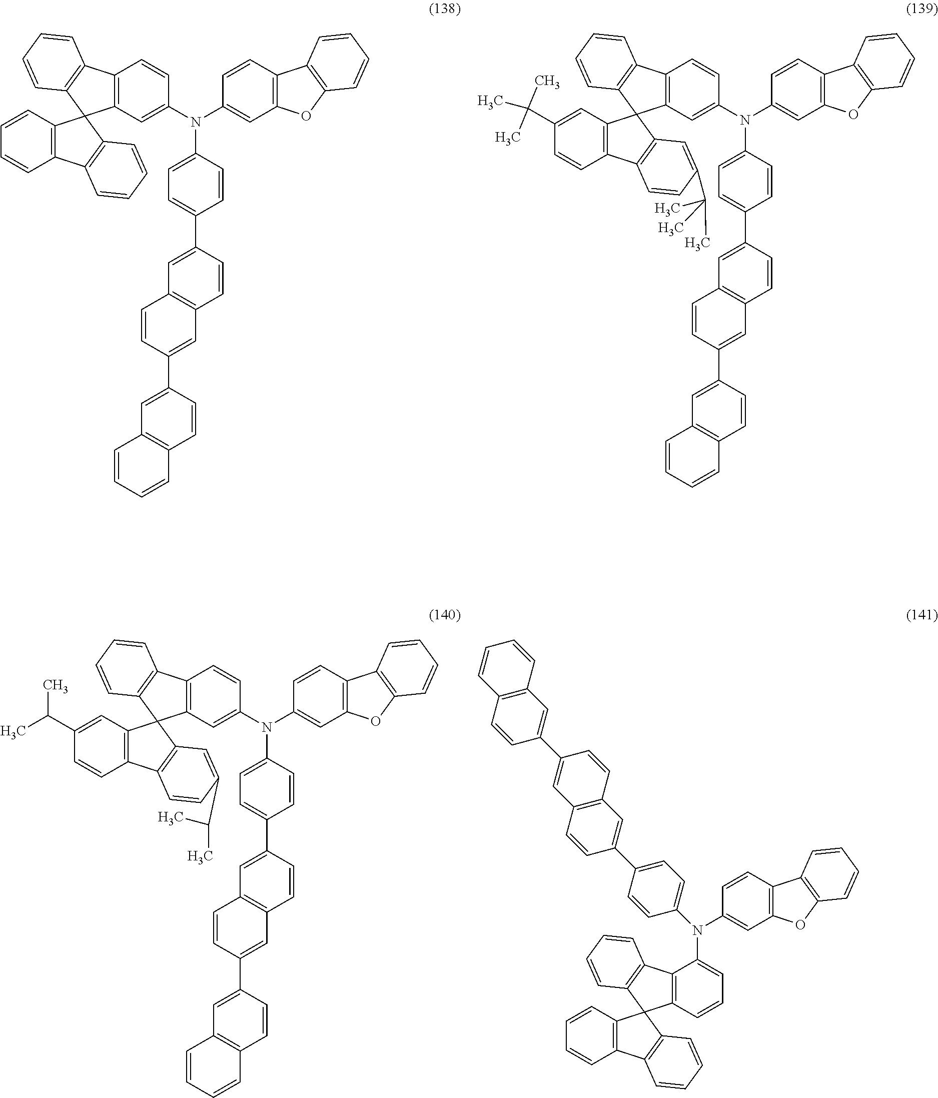

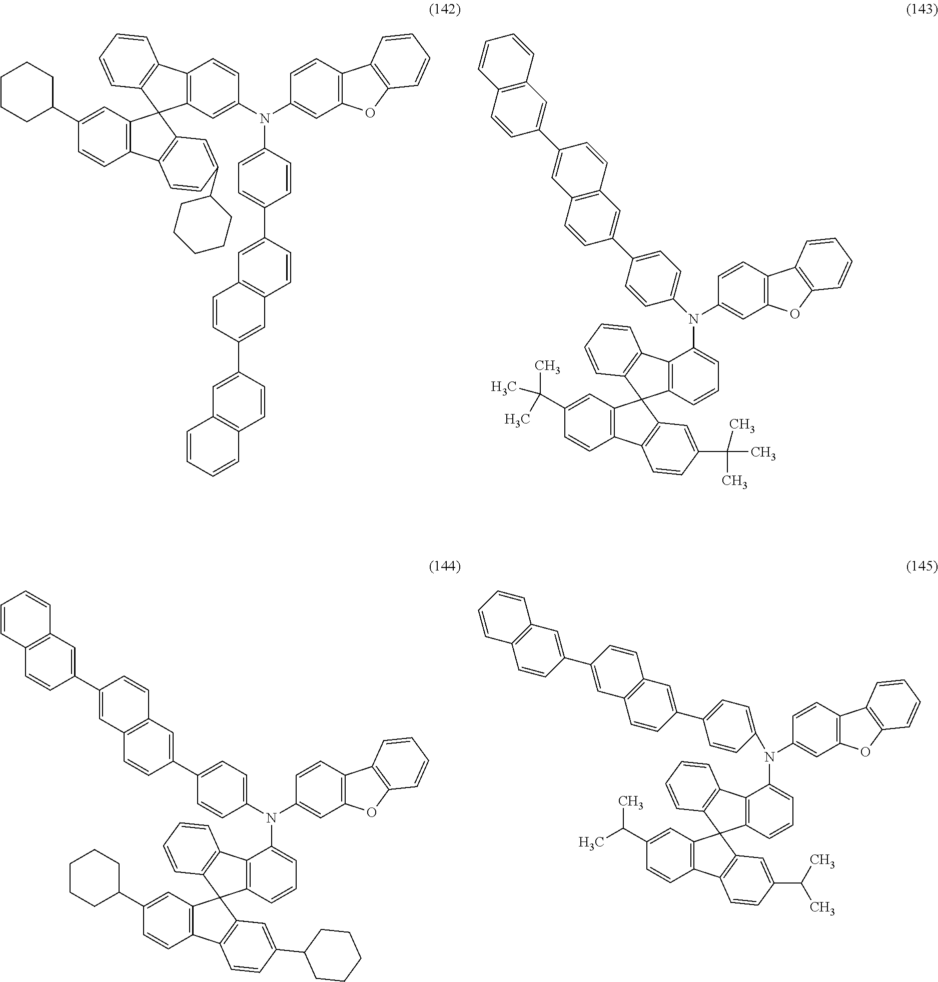

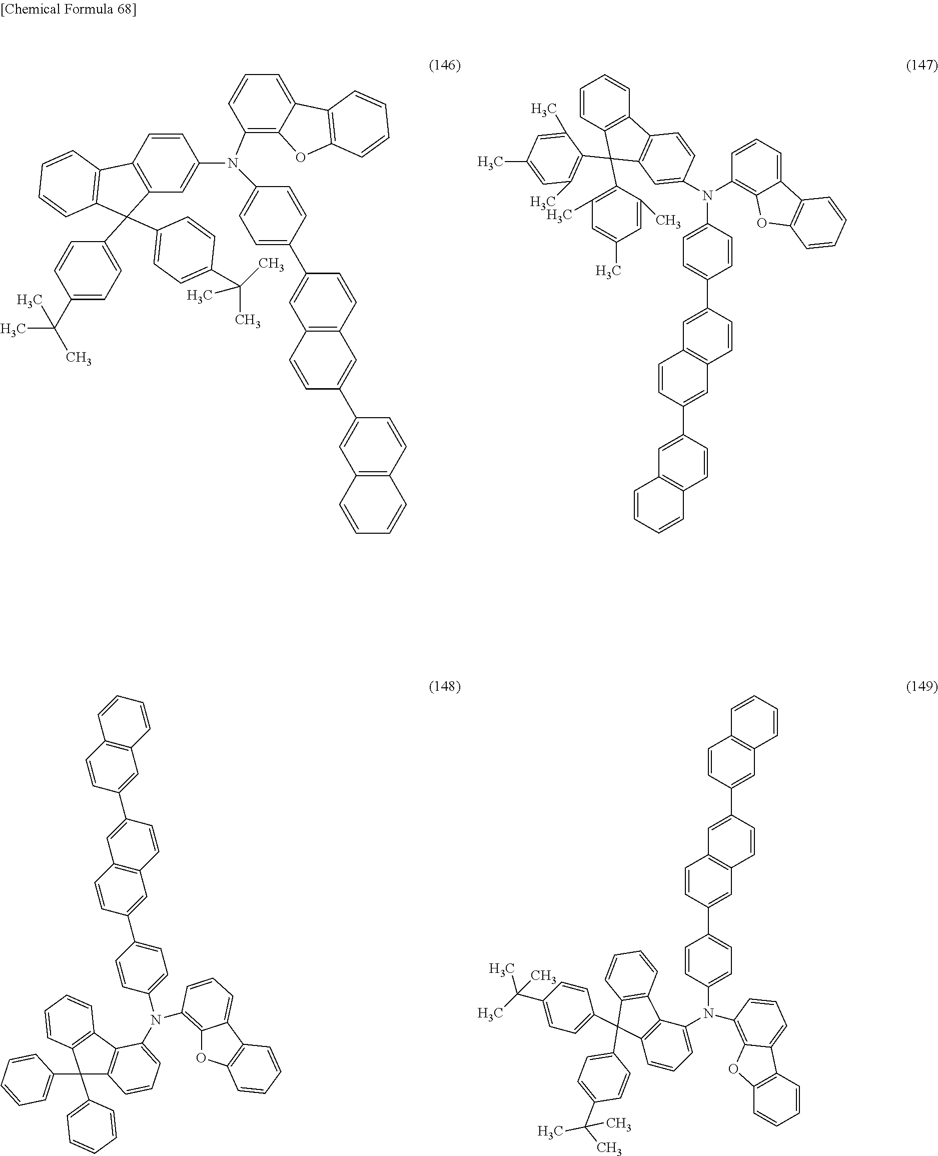 [SMILES: CC(C)(C)c1ccc(C2(c3ccc(C(C)(C)C)cc3)c3ccccc3-c3ccc(N(c4ccc(-c5ccc6cc(-c7ccc8ccccc8c7)ccc6c5)cc4)c4cccc5c4oc4ccccc45)cc32)cc1 ; Cc1cc(C)c(C2(c3c(C)cc(C)cc3C)c3ccccc3-c3ccc(N(c4ccc(-c5ccc6cc(-c7ccc8ccccc8c7)ccc6c5)cc4)c4cccc5c4oc4ccccc45)cc32)c(C)c1 ; c1ccc(C2(c3ccccc3)c3ccccc3-c3c(N(c4ccc(-c5ccc6cc(-c7ccc8ccccc8c7)ccc6c5)cc4)c4cccc5c4oc4ccccc45)cccc32)cc1 ; CC(C)(C)c1ccc(C2(c3ccc(C(C)(C)C)cc3)c3ccccc3-c3c(N(c4ccc(-c5ccc6cc(-c7ccc8ccccc8c7)ccc6c5)cc4)c4cccc5c4oc4ccccc45)cccc32)cc1]

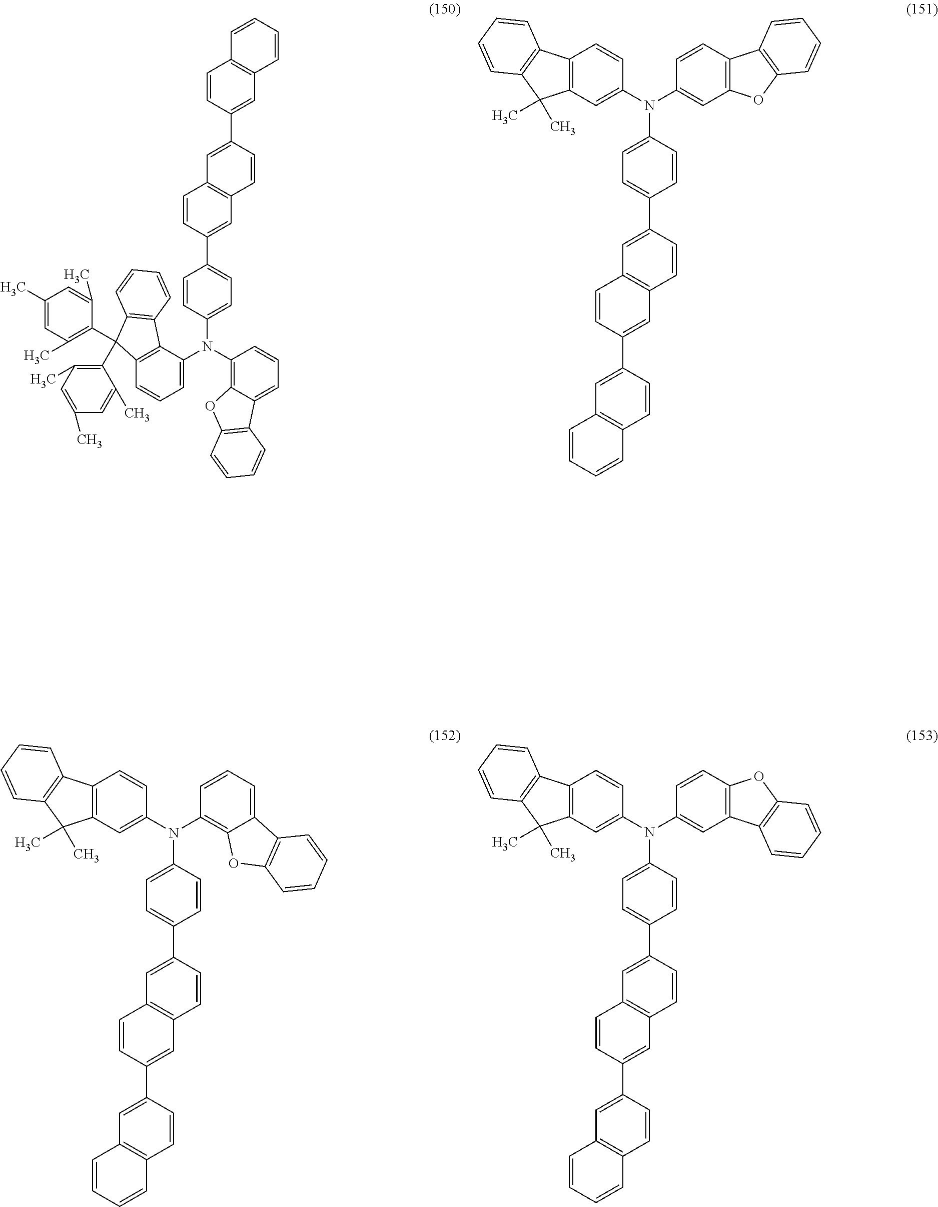 [SMILES: Cc1cc(C)c(C2(c3c(C)cc(C)cc3C)c3ccccc3-c3c(N(c4ccc(-c5ccc6cc(-c7ccc8ccccc8c7)ccc6c5)cc4)c4cccc5c4oc4ccccc45)cccc32)c(C)c1 ; CC1(C)c2ccccc2-c2ccc(N(c3ccc(-c4ccc5cc(-c6ccc7ccccc7c6)ccc5c4)cc3)c3ccc4c(c3)oc3ccccc34)cc21 ; CC1(C)c2ccccc2-c2ccc(N(c3ccc(-c4ccc5cc(-c6ccc7ccccc7c6)ccc5c4)cc3)c3cccc4c3oc3ccccc34)cc21 ; CC1(C)c2ccccc2-c2ccc(N(c3ccc(-c4ccc5cc(-c6ccc7ccccc7c6)ccc5c4)cc3)c3ccc4oc5ccccc5c4c3)cc21]

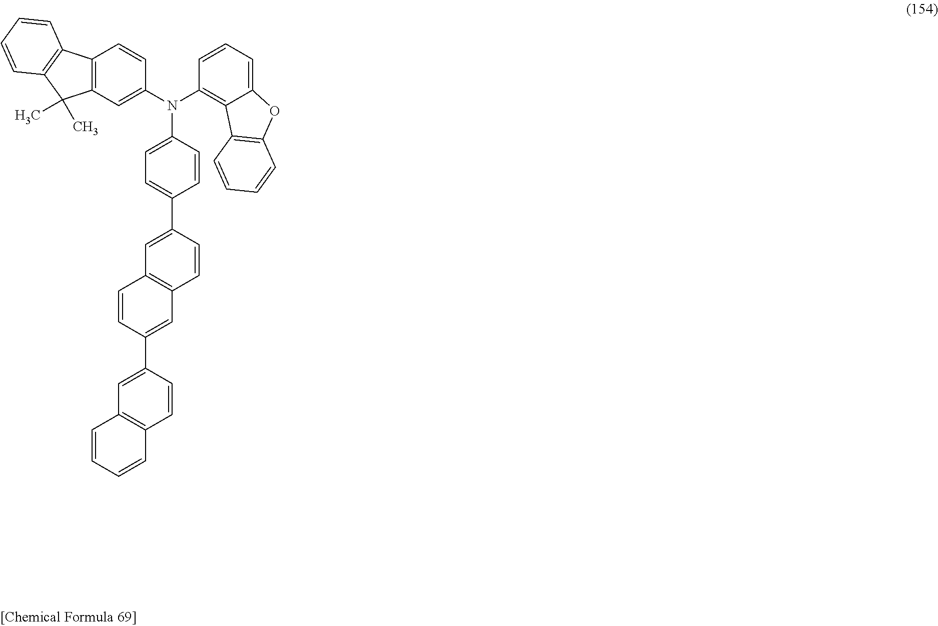 [SMILES: CC1(C)c2ccccc2-c2ccc(N(c3ccc(-c4ccc5cc(-c6ccc7ccccc7c6)ccc5c4)cc3)c3cccc4oc5ccccc5c34)cc21]

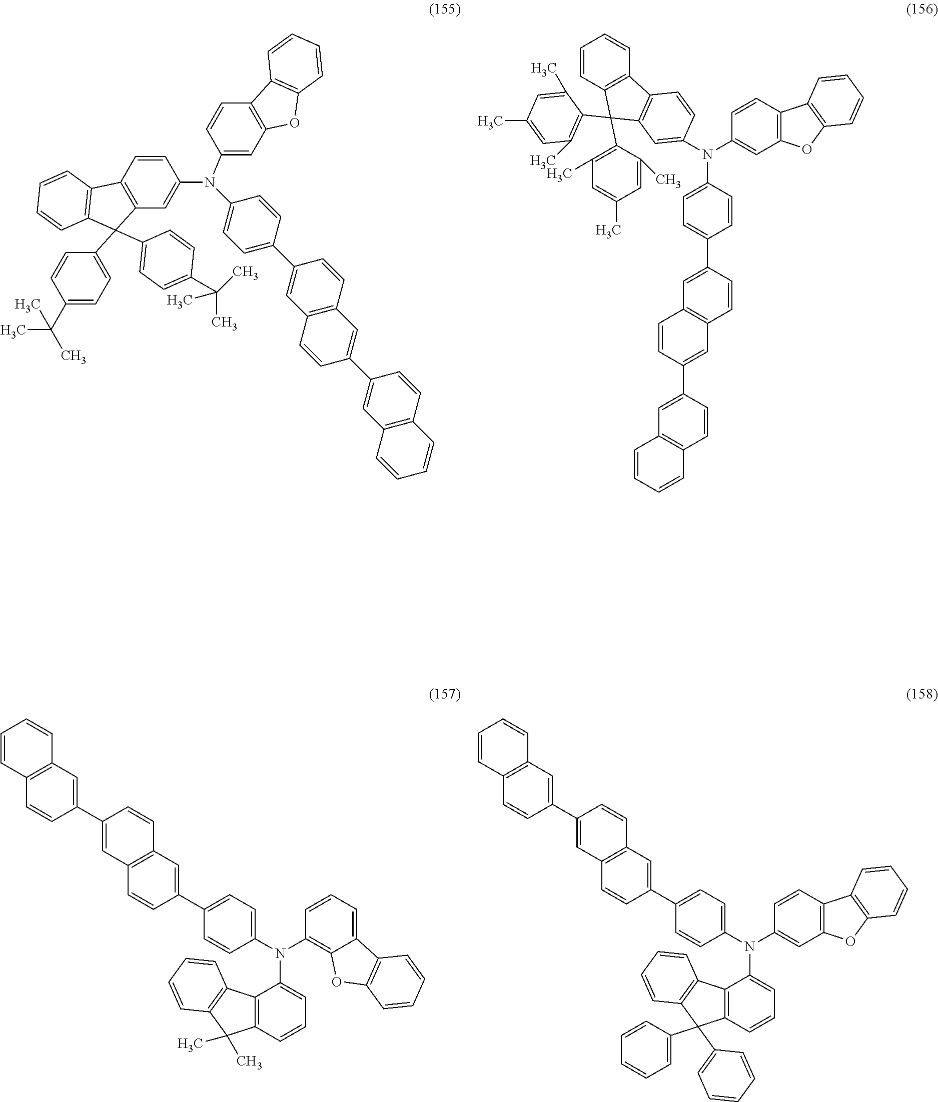 [SMILES: CC(C)(C)c1ccc(C2(c3ccc(C(C)(C)C)cc3)c3ccccc3-c3ccc(N(c4ccc(-c5ccc6cc(-c7ccc8ccccc8c7)ccc6c5)cc4)c4ccc5c(c4)oc4ccccc45)cc32)cc1 ; Cc1cc(C)c(C2(c3c(C)cc(C)cc3C)c3ccccc3-c3ccc(N(c4ccc(-c5ccc6cc(-c7ccc8ccccc8c7)ccc6c5)cc4)c4ccc5c(c4)oc4ccccc45)cc32)c(C)c1 ; CC1(C)c2ccccc2-c2c(N(c3ccc(-c4ccc5cc(-c6ccc7ccccc7c6)ccc5c4)cc3)c3cccc4c3oc3ccccc34)cccc21 ; c1ccc(C2(c3ccccc3)c3ccccc3-c3c(N(c4ccc(-c5ccc6cc(-c7ccc8ccccc8c7)ccc6c5)cc4)c4ccc5c(c4)oc4ccccc45)cccc32)cc1]

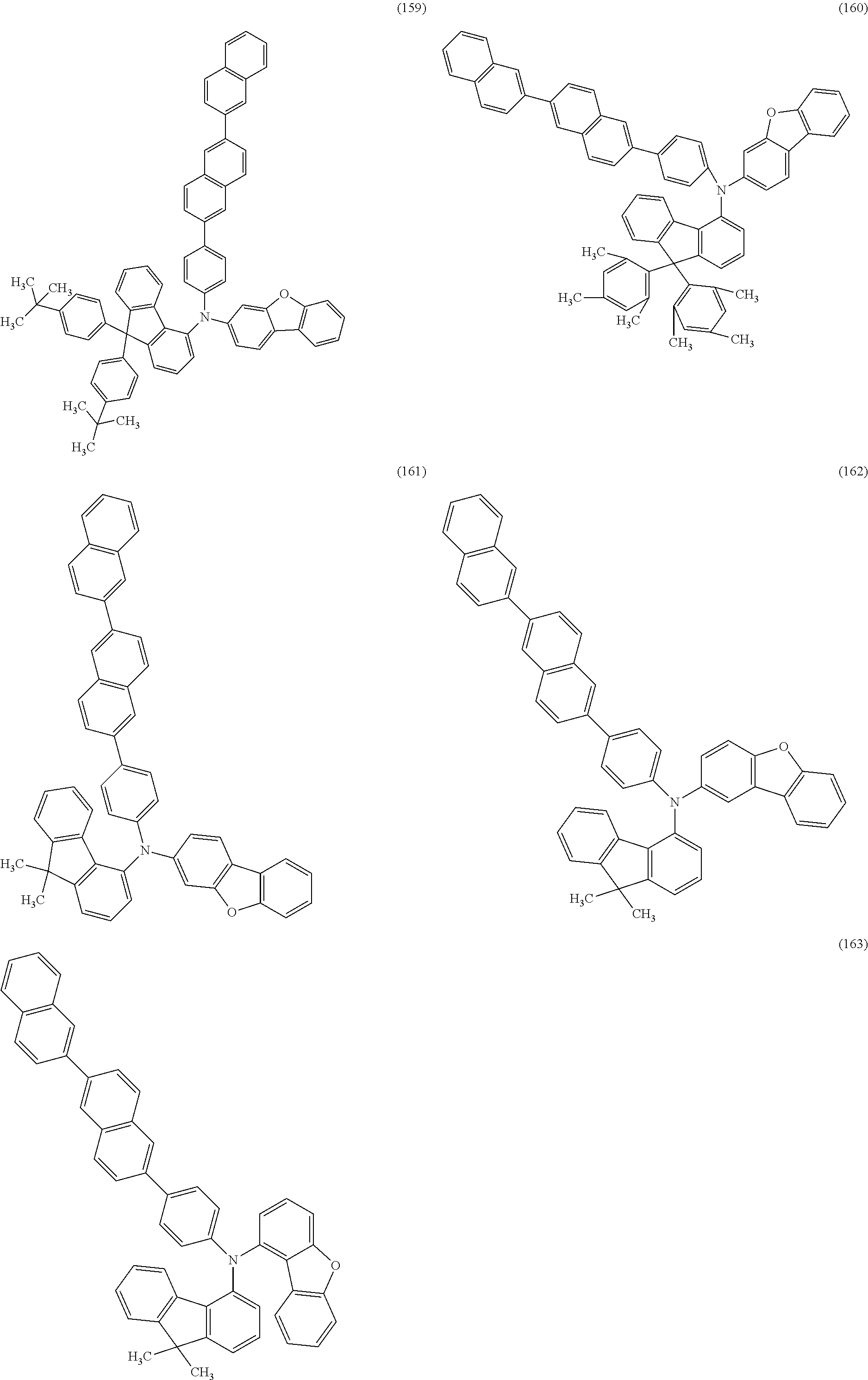

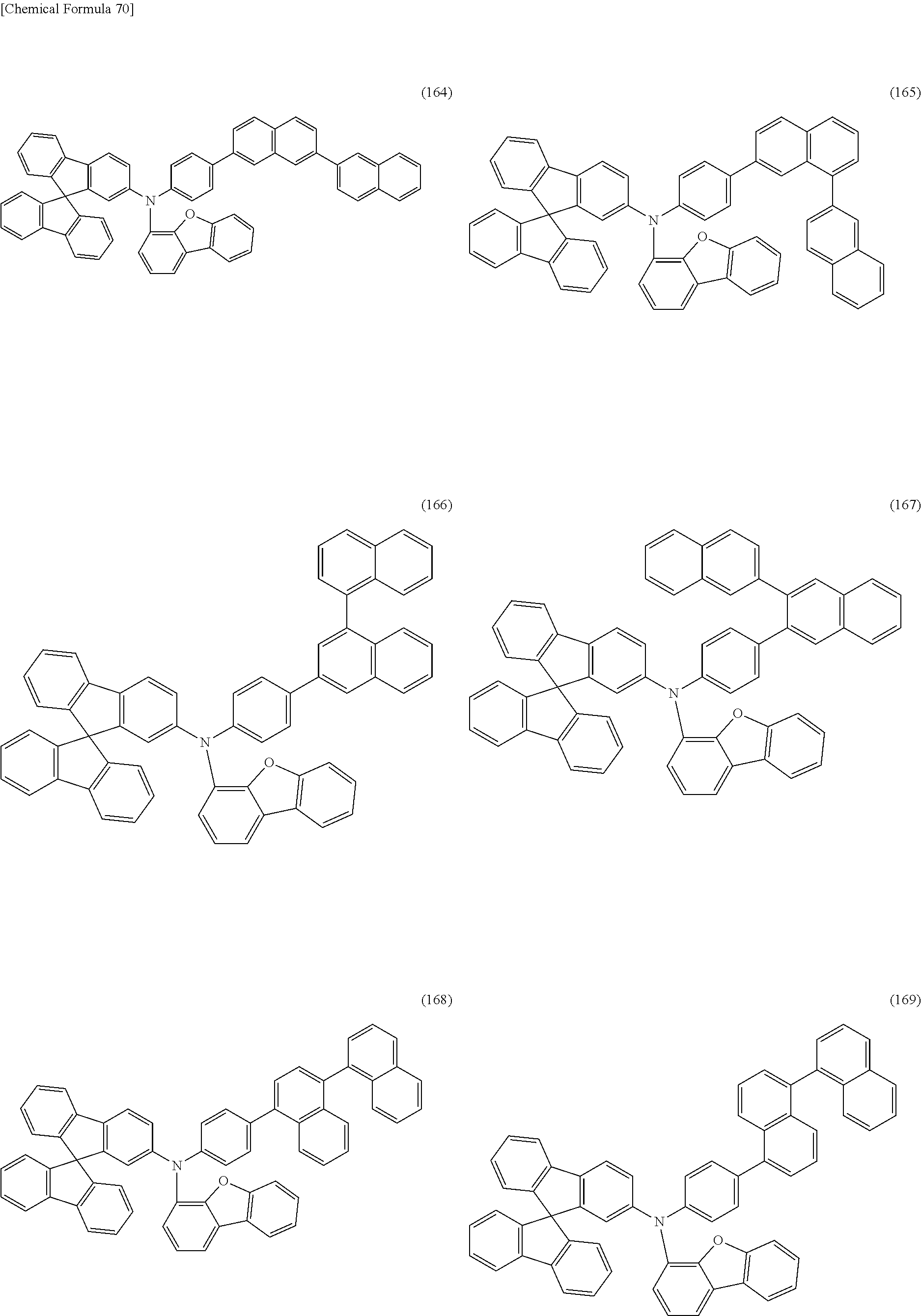

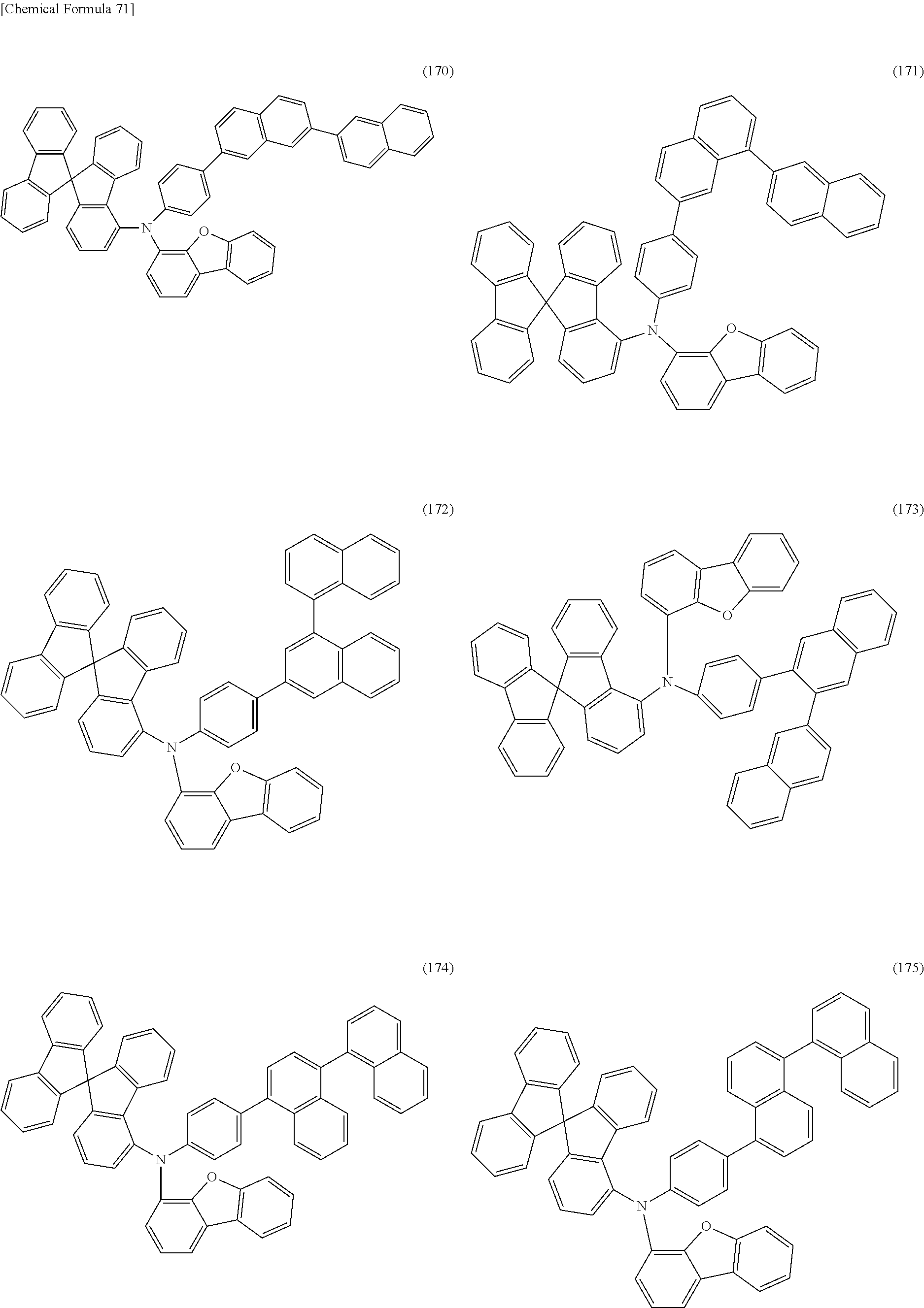

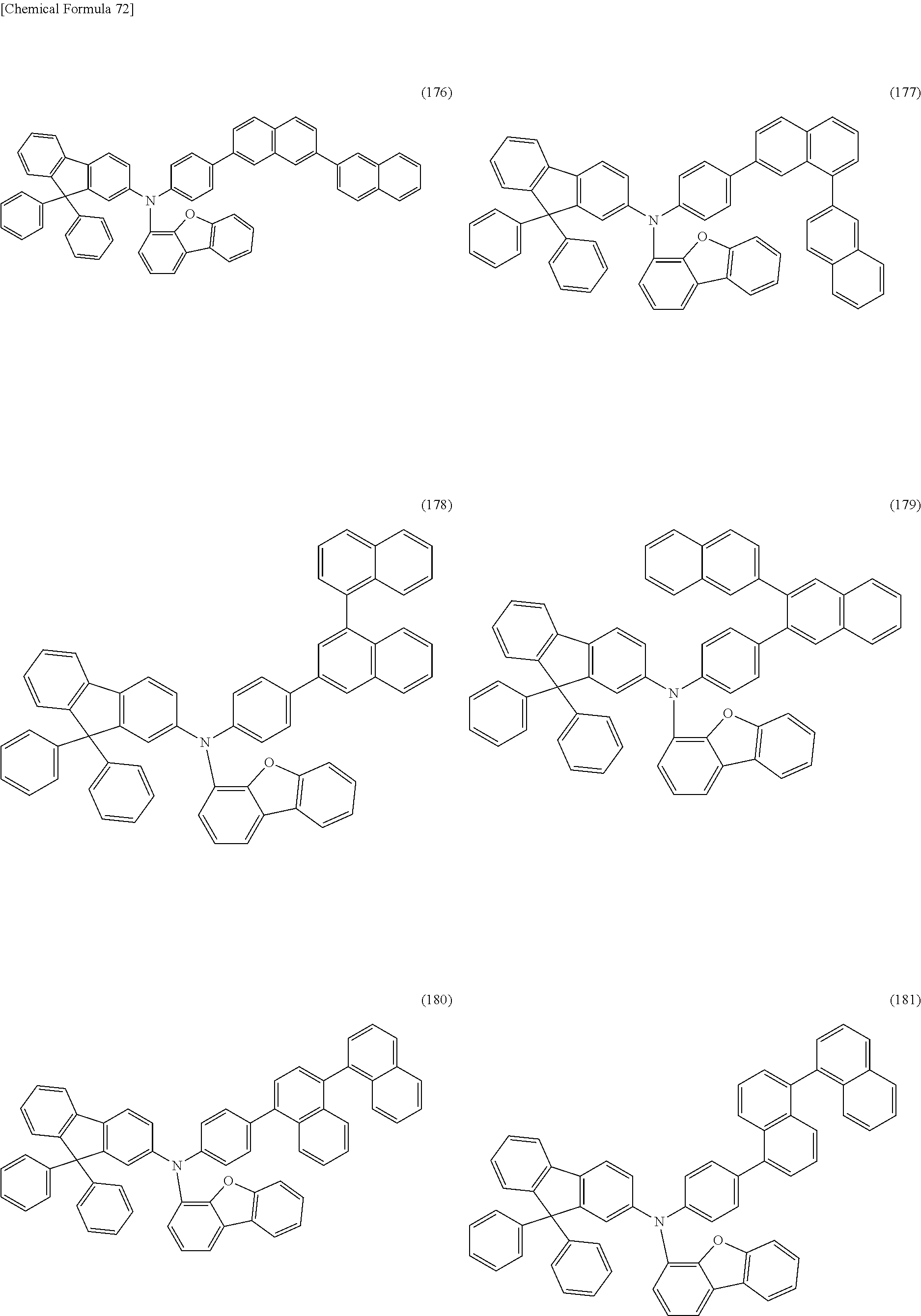

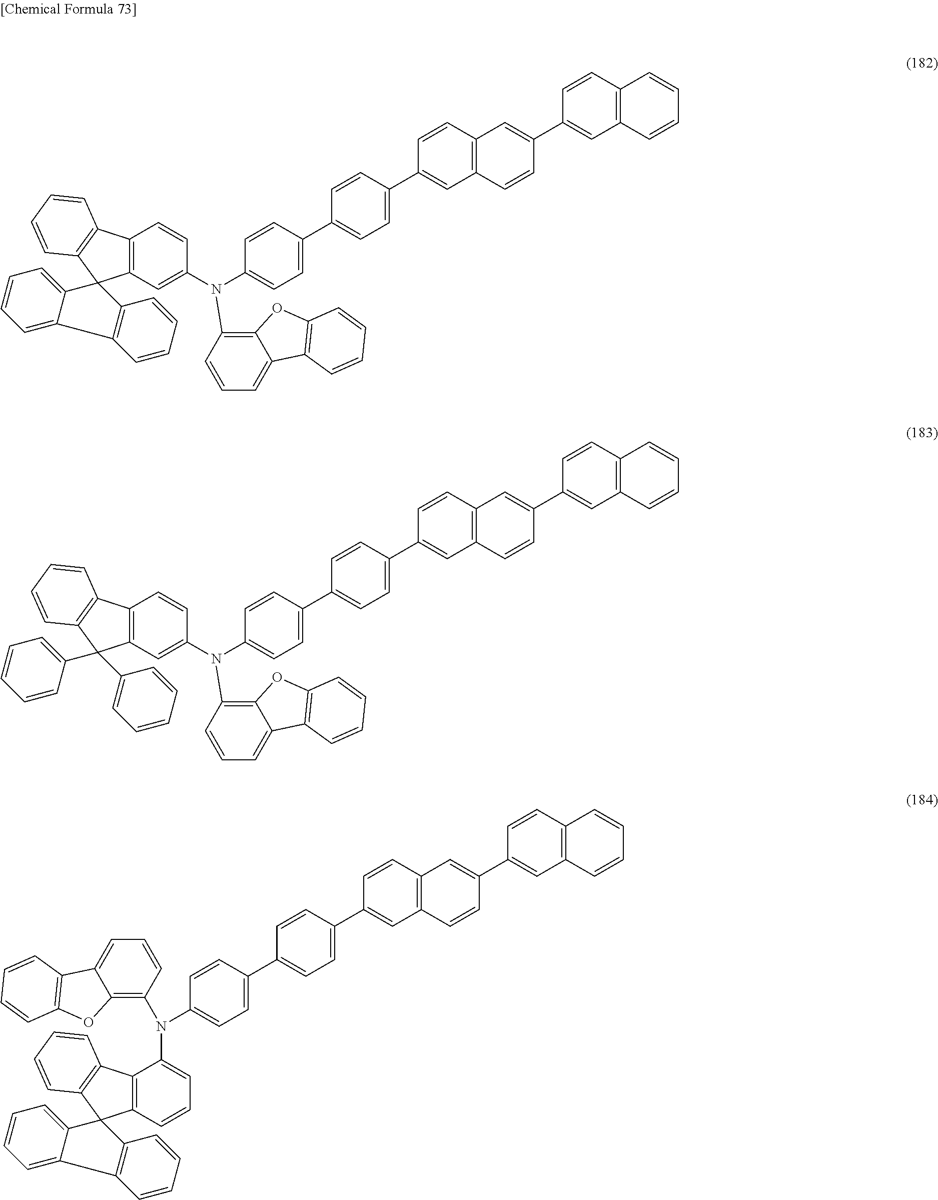 [SMILES: c1ccc2c(c1)-c1ccccc1C21c2ccccc2-c2ccc(N(c3ccc(-c4ccc(-c5ccc6cc(-c7ccc8ccccc8c7)ccc6c5)cc4)cc3)c3cccc4c3oc3ccccc34)cc21 ; c1ccc(C2(c3ccccc3)c3ccccc3-c3ccc(N(c4ccc(-c5ccc(-c6ccc7cc(-c8ccc9ccccc9c8)ccc7c6)cc5)cc4)c4cccc5c4oc4ccccc45)cc32)cc1 ; c1ccc2c(c1)-c1ccccc1C21c2ccccc2-c2c(N(c3ccc(-c4ccc(-c5ccc6cc(-c7ccc8ccccc8c7)ccc6c5)cc4)cc3)c3cccc4c3oc3ccccc34)cccc21]

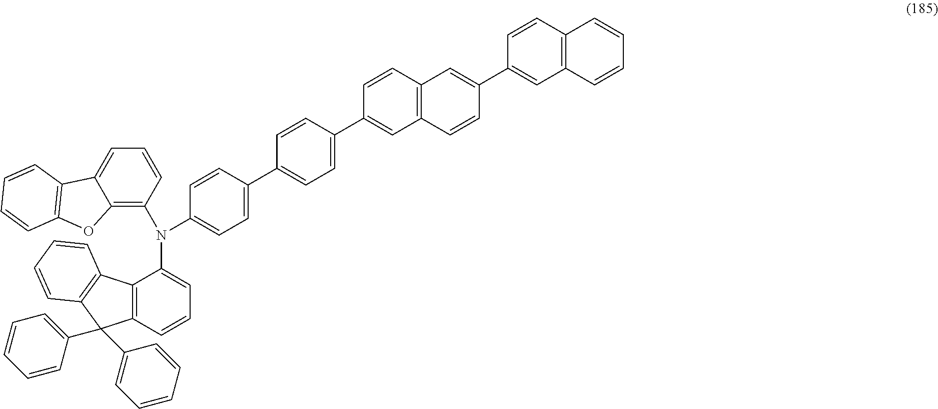 [SMILES: c1ccc(C2(c3ccccc3)c3ccccc3-c3c(N(c4ccc(-c5ccc(-c6ccc7cc(-c8ccc9ccccc9c8)ccc7c6)cc5)cc4)c4cccc5c4oc4ccccc45)cccc32)cc1]

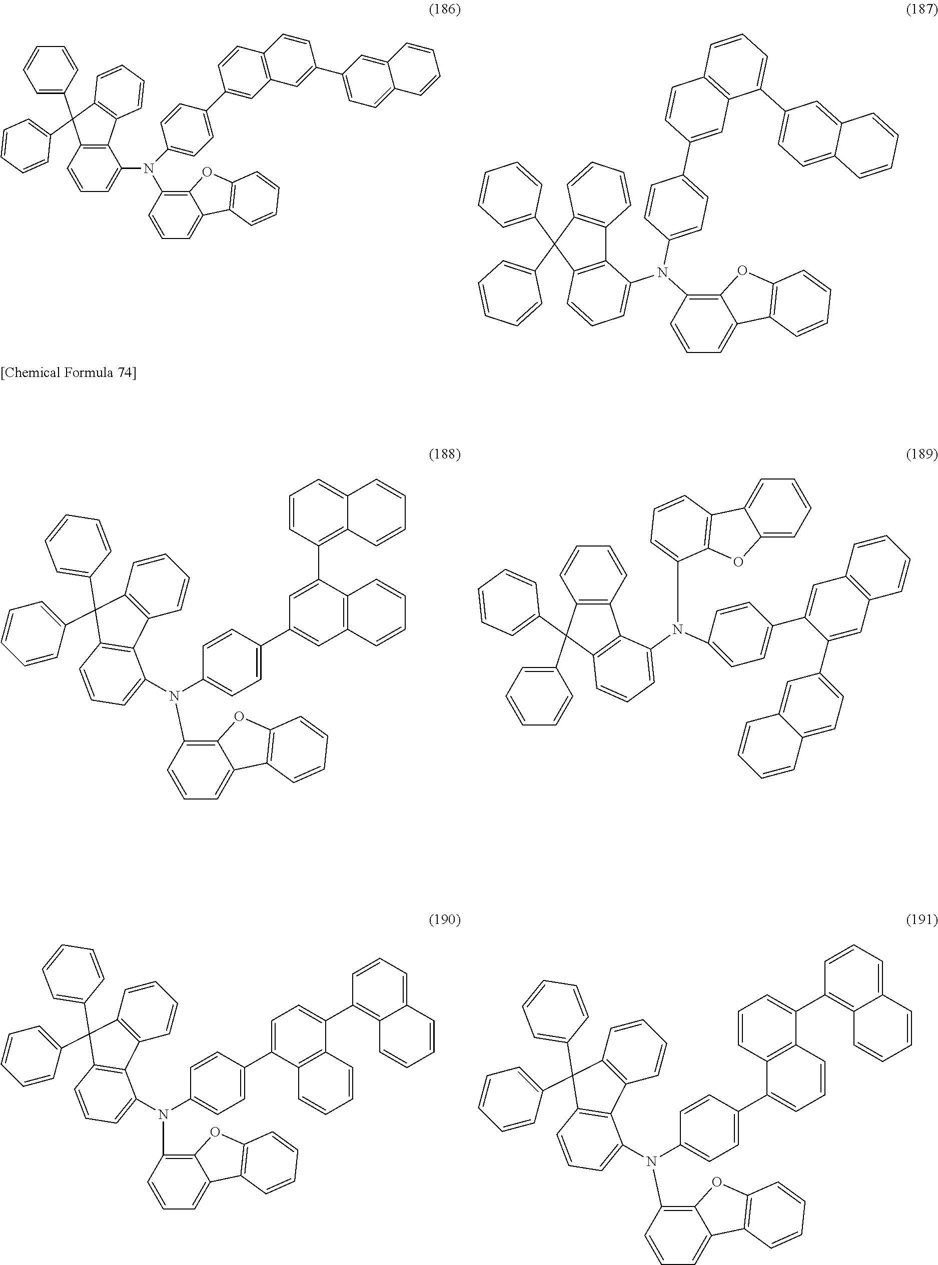

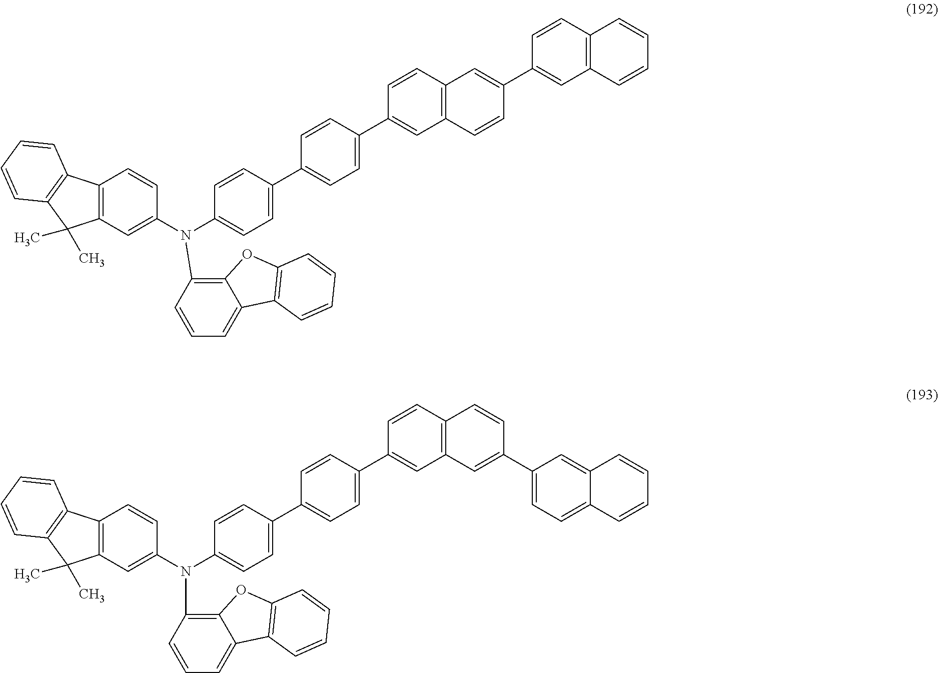 [SMILES: CC1(C)c2ccccc2-c2ccc(N(c3ccc(-c4ccc(-c5ccc6cc(-c7ccc8ccccc8c7)ccc6c5)cc4)cc3)c3cccc4c3oc3ccccc34)cc21 ; CC1(C)c2ccccc2-c2ccc(N(c3ccc(-c4ccc(-c5ccc6ccc(-c7ccc8ccccc8c7)cc6c5)cc4)cc3)c3cccc4c3oc3ccccc34)cc21]

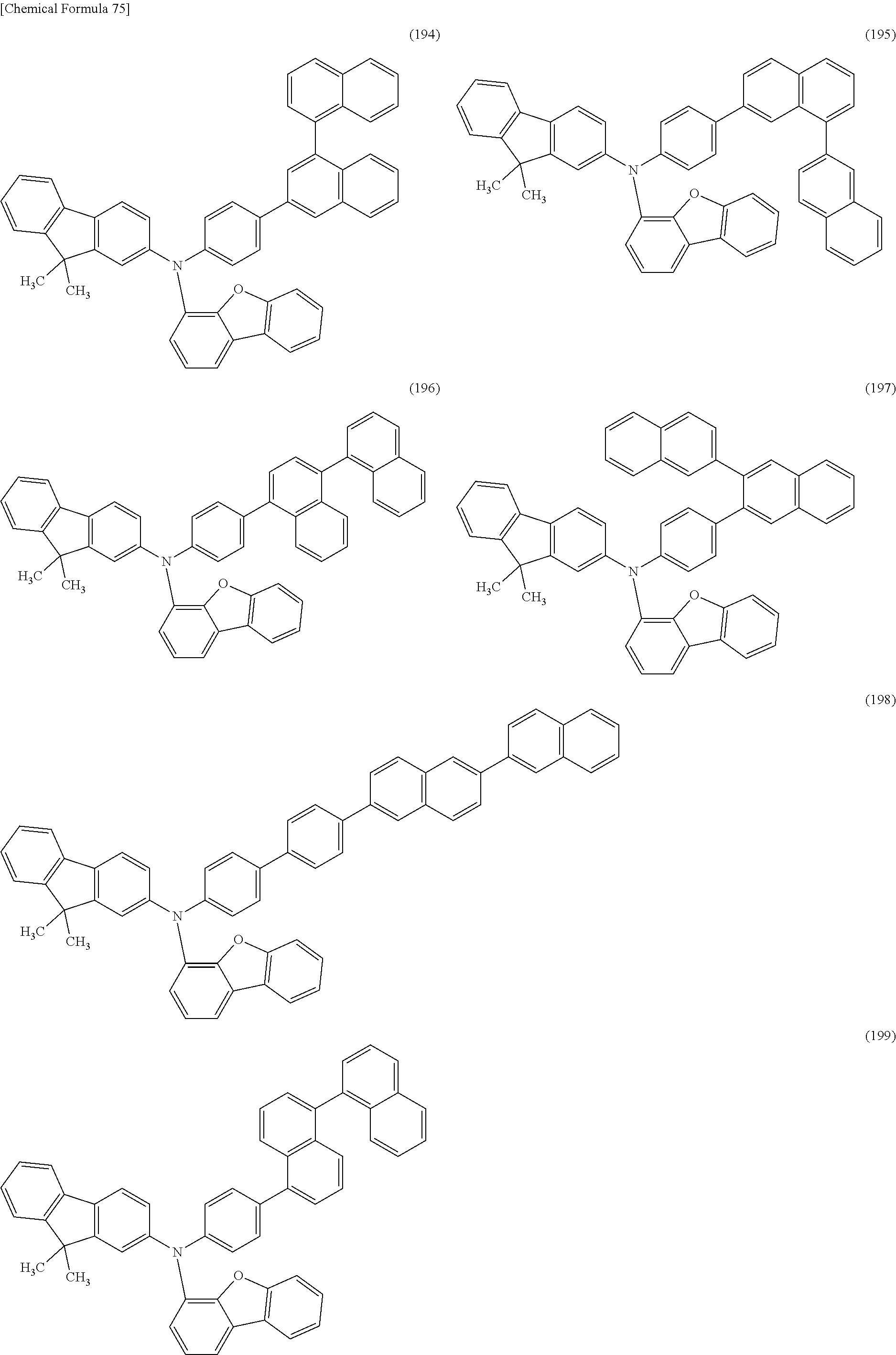

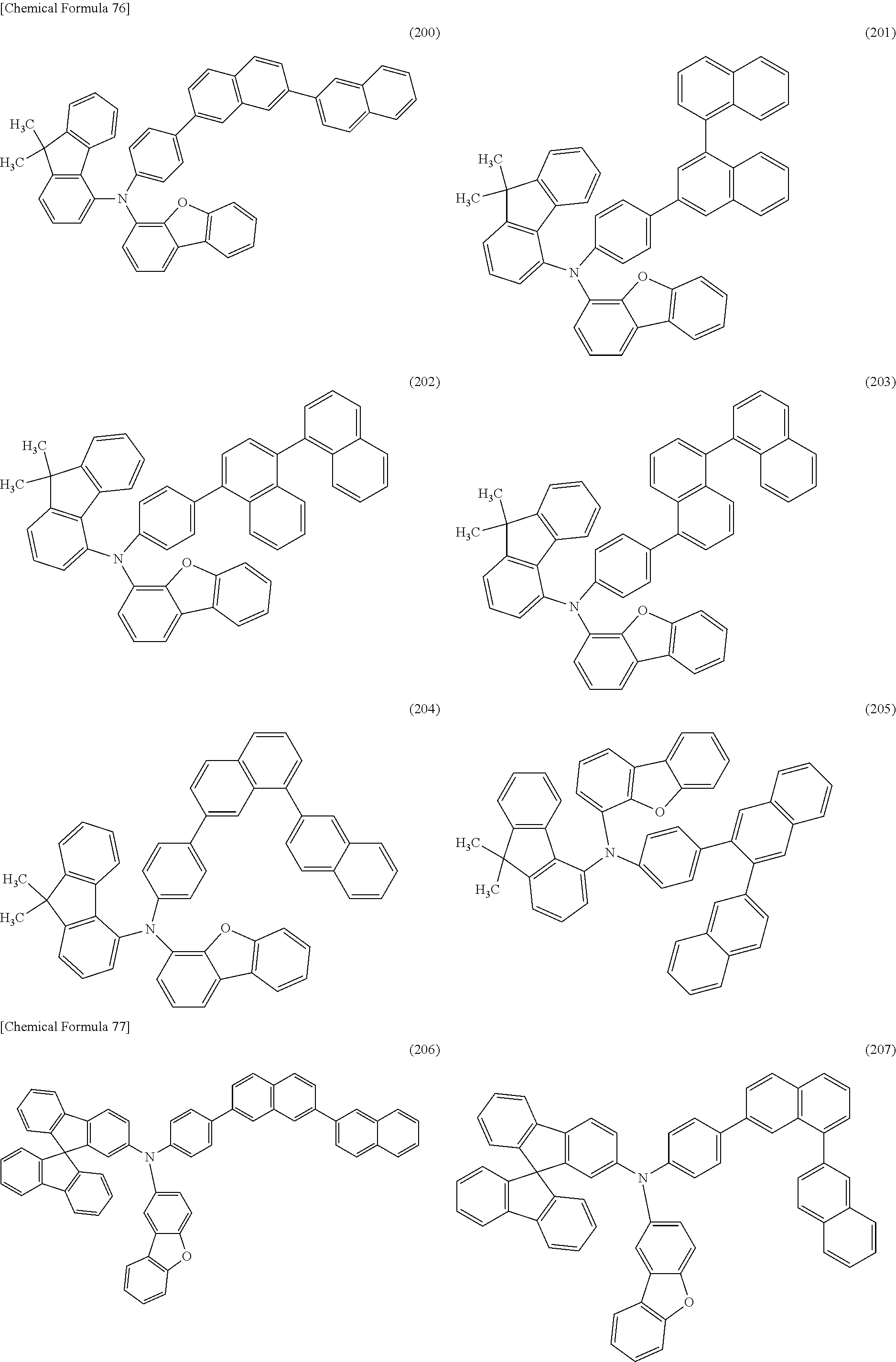

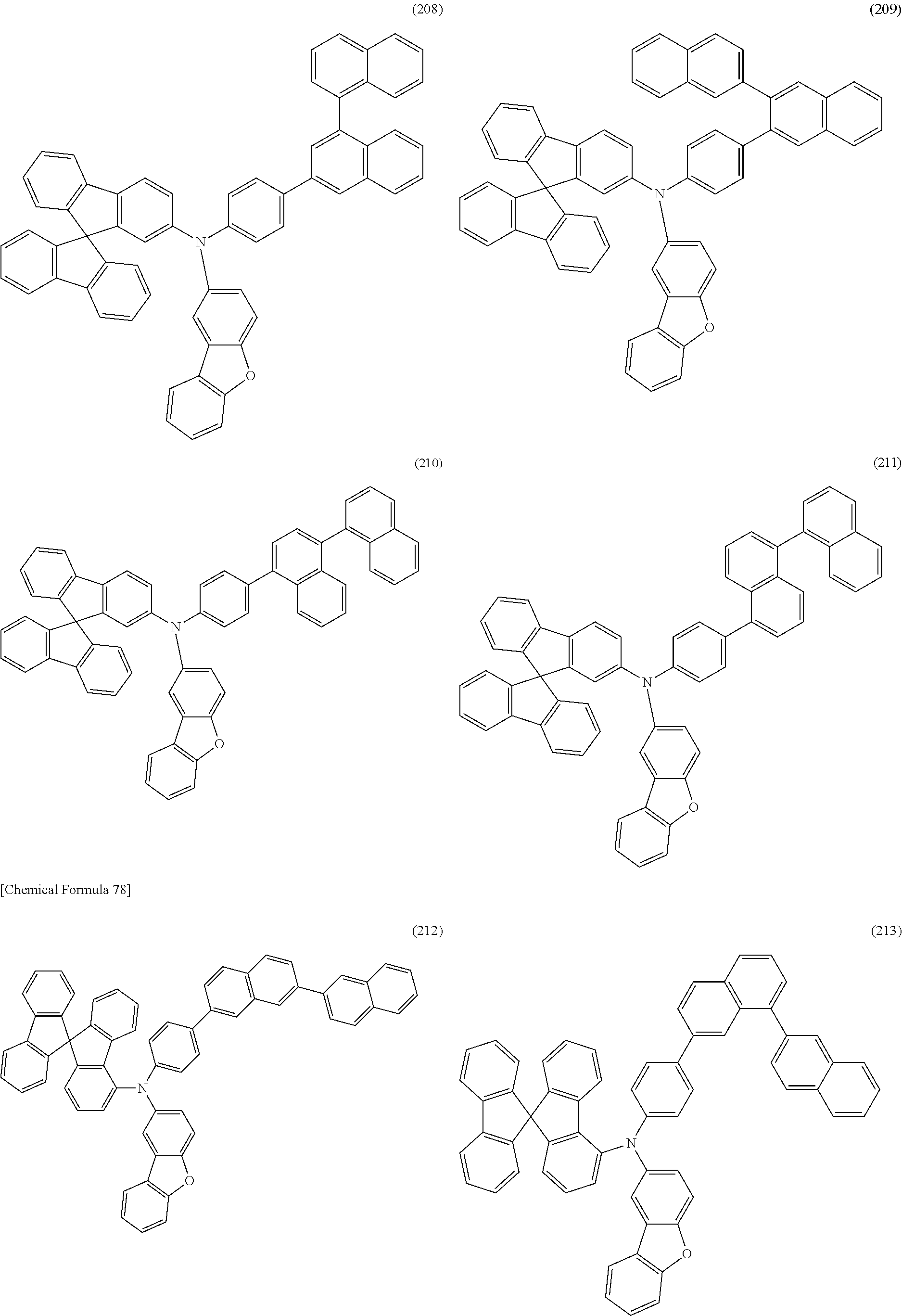

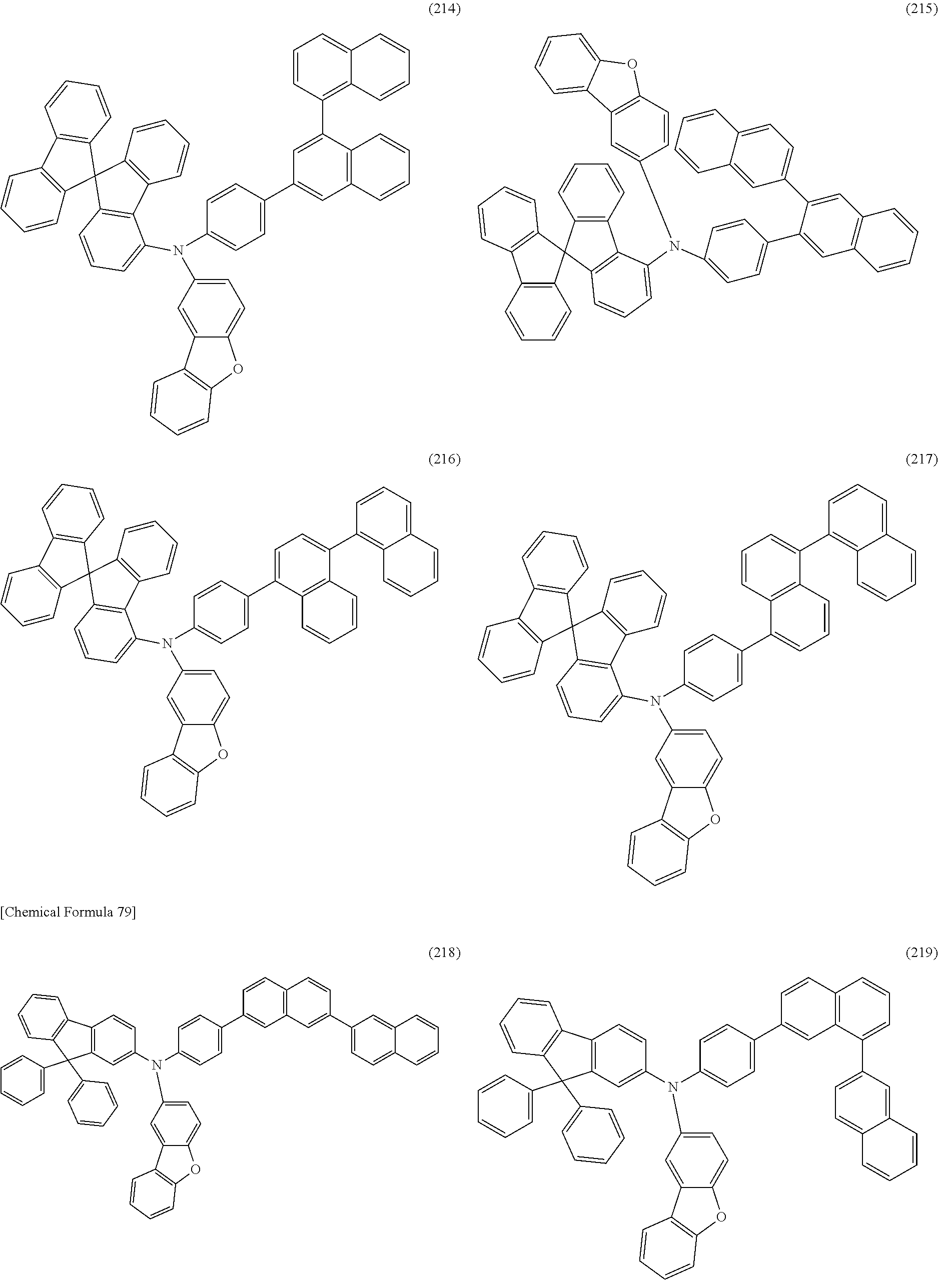

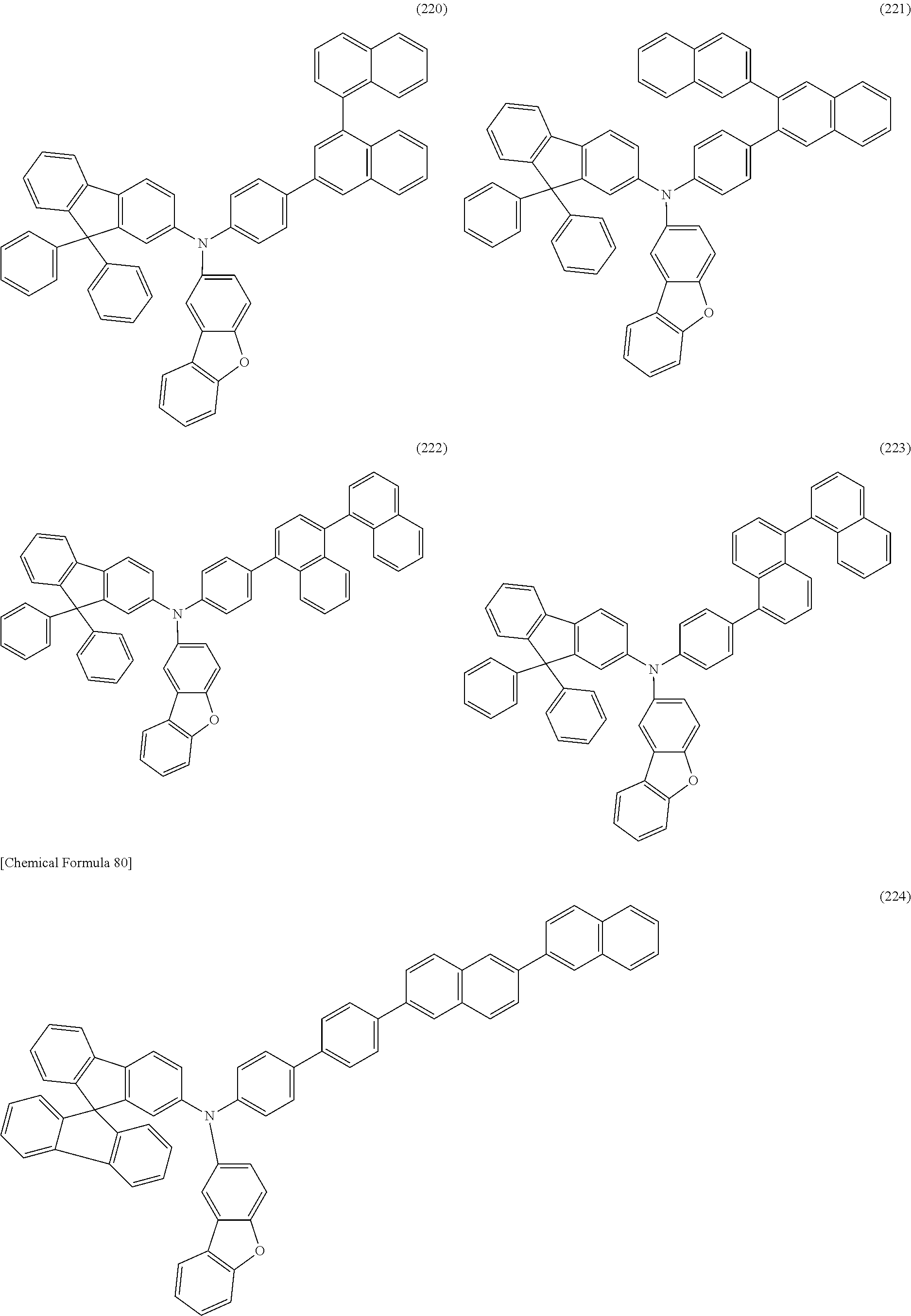

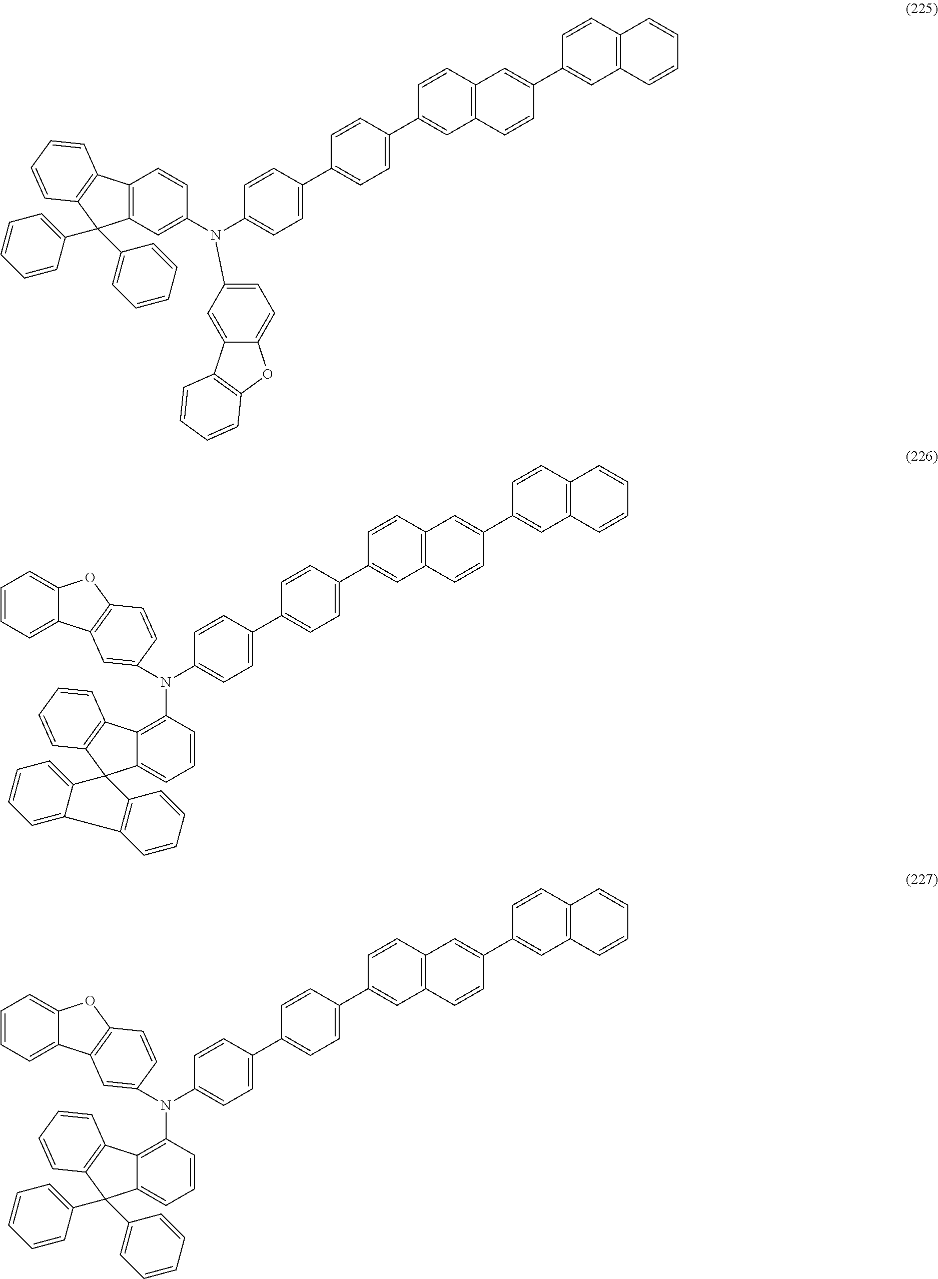 [SMILES: c1ccc(C2(c3ccccc3)c3ccccc3-c3ccc(N(c4ccc(-c5ccc(-c6ccc7cc(-c8ccc9ccccc9c8)ccc7c6)cc5)cc4)c4ccc5oc6ccccc6c5c4)cc32)cc1 ; c1ccc2c(c1)-c1ccccc1C21c2ccccc2-c2c(N(c3ccc(-c4ccc(-c5ccc6cc(-c7ccc8ccccc8c7)ccc6c5)cc4)cc3)c3ccc4oc5ccccc5c4c3)cccc21 ; c1ccc(C2(c3ccccc3)c3ccccc3-c3c(N(c4ccc(-c5ccc(-c6ccc7cc(-c8ccc9ccccc9c8)ccc7c6)cc5)cc4)c4ccc5oc6ccccc6c5c4)cccc32)cc1]

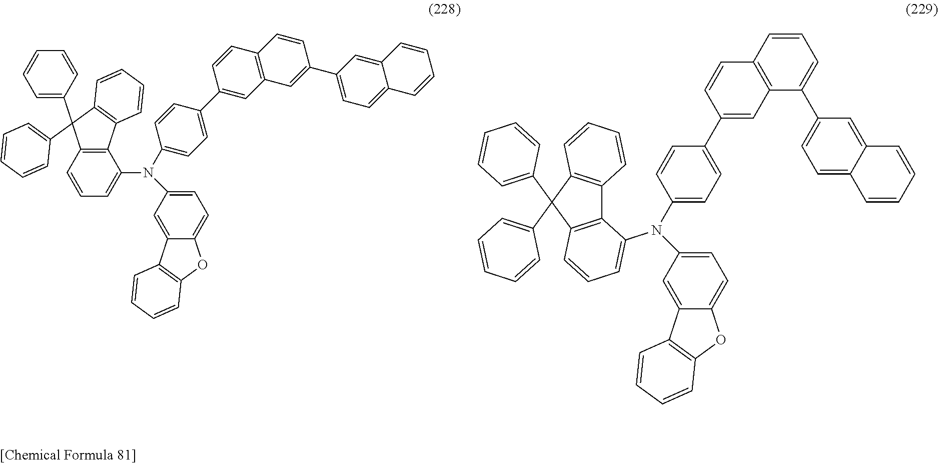 [SMILES: c1ccc(C2(c3ccccc3)c3ccccc3-c3c(N(c4ccc(-c5ccc6ccc(-c7ccc8ccccc8c7)cc6c5)cc4)c4ccc5oc6ccccc6c5c4)cccc32)cc1 ; c1ccc(C2(c3ccccc3)c3ccccc3-c3c(N(c4ccc(-c5ccc6cccc(-c7ccc8ccccc8c7)c6c5)cc4)c4ccc5oc6ccccc6c5c4)cccc32)cc1]

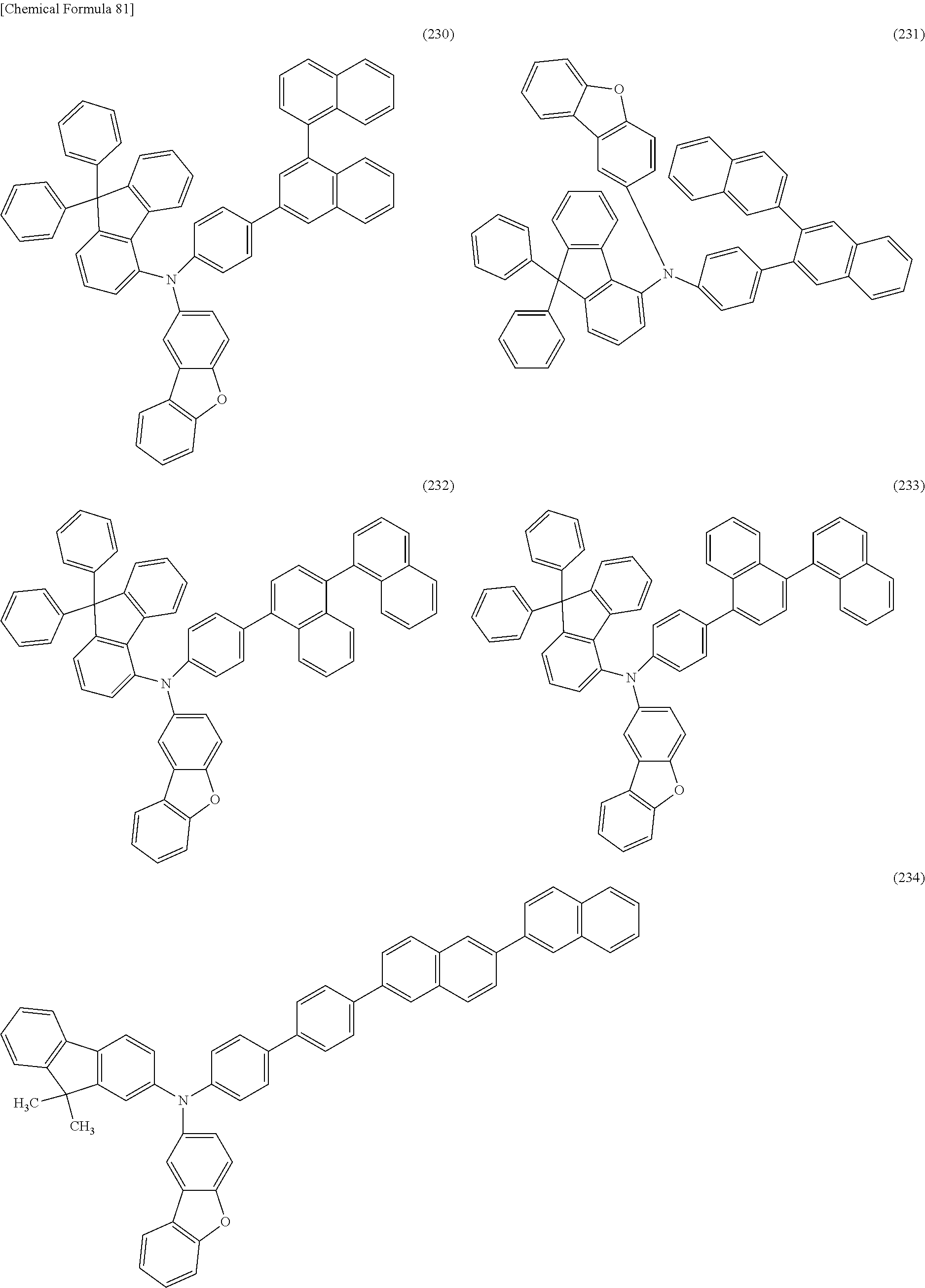

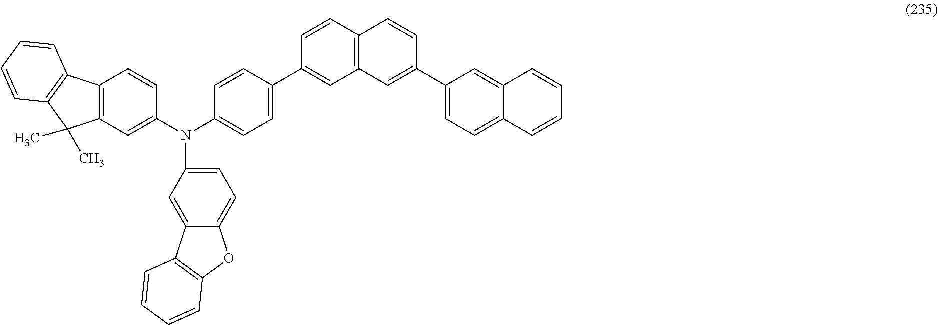 [SMILES: CC1(C)c2ccccc2-c2ccc(N(c3ccc(-c4ccc5ccc(-c6ccc7ccccc7c6)cc5c4)cc3)c3ccc4oc5ccccc5c4c3)cc21]

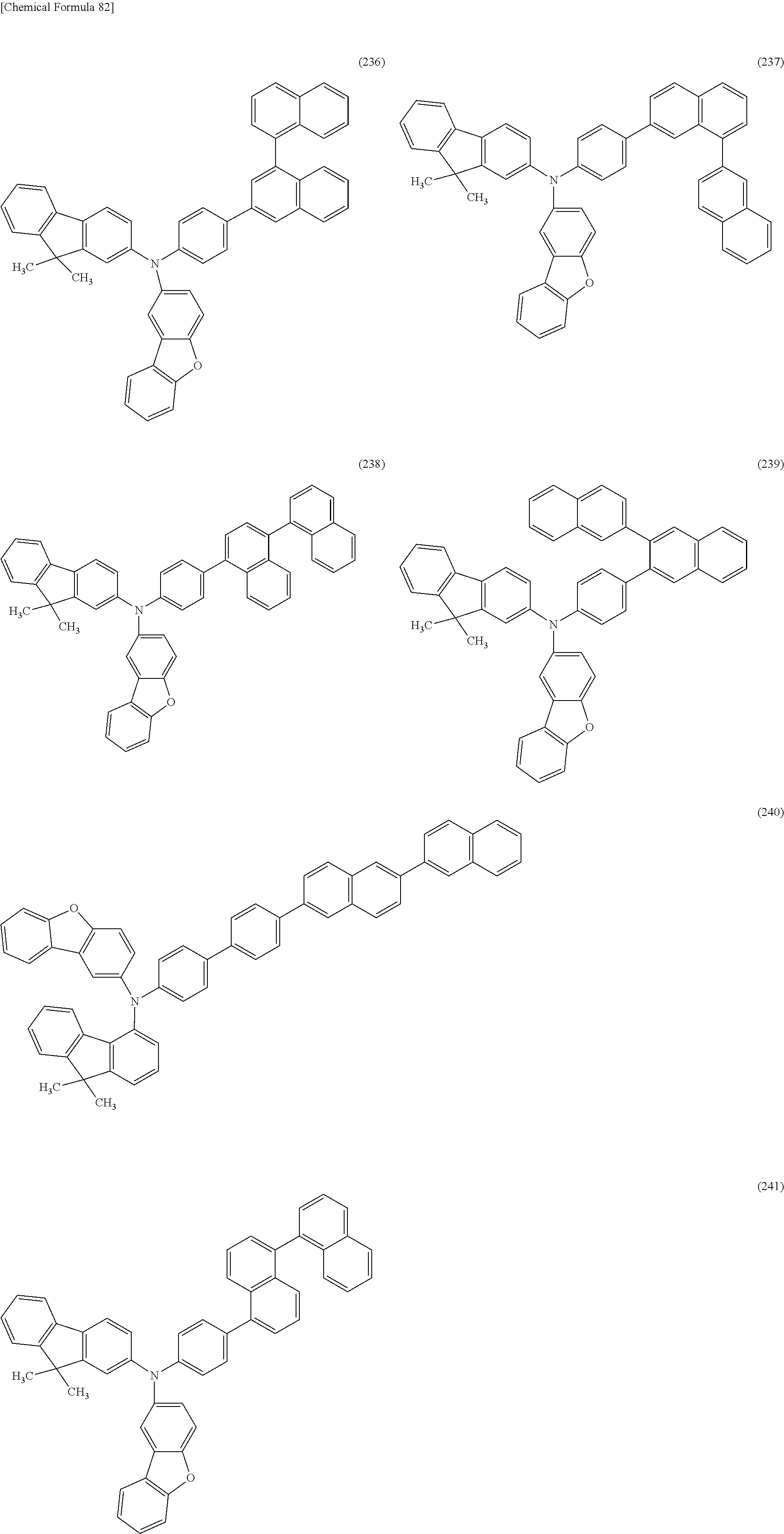

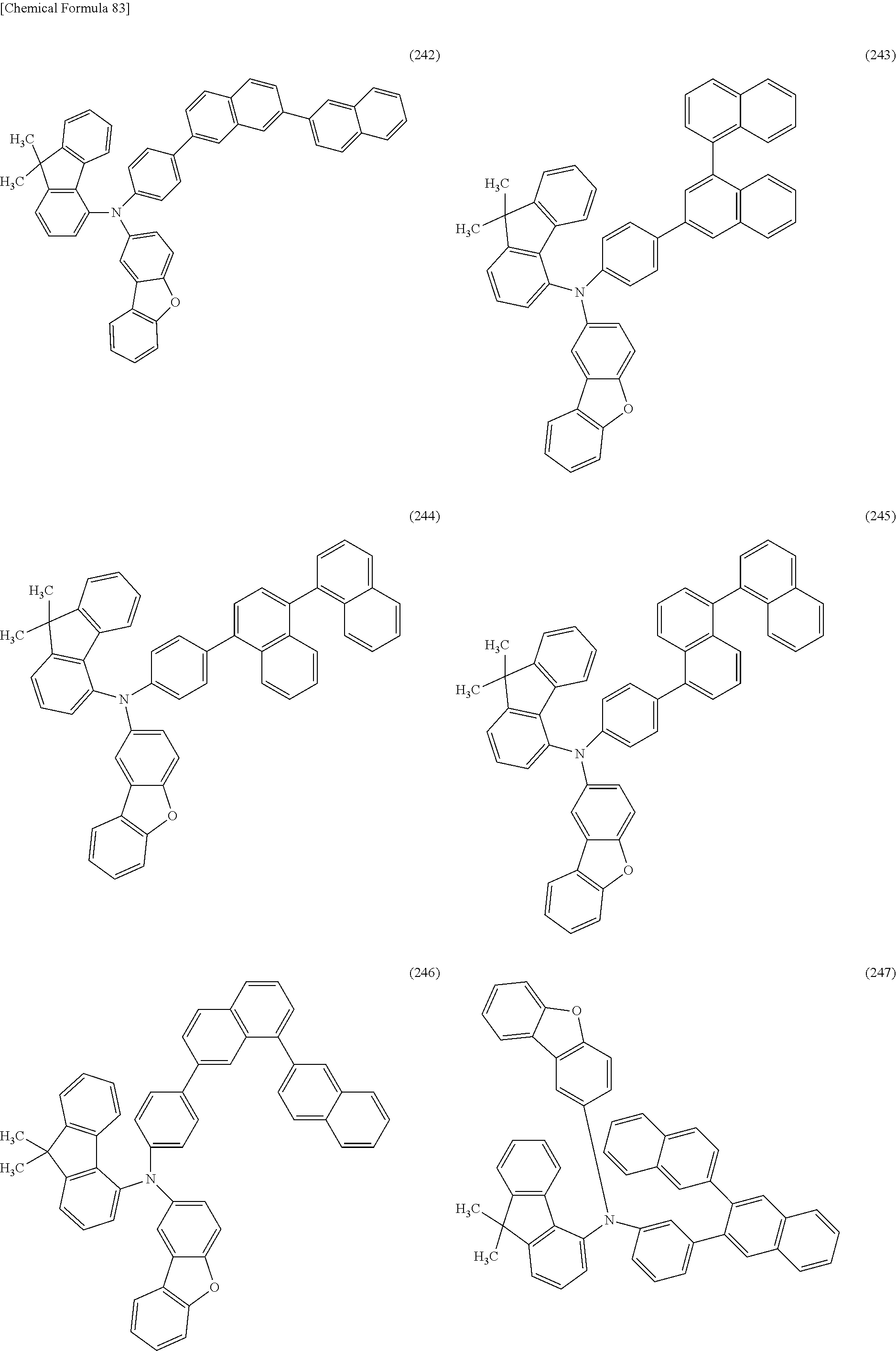

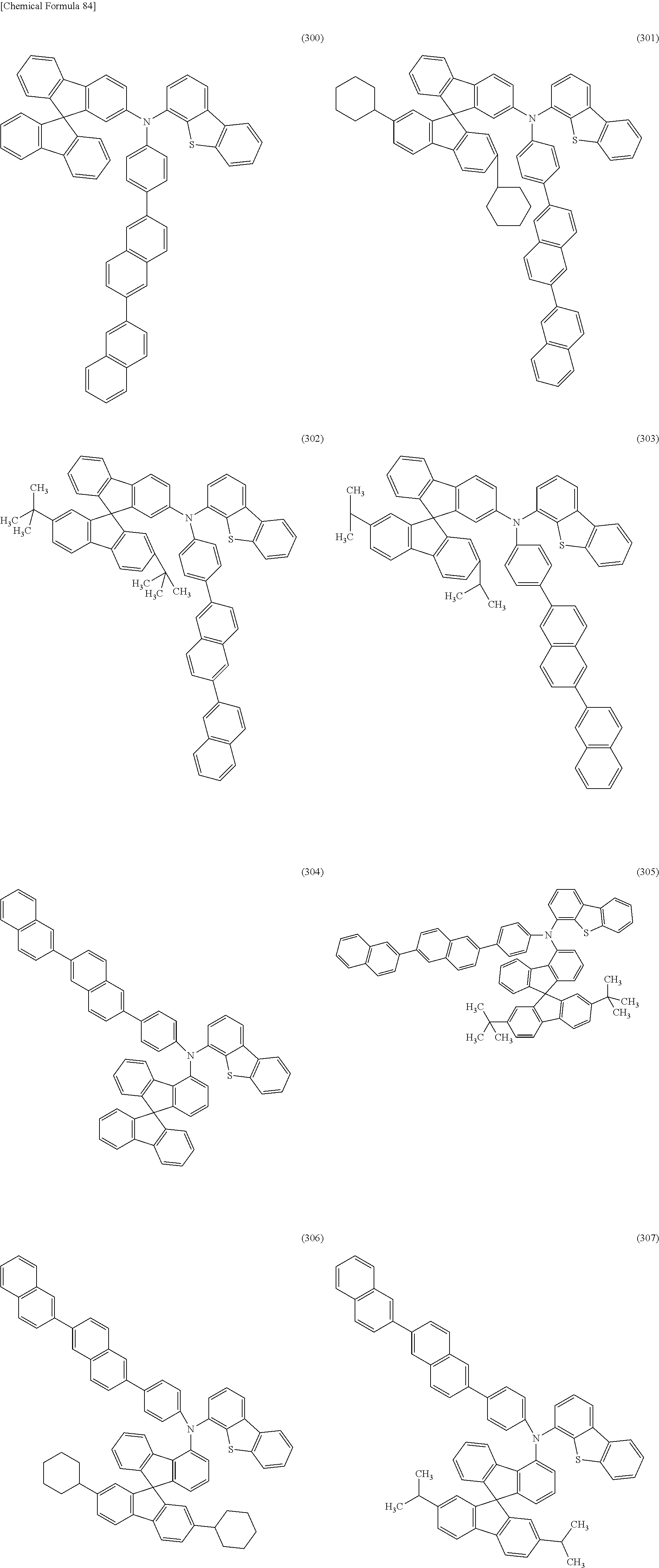

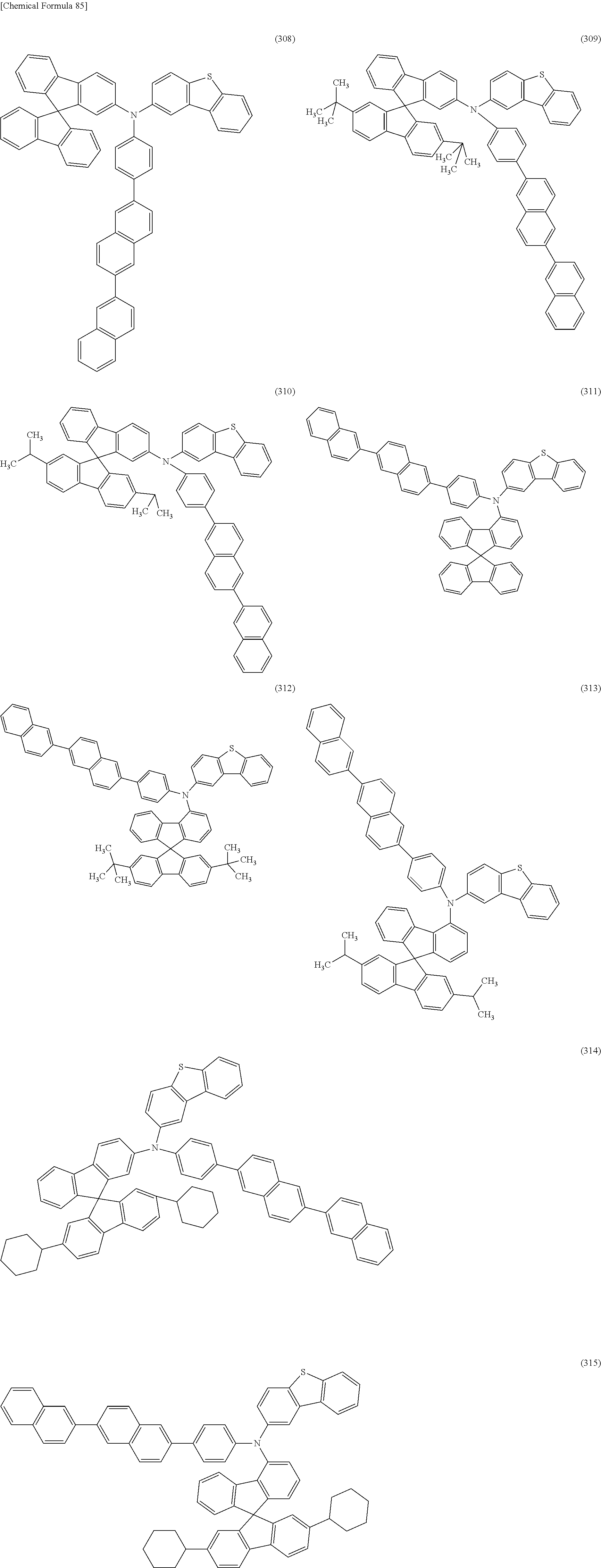

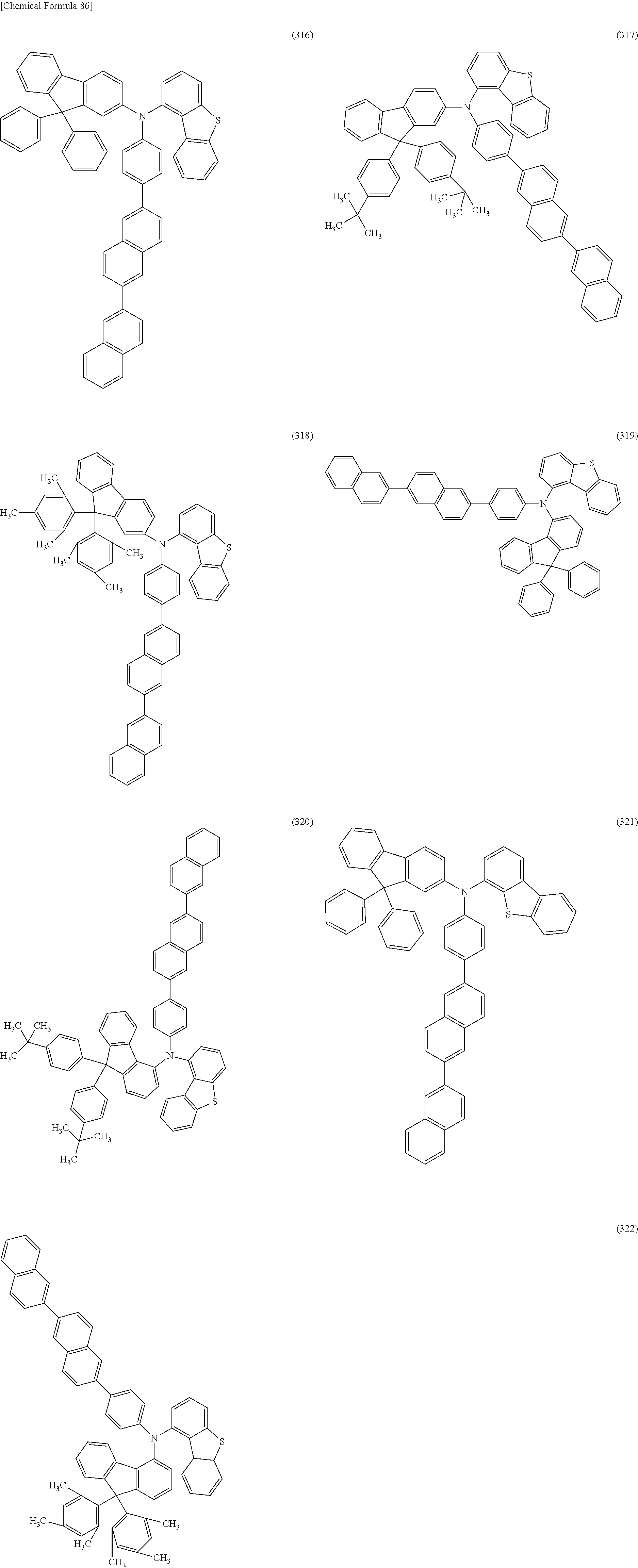

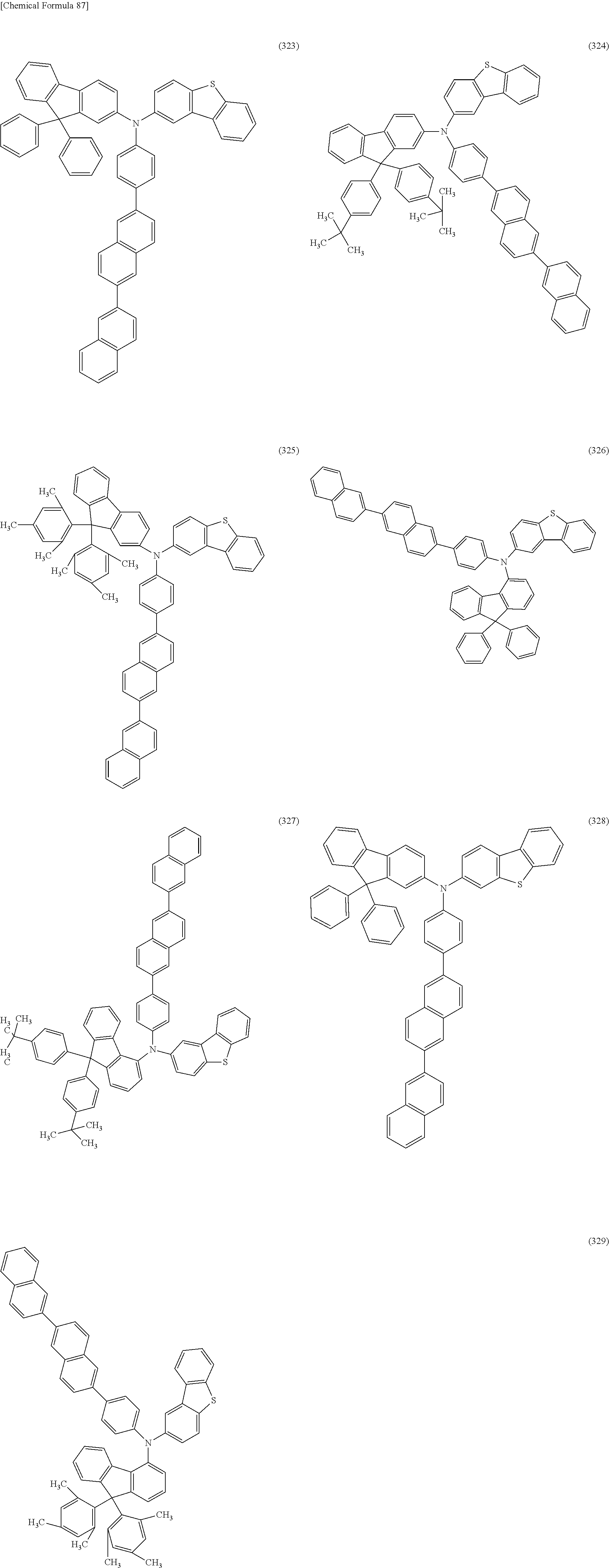

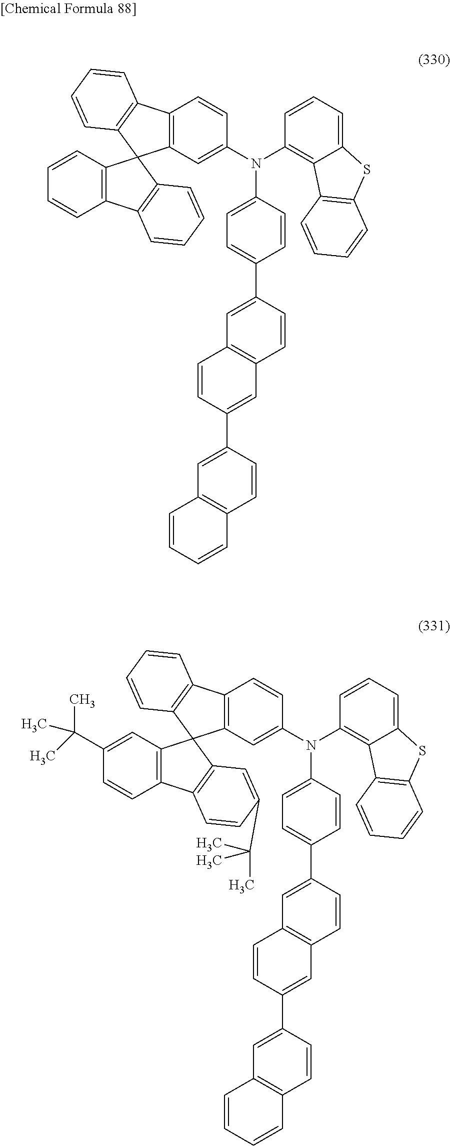 [SMILES: c1ccc2c(c1)-c1ccccc1C21c2ccccc2-c2ccc(N(c3ccc(-c4ccc5cc(-c6ccc7ccccc7c6)ccc5c4)cc3)c3cccc4sc5ccccc5c34)cc21 ; CC(C)(C)c1ccc2c(c1)C1(c3ccccc3-c3ccc(N(c4ccc(-c5ccc6cc(-c7ccc8ccccc8c7)ccc6c5)cc4)c4cccc5sc6ccccc6c45)cc31)c1cc(C(C)(C)C)ccc1-2]

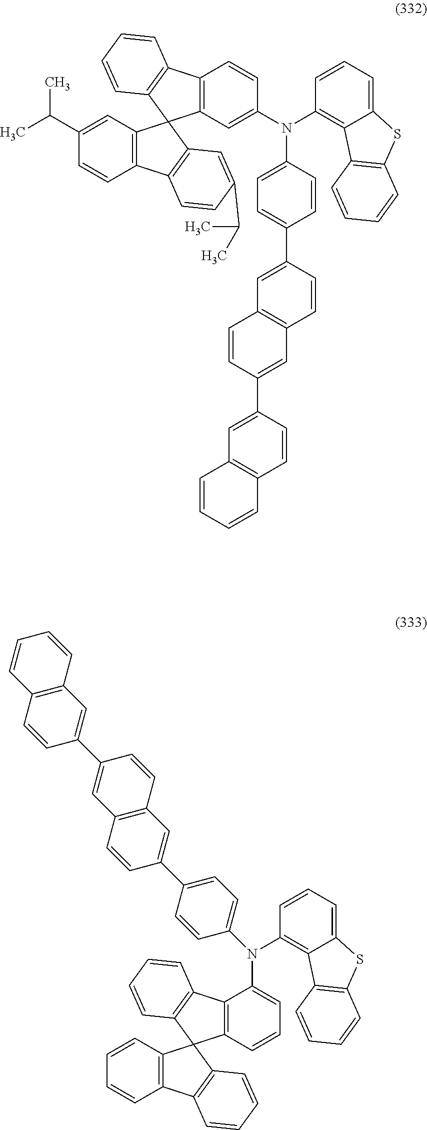 [SMILES: CC(C)c1ccc2c(c1)C1(c3ccccc3-c3ccc(N(c4ccc(-c5ccc6cc(-c7ccc8ccccc8c7)ccc6c5)cc4)c4cccc5sc6ccccc6c45)cc31)c1cc(C(C)C)ccc1-2 ; c1ccc2c(c1)-c1ccccc1C21c2ccccc2-c2c(N(c3ccc(-c4ccc5cc(-c6ccc7ccccc7c6)ccc5c4)cc3)c3cccc4sc5ccccc5c34)cccc21]

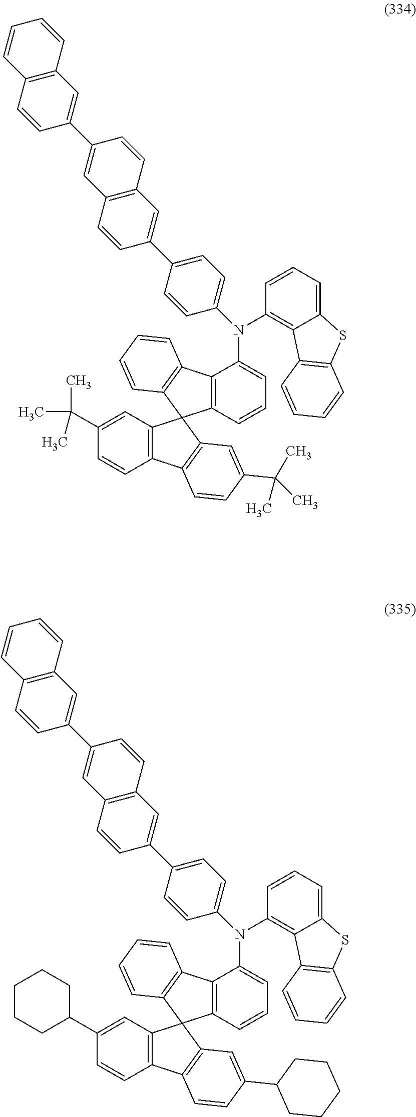 [SMILES: CC(C)(C)c1ccc2c(c1)C1(c3cc(C(C)(C)C)ccc3-2)c2ccccc2-c2c(N(c3ccc(-c4ccc5cc(-c6ccc7ccccc7c6)ccc5c4)cc3)c3cccc4sc5ccccc5c34)cccc21 ; c1ccc2c(c1)-c1c(N(c3ccc(-c4ccc5cc(-c6ccc7ccccc7c6)ccc5c4)cc3)c3cccc4sc5ccccc5c34)cccc1C21c2cc(C3CCCCC3)ccc2-c2ccc(C3CCCCC3)cc21]

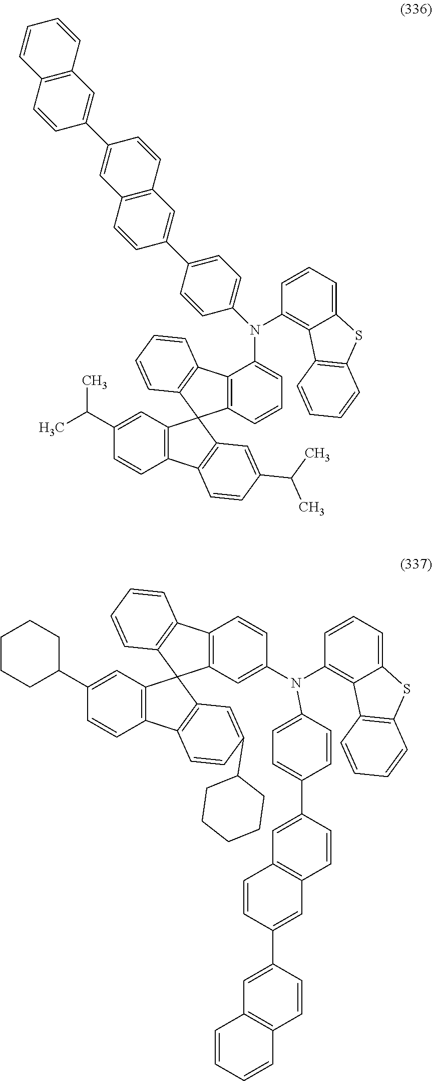 [SMILES: CC(C)c1ccc2c(c1)C1(c3cc(C(C)C)ccc3-2)c2ccccc2-c2c(N(c3ccc(-c4ccc5cc(-c6ccc7ccccc7c6)ccc5c4)cc3)c3cccc4sc5ccccc5c34)cccc21 ; c1ccc2c(c1)-c1ccc(N(c3ccc(-c4ccc5cc(-c6ccc7ccccc7c6)ccc5c4)cc3)c3cccc4sc5ccccc5c34)cc1C21c2cc(C3CCCCC3)ccc2-c2ccc(C3CCCCC3)cc21]

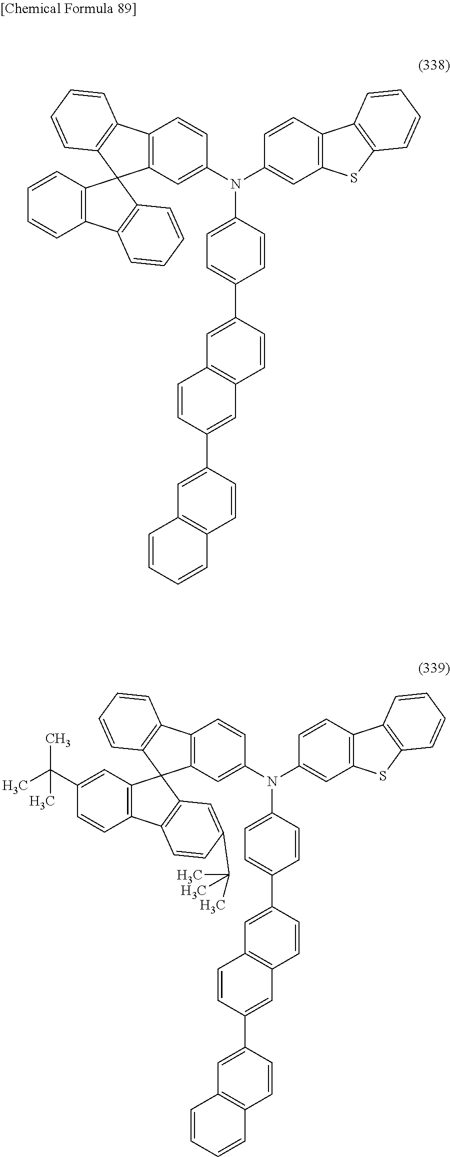 [SMILES: c1ccc2c(c1)-c1ccccc1C21c2ccccc2-c2ccc(N(c3ccc(-c4ccc5cc(-c6ccc7ccccc7c6)ccc5c4)cc3)c3ccc4c(c3)sc3ccccc34)cc21 ; CC(C)(C)c1ccc2c(c1)C1(c3ccccc3-c3ccc(N(c4ccc(-c5ccc6cc(-c7ccc8ccccc8c7)ccc6c5)cc4)c4ccc5c(c4)sc4ccccc45)cc31)c1cc(C(C)(C)C)ccc1-2]

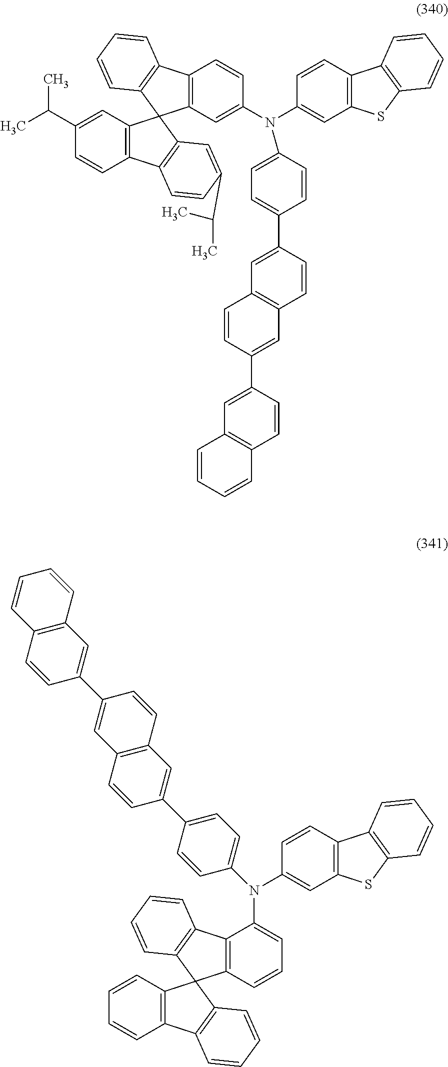 [SMILES: CC(C)c1ccc2c(c1)C1(c3ccccc3-c3ccc(N(c4ccc(-c5ccc6cc(-c7ccc8ccccc8c7)ccc6c5)cc4)c4ccc5c(c4)sc4ccccc45)cc31)c1cc(C(C)C)ccc1-2 ; c1ccc2c(c1)-c1ccccc1C21c2ccccc2-c2c(N(c3ccc(-c4ccc5cc(-c6ccc7ccccc7c6)ccc5c4)cc3)c3ccc4c(c3)sc3ccccc34)cccc21]

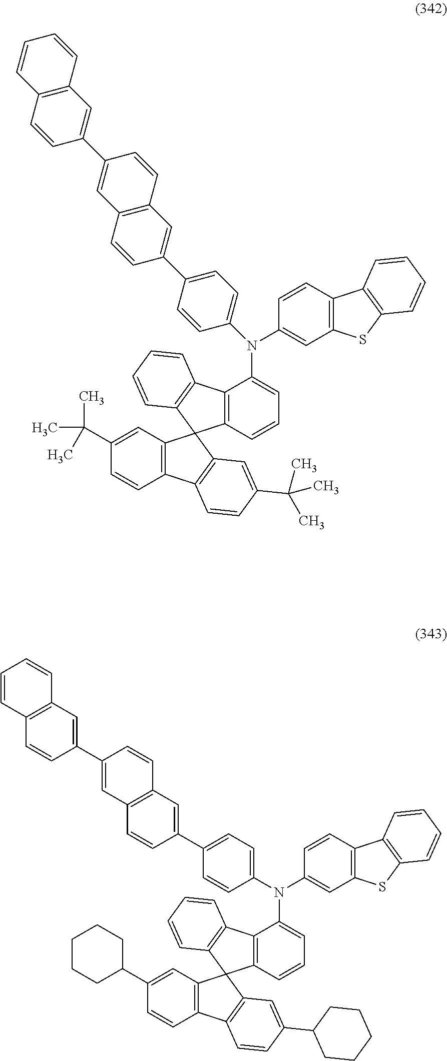 [SMILES: CC(C)(C)c1ccc2c(c1)C1(c3cc(C(C)(C)C)ccc3-2)c2ccccc2-c2c(N(c3ccc(-c4ccc5cc(-c6ccc7ccccc7c6)ccc5c4)cc3)c3ccc4c(c3)sc3ccccc34)cccc21 ; c1ccc2c(c1)-c1c(N(c3ccc(-c4ccc5cc(-c6ccc7ccccc7c6)ccc5c4)cc3)c3ccc4c(c3)sc3ccccc34)cccc1C21c2cc(C3CCCCC3)ccc2-c2ccc(C3CCCCC3)cc21]

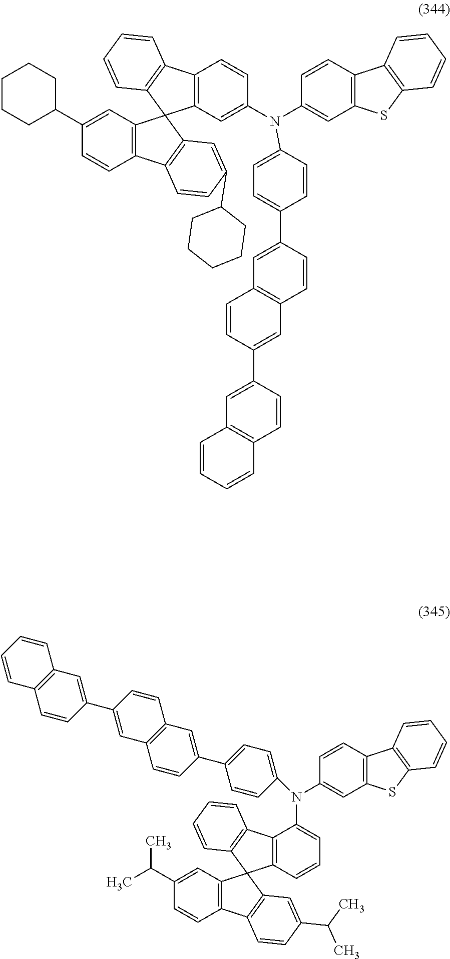 [SMILES: c1ccc2c(c1)-c1ccc(N(c3ccc(-c4ccc5cc(-c6ccc7ccccc7c6)ccc5c4)cc3)c3ccc4c(c3)sc3ccccc34)cc1C21c2cc(C3CCCCC3)ccc2-c2ccc(C3CCCCC3)cc21 ; CC(C)c1ccc2c(c1)C1(c3cc(C(C)C)ccc3-2)c2ccccc2-c2c(N(c3ccc(-c4ccc5cc(-c6ccc7ccccc7c6)ccc5c4)cc3)c3ccc4c(c3)sc3ccccc34)cccc21]

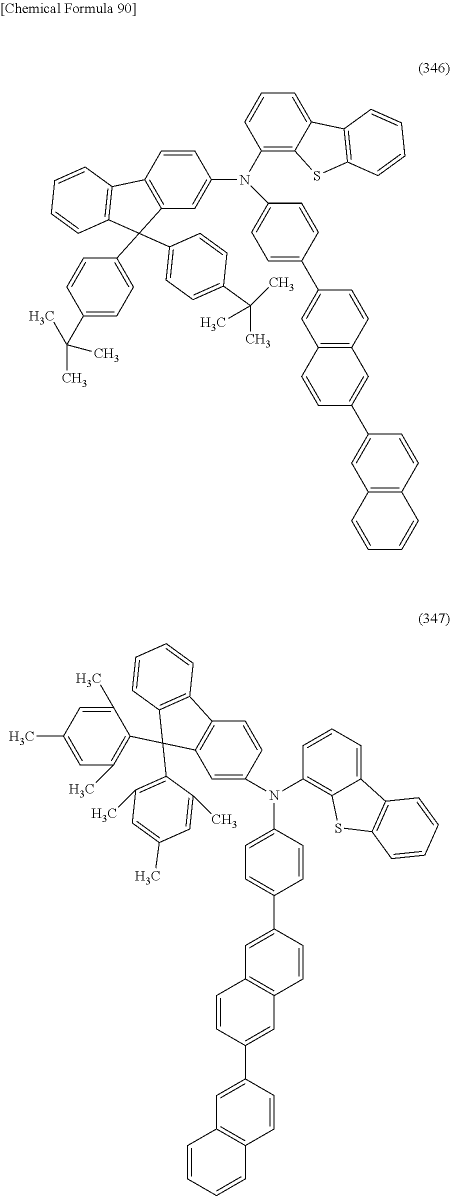 [SMILES: CC(C)(C)c1ccc(C2(c3ccc(C(C)(C)C)cc3)c3ccccc3-c3ccc(N(c4ccc(-c5ccc6cc(-c7ccc8ccccc8c7)ccc6c5)cc4)c4cccc5c4sc4ccccc45)cc32)cc1 ; Cc1cc(C)c(C2(c3c(C)cc(C)cc3C)c3ccccc3-c3ccc(N(c4ccc(-c5ccc6cc(-c7ccc8ccccc8c7)ccc6c5)cc4)c4cccc5c4sc4ccccc45)cc32)c(C)c1]

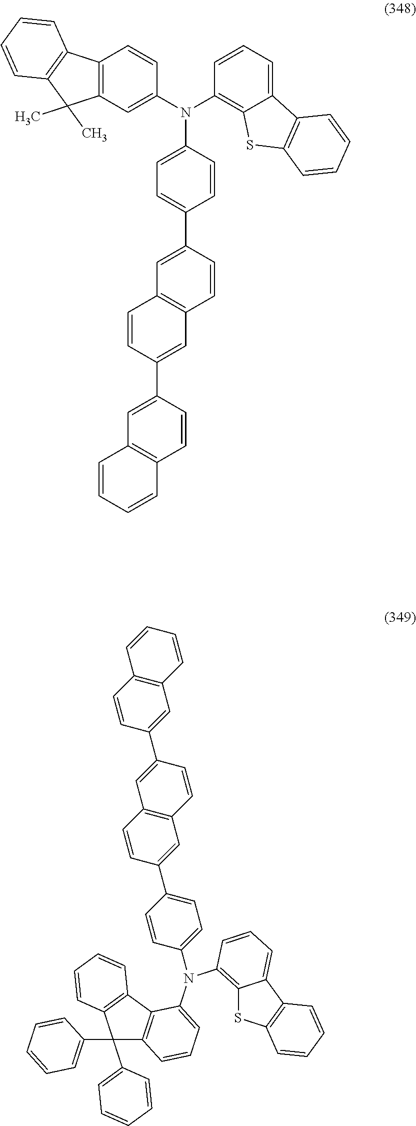 [SMILES: CC1(C)c2ccccc2-c2ccc(N(c3ccc(-c4ccc5cc(-c6ccc7ccccc7c6)ccc5c4)cc3)c3cccc4c3sc3ccccc34)cc21 ; c1ccc(C2(c3ccccc3)c3ccccc3-c3c(N(c4ccc(-c5ccc6cc(-c7ccc8ccccc8c7)ccc6c5)cc4)c4cccc5c4sc4ccccc45)cccc32)cc1]

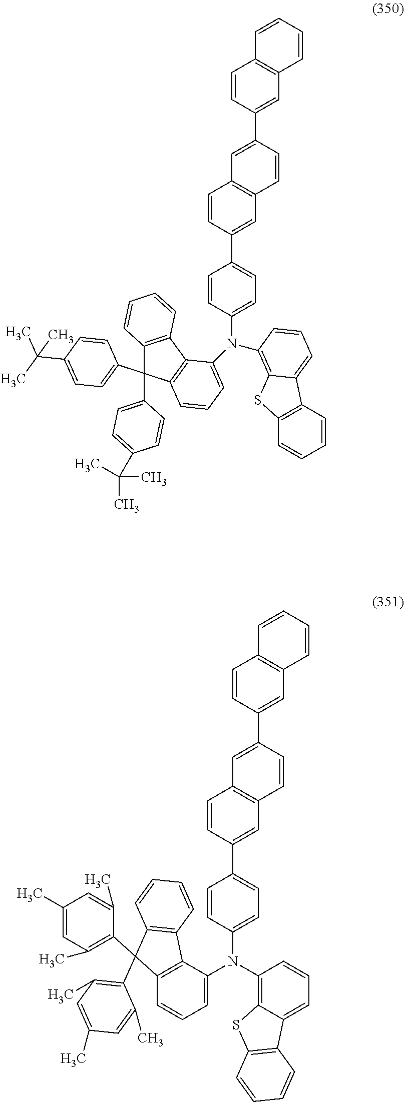 [SMILES: CC(C)(C)c1ccc(C2(c3ccc(C(C)(C)C)cc3)c3ccccc3-c3c(N(c4ccc(-c5ccc6cc(-c7ccc8ccccc8c7)ccc6c5)cc4)c4cccc5c4sc4ccccc45)cccc32)cc1 ; Cc1cc(C)c(C2(c3c(C)cc(C)cc3C)c3ccccc3-c3c(N(c4ccc(-c5ccc6cc(-c7ccc8ccccc8c7)ccc6c5)cc4)c4cccc5c4sc4ccccc45)cccc32)c(C)c1]

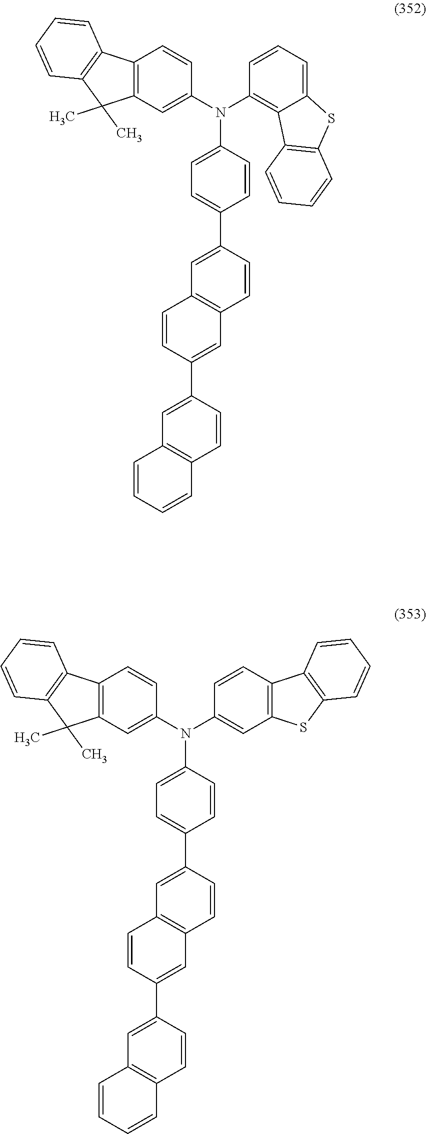 [SMILES: CC1(C)c2ccccc2-c2ccc(N(c3ccc(-c4ccc5cc(-c6ccc7ccccc7c6)ccc5c4)cc3)c3cccc4sc5ccccc5c34)cc21 ; CC1(C)c2ccccc2-c2ccc(N(c3ccc(-c4ccc5cc(-c6ccc7ccccc7c6)ccc5c4)cc3)c3ccc4c(c3)sc3ccccc34)cc21]

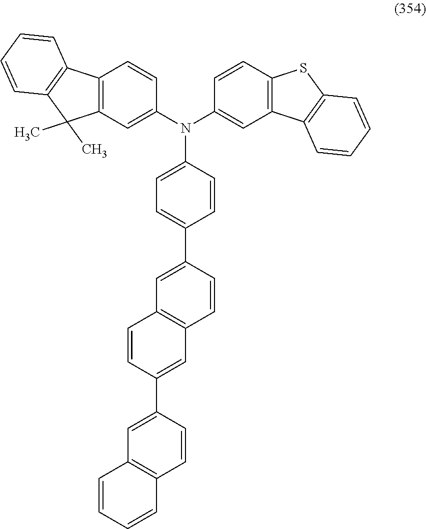 [SMILES: CC1(C)c2ccccc2-c2ccc(N(c3ccc(-c4ccc5cc(-c6ccc7ccccc7c6)ccc5c4)cc3)c3ccc4sc5ccccc5c4c3)cc21]

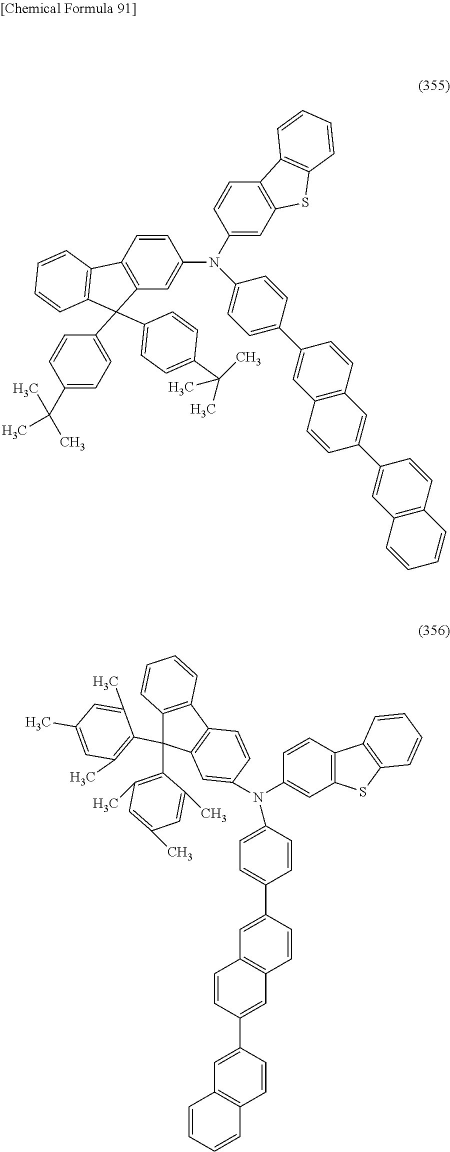 [SMILES: CC(C)(C)c1ccc(C2(c3ccc(C(C)(C)C)cc3)c3ccccc3-c3ccc(N(c4ccc(-c5ccc6cc(-c7ccc8ccccc8c7)ccc6c5)cc4)c4ccc5c(c4)sc4ccccc45)cc32)cc1 ; Cc1cc(C)c(C2(c3c(C)cc(C)cc3C)c3ccccc3-c3ccc(N(c4ccc(-c5ccc6cc(-c7ccc8ccccc8c7)ccc6c5)cc4)c4ccc5c(c4)sc4ccccc45)cc32)c(C)c1]

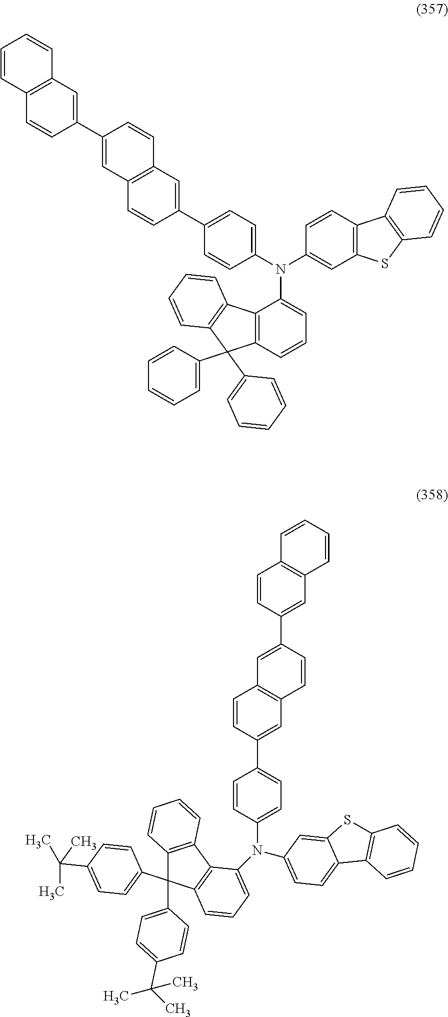 [SMILES: c1ccc(C2(c3ccccc3)c3ccccc3-c3c(N(c4ccc(-c5ccc6cc(-c7ccc8ccccc8c7)ccc6c5)cc4)c4ccc5c(c4)sc4ccccc45)cccc32)cc1 ; CC(C)(C)c1ccc(C2(c3ccc(C(C)(C)C)cc3)c3ccccc3-c3c(N(c4ccc(-c5ccc6cc(-c7ccc8ccccc8c7)ccc6c5)cc4)c4ccc5c(c4)sc4ccccc45)cccc32)cc1]

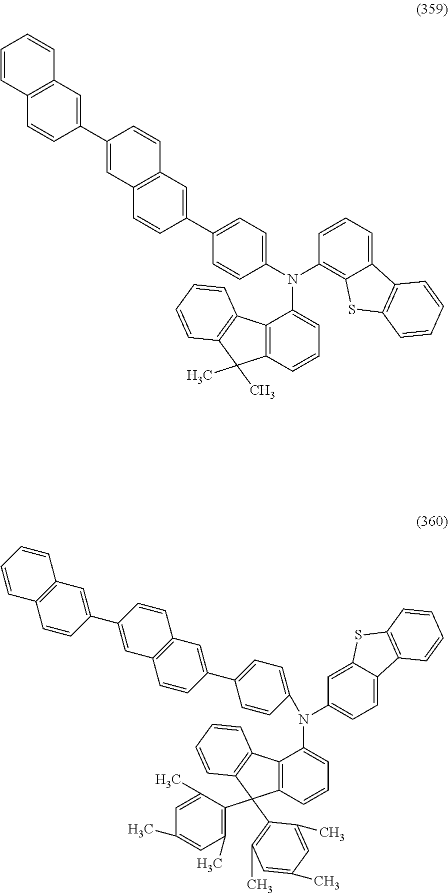 [SMILES: CC1(C)c2ccccc2-c2c(N(c3ccc(-c4ccc5cc(-c6ccc7ccccc7c6)ccc5c4)cc3)c3cccc4c3sc3ccccc34)cccc21 ; Cc1cc(C)c(C2(c3c(C)cc(C)cc3C)c3ccccc3-c3c(N(c4ccc(-c5ccc6cc(-c7ccc8ccccc8c7)ccc6c5)cc4)c4ccc5c(c4)sc4ccccc45)cccc32)c(C)c1]

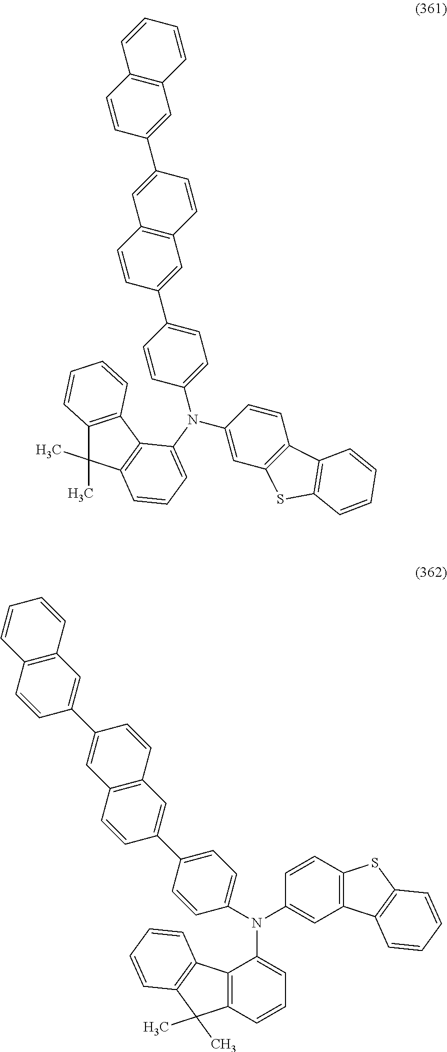 [SMILES: CC1(C)c2ccccc2-c2c(N(c3ccc(-c4ccc5cc(-c6ccc7ccccc7c6)ccc5c4)cc3)c3ccc4c(c3)sc3ccccc34)cccc21 ; CC1(C)c2ccccc2-c2c(N(c3ccc(-c4ccc5cc(-c6ccc7ccccc7c6)ccc5c4)cc3)c3ccc4sc5ccccc5c4c3)cccc21]

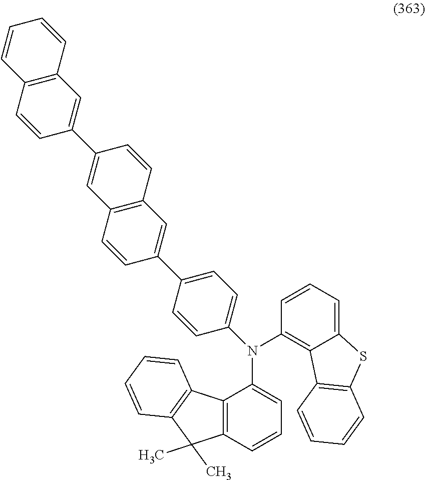 [SMILES: CC1(C)c2ccccc2-c2c(N(c3ccc(-c4ccc5cc(-c6ccc7ccccc7c6)ccc5c4)cc3)c3cccc4sc5ccccc5c34)cccc21]

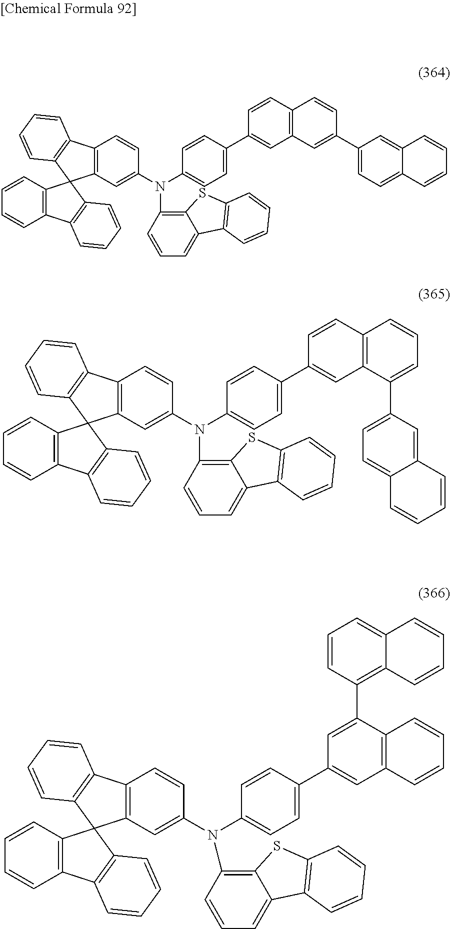 [SMILES: c1ccc2c(c1)-c1ccccc1C21c2ccccc2-c2ccc(N(c3ccc(-c4ccc5ccc(-c6ccc7ccccc7c6)cc5c4)cc3)c3cccc4c3sc3ccccc34)cc21 ; c1ccc2c(c1)-c1ccccc1C21c2ccccc2-c2ccc(N(c3ccc(-c4ccc5cccc(-c6ccc7ccccc7c6)c5c4)cc3)c3cccc4c3sc3ccccc34)cc21 ; c1ccc2c(c1)-c1ccccc1C21c2ccccc2-c2ccc(N(c3ccc(-c4cc(-c5cccc6ccccc56)c5ccccc5c4)cc3)c3cccc4c3sc3ccccc34)cc21]

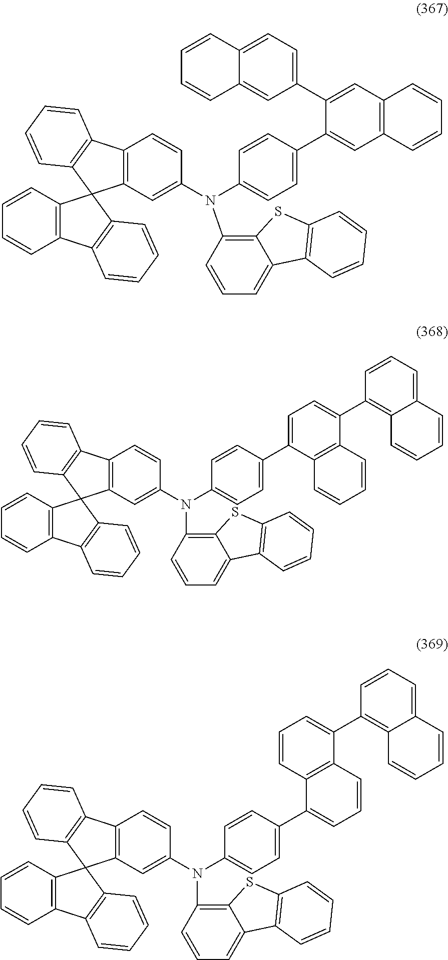 [SMILES: c1ccc2c(c1)-c1ccccc1C21c2ccccc2-c2ccc(N(c3ccc(-c4cc5ccccc5cc4-c4ccc5ccccc5c4)cc3)c3cccc4c3sc3ccccc34)cc21 ; c1ccc2c(c1)-c1ccccc1C21c2ccccc2-c2ccc(N(c3ccc(-c4ccc(-c5cccc6ccccc56)c5ccccc45)cc3)c3cccc4c3sc3ccccc34)cc21 ; c1ccc2c(c1)-c1ccccc1C21c2ccccc2-c2ccc(N(c3ccc(-c4cccc5c(-c6cccc7ccccc67)cccc45)cc3)c3cccc4c3sc3ccccc34)cc21]

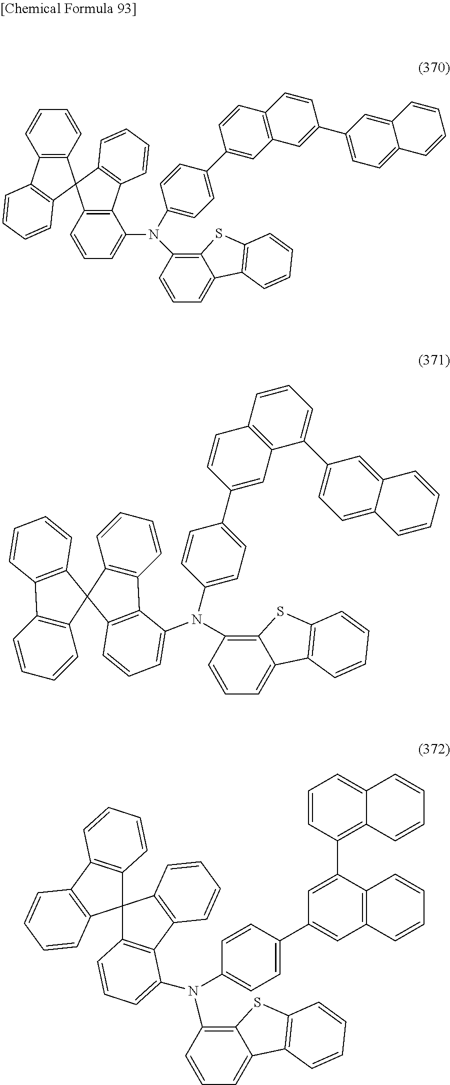 [SMILES: c1ccc2c(c1)-c1ccccc1C21c2ccccc2-c2c(N(c3ccc(-c4ccc5ccc(-c6ccc7ccccc7c6)cc5c4)cc3)c3cccc4c3sc3ccccc34)cccc21 ; c1ccc2c(c1)-c1ccccc1C21c2ccccc2-c2c(N(c3ccc(-c4ccc5cccc(-c6ccc7ccccc7c6)c5c4)cc3)c3cccc4c3sc3ccccc34)cccc21 ; c1ccc2c(c1)-c1ccccc1C21c2ccccc2-c2c(N(c3ccc(-c4cc(-c5cccc6ccccc56)c5ccccc5c4)cc3)c3cccc4c3sc3ccccc34)cccc21]

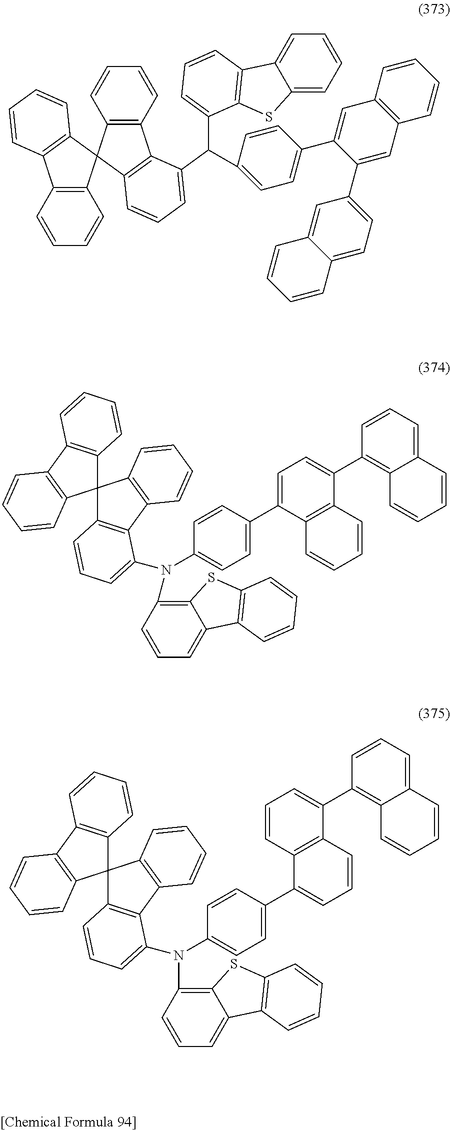 [SMILES: c1ccc2c(c1)-c1ccccc1C21c2ccccc2-c2c(C(c3ccc(-c4cc5ccccc5cc4-c4ccc5ccccc5c4)cc3)c3cccc4c3sc3ccccc34)cccc21 ; c1ccc2c(c1)-c1ccccc1C21c2ccccc2-c2c(N(c3ccc(-c4ccc(-c5cccc6ccccc56)c5ccccc45)cc3)c3cccc4c3sc3ccccc34)cccc21 ; c1ccc2c(c1)-c1ccccc1C21c2ccccc2-c2c(N(c3ccc(-c4cccc5c(-c6cccc7ccccc67)cccc45)cc3)c3cccc4c3sc3ccccc34)cccc21]

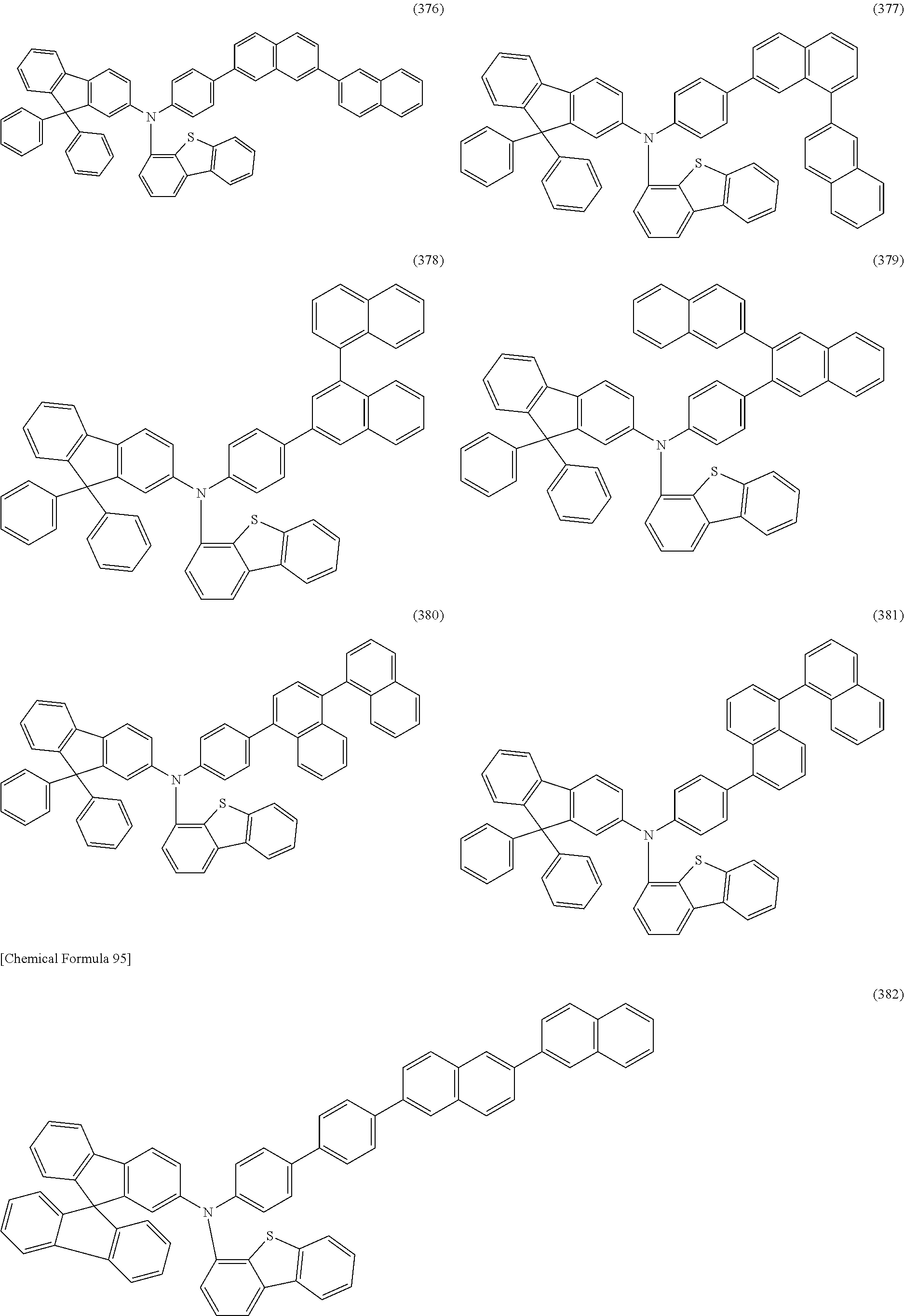

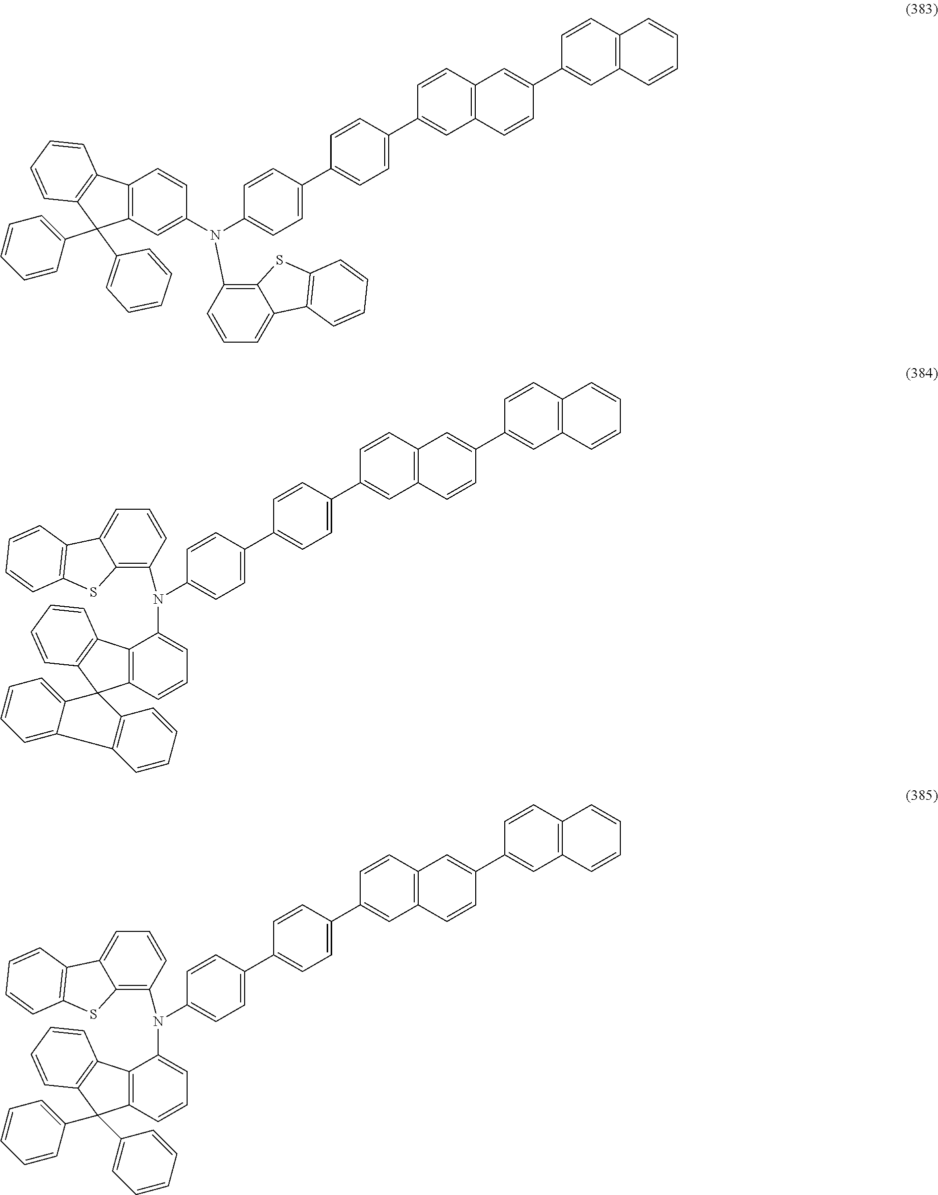 [SMILES: c1ccc(C2(c3ccccc3)c3ccccc3-c3ccc(N(c4ccc(-c5ccc(-c6ccc7cc(-c8ccc9ccccc9c8)ccc7c6)cc5)cc4)c4cccc5c4sc4ccccc45)cc32)cc1 ; c1ccc2c(c1)-c1ccccc1C21c2ccccc2-c2c(N(c3ccc(-c4ccc(-c5ccc6cc(-c7ccc8ccccc8c7)ccc6c5)cc4)cc3)c3cccc4c3sc3ccccc34)cccc21 ; c1ccc(C2(c3ccccc3)c3ccccc3-c3c(N(c4ccc(-c5ccc(-c6ccc7cc(-c8ccc9ccccc9c8)ccc7c6)cc5)cc4)c4cccc5c4sc4ccccc45)cccc32)cc1]

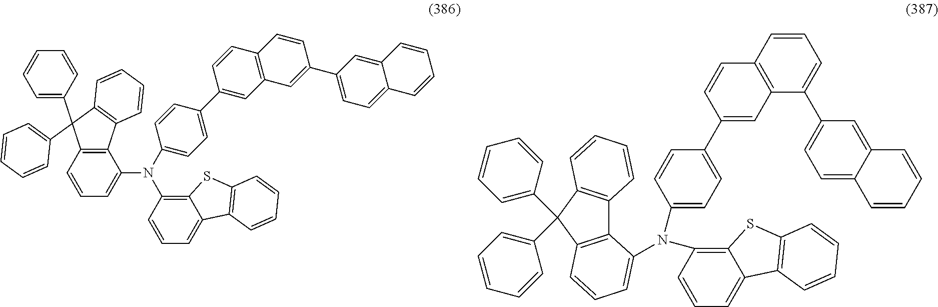 [SMILES: c1ccc(C2(c3ccccc3)c3ccccc3-c3c(N(c4ccc(-c5ccc6ccc(-c7ccc8ccccc8c7)cc6c5)cc4)c4cccc5c4sc4ccccc45)cccc32)cc1 ; c1ccc(C2(c3ccccc3)c3ccccc3-c3c(N(c4ccc(-c5ccc6cccc(-c7ccc8ccccc8c7)c6c5)cc4)c4cccc5c4sc4ccccc45)cccc32)cc1]

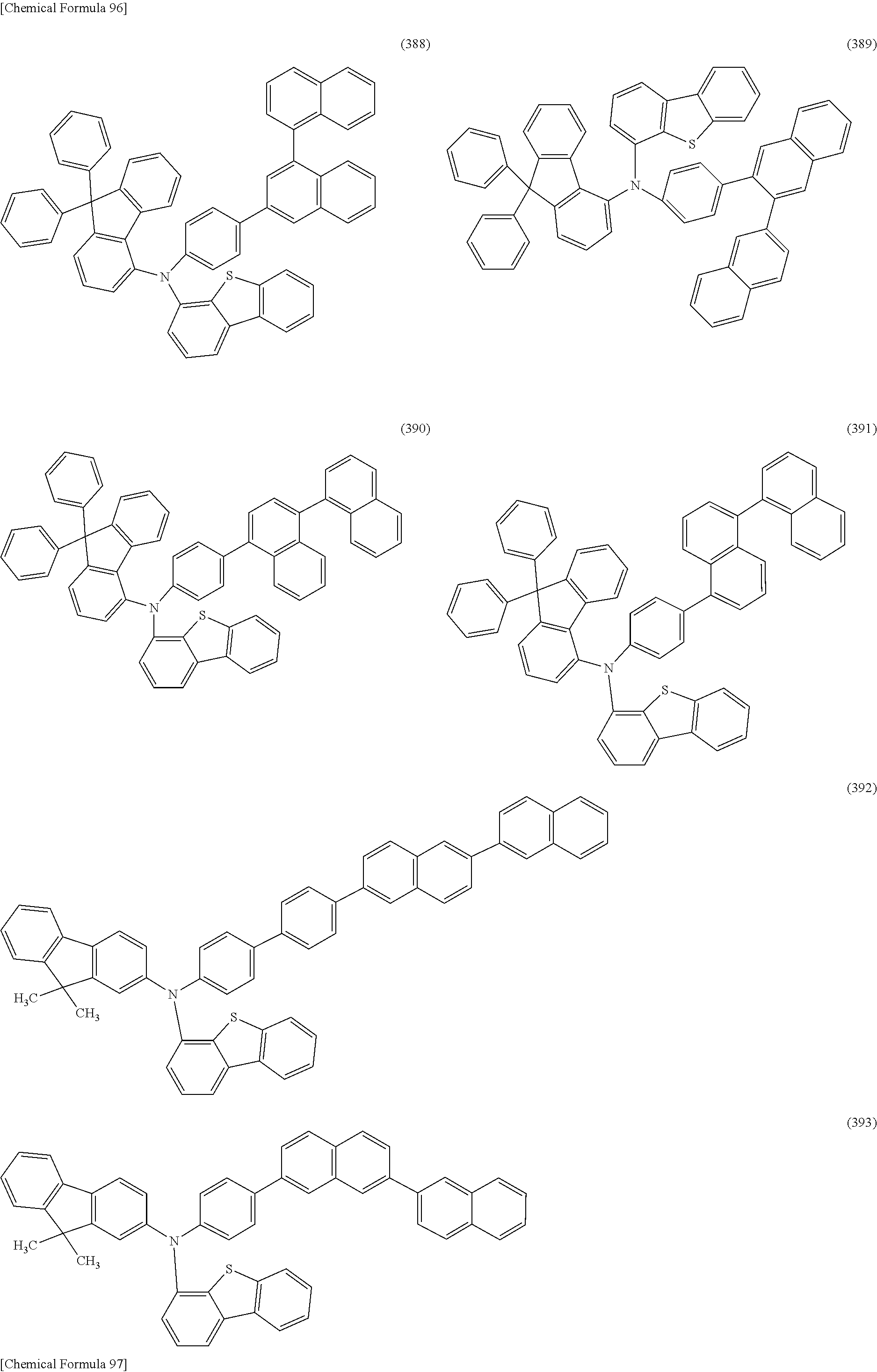

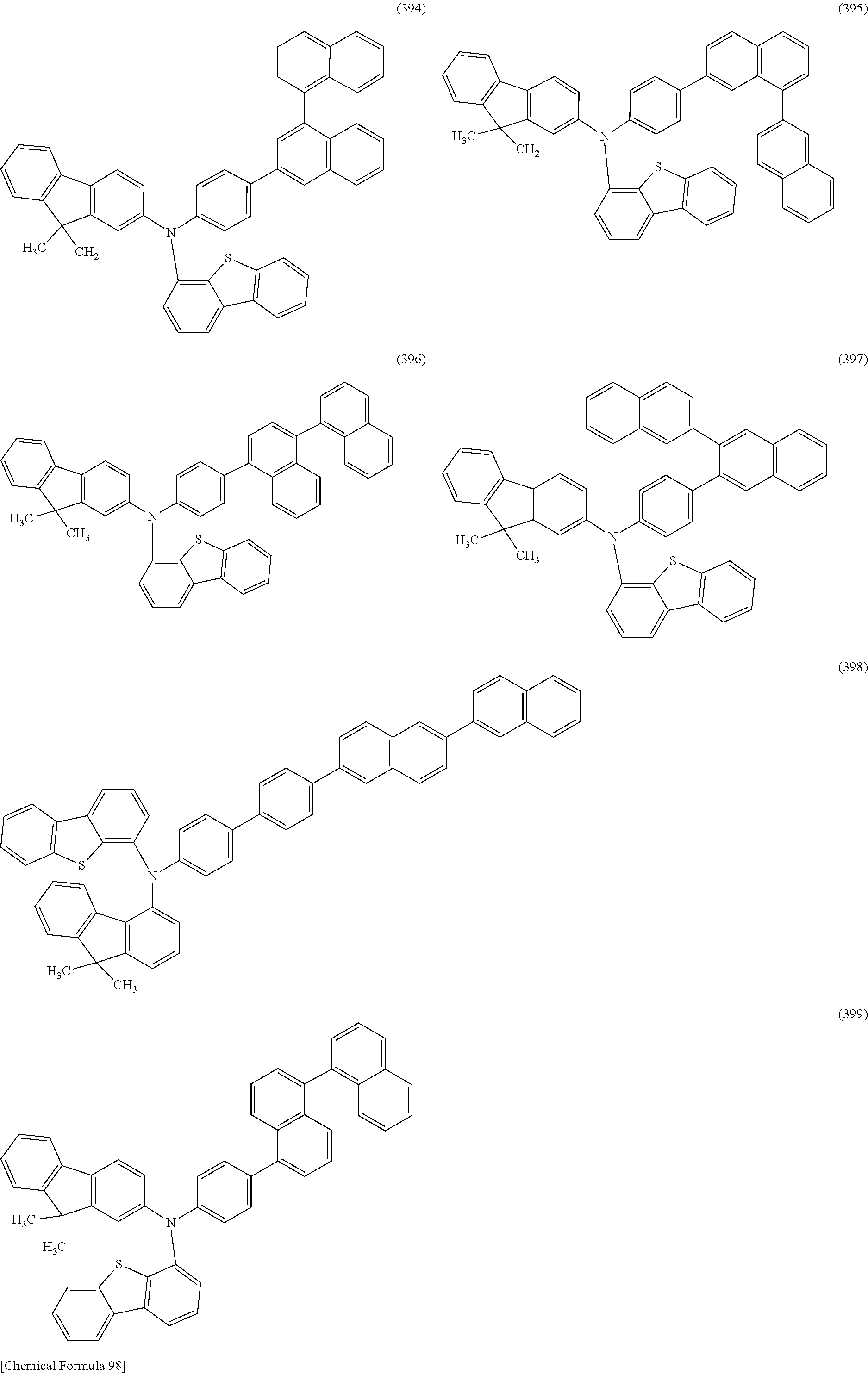

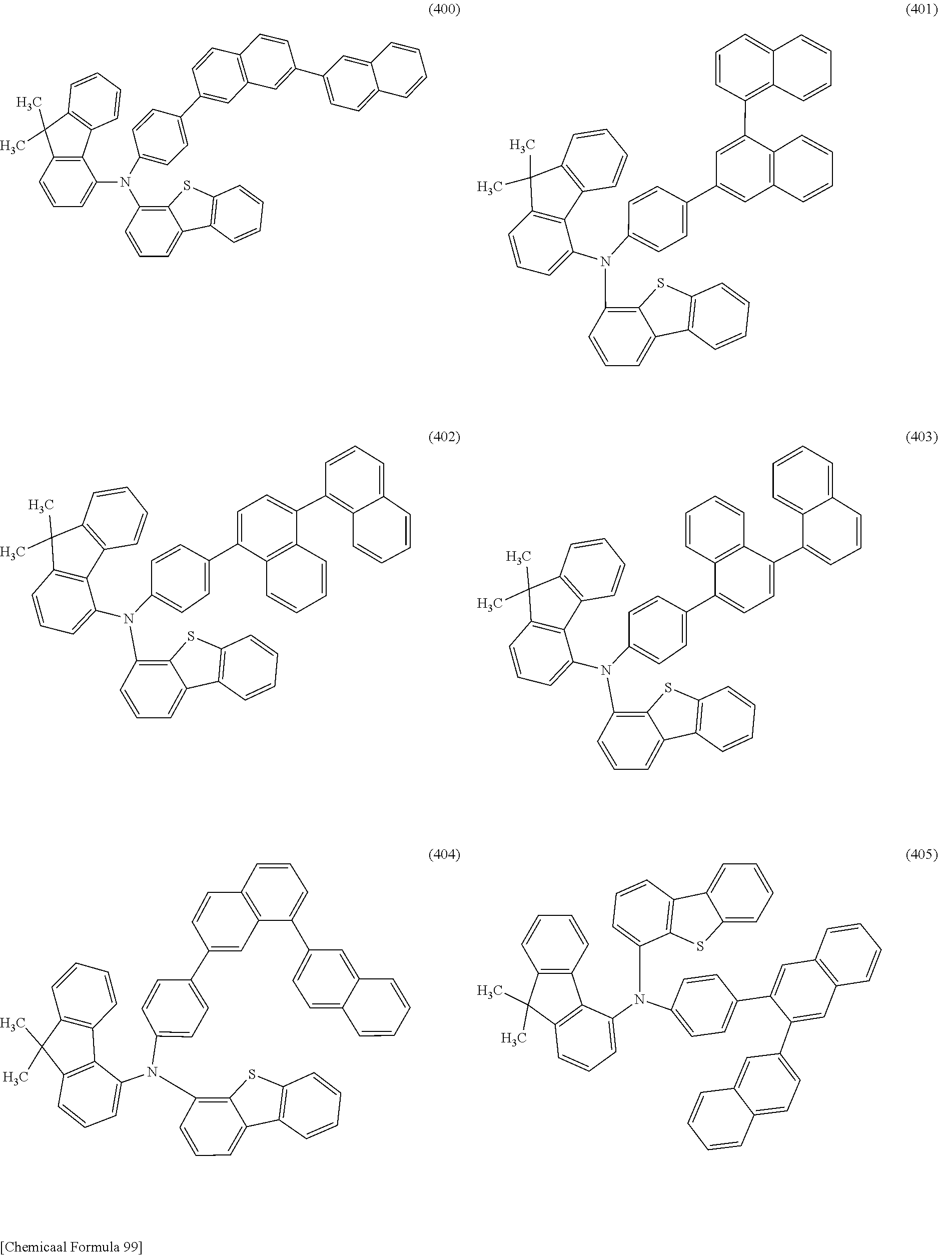

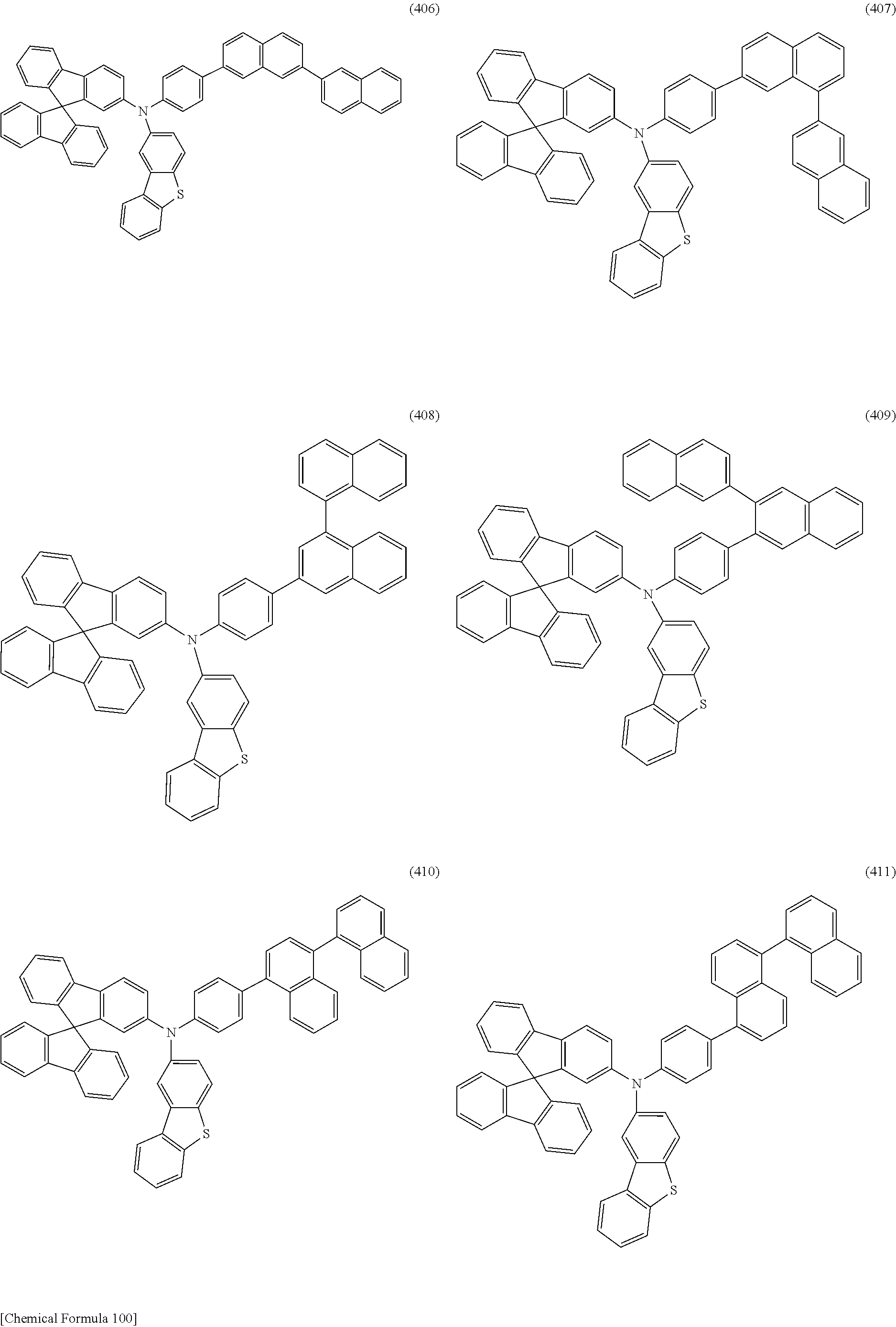

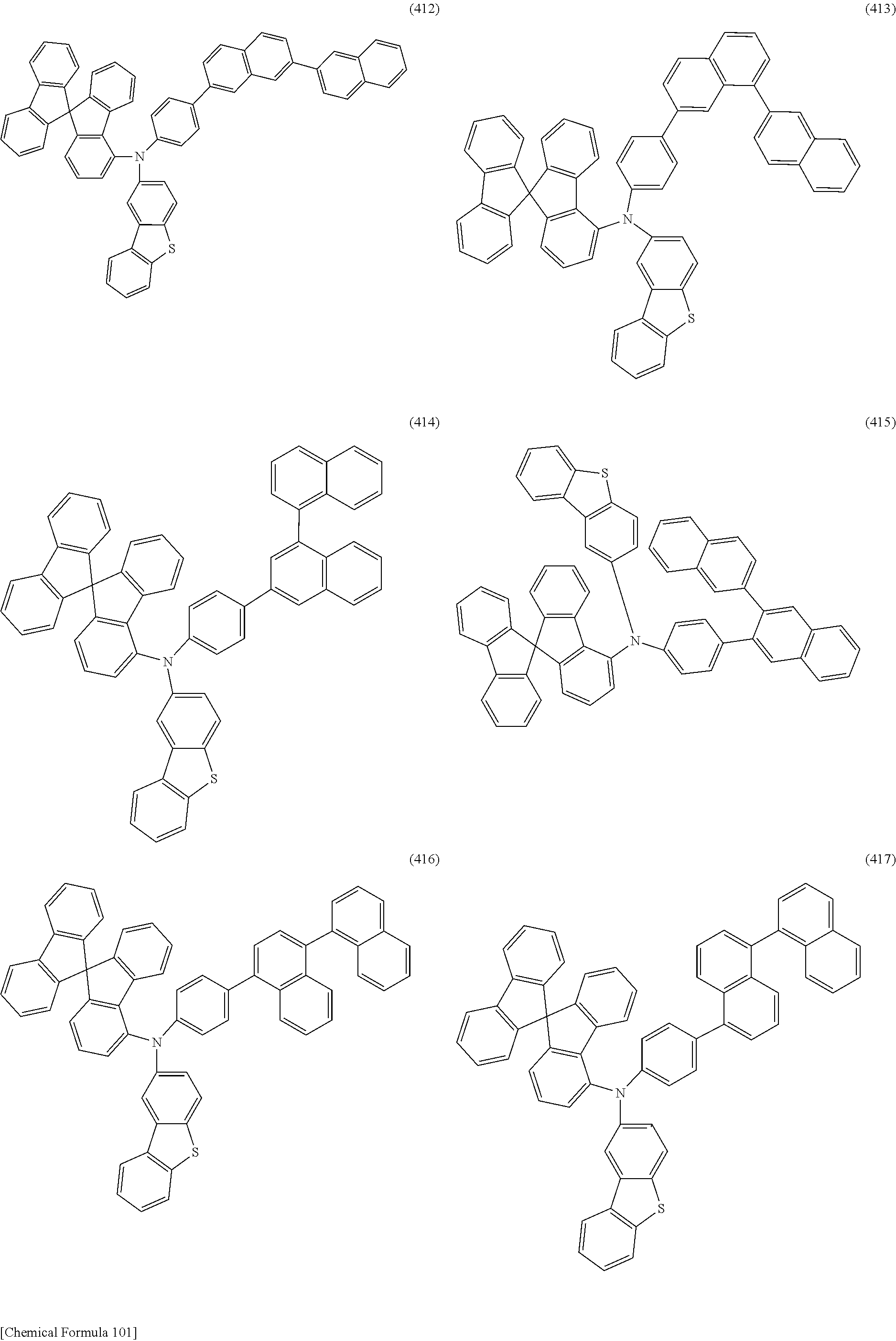

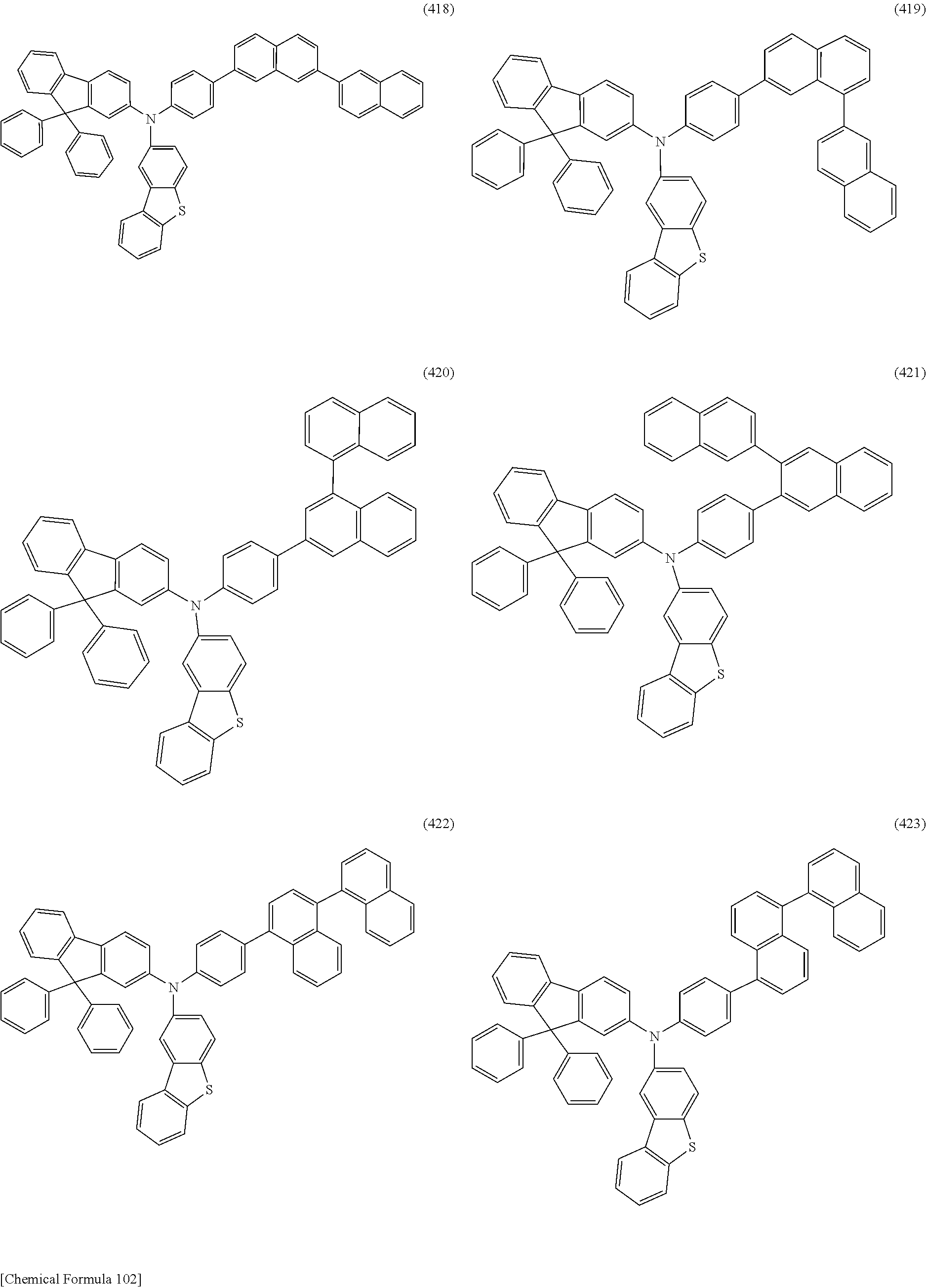

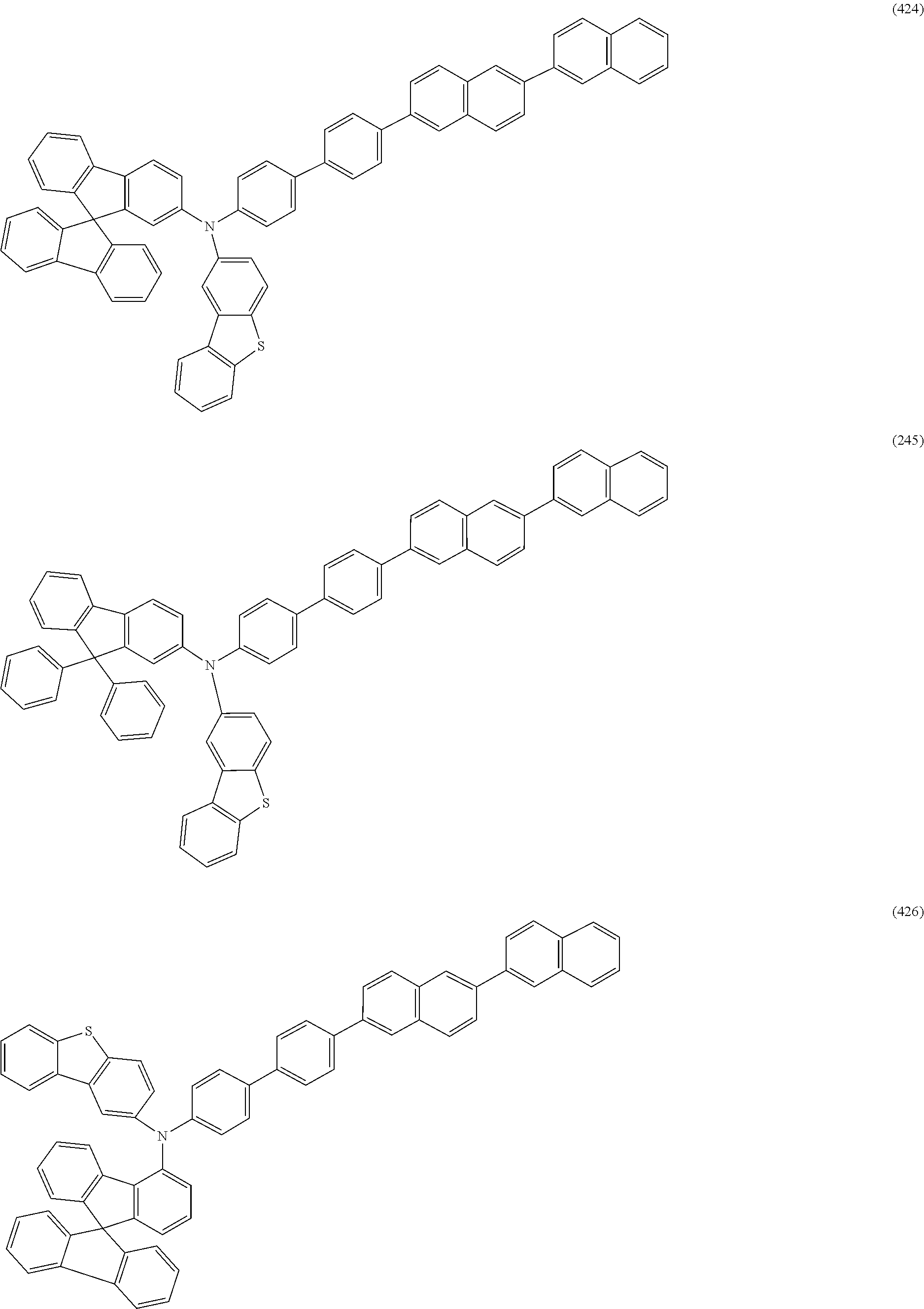 [SMILES: c1ccc2c(c1)-c1ccccc1C21c2ccccc2-c2ccc(N(c3ccc(-c4ccc(-c5ccc6cc(-c7ccc8ccccc8c7)ccc6c5)cc4)cc3)c3ccc4sc5ccccc5c4c3)cc21 ; c1ccc(C2(c3ccccc3)c3ccccc3-c3ccc(N(c4ccc(-c5ccc(-c6ccc7cc(-c8ccc9ccccc9c8)ccc7c6)cc5)cc4)c4ccc5sc6ccccc6c5c4)cc32)cc1 ; c1ccc2c(c1)-c1ccccc1C21c2ccccc2-c2c(N(c3ccc(-c4ccc(-c5ccc6cc(-c7ccc8ccccc8c7)ccc6c5)cc4)cc3)c3ccc4sc5ccccc5c4c3)cccc21]

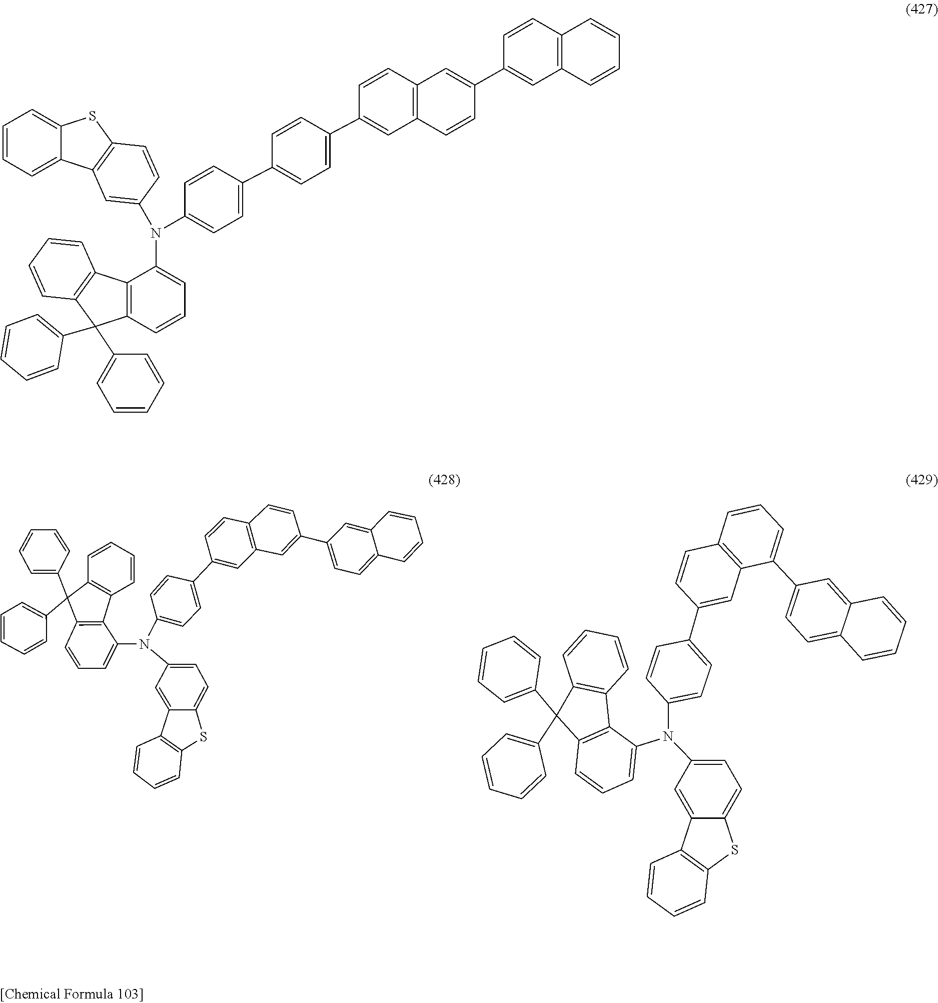 [SMILES: c1ccc(C2(c3ccccc3)c3ccccc3-c3c(N(c4ccc(-c5ccc(-c6ccc7cc(-c8ccc9ccccc9c8)ccc7c6)cc5)cc4)c4ccc5sc6ccccc6c5c4)cccc32)cc1 ; c1ccc(C2(c3ccccc3)c3ccccc3-c3c(N(c4ccc(-c5ccc6ccc(-c7ccc8ccccc8c7)cc6c5)cc4)c4ccc5sc6ccccc6c5c4)cccc32)cc1 ; c1ccc(C2(c3ccccc3)c3ccccc3-c3c(N(c4ccc(-c5ccc6cccc(-c7ccc8ccccc8c7)c6c5)cc4)c4ccc5sc6ccccc6c5c4)cccc32)cc1]

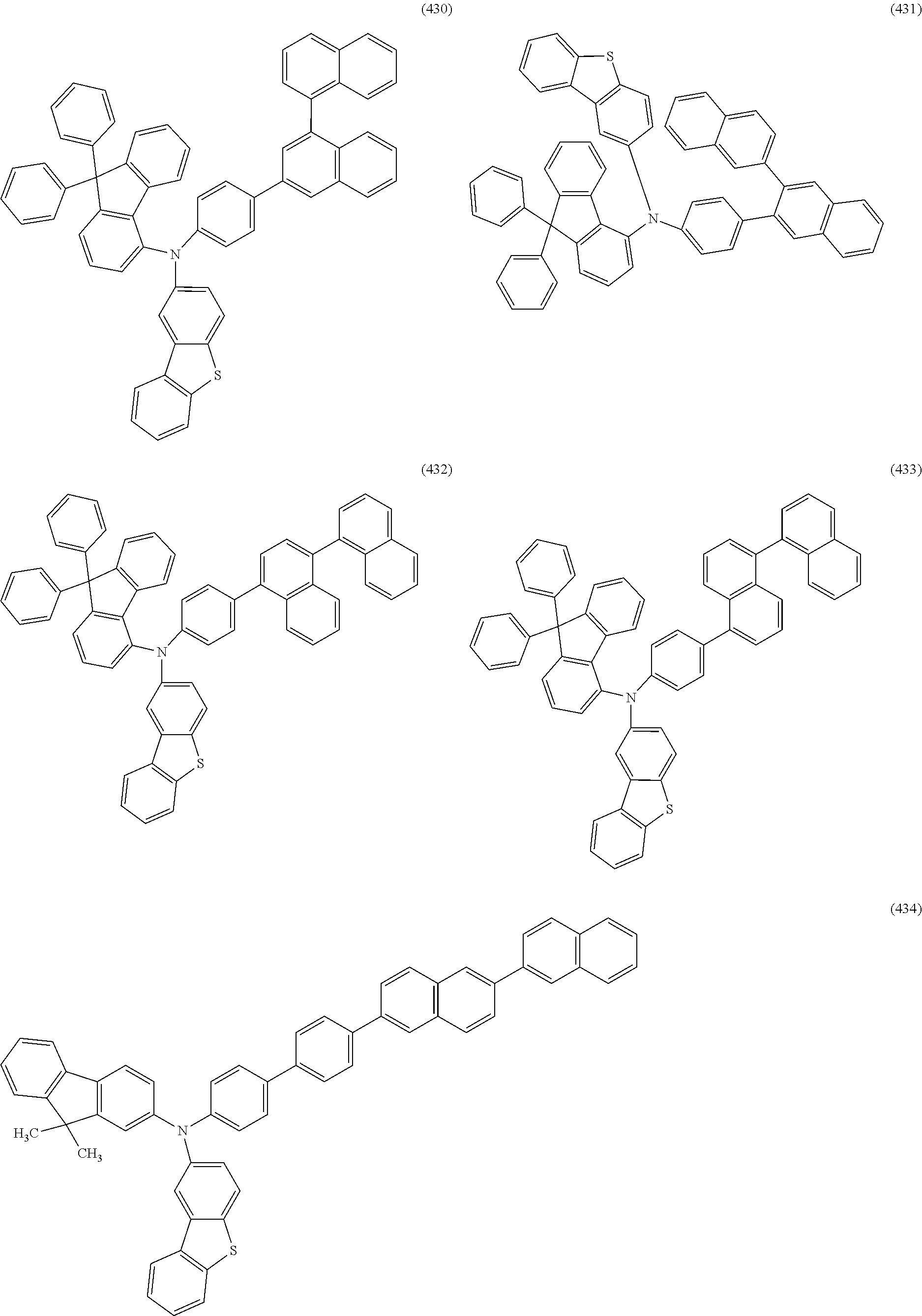

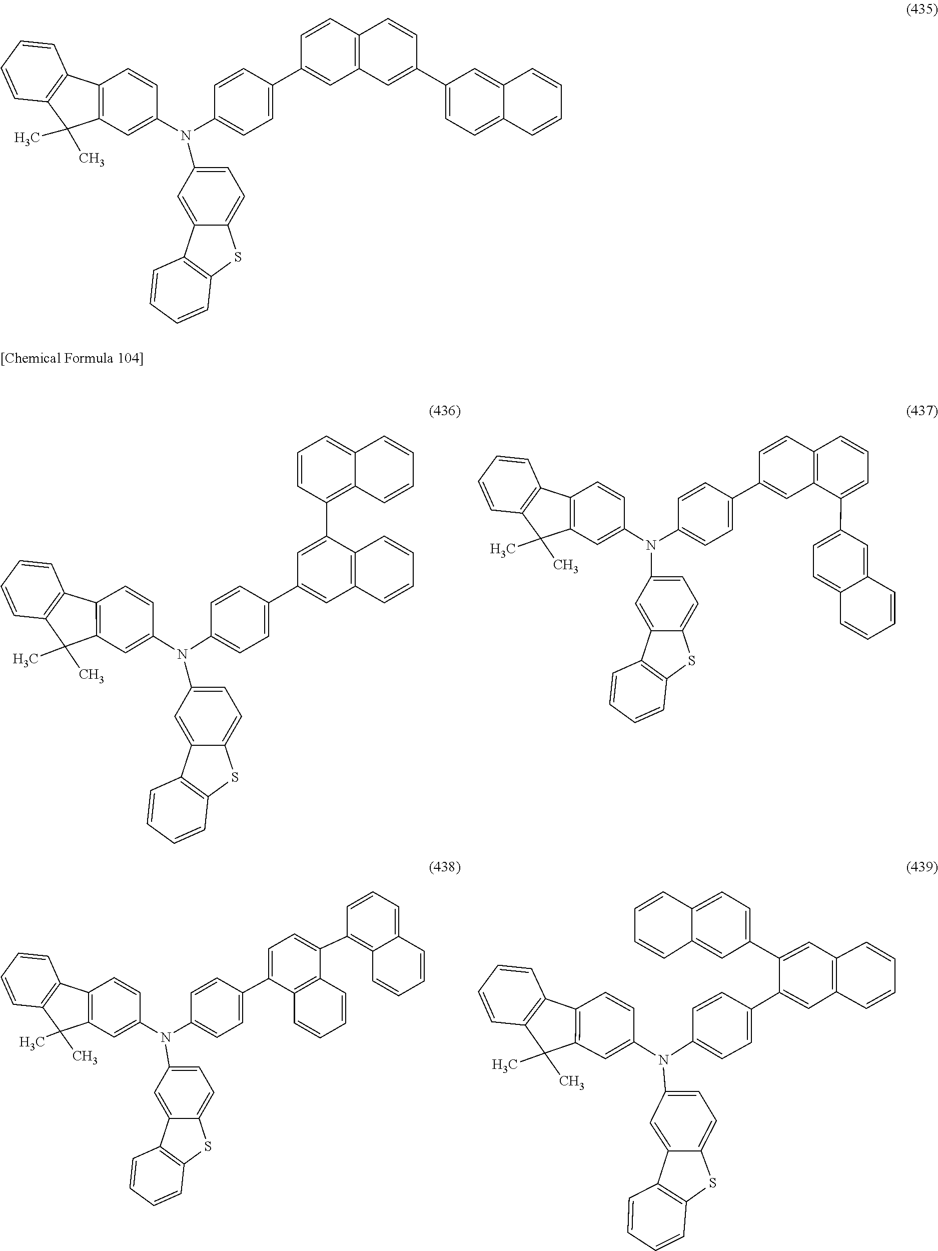 [SMILES: CC1(C)c2ccccc2-c2ccc(N(c3ccc(-c4ccc5ccc(-c6ccc7ccccc7c6)cc5c4)cc3)c3ccc4sc5ccccc5c4c3)cc21 ; CC1(C)c2ccccc2-c2ccc(N(c3ccc(-c4cc(-c5cccc6ccccc56)c5ccccc5c4)cc3)c3ccc4sc5ccccc5c4c3)cc21 ; CC1(C)c2ccccc2-c2ccc(N(c3ccc(-c4ccc5cccc(-c6ccc7ccccc7c6)c5c4)cc3)c3ccc4sc5ccccc5c4c3)cc21 ; CC1(C)c2ccccc2-c2ccc(N(c3ccc(-c4ccc(-c5cccc6ccccc56)c5ccccc45)cc3)c3ccc4sc5ccccc5c4c3)cc21 ; CC1(C)c2ccccc2-c2ccc(N(c3ccc(-c4cc5ccccc5cc4-c4ccc5ccccc5c4)cc3)c3ccc4sc5ccccc5c4c3)cc21]

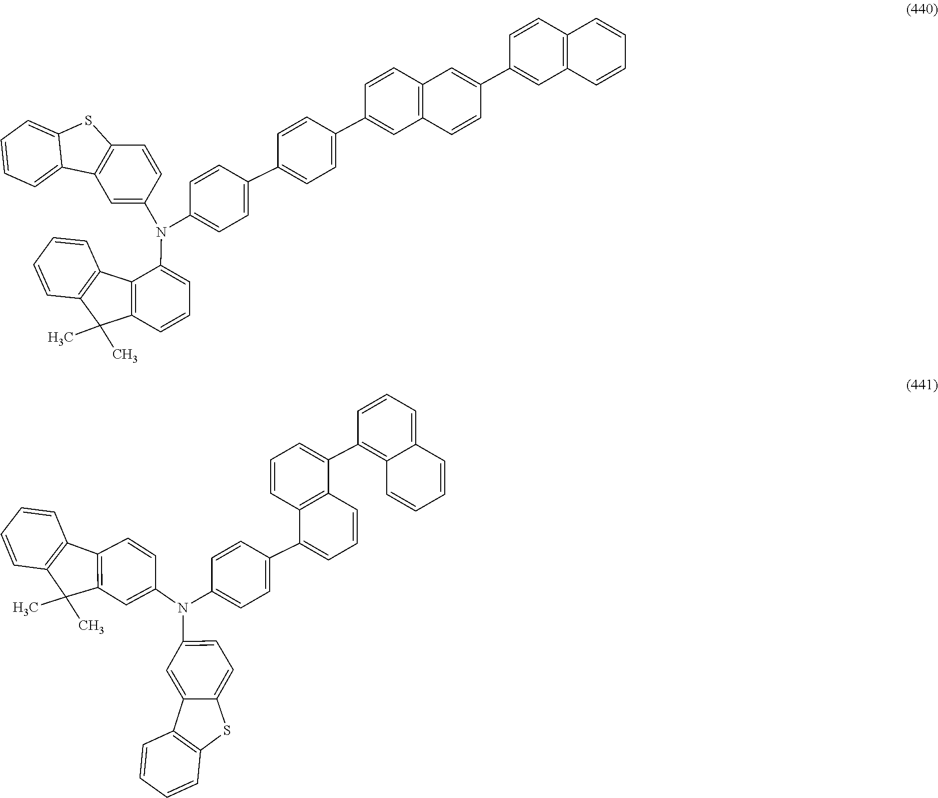 [SMILES: CC1(C)c2ccccc2-c2c(N(c3ccc(-c4ccc(-c5ccc6cc(-c7ccc8ccccc8c7)ccc6c5)cc4)cc3)c3ccc4sc5ccccc5c4c3)cccc21 ; CC1(C)c2ccccc2-c2ccc(N(c3ccc(-c4cccc5c(-c6cccc7ccccc67)cccc45)cc3)c3ccc4sc5ccccc5c4c3)cc21]

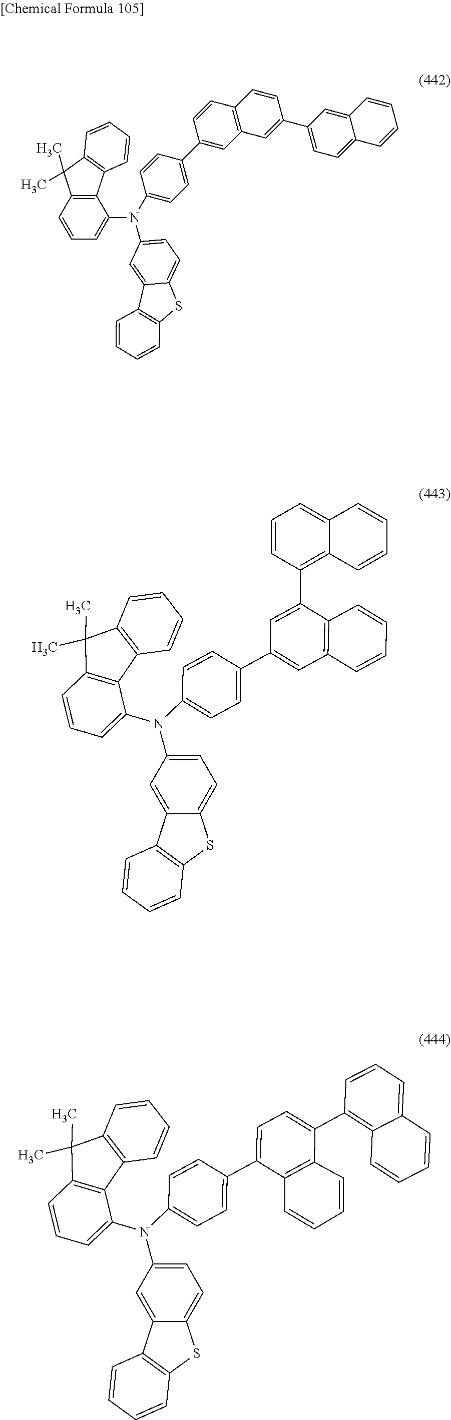 [SMILES: CC1(C)c2ccccc2-c2c(N(c3ccc(-c4ccc5ccc(-c6ccc7ccccc7c6)cc5c4)cc3)c3ccc4sc5ccccc5c4c3)cccc21 ; CC1(C)c2ccccc2-c2c(N(c3ccc(-c4cc(-c5cccc6ccccc56)c5ccccc5c4)cc3)c3ccc4sc5ccccc5c4c3)cccc21 ; CC1(C)c2ccccc2-c2c(N(c3ccc(-c4ccc(-c5cccc6ccccc56)c5ccccc45)cc3)c3ccc4sc5ccccc5c4c3)cccc21]

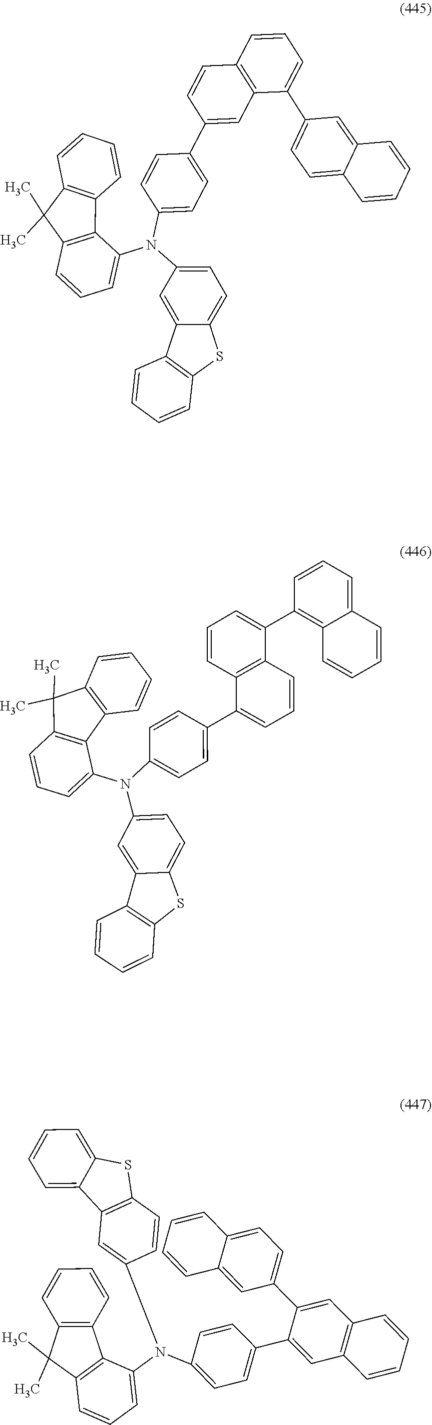 [SMILES: CC1(C)c2ccccc2-c2c(N(c3ccc(-c4ccc5cccc(-c6ccc7ccccc7c6)c5c4)cc3)c3ccc4sc5ccccc5c4c3)cccc21 ; CC1(C)c2ccccc2-c2c(N(c3ccc(-c4cccc5c(-c6cccc7ccccc67)cccc45)cc3)c3ccc4sc5ccccc5c4c3)cccc21 ; CC1(C)c2ccccc2-c2c(N(c3ccc(-c4cc5ccccc5cc4-c4ccc5ccccc5c4)cc3)c3ccc4sc5ccccc5c4c3)cccc21]

### Embodiment 2

In this embodiment, an organic semiconductor device of one embodiment of the present invention is described in detail.

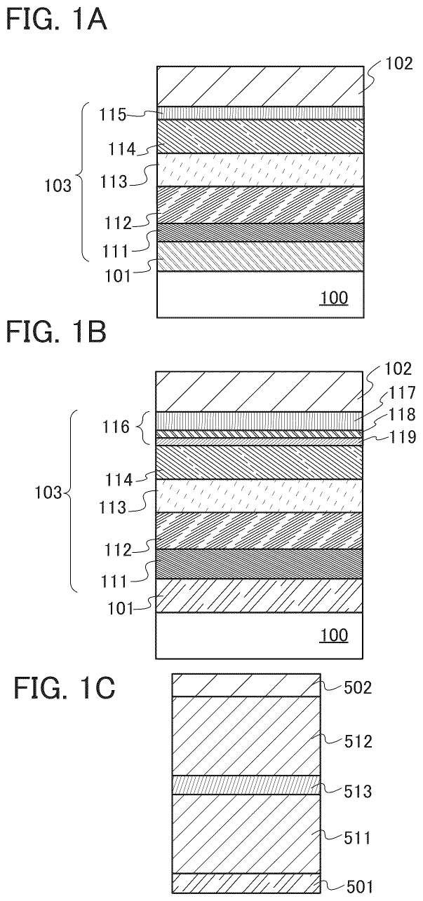

FIGS. 1A to 1C are schematic diagrams of light-emitting devices of embodiments of the present invention. Each of the light-emitting devices includes a first electrode 101 over an insulator 100, and an organic compound layer 103 between the first electrode 101 and a second electrode 102. The organic compound layer 103 includes at least one of the organic compounds represented by General Formulae (G1) to (G6) described in Embodiment 1. The organic semiconductor device of one embodiment of the present invention includes an active layer (e.g., a light-emitting layer 113 in a light-emitting device or a photoelectric conversion layer in a photosensor). The light-emitting layer 113 in the light-emitting device contains an emission center substance that emits light when voltage is applied between the first electrode 101 and the second electrode 102.

The organic compound layer 103 preferably includes, besides the light-emitting layer 113, functional layers such as a hole-injection layer 111, a hole-transport layer 112, an electron-transport layer 114, and an electron-injection layer 115, as shown in FIG. 1A. Note that the organic compound layer 103 may include functional layers other than the above functional layers, such as a hole-blocking layer, an electron-blocking layer, an exciton-blocking layer, and a charge-generation layer. Alternatively, any of the above layers may be omitted.

Note that the organic compounds represented by General Formulae (G1) to (G6) in Embodiment 1 are preferably contained in a layer where holes are moved. Examples of the layer where holes are moved include a hole-injection layer, a hole-transport layer, an electron-blocking layer, and a light-emitting layer.

Although the first electrode 101 includes an anode and the second electrode 102 includes a cathode in this embodiment, the first electrode 101 may include a cathode and the second electrode 102 may include an anode. The first electrode 101 and the second electrode 102 each have a single-layer structure or a stacked-layer structure. In the case of the stacked-layer structure, a layer in contact with the organic compound layer 103 serves as an anode or a cathode. In the case where the electrodes each have the stacked-layer structure, there is no limitation on work functions of materials for layers other than the layer in contact with the organic compound layer 103, and the materials can be selected in accordance with required properties such as a resistance value, processing easiness, reflectivity, light-transmitting property, and stability.

The anode is preferably formed using any of metals, alloys, and conductive compounds with a high work function (specifically, higher than or equal to 4.0 eV), mixtures thereof, and the like. Specific examples include indium oxide-tin oxide (ITO: indium tin oxide), indium oxide-tin oxide containing silicon or silicon oxide (ITSO: indium tin silicon oxide), indium oxide-zinc oxide, and indium oxide containing tungsten oxide and zinc oxide (IWZO). Films of such conductive metal oxides are usually formed by a sputtering method, but may be formed by application of a sol-gel method or the like. For example, a film of indium oxide-zinc oxide is formed by a sputtering method using a target in which 1 wt % to 20 wt % zinc oxide is added to indium oxide. Furthermore, a film of indium oxide containing tungsten oxide and zinc oxide (IWZO) can be formed by a sputtering method using a target in which 0.5 wt % to 5 wt % tungsten oxide and 0.1 wt % to 1 wt % zinc oxide are added to indium oxide. Alternatively, gold (Au), platinum (Pt), nickel (Ni), tungsten (W), chromium (Cr), molybdenum (Mo), iron (Fe), cobalt (Co), copper (Cu), palladium (Pd), titanium (Ti), aluminum (Al), nitride of a metal material (e.g., titanium nitride), or the like can be used for the anode. The anode may be a stack of layers formed of any of these materials. For example, a film in which Al, Ti, and ITSO are stacked in this order over Ti is preferable because the film has high efficiency owing to high reflectivity and enables high resolution of several thousand ppi. Graphene can also be used for the anode. When a composite material that can be included in the hole-injection layer 111 described later is used for a layer (typically, the hole-injection layer) in contact with the anode, an electrode material can be selected regardless of its work function.

The hole-injection layer 111 is provided in contact with the anode and has a function of facilitating injection of holes into the organic compound layer 103. The hole-injection layer 111 can be formed using phthalocyanine (abbreviation: H₂Pc), a phthalocyanine compound or a phthalocyanine-based complex compound such as copper phthalocyanine (abbreviation: CuPc), an aromatic amine compound such as 4,4′-bis[N-(4-diphenylaminophenyl)-N-phenylamino]biphenyl (abbreviation: DPAB) or 4,4′-bis(N-{4-[N′-(3-methylphenyl)-N′-phenylamino]phenyl}-N-phenylamino)biphenyl (abbreviation: DNTPD), or a high molecular compound such as poly(3,4-ethylenedioxythiophene)/poly(styrenesulfonic acid) (abbreviation: PEDOT/PSS).

The hole-injection layer 111 may be formed using a substance having an electron-accepting property. Examples of the substance having an acceptor property include organic compounds having an electron-withdrawing group (a halogen group or a cyano group), such as 7,7,8,8-tetracyano-2,3,5,6-tetrafluoroquinodimethane (abbreviation: F4-TCNQ), chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), and 2-(7-dicyanomethylene-1,3,4,5,6,8,9,10-octafluoro-7H-pyren-2-ylidene)malononitrile. A compound in which electron-withdrawing groups are bonded to a fused aromatic ring having a plurality of heteroatoms, such as HAT-CN, is particularly preferable because it is thermally stable. A [3]radialene derivative having an electron-withdrawing group (in particular, a cyano group, a halogen group such as a fluoro group, or the like) has a significantly high electron-accepting property and thus is preferable. Specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile]. As the substance having an acceptor property, a transition metal oxide such as molybdenum oxide, vanadium oxide, ruthenium oxide, tungsten oxide, or manganese oxide can be used, other than the above-described organic compounds.

The hole-injection layer 111 is preferably formed using a composite material containing any of the aforementioned materials having an acceptor property and an organic compound having a hole-transport property.

As the organic compound having a hole-transport property used in the composite material, any of a variety of organic compounds such as aromatic amine compounds, heteroaromatic compounds, aromatic hydrocarbons, and high molecular compounds (e.g., oligomers, dendrimers, and polymers) can be used. Note that the organic compound having a hole-transport property used in the composite material preferably has a hole mobility higher than or equal to 1×10⁻⁶ cm²/Vs. The organic compound having a hole-transport property used in the composite material preferably has a fused aromatic hydrocarbon ring or a π-electron rich heteroaromatic ring. As the fused aromatic hydrocarbon ring, an anthracene ring, a naphthalene ring, or the like is preferable. As the π-electron rich heteroaromatic ring, a fused aromatic ring having at least one of a pyrrole skeleton, a furan skeleton, and a thiophene skeleton is preferable; specifically, a carbazole ring, a dibenzothiophene ring, or a ring in which an aromatic ring or a heteroaromatic ring is further fused to a carbazole ring or a dibenzothiophene ring is preferable.

Such an organic compound having a hole-transport property further preferably has any of a carbazole skeleton, a dibenzofuran skeleton, a dibenzothiophene skeleton, and an anthracene skeleton. In particular, an aromatic amine having a substituent that includes a dibenzofuran ring or a dibenzothiophene ring, an aromatic monoamine that has a naphthalene ring, or an aromatic monoamine in which a 9-fluorenyl group is bonded to nitrogen of an amine through an arylene group may be used. Note that the organic compound having a hole-transport property preferably has an N,N-bis(4-biphenyl)amino group to enable fabricating a light-emitting device with a long lifetime.

Specific examples of the organic compound having a hole-transport property include N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6;1′-binaphthyl-2-yl)triphenylamine (abbreviation: BBAαNβNB), 4,4′-diphenyl-4″-(7;1′-binaphthyl-2-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), NN-bis(biphenyl-4-yl)-9,9′-spirobi[9H-fluoren]-2-amine (abbreviation: BBASF), N,N-bis(biphenyl-4-yl)-9,9′-spirobi[9H-fluoren]-4-amine (abbreviation: BBASF(4)), N-(biphenyl-2-yl)-N-(9,9-dimethyl-91-fluoren-2-yl)-9,9′-spirobi[9H-fluoren]-4-amine (abbreviation: oFBiSF), N-(biphenyl-4-yl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-(9-phenylfluoren-9-yl)triphenylamine (abbreviation: BPAFLP), 4-phenyl-3′-(9-phenylfluoren-9-yl)triphenylanmine (abbreviation: mBPAFLP), 4-phenyl-4′-[4-(9-phenylfluoren-9-yl)phenyl]triphenylamine (abbreviation: BPAFLBi), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), N-phenyl-N-[4-(9-phenyl-9H-carbazol-3-yl)phenyl]-9,9′-spirobi[9H-fluoren]-2-amine (abbreviation: PCBASF), N-(biphenyl-4-yl)-N-1[4-(9-phenyl-9H-carbazol-3-yl)phenyl]-9,9-dimethyl-9H-fluoren-2-amine (abbreviation: PCBBiF), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

Examples of the aromatic amine compounds that can be used as the material having a hole-transport property include N,N′-di(p-tolyl)-N,N′-diphenyl-p-phenylenedianmine (abbreviation: DTDPPA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), and 1,3,5-tris[N-(4-diphenylaminophenyl)-N-phenylamino]benzene (abbreviation: DPA3B). The organic compounds represented by General Formulae (G1) to (G6) in Embodiment 1 can also be suitably used.

The formation of the hole-injection layer 111 can improve the hole-injection property, which allows the light-emitting device to be driven at a low voltage.

Among substances having an acceptor property, the organic compound having an acceptor property is easy to use because it is easily deposited by evaporation.

The hole-transport layer 112 is formed using an organic compound having a hole-transport property. The organic compound having a hole-transport property preferably has a hole mobility higher than or equal to 1×10⁻⁶ cm²/Vs.

Examples of the material having a hole-transport property include compounds having an aromatic amine skeleton, such as 4,4′-bis[N-(1-naphthyl)-N-phenylamino]biphenyl (abbreviation: NPB), N,N′-diphenyl-N,N′-bis(3-methylphenyl)-4,4′-diaminobiphenyl (abbreviation: TPD), N,N′-bis(9,9′-spirobi[9H-fluoren]-2-yl)-N,N′-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), and N-phenyl-N-[4-(9-phenyl-9H-carbazol-3-yl)phenyl]-9,9′-spirobi[9H-fluoren]-2-amine (abbreviation: PCBASF); compounds having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 3,3′-bis(9-phenyl-9H-carbazole) (abbreviation: PCCP), 9,9′-bis(biphenyl-4-yl)-3,3′-bi-9H-carbazole (abbreviation: BisBPCz), 9,9′-bis(biphenyl-3-yl)-3,3′-bi-9H-carbazole (abbreviation: BismBPCz), and 9-(biphenyl-3-yl)-9′-(biphenyl-4-yl)-9H,9′H-3,3′-bicarbazole (abbreviation: mBPCCBP), 9-(2-naphthyl)-9′-phenyl-9H,9′H-3,3′-bicarbazole (abbreviation: βNCCP), 9-(3-biphenyl)-9′-(2-naphthyl)-3,3′-bi-9H-carbazole (abbreviation: βNCCmBP), 9-(4-biphenyl)-9′-(2-naphthyl)-3,3′-bi-9H-carbazole (abbreviation: PβNCCBP), 9,9′-di-2-naphthyl-3,3′-9H,9′H-bicarbazole (abbreviation: BisβNCz), 9-(2-naphthyl)-9′-[1,1′: 4′, 1″-terphenyl]-3-yl-3,3′-9H,9′H-bicarbazole, 9-(2-naphthyl)-9′-[1,1′: 3′, 1″-terphenyl]-3-yl-3,3′-9H,9′H-bicarbazole, 9-(2-naphthyl)-9′-[1,1′: 3′, 1″-terphenyl]-5′-yl-3,3′-9H,9′H-bicarbazole, 9-(2-naphthyl)-9′-[1,1′: 4′, 1″-terphenyl]-4-yl-3,3′-9H,9′H-bicarbazole, 9-(2-naphthyl)-9′-[1,1′: 3′, 1″-terphenyl]-4-yl-3,3′-9H,9′H-bicarbazole, 9-(2-naphthyl)-9′-(triphenylen-2-yl)-3,3′-9H,9′H-bicarbazole, 9-phenyl-9′-(triphenylen-2-yl)-3,3′-9H,9′H-bicarbazole (abbreviation: PCCzTp), 9,9′-bis(triphenylen-2-yl)-3,3′-9H,9′H-bicarbazole, 9-(4-biphenyl)-9′-(triphenylen-2-yl)-3,3′-91H,9′H-bicarbazole, and 9-(triphenylen-2-yl)-9′-[1,1′: 3′, 1″-terphenyl]-4-yl-3,3′-9H,9′H-bicarbazole; compounds having a thiophene skeleton, such as 4,4′, 4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV); and compounds having a furan skeleton, such as 4,4′, 4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II). Among the above materials, the compound having an aromatic amine skeleton or the compound having a carbazole skeleton is preferable because the compound is highly reliable and has a high hole-transport property to contribute to a reduction in driving voltage. Note that any of the substances given as examples of the material having a hole-transport property used in the composite material for the hole-injection layer 111 can also be suitably used as the material contained in the hole-transport layer 112. The organic compounds represented by General Formulae (G1) to (G6) in Embodiment 1 can also be suitably used.

The emission center substance may be a fluorescent substance, a phosphorescent substance, a substance exhibiting thermally activated delayed fluorescence (TADF), or any other light-emitting substance.

Examples of the material that can be used as a fluorescent substance in the light-emitting layer are as follows. Other fluorescent substances can also be used.

The examples include 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N′-diphenyl-N,N′-bis[4-(9-phenyl-9H-fluoren-9-yl)phenyl]pyrene-1,6-diamine (abbreviation: 1,6FLPAPrn), N,N′-bis(3-methylphenyl)-N,N-bis[3-(9-phenyl-9H-fluoren-9-yl)phenyl]pyrene-1,6-diamine (abbreviation: 1,6mMemFLPAPrn), N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra-tert-butylperylene (abbreviation: TBP), 4-(10-phenyl-9-anthryl)-4′-(9-phenyl-9H-carbazol-3-yl)triphenylamine (abbreviation: PCBAPA), N,N″-(2-tert-butylanthracene-9,10-diyldi-4,1-phenylene)bis(N,N′,N″-triphenyl-1,4-phenylenediamine) (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenediamine (abbreviation: 2DPAPPA), N,N,N′,N′,N″,N″,N′″,N′″-octaphenyldibenzo[g,p]chrysene-2,7,10,15-tetraamine (abbreviation: DBC1), coumarin 30, N-(9,10-diphenyl-2-anthryl)-N,9-diphenyl-9H-carbazol-3-amine (abbreviation: 2PCAPA), N-[9,10-bis(biphenyl-2-yl)-2-anthryl]-N,9-diphenyl-9H-carbazol-3-amine (abbreviation: 2PCABPhA), N (9,10-diphenyl-2-anthryl)-N,N′,N′-triphenyl-1,4-phenylenediamine (abbreviation: 2DPAPA), N′-[9,10-bis(biphenyl-2-yl)-2-anthryl]-N,N′,N′-triphenyl-1,4-phenylenediamine (abbreviation: 2DPABPhA), 9,10-bis(biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), N,N′-diphenyl-N,N′-(1,6-pyrene-diyl)bis[(6-phenylbenzo[b]naphtho[1,2-d]furan)-8-amine](abbreviation: 1,6BnfAPrn-03), 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02), and 3,10-bis[/N-(dibenzofuran-3-yl)-N-phenylamino]naphtho[2,3-b;6,7-b′]bisbenzofuran (abbreviation: 3,10FrA2PNbf(IV)-02). Fused aromatic diamine compounds typified by pyrenediamine compounds such as 1,6FLPAPrn, 1,6mMemFLPAPrn, and 1,6BnfAPrn-03 are particularly preferable because of their high hole-trapping properties, high emission efficiency, or high reliability.

A fused heteroaromatic compound containing nitrogen and boron, especially a compound having a diaza-boranaphtho-anthracene skeleton, exhibits a narrow emission spectrum, emits blue light with high color purity, and can thus be suitably used. Examples of the compound include 5,9-diphenyl-5,9-diaza-13b-boranaphtho[3,2,1-de]anthracene (abbreviation: DABNA1), 9-(diphenyl-3-yl)-N,N,5,11-tetraphenyl-5,9-dihydro-5,9-diaza-13b-boranaphtho[3,2,1-de]anthracen-3-amine (abbreviation: DABNA2), 2,12-di(tert-butyl)-5,9-di(4-tert-butylphenyl)-N,N-diphenyl-5H,9H-[11,4]benzazaborino[2,3,4-kl]phenazaborin-7-amine (abbreviation: DPhA-tBu4DABNA), 2,12-di(tert-butyl)-N,N,5,9-tetra(4-tert-butylphenyl)-5H,9H-[1,4]benzazaborino[2,3,4-kl]phenazaborin-7-amine (abbreviation: tBuDPhA-tBu4DABNA), 2,12-di(tert-butyl)-5,9-di(4-tert-butylphenyl)-7-methyl-5H,9H-[1,4]benzazaborino[2,3,4-kl]phenazaborine (abbreviation: Me-tBu4DABNA), N⁷,N⁷,N¹³,N¹³,5,9,11,15-octaphenyl-5H,9H,11H,15H-[1,4]benzazaborino[2,3,4-kl][1,4]benzazaborino[4′, 3′, 2′: 4,5][1,4]benzazaborino[3,2-b]phenazaborine-7,13-diamine (abbreviation: v-DABNA), and 2-(4-tert-butylphenyl)benz[5,6]indolo[3,2,1-jk]benzo[b]carbazole (abbreviation: tBuPBibc).

Besides the above compounds, 9,10,11-tris[3,6-bis(1,1-dimethylethyl)-9H-carbazolyl-9-yl]-2,5,15,18-tetrakis(1,1-dimethylethyl)indolo[3,2,1-de]indolo[3′, 2′, 1′: 8,1][1,4]benzazaborino[2,3,4-kl]phenazaborine (abbreviation: BBCz-G), 9,11-bis[3,6-bis(1,1-dimethylethyl)-9H-carbazolyl-9-yl]-2,5,15,18-tetrakis(1,1-dimethylethyl)indolo[3,2,1-de]indolo[3′, 2′, 1′: 8,1][1,4]benzazaborino[2,3,4-kl]phenazaborine (abbreviation: BBCz-Y), or the like can be suitably used.

Examples of the material that can be used when a phosphorescent substance is used as the light-emitting substance in the light-emitting layer are as follows.

The examples include an organometallic iridium complex having a 4H-triazole skeleton, such as tris{2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]), and tris(5-methyl-3,4-diphenyl-4H-1,2,4-triazolato)iridium(III) (abbreviation: [Ir(Mptz)₃]); an organometallic iridium complex having a 1H-triazole skeleton, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]): an organometallic iridium complex having an imidazole skeleton, such as fac-tris[1-(2,6-diisopropylphenyl)-2-phenyl-1H-imidazole]iridium(III) (abbreviation: [Ir(iPrpim)₃]), tris[3-(2,6-dimethylphenyl)-7-methylimidazo[1,2-f]phenanthridinato]iridium(III) (abbreviation: [Ir(dmpimpt-Me)₃]), and tris(2-{1-[2,6-bis(1-methylethyl)phenyl]-1H-imidazol-2-yl-κN³}-4-cyanophenyl-κC)iridium(III) (abbreviation: CNImfr); an organometallic complex having a benzimidazolidene skeleton, such as tris[(6-tert-butyl-3-phenyl-2H-imidazo[4,5-b]pyrazin-1-yl-κC²)phenyl-κC]iridium(III) (abbreviation: [Ir(cb)₃]); and an organometallic iridium complex in which a phenylpyridine derivative having an electron-withdrawing group is a ligand, such as bis[2-(4′, 6′-difluorophenyl)pyridinato-N,C^{2′}]iridium(III) tetrakis(1-pyrazolyl)borate (abbreviation: FIr6), bis[2-(4′, 6′-difluorophenyl)pyridinato-N,C^{2′}]iridium(III) picolinate (abbreviation: FIrpic), bis{2-[3′, 5′-bis(trifluoromethyl)phenyl]pyridinato-N,C^{2′}}iridium(III) picolinate (abbreviation: [Ir(CF₃ppy)₂(pic)]), and bis[2-(4′, 6′-difluorophenyl)pyridinato-N,C^{2′}]iridium(III) acetylacetonate (abbreviation: FIracac). These compounds emit blue phosphorescent light and have an emission peak in the wavelength range from 450 nm to 520 nm.

Other examples include an organometallic iridium complex having a pyrimidine skeleton, such as tris(4-methyl-6-phenylpyrimidinato)iridium(III) (abbreviation: [Ir(mppi)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppn)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(II) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), and (acetylacetonato)bis(4,6-diphenylpyrimidinato)iridium(III) (abbreviation: [Ir(dppm)₂(acac)]); an organometallic iridium complex having a pyrazine skeleton, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); an organometallic iridium complex having a pyridine skeleton, such as tris(2-phenylpyridinato-N,C^{2′})iridium(II) (abbreviation: [Ir(ppy)₃]), bis(2-phenylpyridinato-N′, C^{2′})iridium(11) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C″)iridium(III) (abbreviation: [Ir(pq)₃]), bis(2-phenylquinolinato-N,C²)iridium(Ill) acetylacetonate (abbreviation: [Ir(pq)₂(acac)]), [2-d₃-methyl-8-(2-pyridinyl-KN)benzofuro[2,3-b]pyridine-κC]bis[2-(5-d₃-methyl-2-pyridinyl-κN²)phenyl-κC]iridium(III) (abbreviation: [Ir(5mppy-d₃)₂(mbfpypy-d₃)]), [2-(methyl-d₃)-8-[4-(1-methylethyl-1-d)-2-pyridinyl-κN]benzofuro[2,3-b]pyridin-7-yl-κC]bis[5-(methyl-d₃)-2-[5-(methyl-d₃)-2-pyridinyl-κN)phenyl-κC]iridium(III) (abbreviation: Ir(5mtpy-d₆)₂(mbfpypy-iPr-d₄)), [2-d₃-methyl-(2-pyridinyl-κN)benzofuro[2,3-b]pyridine-κC]bis[2-(2-pyridinyl-κN)phenyl-κC]iridium(III) (abbreviation: [Ir(ppy)₂(mbfpypy-d₃)]), [2-(4-methyl-5-phenyl-2-pyridinyl-κN)phenyl-κC]bis[2-(2-pyridinyl-κN)phenyl-κC]iridium(III) (abbreviation: [Ir(ppy)₂(mdppy)]) [2-(4-d₃-methyl-5-phenyl-2-pyridinyl-κN²)phenyl-κC]bis[2-(5-d₃-methyl-2-pyridinyl-κN²)phenyl-κC]iridium(III) (abbreviation: [Ir(5mppy-d₃)₂(mdppy-d₃)]), and [2-methyl-(2-pyridinyl-κN)benzofuro[2,3-b]pyridine-κC]bis[2-(2-pyridinyl-κN)phenyl-κC]iridium(III) (abbreviation: [Ir(ppy)₂(mbfpypy)]); and a rare earth metal complex such as tris(acetylacetonato) (monophenanthroline)terbium(III) (abbreviation: [Tb(acac)₃(Phen)]). These are mainly compounds that emit green phosphorescent light and have an emission peak in the wavelength range from 500 nm to 600 nm. Note that organometallic iridium complexes including a pyrimidine skeleton have distinctively high reliability or emission efficiency and thus are particularly preferable.

Other examples include an organometallic iridium complex having a pyrimidine skeleton, such as (diisobutyrylmethanato)bis[4,6-bis(3-methylphenyl)pyriniidinato]iridium(III) (abbreviation: [Ir(5mdppm)₂(dibm)]), bis[4,6-bis(3-methylphenyl)pyrimidinato](dipivaloylnethanato)iridiurn(III) (abbreviation: [Ir(5mdppm)₂(dpm)]), and bis[4,6-di(naphthalen-1-yl)pyrimidinato](dipivaloylmethanato)iridium(III) (abbreviation: [Ir(d1npm)₂(dpm)]); an organometallic iridium complex having a pyrazine skeleton, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), and (acetylacetonato)bis[2,3-bis(4-fluorophenyl)quinoxalinato]iridium(III) (abbreviation: [Ir(Fdpq)₂(acac)]); an organometallic iridium complex having a pyridine skeleton, such as tris(1-phenylisoquinolinato-N,C^{2′})iridium(III) (abbreviation: [Ir(piq)₃]), bis(1-phenylisoquinolinato-N,C^{2′})iridium(III) acetylacetonate (abbreviation: [Ir(piq)₂(acac)]), (3,7-diethyl-4,6-nonanedionato-κO⁴,κO⁶)bis[2,4-dimethyl-6-[7-(1-methylethyl)-1-isoquinolinyl-κN]phenyl-κC]iridium(III), and (3,7-diethyl-4,6-nonanedionato-κO⁴,κO⁶)bis[2,4-dimethyl-6-[5-(1-methylethy])-2-quinolinyl-N]phenyl-κC^{2′})iridium(III); a platinum complex such as 2,3,7,8,12,13,17,18-octaethyl-21H,23H-porphyrinplatinum(II) (abbreviation: PtOEP); and a rare earth metal complex such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]). These compounds emit red phosphorescent light and have an emission peak in the wavelength range from 600 nm to 700 nm. Furthermore, the organometallic iridium complexes having a pyrazine skeleton can provide red light emission with favorable chromaticity.

Besides the above phosphorescent compounds, known phosphorescent compounds may be selected and used.

Examples of the TADF material include a fullerene, a derivative thereof, an acridine, a derivative thereof, and an eosin derivative. Furthermore, a metal-containing porphyrin, such as a porphyrin containing magnesium (Mg), zinc (Zn), cadmium (Cd), tin (Sn), platinum (Pt), indium (In), or palladium (Pd), can be given. Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), and an octaethylporphyrin-platinum chloride complex (PtCl₂OEP), which are represented by the following structural formulae.

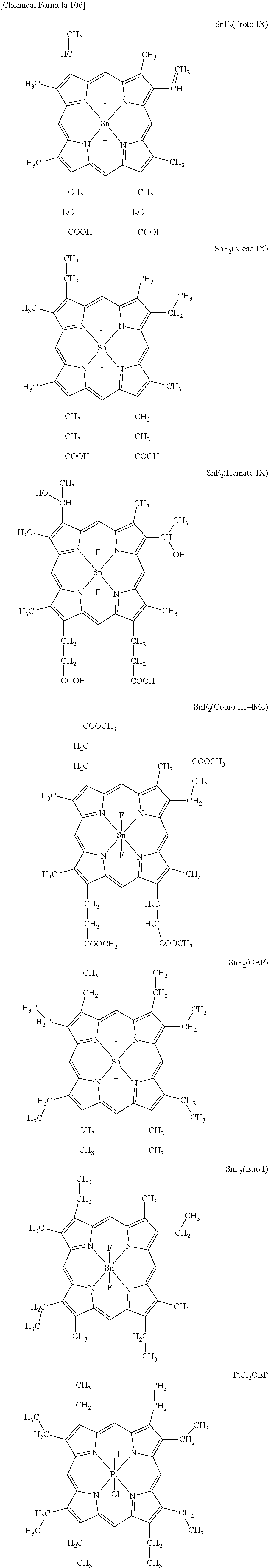

Alternatively, it is possible to use a heterocyclic compound having one or both of a π-electron rich heteroaromatic ring and a π-electron deficient heteroaromatic ring that is represented by the following structural formulae, such as 2-(biphenyl-4-yl)-4,6-bis(12-phenylindolo[2,3-a]carbazol-11-yl)-1,3,5-triazine (abbreviation: PIC-TRZ), 9-(4,6-diphenyl-1,3,5-triazin-2-yl)-9′-phenyl-9H,9′H-3,3′-bicarbazole (abbreviation: PCCzTzn), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), or 10-phenyl-10H,10′H-spiro[acridin-9,9′-anthracen]-10′-one (abbreviation: ACRSA). Such a heterocyclic compound is preferable because of having high electron-transport and hole-transport properties owing to a π-electron rich heteroaromatic ring and a π-electron deficient heteroaromatic ring. Among skeletons having the π-electron deficient heteroaromatic ring, a pyridine skeleton, a diazine skeleton (a pyrimidine skeleton, a pyrazine skeleton, and a pyridazine skeleton), and a triazine skeleton are preferable because of their high stability and reliability. In particular, a benzofuropyrimidine skeleton, a benzothienopyrimidine skeleton, a benzofuropyrazine skeleton, and a benzothienopyrazine skeleton are preferable because of their high acceptor properties and high reliability. Among skeletons having the π-electron rich heteroaromatic ring, an acridine skeleton, a phenoxazine skeleton, a phenothiazine skeleton, a furan skeleton, a thiophene skeleton, and a pyrrole skeleton have high stability and reliability; thus, at least one of these skeletons is preferably included. A dibenzofuran skeleton is preferable as a furan skeleton, and a dibenzothiophene skeleton is preferable as a thiophene skeleton. As a pyrrole skeleton, an indole skeleton, a carbazole skeleton, an indolocarbazole skeleton, a bicarbazole skeleton, and a 3-(9-phenyl-91H-carbazol-3-yl)-9H-carbazole skeleton are particularly preferable. Note that a substance in which the π-electron rich heteroaromatic ring is directly bonded to the π-electron deficient heteroaromatic ring is particularly preferable because the electron-donating property of the π-electron rich heteroaromatic ring and the electron-accepting property of the π-electron deficient heteroaromatic ring are both improved, the energy difference between the S₁ level and the T₁ level becomes small, and thus thermally activated delayed fluorescence can be obtained with high efficiency. Note that an aromatic ring to which an electron-withdrawing group such as a cyano group is bonded may be used instead of the π-electron deficient heteroaromatic ring. As a π-electron rich skeleton, an aromatic amine skeleton, a phenazine skeleton, or the like can be used. As a π-electron deficient skeleton, a xanthene skeleton, a thioxanthene dioxide skeleton, an oxadiazole skeleton, a triazole skeleton, an imidazole skeleton, an anthraquinone skeleton, a skeleton containing boron such as phenylborane or boranthrene, an aromatic ring or a heteroaromatic ring having a cyano group or a nitrile group such as benzonitrile or cyanobenzene, a carbonyl skeleton such as benzophenone, a phosphine oxide skeleton, a sulfone skeleton, or the like can be used. As described above, a π-electron deficient skeleton and a π-electron rich skeleton can be used instead of at least one of the π-electron deficient heteroaromatic ring and the π-electron rich heteroaromatic ring.

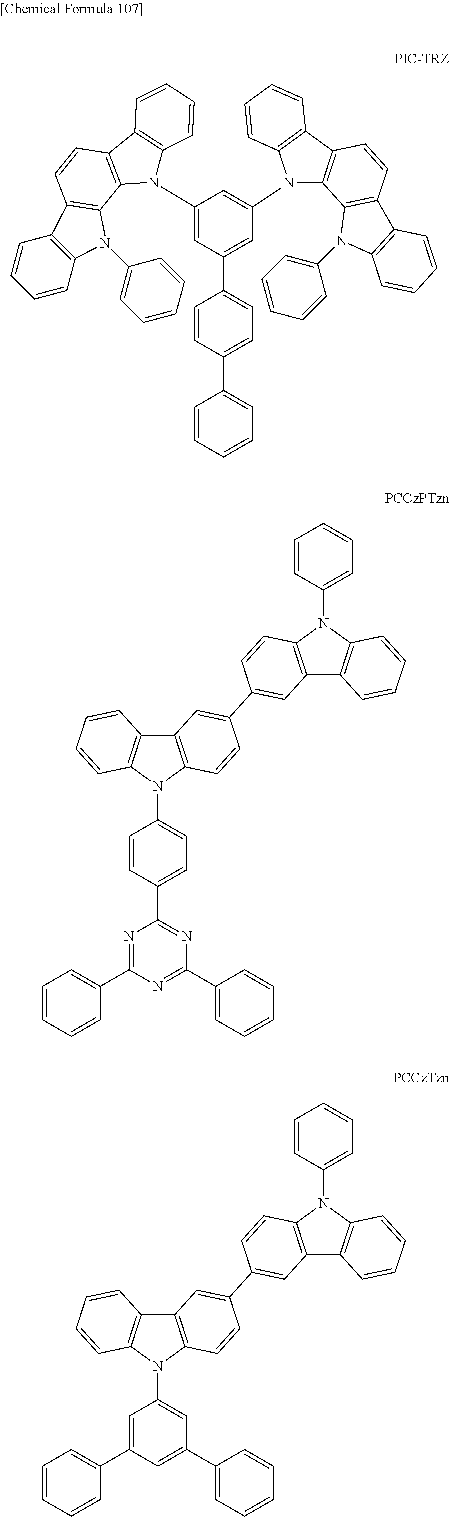 [SMILES: c1ccc(-c2ccc(-c3cc(-n4c5ccccc5c5ccc6c7ccccc7n(-c7ccccc7)c6c54)cc(-n4c5ccccc5c5ccc6c7ccccc7n(-c7ccccc7)c6c54)c3)cc2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; c1ccc(-c2cc(-c3ccccc3)cc(-n3c4ccccc4c4cc(-c5ccc6c(c5)c5ccccc5n6-c5ccccc5)ccc43)c2)cc1]

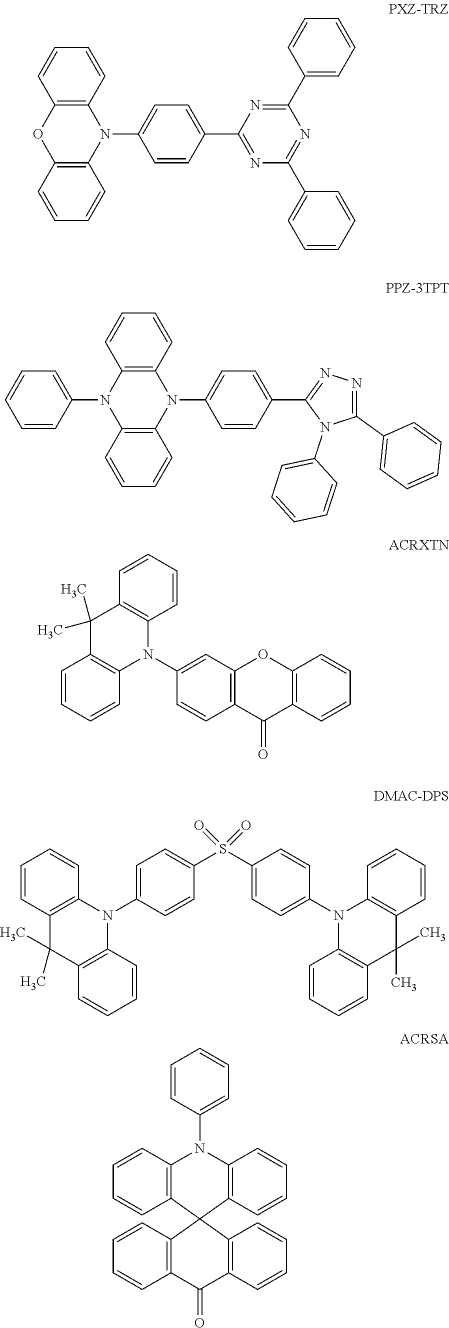 [SMILES: c1ccc(-c2nc(-c3ccccc3)nc(-c3ccc(N4c5ccccc5Oc5ccccc54)cc3)n2)cc1 ; c1ccc(-c2nnc(-c3ccc(N4c5ccccc5N(c5ccccc5)c5ccccc54)cc3)n2-c2ccccc2)cc1 ; CC1(C)c2ccccc2N(c2ccc3c(=O)c4ccccc4oc3c2)c2ccccc21 ; CC1(C)c2ccccc2N(c2ccc(S(=O)(=O)c3ccc(N4c5ccccc5C(C)(C)c5ccccc54)cc3)cc2)c2ccccc21 ; O=C1c2ccccc2C2(c3ccccc31)c1ccccc1N(c1ccccc1)c1ccccc12]

Note that a TADF material is a material having a small difference between the S₁ level and the T₁ level and a function of converting triplet excitation energy into singlet excitation energy by reverse intersystem crossing. Thus, a TADF material can upconvert triplet excitation energy into singlet excitation energy (i.e., reverse intersystem crossing) using a small amount of thermal energy and efficiently generate a singlet excited state. In addition, the triplet excitation energy can be converted into light emission.

An exciplex whose excited state is formed of two kinds of substances has an extremely small difference between the S₁ level and the T₁ level and functions as a TADF material capable of converting triplet excitation energy into singlet excitation energy.

A phosphorescent spectrum observed at a low temperature (e.g., 77 K to 10 K) is used for an index of the T₁ level. When the level of energy with a wavelength of the line obtained by extrapolating a tangent to the fluorescent spectrum at a tail on the short wavelength side is the S₁ level and the level of energy with a wavelength of the line obtained by extrapolating a tangent to the phosphorescent spectrum at a tail on the short wavelength side is the T₁ level, the difference between the S₁ level and the T₁ level of the T ADF material is preferably smaller than or equal to 0.3 eV, further preferably smaller than or equal to 0.2 eV.

When a TADF material is used as the light-emitting substance, the S₁ level of the host material is preferably higher than that of the TADF material. In addition, the T₁ level of the host material is preferably higher than that of the T ADF material.

As the host material in the light-emitting layer, various carrier-transport materials such as materials having an electron-transport property and/or materials having a hole-transport property, and the TADF materials can be used.

The material with a hole-transport property is preferably an organic compound having an amine skeleton or a π-electron rich heteroaromatic ring skeleton, for example. As the π-electron rich heteroaromatic ring, a fused aromatic ring having at least one of an acridine skeleton, a phenoxazine skeleton, a phenothiazine skeleton, a furan skeleton, a thiophene skeleton, and a pyrrole skeleton is preferable; specifically, a carbazole ring, a dibenzothiophene ring, or a ring in which an aromatic ring or a heteroaromatic ring is further fused to a carbazole ring or a dibenzothiophene ring is preferable.

Such an organic compound with a hole-transport property further preferably has any of a carbazole skeleton, a dibenzofuran skeleton, a dibenzothiophene skeleton, and an anthracene skeleton. In particular, an aromatic amine having a substituent that includes a dibenzofuran ring or a dibenzothiophene ring, an aromatic monoamine that has a naphthalene ring, or an aromatic monoamine in which a 9-fluorenyl group is bonded to nitrogen of an amine through an arylene group may be used. Note that the organic compound with a hole-transport property preferably has an N,N-bis(4-biphenyl)amino group to enable fabricating a light-emitting device having a long lifetime.

Examples of such an organic compound include a compound having an aromatic amine skeleton, such as 4,4′-bis[N-(1-naphthyl)-A-phenylamino]biphenyl (abbreviation: NPB), N,N′-diphenyl-N,N′-bis(3-methylphenyl)-4,4′-diaminobiphenyl (abbreviation: TPD), N,N′-bis(9,9′-spirobi[9H-fluoren]-2-yl)-N,N′-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), and N-phenyl-N-[4-(9-phenyl-9H-carbazol-3-yl)phenyl]-9,9′-spirobi[9H-fluoren]-2-amine (abbreviation: PCBASF); a compound having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), and 3,3′-bis(9-phenyl-9H-carbazole) (abbreviation: PCCP); a compound having a thiophene skeleton, such as 4,4′, 4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV); and a compound having a furan skeleton, such as 4,4′, 4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II). Among the above materials, the compound having an aromatic amine skeleton and the compound having a carbazole skeleton are preferable because these compounds are highly reliable and have high hole-transport properties to contribute to a reduction in driving voltage. In addition, the organic compounds given as examples of the material having a hole-transport property that can be used for the hole-transport layer can also be used. The organic compounds represented by General Formulae (G1) to (G6) in Embodiment 1 can also be suitably used.

The material having an electron-transport property preferably has an electron mobility higher than or equal to 1×10⁻⁷ cm²/Vs, further preferably higher than or equal to 1×10⁻⁶ cm²/Vs in the case where the square root of the electric field strength [V/cm] is 600. Note that any other substance can also be used as long as the substance has an electron-transport property higher than a hole-transport property.

As the material having an electron-transport property, for example, a metal complex such as bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), bis(8-quinolinolato)zinc(II) (abbreviation: Zng), bis[2-(2-benzoxazolyl)phenolato]zinc(II) (abbreviation: ZnPBO), or bis[2-(2-benzothiazolyl)phenolato]zinc(II) (abbreviation: ZnBTZ); or an organic compound having a π-electron deficient heteroaromatic ring is preferably used. Examples of the organic compound having a π-electron deficient heteroaromatic ring skeleton include an organic compound that includes a heteroaromatic ring having a polyazole skeleton, an organic compound that includes a heteroaromatic ring having a pyridine skeleton, an organic compound that includes a heteroaromatic ring having a diazine skeleton, and an organic compound that includes a heteroaromatic ring having a triazine skeleton.

Among the above materials, the organic compound that includes a heteroaromatic ring having a diazine skeleton (a pyrimidine skeleton, a pyrazine skeleton, or a pyridazine skeleton), the organic compound that includes a heteroaromatic ring having a pyridine skeleton, and the organic compound that includes a heteroaromatic ring having a triazine skeleton have high reliability and thus are preferable. In particular, the organic compound that includes a heteroaromatic ring having a diazine (pyrimidine or pyrazine) skeleton and the organic compound that includes a heteroaromatic ring having a triazine skeleton have a high electron-transport property to contribute to a reduction in driving voltage. A benzofuropyrimidine skeleton, a benzothienopyrimidine skeleton, a benzofuropyrazine skeleton, and a benzothienopyrazine skeleton are preferable because of their high acceptor properties and high reliability.

Examples of the organic compound having a π-electron deficient heteroaromatic ring skeleton include an organic compound having an azole skeleton, such as 2-(4-biphenylyl)-5-(4-tert-butylphenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: Col 1), 2,2,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), or 4,4′-bis(5-methylbenzoxazol-2-yl)stilbene (abbreviation: BzOS); an organic compound having a heteroaromatic ring having a pyridine skeleton, such as 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), bathophenanthroline (abbreviation: BPhen), bathocuproine (abbreviation: BCP), 2,9-di(naphthalen-2-yl)-4,7-diphenyl-1,10-phenanthroline (abbreviation: NBPhen), 2,2′-(1,3-phenylene)bis(9-phenyl-1,10-phenanthroline) (abbreviation: mPPhen2P), 2-[3-(2-triphenylenyl)phenyl]-1,10-phenanthroline (abbreviation: mTpPPhen), 2-phenyl-9-(2-triphenylenyl)-1,10-phenanthroline (abbreviation: Ph-TpPhen), 2-[4-(9-phenanthrenyl)-1-naphthalenyl]-1,10-phenanthroline (abbreviation: PnNPhen), or 2-[4-(2-triphenylenyl)phenyl]-1,10-phenanthroline (abbreviation: pTpPPhen); an organic compound having a diazine skeleton, such as 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4′-(9-phenyl-9H-carbazol-3-yl)-3,1′-biphenyl-1-yl]dibenzo[f,h]quinoxaline (abbreviation: 2mpPCBPDBq), 2-[4-(3,6-diphenyl-91-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), 9-[3′-(dibenzothiophen-4-yl)biphenyl-3-yl]naphtho[1′, 2′: 4,5]furo[2,3-b]pyrazine (abbreviation: 9mDBtBPNfpr), 9-[3′-(dibenzothiophen-4-yl)biphenyl-4-yl]naphtho[1′, 2′: 4,5]furo[2,3-b]pyrazine (abbreviation: 9pmDBtBPNfpr), 4,6-bis[3-(phenanthren-9-yl)phenyl]pyrimidine (abbreviation: 4,6mPnP2Pm), 4,6-bis[3-(dibenzothiophen-4-yl)phenyl]pyrimidine (abbreviation: 4,6mDBTP2Pm-II), 4,6-bis[3-(9H-carbazol-9-yl)phenyl]pyrimidine (abbreviation: 4,6nCzP2Pn), 9,9′-[pyrimidine-4,6-diylbis(biphenyl-3,3′-diyl)]bis(9H-carbazole) (abbreviation: 4,6rnCzBP2Prn), 8-(biphenyl-4-yl)-4-[3-(dibenzothiophen-4-yl)phenyl]-[1]benzofuro[3,2-d]pyrimidine (abbreviation: 8BP-4mDBtPBfprn), 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-[1]benzofuro[3,2-d]pyrimidine (abbreviation: 4,8mDBtP2Bfprn), 8-[3′-(dibenzothiophen-4-yl)(biphenyl-3-yl)]naphtho[1′, 2′: 4,5]furo[3,2-d]pyrinidine (abbreviation: 8mDBtBPNfpm), 8-[(2,2′-binaphthalen)-6-yl]-4-[3-(dibenzothiophen-4-yl)phenyl]-[1]benzofuro[3,2-d]pyrimidine (abbreviation: 8(PN2)-4mDBtPBfpm), 2,2′-(pyridine-2,6-diyl)bis(4-phenylbenzo[h]quinazoline) (abbreviation: 2,6(P-Bqn)₂Py), 2,2′-(pyridine-2,6-diyl)bis{4-[4-(2-naphthyl)phenyl]-6-phenylpyrimidine}(abbreviation: 2,6(NP-PPrn)₂Py), 6-(biphenyl-3-yl)-4-[3,5-bis(9H-carbazol-9-yl)phenyl]-2-phenylpyrimidine (abbreviation: 6mBP-4Cz2PPrn), 2,6-bis(4-naphthalen-1-ylphenyl)-4-[4-(3-pyridyl)phenyl]pyrimidine (abbreviation: 2,4NP-6PyPPm), 4-[3,5-bis(9H-carbazol-9-yl)phenyl]-2-phenyl-6-(1,1′-biphenyl-4-yl)pyrimidine (abbreviation: 6BP-4Cz2PPn), or 7-[4-(9-phenyl-9H-carbazol-2-yl)quinazolin-2-yl]-7H-dibenzo[c,g]carbazole (abbreviation: PC-cgDBCzQz); and an organic compound having a heteroaromatic ring having a triazine skeleton, such as 2-(biphenyl-4-yl)-4-phenyl-6-(9,9′-spirobi[9H-fluoren]-2-yl)-1,3,5-triazine (abbreviation: BP-SFTzn), 2-{3-[3-(benzo[b]naphtho[1,2-d]furan-8-yl)phenyl]phenyl}-4,6-diphenyl-1,3,5-triazine (abbreviation: mBnfBPTzn), 2-{3-[3-(benzo[b]naphtho[1,2-d]furan-6-yl)phenyl]phenyl}-4,6-diphenyl-1,3,5-triazine (abbreviation: mBnfBPTzn-02), 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 2-[3′-(9,9-dimethyl-9[H-fluoren-2-yl)biphenyl-3-yl]-4,6-diphenyl-1,3,5-triazine (abbreviation: mFBPTzn), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-{3-[3-(dibenzothiophen-4-yl)phenyl]phenyl}-4,6-diphenyl-1,3,5-triazine (abbreviation: mDBtBPTzn), 2,4,6-tris(3′-(pyridin-3-yl)biphenyl-3-yl)-1,3,5-triazine (abbreviation: TmPPPyTz), 2-[3-(2,6-dimethyl-3-pyridinyl)-5-(9-phenanthrenyl)phenyl]-4,6-diphenyl-1,3,5-triazine (abbreviation: mPn-mDMePyPTzn), 11-[4-(biphenyl-4-yl)-6-phenyl-1,3,5-triazin-2-yl]-11,12-dihydro-12-phenylindolo[2,3-a]carbazole (abbreviation: BP-Icz(II)Tzn), 2-[3′-(triphenylen-2-yl)biphenyl-3-yl]-4,6-diphenyl-1,3,5-triazine (abbreviation: mTpBPTzn), 3-[9-(4,6-diphenyl-1,3,5-triazin-2-yl)-2-dibenzofuranyl]-9-phenyl-9H-carbazole (abbreviation: PCDBfTzn), or 2-(biphenyl-3-yl)-4-phenyl-6-{8-[(1,1′: 4′, 1″-terphenyl)-4-yl]-1-dibenzofuranyl}-1,3,5-triazine (abbreviation: mBP-TPDBIfTzn). The organic compound that includes a heteroaromatic ring having a diazine skeleton, the organic compound that includes a heteroaromatic ring having a pyridine skeleton, and the organic compound that includes a heteroaromatic ring having a triazine skeleton are preferable because of having high reliability. In particular, the organic compound that includes a heteroaromatic ring having a diazine (pyrimidine or pyrazine) skeleton and the organic compound that includes a heteroaromatic ring having a triazine skeleton have a high electron-transport property to contribute to a reduction in driving voltage.

As the TADF material that can be used as the host material, the above materials mentioned as the TADF material can also be used. When the TADF material is used as the host material, triplet excitation energy generated in the TADF material is converted into singlet excitation energy by reverse intersystem crossing and transferred to the light-emitting substance, whereby the emission efficiency of the light-emitting device can be increased. Here, the TADF material functions as an energy donor, and the light-emitting substance functions as an energy acceptor.

This is very effective in the case where the light-emitting substance is a fluorescent substance. In that case, the S₁ level of the TADF material is preferably higher than that of the fluorescent substance in order that high emission efficiency can be achieved. Furthermore, the T₁ level of the TADF material is preferably higher than the S₁ level of the fluorescent substance. Therefore, the T₁ level of the TADF material is preferably higher than that of the fluorescent substance.

It is also preferable to use a TADF material that emits light whose wavelength overlaps with the wavelength on a lowest-energy-side absorption band of the fluorescent substance, in which case excitation energy is transferred smoothly from the TADF material to the fluorescent substance and light emission can be obtained efficiently.

In addition, in order to efficiently generate singlet excitation energy from the triplet excitation energy by reverse intersystem crossing, carrier recombination preferably occurs in the TADF material. It is also preferable that the triplet excitation energy generated in the TADF material not be transferred to the triplet excitation energy of the fluorescent substance. For that reason, the fluorescent substance preferably has a protective group around a luminophore (a skeleton which causes light emission) of the fluorescent substance. As the protective group, a substituent having no r bond and a saturated hydrocarbon are preferably used. Specific examples include an alkyl group having 3 to 10 carbon atoms, a substituted or unsubstituted cycloalkyl group having 3 to 10 carbon atoms, and a trialkylsilyl group having 3 to 10 carbon atoms. It is further preferable that the fluorescent substance have a plurality of protective groups. The substituents having no r bond are poor in carrier transport performance, whereby the TADF material and the luminophore of the fluorescent substance can be made away from each other with little influence on carrier transportation or carrier recombination. Here, the luminophore refers to an atomic group (skeleton) that causes light emission in a fluorescent substance. The luminophore is preferably a skeleton having a Tr bond, further preferably includes an aromatic ring, and still further preferably includes a fused aromatic ring or a fused heteroaromatic ring. Examples of such a luminophore include a phenanthrene skeleton, a stilbene skeleton, an acridone skeleton, a phenoxazine skeleton, a phenothiazine skeleton, a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton. Specifically, a fluorescent substance having any of a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton is preferable because of its high fluorescence quantum yield.

In the case where a fluorescent substance is used as the light-emitting substance, a material having an acene skeleton, especially an anthracene skeleton is suitably used as the host material. The use of a substance having an anthracene skeleton as the host material for the fluorescent substance makes it possible to obtain a light-emitting layer with high emission efficiency and high durability. Among the substances having an anthracene skeleton, a substance having a diphenylanthracene skeleton, in particular, a substance having a 9,10-diphenylanthracene skeleton, is chemically stable and thus is preferably used as the host material. The host material preferably has a carbazole skeleton to have higher hole-injection and hole-transport properties; further preferably, the host material has a benzocarbazole skeleton in which a benzene ring is further condensed to a carbazole skeleton, because the HOMO level of the host material having a benzocarbazole skeleton is higher than that of the host material having a carbazole skeleton by approximately 0.1 eV and the host material having a benzocarbazole skeleton is thus easier for holes to enter than the host material having a carbazole skeleton. In particular, the host material preferably has a dibenzocarbazole skeleton, because the HOMO level of the host material having a dibenzocarbazole skeleton is higher than that of the host material having a carbazole skeleton by approximately 0.1 eV, the host material having a dibenzocarbazole skeleton is thus easier for holes to enter than the host material having a carbazole skeleton, and the host material having a dibenzocarbazole skeleton has a higher hole-transport property and higher heat resistance than the host material having a carbazole skeleton. Accordingly, a substance that has both a 9,10-diphenylanthracene skeleton and a carbazole skeleton (or a benzocarbazole or dibenzocarbazole skeleton) is further preferable as the host material. Note that in terms of the hole-injection and hole-transport properties described above, instead of a carbazole skeleton, a benzofluorene skeleton or a dibenzofluorene skeleton may be used.

Examples of such a substance include 9-phenyl-3-[4-(10-phenyl-9-anthryl)phenyl]-9H-carbazole (abbreviation: PCzPA), 3-[4-(1-naphthyl)phenyl]-9-phenyl-9H-carbazole (abbreviation: PCPN), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]benzo[b]naphtho[1,2-d]furan (abbreviation: 2mBnfPPA), 9-phenyl-10-[4-(9-phenyl-9H-fluoren-9-yl)biphenyl-4′-yl]anthracene (abbreviation: FLPPA), 9-(1-naphthyl)-10-[4-(2-naphthyl)phenyl]anthracene (abbreviation: αN-βNPAnth), 9-(1-naphthyl)-10-(2-naphthyl)anthracene (abbreviation: α,βADN), 2-(10-phenylanthracen-9-yl)dibenzofuran, 2-(10-phenyl-9-anthracenyl)benzo[b]naphtho[2,3-d]furan (abbreviation: Bnf(II)PhA), 9-(2-naphthyl)-10-[3-(2-naphthyl)phenyl]anthracene (abbreviation: βN-mβNPAnth), and 1-{4-[10-(biphenyl-4-yl)-9-anthracenyl]phenyl}-2-ethyl-1H-benzimidazole (abbreviation: EtBImPBPhA). In particular, CzPA, cgDBCzPA, 2mBnfPPA, and PCzPA exhibit excellent properties and thus are preferably selected.

Note that the host material may be a mixture of a plurality of kinds of substances; in the case of using a mixed host material, it is preferable to mix a material having an electron-transport property with a material having a hole-transport property. By mixing the material having an electron-transport property with the material having a hole-transport property, the transport property of the light-emitting layer 113 can be easily adjusted and a recombination region can be easily controlled. The weight ratio of the content of the material having a hole-transport property to the content of the material having an electron-transport property can be 1:19 to 19:1.

Note that a phosphorescent substance can be used as part of the mixed material. When a fluorescent substance is used as the light-emitting substance, a phosphorescent substance can be used as an energy donor for supplying excitation energy to the fluorescent substance.

An exciplex may be formed of these mixed materials. These mixed materials are preferably selected so as to form an exciplex that exhibits light emission whose wavelength overlaps with the wavelength on a lowest-energy-side absorption band of the light-emitting substance, in which case energy can be transferred smoothly and light emission can be obtained efficiently. The use of such a structure is preferable because the driving voltage can also be reduced.

Note that at least one of the materials forming an exciplex may be a phosphorescent substance. In this case, triplet excitation energy can be efficiently converted into singlet excitation energy by reverse intersystem crossing.

In order to form an exciplex efficiently, a material having an electron-transport property is preferably combined with a material having a hole-transport property and a HOMO level higher than or equal to that of the material having an electron-transport property. In addition, the lowest unoccupied molecular orbital (LUMO) level of the material having a hole-transport property is preferably higher than or equal to that of the material having an electron-transport property. Note that the LUMO levels and the HOMO levels of the materials can be derived from the electrochemical characteristics (the reduction potentials and the oxidation potentials) of the materials that are measured by cyclic voltammetry (CV).

The formation of an exciplex can be confirmed by a phenomenon in which the emission spectrum of the mixed film in which the material having a hole-transport property and the material having an electron-transport property are mixed is shifted to the longer wavelength side than the emission spectrum of each of the materials (or has another peak on the longer wavelength side) observed by comparison of the emission spectra of the material having a hole-transport property, the material having an electron-transport property, and the mixed film of these materials, for example. Alternatively, the formation of an exciplex can be confirmed by a difference in transient response, such as a phenomenon in which the transient photoluminescence (PL) lifetime of the mixed film has a longer lifetime component or has a larger proportion of delayed component than that of each of the materials, observed by comparison of transient PL of the material having a hole-transport property, the material having an electron-transport property, and the mixed film of these materials. The transient PL can be rephrased as transient electroluminescence (EL). That is, the formation of an exciplex can also be confirmed by a difference in transient response observed by comparison of the transient EL of the material having a hole-transport property, the material having an electron-transport property, and the mixed film of these materials.

The electron-transport layer 114 contains a material having an electron-transport property. The material having an electron-transport property preferably has an electron mobility higher than or equal to 1×10⁻⁷ cm²/Vs, further preferably higher than or equal to 1×10⁻⁶ cm²/Vs in the case where the square root of the electric field strength [V/cm] is 600. Note that any other substance can also be used as long as the substance has an electron-transport property higher than a hole-transport property. An organic compound including a π-electron deficient heteroaromatic ring is preferable as the above organic compound. The organic compound including a π-electron deficient heteroaromatic ring is preferably one or more of an organic compound including a heteroaromatic ring having a polyazole skeleton, an organic compound including a heteroaromatic ring having a pyridine skeleton, an organic compound including a heteroaromatic ring having a diazine skeleton, and an organic compound including a heteroaromatic ring having a triazine skeleton.

As the organic compound having an electron-transport property that can be used for the electron-transport layer 114, any of the aforementioned organic compounds that can be given as the organic compound having an electron-transport property in the light-emitting layer 113 can be used. Among the above materials, the organic compound that includes a heteroaromatic ring having a diazine skeleton, the organic compound that includes a heteroaromatic ring having a pyridine skeleton, and the organic compound that includes a heteroaromatic ring having a triazine skeleton are preferable because of having high reliability. In particular, the organic compound that includes a heteroaromatic ring having a diazine (pyrimidine or pyrazine) skeleton and the organic compound that includes a heteroaromatic ring having a triazine skeleton have a high electron-transport property to contribute to a reduction in driving voltage. In particular, an organic compound having a phenanthroline skeleton such as mTpPPhen, PnNPhen, or mPPhen2P is preferable, and an organic compound having a phenanthroline dimer structure such as mPPhen2P is further preferable because of high stability.

Note that the electron-transport layer 114 may have a stacked-layer structure. A layer in the stacked-layer structure of the electron-transport layer 114, which is in contact with the light-emitting layer 113, may function as a hole-blocking layer. In the case where the electron-transport layer in contact with the light-emitting layer functions as a hole-blocking layer, the electron-transport layer is preferably formed using a material having a lower HOMO level than a material contained in the light-emitting layer 113 by greater than or equal to 0.5 eV.

A layer that contains a compound or a complex of an alkali metal or an alkaline earth metal such as 8-hydroxyquinolinato-lithium (abbreviation: Liq), 1,1′-pyridine-2,6-diyl-bis(1,3,4,6,7,8-hexahydro-2H-pyrimido[1,2-a]pyrimidine) (abbreviation: hpp2Py), or the like may be provided as the electron-injection layer 115. As the electron-injection layer 115, an alkali metal, an alkaline earth metal, or a compound thereof may be contained in a layer formed using a substance having an electron-transport property.

Instead of the electron-injection layer 115, a charge-generation layer 116 may be provided (FIG. 1B). The charge-generation layer 116 refers to a layer capable of injecting holes into a layer in contact with the cathode side of the charge-generation layer 116 and electrons into a layer in contact with the anode side thereof when a potential is applied. The charge-generation layer 116 includes at least a p-type layer 117. The p-type layer 117 is preferably formed using any of the composite materials given above as examples of materials that can be used for the hole-injection layer 111. The p-type layer 117 may be formed by stacking a film containing the above-described acceptor material as a material included in the composite material and a film containing a hole-transport material. When a potential is applied to the p-type layer 117, electrons are injected into the electron-transport layer 114 and holes are injected into the cathode; thus, the light-emitting device operates. Since the organic compound of one embodiment of the present invention has a low refractive index, using the organic compound for the p-type layer 117 enables the light-emitting device to have high external quantum efficiency.

Note that the charge-generation layer 116 preferably includes one or both of an electron-relay layer 118 and an electron-injection buffer layer 119 in addition to the p-type layer 117.

The electron-relay layer 118 includes at least the substance having an electron-transport property and has a function of preventing an interaction between the electron-injection buffer layer 119 and the p-type layer 117 and smoothly transferring electrons. The LUMO level of the substance having an electron-transport property included in the electron-relay layer 118 is preferably between the LUMO level of the acceptor substance in the p-type layer 117 and the LUMO level of a substance included in a layer of the electron-transport layer 114 that is in contact with the charge-generation layer 116. As a specific value of the energy level, the LUMO level of the substance having an electron-transport property in the electron-relay layer 118 is preferably higher than or equal to −5.0 eV, further preferably higher than or equal to −5.0 eV and lower than or equal to −3.0 eV. Note that as the substance having an electron-transport property in the electron-relay layer 118, a phthalocyanine-based material or a metal complex having a metal-oxygen bond and an aromatic ligand is preferably used.

The electron-injection buffer layer 119 can be formed using a substance having a high electron-injection property, e.g., an alkali metal, an alkaline earth metal, a rare earth metal, or a compound thereof (an alkali metal compound (including an oxide such as lithium oxide, a halide, and a carbonate such as lithium carbonate or cesium carbonate), an alkaline earth metal compound (including an oxide, a halide, and a carbonate), or a rare earth metal compound (including an oxide, a halide, and a carbonate)).

In the case where the electron-injection buffer layer 119 contains a substance having an electron-transport property and a donor substance, the donor substance can be an organic compound such as tetrathianaphthacene (abbreviation: TTN), nickelocene, or decamethylnickelocene, as well as an alkali metal, an alkaline earth metal, a rare earth metal, or a compound thereof (e.g., an alkali metal compound (including an oxide such as lithium oxide, a halide, and a carbonate such as lithium carbonate or cesium carbonate), an alkaline earth metal compound (including an oxide, a halide, and a carbonate), or a rare earth metal compound (including an oxide, a halide, and a carbonate)). As the substance having an electron-transport property, a material similar to the above-described material for the electron-transport layer 114 can be used.

The second electrode 102 is an electrode including a cathode. The second electrode 102 may have a stacked-layer structure, in which case a layer in contact with the organic compound layer 103 functions as a cathode. For the cathode, a metal, an alloy, an electrically conductive compound, or a mixture thereof each having a low work function (specifically, lower than or equal to 3.8 eV) can be used, for example. Specific examples of such a cathode material include elements belonging to Group 1 or 2 of the periodic table, such as alkali metals (e.g., lithium (Li) or cesium (Cs)), magnesium (Mg), calcium (Ca), and strontium (Sr), alloys containing these elements (e.g., MgAg and AlLi), compounds containing these elements (e.g., lithium fluoride (LiF), cesium fluoride (CsF), and calcium fluoride (CaF₂)), rare earth metals such as europium (Eu) and ytterbium (Yb), and alloys containing these rare earth metals. However, when the electron-injection layer 115 or a thin film formed using any of the above materials having a low work function is provided between the second electrode 102 and the electron-transport layer, a variety of conductive materials such as Al, Ag, ITO, or indium oxide-tin oxide containing silicon or silicon oxide can be used for the cathode regardless of the work function.

When the second electrode 102 is formed using a material that transmits visible light, the light-emitting device can emit light from the second electrode 102 side.

Films of these conductive materials can be deposited by a dry process such as a vacuum evaporation method or a sputtering method, an ink-jet method, a spin coating method, or the like. Alternatively, a wet process using a sol-gel method or a wet process using a paste of a metal material may be employed.

The organic compound layer 103 can be formed by any of a variety of methods, including a dry process and a wet process. For example, a vacuum evaporation method, a gravure printing method, an offset printing method, a screen printing method, an ink-jet method, a spin coating method, or the like may be used.

Different deposition methods may be used to form the electrodes or the layers described above.

Next, an embodiment of a light-emitting device with a structure in which a plurality of light-emitting units are stacked (this type of light-emitting device is also referred to as a stacked or tandem device) is described with reference to FIG. 1C. This light-emitting device includes a plurality of light-emitting units between an anode and a cathode. One light-emitting unit has substantially the same structure as the organic compound layer 103 illustrated in FIG. 1A. In other words, the light-emitting device illustrated in FIG. 1C includes a plurality of light-emitting units, and the light-emitting device illustrated in FIG. 1A or 1B includes a single light-emitting unit.

In FIG. 1C, a first light-emitting unit 511 and a second light-emitting unit 512 are stacked between a first electrode 501 and a second electrode 502, and a charge-generation layer 513 is provided between the first light-emitting unit 511 and the second light-emitting unit 512. The first electrode 501 and the second electrode 502 correspond, respectively, to the first electrode 101 and the second electrode 102 illustrated in FIG. 1A, and the materials given in the description for FIG. 1A can be used. Furthermore, the first light-emitting unit 511 and the second light-emitting unit 512 may have the same structure or different structures.

The charge-generation layer 513 has a function of injecting electrons into one of the light-emitting units and injecting holes into the other of the light-emitting units when voltage is applied between the first electrode 501 and the second electrode 502. That is, in FIG. 1C, the charge-generation layer 513 injects electrons into the first light-emitting unit 511 and holes into the second light-emitting unit 512 when voltage is applied such that the potential of the anode becomes higher than the potential of the cathode.

The charge-generation layer 513 preferably has a structure similar to that of the charge-generation layer 116 described with reference to FIG. 1B. A composite material of an organic compound and a metal oxide enables low-voltage driving and low-current driving because of having an excellent carrier-injection property and an excellent carrier-transport property. In the case where the anode-side surface of a light-emitting unit is in contact with the charge-generation layer 513, the charge-generation layer 513 can also function as a hole-injection layer of the light-emitting unit; therefore, a hole-injection layer is not necessarily provided in the light-emitting unit.

In the case where the electron-injection buffer layer 119 is provided in the charge-generation layer 513, the electron-injection buffer layer 119 functions as the electron-injection layer in the light-emitting unit on the anode side; thus, an electron-injection layer is not necessarily formed in the light-emitting unit on the anode side.

The light-emitting device having two light-emitting units is described with reference to FIG. 1C; however, one embodiment of the present invention can also be applied to a light-emitting device in which three or more light-emitting units are stacked. With a plurality of light-emitting units partitioned by the charge-generation layer 513 between a pair of electrodes as in the light-emitting device of this embodiment, it is possible to provide a long-life device that can emit light with high luminance at a low current density. A light-emitting apparatus that can be driven at a low voltage and has low power consumption can also be provided.

When the emission colors of the light-emitting units are different, light emission of a desired color can be obtained from the light-emitting device as a whole. For example, in a light-emitting device having two light-emitting units, the emission colors of the first light-emitting unit may be red and green and the emission color of the second light-emitting unit may be blue, so that the light-emitting device can emit white light as the whole.

The organic compound layer 103, the first light-emitting unit 511, the second light-emitting unit 512, the layers such as the charge-generation layer, and the electrodes that are described above can be formed by a method such as an evaporation method (including a vacuum evaporation method), a droplet discharge method (also referred to as an ink-jet method), a coating method, or a gravure printing method. A low molecular material, a middle molecular material (including an oligomer and a dendrimer), or a high molecular material may be included in the above components.

Next, an organic semiconductor device of one embodiment of the present invention is described.

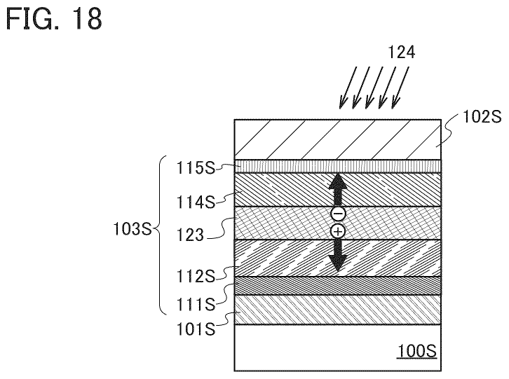

FIG. 18 is a schematic diagram of a photosensor of one embodiment of the present invention. The photosensor includes a first electrode 101S over an insulator 1008 and an organic compound layer 103S between the first electrode 1018 and a second electrode 102S. The organic compound layer 103S includes at least a photoelectric conversion layer 123 and may further include a layer having a different function. The organic compound layer 103S includes the organic compound represented by General Formula (G1) in Embodiment 1.

The photoelectric conversion layer 123 generates carriers and includes a p-type semiconductor and an n-type semiconductor. Charges are generated by light 124 entering the photoelectric conversion layer 123 and can be extracted as current.

The organic compound layer 103S preferably includes, besides the photoelectric conversion layer 123, functional layers such as a hole-injection layer 111S, a hole-transport layer 112S, an electron-transport layer 114S, and an electron-injection layer 115S, as shown in FIG. 18. Note that the organic compound layer 103S may include functional layers other than the above functional layers. Alternatively, any of the above layers may be omitted.

Note that the organic compound represented by General Formula (G1) in Embodiment 1 is preferably contained in a layer where holes are moved. Examples of the layer where holes are moved include a hole-injection layer, a hole-transport layer, an electron-blocking layer, and a photoelectric conversion layer.

In this embodiment, the first electrode 101S and the second electrode 102S each have a single-layer structure or a stacked-layer structure. In the case of the stacked-layer structure, a layer in contact with the organic compound layer 103 serves as an anode or a cathode. In the case where the electrodes each have the stacked-layer structure, there is no limitation on work functions of materials for layers other than the layer in contact with the organic compound layer 103, and the materials can be selected in accordance with required properties such as a resistance value, processing easiness, reflectivity, light-transmitting property, and stability. The first electrode 1018 and the second electrode 102S can be formed using materials similar to those for the first electrode 101 and the second electrode 102, respectively. Note that the electrode which light enters is preferably formed using a material transmitting light with a wavelength that can be converted into current in a photoelectric conversion layer, further preferably formed using a material with a transmittance of 50% or more, still further preferably 70% or more. Of the first electrode 101S and the second electrode 102S, the electrode that receives holes is preferably formed using any of the materials that are given as materials suitable for the anode of the light-emitting device; the electrode that receives electrons is preferably formed using any of the materials that are given as materials suitable for the cathode of the light-emitting device.

The hole-injection layer 111S, the hole-transport layer 112S, the electron-transport layer 114S, the electron-injection layer 115S, and other functional layers can be formed using any of the materials that are given as materials for the functional layers of the light-emitting device. Note that the layer having a function of transporting holes preferably contains the organic compound of one embodiment of the present invention.

The photoelectric conversion layer 123 generates carriers on the basis of incident light and contains a semiconductor. Examples of the semiconductor include an inorganic semiconductor such as silicon and an organic semiconductor including an organic compound. This embodiment shows an example where an organic semiconductor is used as the semiconductor included in the active layer. The use of an organic semiconductor is preferable because the light-emitting layer and the active layer can be formed by the same method (e.g., a vacuum evaporation method) and thus the same manufacturing apparatus can be used.

The photoelectric conversion layer 123 contains at least a p-type semiconductor material and an n-type semiconductor material.

Examples of the p-type semiconductor material include electron-donating organic semiconductor materials such as copper(II) phthalocyanine (CuPc), tetraphenyldibenzoperiflanthene (DBP), zinc phthalocyanine (ZnPc), tin phthalocyanine (SnPc), and quinacridone.

Other examples of the p-type semiconductor material include a carbazole derivative, a thiophene derivative, a furan derivative, and a compound having an aromatic amine skeleton. Other examples of the p-type semiconductor material include a naphthalene derivative, an anthracene derivative, a pyrene derivative, a triphenylene derivative, a fluorene derivative, a pyrrole derivative, a benzofuran derivative, a benzothiophene derivative, an indole derivative, a dibenzofuran derivative, a dibenzothiophene derivative, an indolocarbazole derivative, a porphyrin derivative, a phthalocyanine derivative, a naphthalocyanine derivative, a quinacridone derivative, a polyphenylene vinylene derivative, a polyparaphenylene derivative, a polyfluorene derivative, a polyvinylcarbazole derivative, and a polythiophene derivative.

Examples of the n-type semiconductor material include electron-accepting organic semiconductor materials such as fullerene (e.g., C₆₀ and C₇₀) and fullerene derivatives. Fullerene has a soccer ball-like shape, which is energetically stable. Both the HOMO level and the LUMO level of fullerene are deep (low). Having a deep LUMO level, fullerene has an extremely high electron-accepting property (acceptor property). When π-electron conjugation (resonance) spreads on a plane as in benzene, an electron-donating property (donor property) usually increases; however, fullerene has a spherical shape, and thus has a high electron-accepting property although π-electron conjugation widely spread therein. The high electron-accepting property efficiently causes rapid charge separation and thus is useful for photoelectric conversion devices. Both C₆₀ and C₇₀ have a wide absorption band in the visible light region, and C₇₀ is especially preferable because of having a larger π-electron conjugation system and a wider absorption band in the long wavelength region than C₆₀. Other examples of fullerene derivatives include[6,6]-phenyl-C₇₁-butyric acid methyl ester (abbreviation: PC₇₁BM), [6,6]-phenyl-C₆₁-butyric acid methyl ester (abbreviation: PC₆₁BM), and 1′, 1″, 4′, 4″ tetrahydro-di[1,4]nethanonaphthaleno[1,2:2′, 3′, 56,60:2″, 3″][5,6]fullerene-C₆₀ (abbreviation: ICBA).

Other examples of the n-type semiconductor material include a metal complex having a quinoline skeleton, a metal complex having a benzoquinoline skeleton, a metal complex having an oxazole skeleton, a metal complex having a thiazole skeleton, an oxadiazole derivative, a triazole derivative, an imidazole derivative, an oxazole derivative, a thiazole derivative, a phenanthroline derivative, a quinoline derivative, a benzoquinoline derivative, a quinoxaline derivative, a dibenzoquinoxaline derivative, a pyridine derivative, a bipyridine derivative, a pyrimidine derivative, a naphthalene derivative, an anthracene derivative, a coumarin derivative, a rhodamine derivative, a triazine derivative, and a quinone derivative.

The photoelectric conversion layer 123 is preferably a stacked film of a first layer containing the p-type semiconductor material and a second layer containing the n-type semiconductor material.

In the light-emitting device having any of the aforementioned structures, the photoelectric conversion layer 123 is preferably a mixed film containing the p-type semiconductor material and the n-type semiconductor material.

The HOMO level of the electron-donating organic semiconductor material is preferably shallower (higher) than the HOMO level of the electron-accepting organic semiconductor material. The LUMO level of the electron-donating organic semiconductor material is preferably shallower (higher) than the LUMO level of the electron-accepting organic semiconductor material.

Fullerene having a spherical shape may be used as the electron-accepting organic semiconductor material, and an organic semiconductor material having a substantially planar shape may be used as the electron-donating organic semiconductor material. Molecules of similar shapes tend to aggregate, and aggregated molecules of similar kinds, which have molecular orbital energy levels close to each other, can increase the carrier-transport property.

Since the organic compound represented by General Formula (G1) is a highly heat-resistant material having a favorable hole-transport property, the use of such an organic compound for the light-emitting device and the photosensor of one embodiment of the present invention having the above-described structure can provide a device having a low driving voltage and high reliability at high-temperature driving. A thin film containing the organic compound with such a structure is preferable because it undergoes a small change in quality and can provide a device stable to heat or driving. A device using the organic compound with such a structure has a low driving voltage and a small variation in driving voltage; thus, the device can be highly reliable in voltage and high-temperature driving. Furthermore, a device with low power consumption can be provided, which is preferable. In addition, the organic compound with such a structure is preferable in terms of fabrication costs because it has a high sublimation property, is not decomposed in an evaporation process, and can be produced stably.

### Embodiment 3

Described in this embodiment is a mode in which the organic semiconductor device of one embodiment of the present invention is a light-emitting device that can be used as a display element of a display device. Although this embodiment shows an example in which organic compound layers of light-emitting devices are patterned by a photolithography technique, the light-emitting devices may be formed by evaporation using a mask.

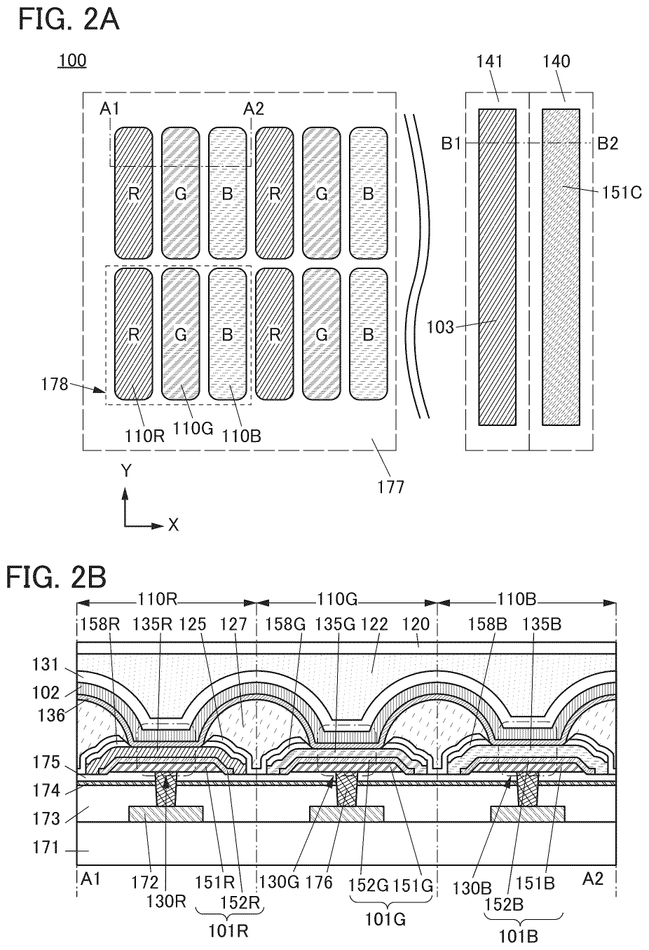

As illustrated in FIGS. 2A and 2B, a plurality of light-emitting devices 130 are formed over an insulating layer 175 to constitute a display device.

A display device includes a pixel portion 177 in which a plurality of pixels 178 are arranged in matrix. The pixel 178 includes a subpixel 110R, a subpixel 110G, and a subpixel 110B.

In this specification and the like, for example, description common to the subpixels 110R, 110G, and 110B is sometimes made using the collective term “subpixel 110”. As for other components that are distinguished from each other using letters of the alphabet, matters common to the components are sometimes described using reference numerals excluding the letters of the alphabet.

The subpixel 110R emits red light, the subpixel 110G emits green light, and the subpixel 110B emits blue light. Thus, an image can be displayed on the pixel portion 177. Note that in this embodiment, three colors of red (R), green (G), and blue (B) are given as examples of colors of light emitted by the subpixels; however, subpixels of a different combination of colors may be employed. The number of subpixels is not limited to three, and may be four or more. Examples of four subpixels include subpixels emitting light of four colors of R, G, B, and white (W), subpixels emitting light of four colors of R, G, B, and yellow (Y), and four subpixels emitting light of R, G, and B and infrared light (IR).

In this specification and the like, the row direction and the column direction are sometimes referred to as the X direction and the Y direction, respectively. The X direction and the Y direction intersect with each other and are perpendicular to each other, for example.

FIG. 2A illustrates an example where subpixels of different colors are arranged in the X direction and subpixels of the same color are arranged in the Y direction. Note that subpixels of different colors may be arranged in the Y direction, and subpixels of the same color may be arranged in the X direction.

Outside the pixel portion 177, a connection portion 140 is provided and a region 141 may also be provided. The region 141 is provided between the pixel portion 177 and the connection portion 140. The organic compound layer 103 is provided in the region 141. A conductive layer 151C is provided in the connection portion 140.

Although FIG. 2A illustrates an example where the region 141 and the connection portion 140 are positioned on the right side of the pixel portion 177, the positions of the region 141 and the connection portion 140 are not particularly limited. The number of regions 141 and the number of connection portions 140 can each be one or more.

FIG. 2B is an example of a cross-sectional view along the dashed-dotted line A1-A2 in FIG. 2A. As illustrated in FIG. 2B, the display device includes an insulating layer 171, a conductive layer 172 over the insulating layer 171, an insulating layer 173 over the insulating layer 171 and the conductive layer 172, an insulating layer 174 over the insulating layer 173, and the insulating layer 175 over the insulating layer 174. The insulating layer 171 is provided over a substrate (not illustrated). An opening reaching the conductive layer 172 is provided in the insulating layers 175, 174, and 173, and a plug 176 is provided to fill the opening.

In the pixel portion 177, the light-emitting device 130 is provided over the insulating layer 175 and the plug 176. A protective layer 131 is provided to cover the light-emitting device 130. A substrate 120 is bonded to the protective layer 131 with a resin layer 122. An inorganic insulating layer 125 and an insulating layer 127 over the inorganic insulating layer 125 are preferably provided between the adjacent light-emitting devices 130.

Although FIG. 2B shows cross sections of a plurality of the inorganic insulating layers 125 and a plurality of the insulating layers 127, it is preferable that the inorganic insulating layers 125 be connected to each other and the insulating layers 127 be connected to each other when the display device is seen from above. In other words, the inorganic insulating layer 125 and the insulating layer 127 each preferably has an opening over a first electrode.

In FIG. 2B, a light-emitting device 130R, a light-emitting device 130G, and a light-emitting device 130B are shown as the light-emitting devices 130. The light-emitting devices 130R, 130G, and 130B emit light of different colors. For example, the light-emitting device 130R can emit red light, the light-emitting device 130G can emit green light, and the light-emitting device 130B can emit blue light. Alternatively, the light-emitting device 130R, 130G, or 130B may emit visible light of another color or infrared light. It can be said that in FIG. 2B, the light-emitting devices 130R and 130G are adjacent light-emitting devices and the light-emitting devices 130G and 130B are adjacent light-emitting devices.

The display device of one embodiment of the present invention can be, for example, a top-emission display device where light is emitted in the direction opposite to a substrate over which light-emitting devices are formed. Note that the display device of one embodiment of the present invention may be of a bottom emission type.

The light-emitting device 130R emits red light (preferably emits phosphorescent light), and preferably has any of the structures shown in Embodiment 1 or 2. The light-emitting device 130R includes a first electrode 101R (pixel electrode) including a conductive layer 151R and a conductive layer 152R, a first layer 135R over the first electrode, a common layer 136 over the first layer 135R, and the second electrode (common electrode) 102 over the common layer 136. The common layer 136 is preferably an electron-injection layer.

The light-emitting device 130G emits green light (preferably emits phosphorescent light), and preferably has any of the structures shown in Embodiment 1 or 2. The light-emitting device 130G includes a first electrode 101G (pixel electrode) including a conductive layer 151G and a conductive layer 152G, a first layer 135G over the first electrode, the common layer 136 over the first layer 135G, and the second electrode (common electrode) 102 over the common layer 136. The common layer 136 is preferably an electron-injection layer.

The light-emitting device 130B emits blue light (preferably emits fluorescent light), and preferably has any of the structures shown in Embodiment 1 or 2. The light-emitting device 130B includes a first electrode 101B (pixel electrode) including a conductive layer 151B and a conductive layer 152B, a first layer 135B over the first electrode, the common layer 136 over the first layer 135B, and the second electrode (common electrode) 102 over the common layer 136. The common layer 136 is preferably an electron-injection layer.

In the light-emitting device, one of the pixel electrode (first electrode) and the common electrode (second electrode) functions as an anode and the other functions as a cathode. In this embodiment, description is made on the assumption that the pixel electrode functions as the anode and the common electrode functions as the cathode unless otherwise specified.

The first layers 135R, 135G, and 135B are island-shaped layers that are independent of each other on a light-emitting device basis or on an emission color basis. It is preferable that the first layers 135R, 135G, and 135B not overlap with one another. The first layers included in the plurality of light-emitting devices 130 formed in the light-emitting apparatus, such as the first layers 135R, 135G, and 135B, are collectively referred to as a first layer group 135A in some cases. Providing the island-shaped first layer group 135A in each of the light-emitting devices 130 can suppress leakage current between the adjacent light-emitting devices 130 even in a high-resolution display device. This can prevent crosstalk, so that a display device with extremely high contrast can be obtained. Specifically, a display device having high current efficiency at low luminance can be obtained.

The island-shaped first layer group 135A is formed by forming an EL film for each emission color and processing the EL film by a photolithography technique.

The first layer 135 is preferably provided to cover the top surface and the side surface of the first electrode (pixel electrode) 101 of the light-emitting device 130. In this case, the aperture ratio of the display device can be easily increased as compared to the structure where an end portion of the first layer 135 is positioned inward from an end portion of the pixel electrode. Covering the side surface of the pixel electrode of the light-emitting device 130 with the first layer 135 can inhibit the first electrode 101 from being in contact with the second electrode 102; hence, a short circuit of the light-emitting device 130 can be inhibited.

In the display device of one embodiment of the present invention, the first electrode (pixel electrode) 101 of the light-emitting device preferably has a stacked-layer structure. For example, in the example illustrated in FIG. 2B, the first electrode 101 of the light-emitting device 130 is a stack of the conductive layer 151 on the insulating layer 171 side and the conductive layer 152 on the organic compound layer side.

A metal material can be used for the conductive layer 151, for example. Specifically, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals, for example.

For the conductive layer 152, an oxide containing one or more selected from indium, tin, zinc, gallium, titanium, aluminum, and silicon can be used. For example, it is preferable to use a conductive oxide containing one or more of indium oxide, indium tin oxide, indium zinc oxide, zinc oxide, zinc oxide containing gallium, titanium oxide, indium zinc oxide containing gallium, indium zinc oxide containing aluminum, indium tin oxide containing silicon, indium zinc oxide containing silicon, and the like. In particular, indium tin oxide containing silicon can be suitably used for the conductive layer 152 because of having a high work function, for example, a work function higher than or equal to 4.0 eV.

The conductive layer 151 and the conductive layer 152 may each be a stack of a plurality of layers containing different materials. In that case, the conductive layer 151 may include a layer formed using a material that can be used for the conductive layer 152, such as a conductive oxide, and the conductive layer 152 may include a layer formed using a material that can be used for the conductive layer 151, such as a metal material. In the case where the conductive layer 151 is a stack of two or more layers, for example, a layer in contact with the conductive layer 152 can be formed using a material that can be used for the conductive layer 152.

The conductive layer 151 preferably has a tapered end portion. Specifically, the conductive layer 151 preferably has a tapered end portion with a taper angle of less than 90°. In that case, the conductive layer 152 provided along the side surface of the conductive layer 151 also has a tapered shape. When the end portion of the conductive layer 152 has a tapered shape, coverage with the first layer 135 provided along the side surface of the conductive layer 152 can be improved.

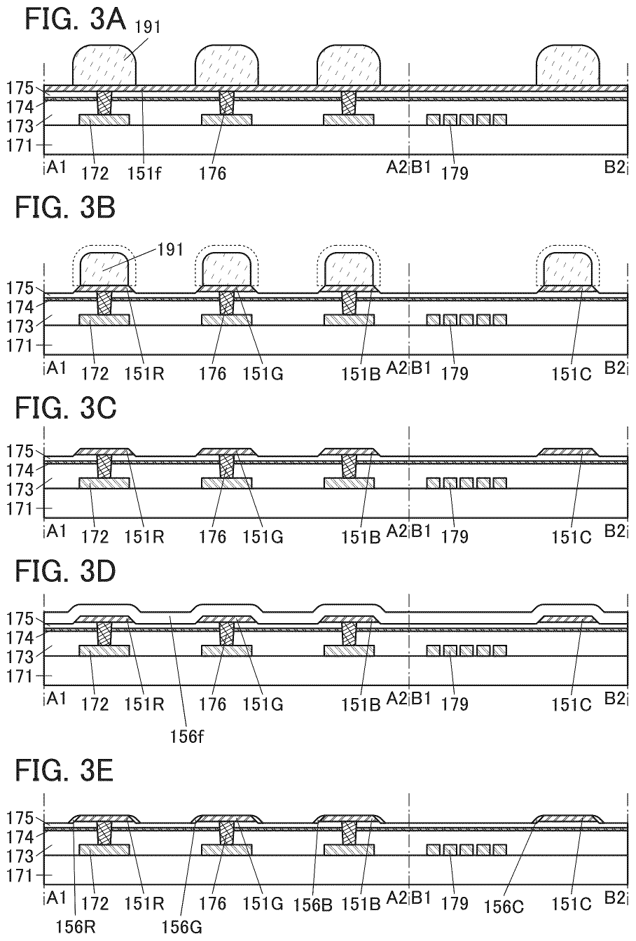

Next, an example of a method for manufacturing the display device having the structure illustrated in FIG. 2A is described with reference to FIGS. 3A to 3E to FIGS. 8A to 8C.

### Manufacturing Method Example

Thin films included in the display device (e.g., insulating films, semiconductor films, and conductive films) can be formed by a sputtering method, a chemical vapor deposition (CVD) method, a vacuum evaporation method, a pulsed laser deposition (PLD) method, an ALD method, or the like.

Thin films included in the display device (e.g., insulating films, semiconductor films, and conductive films) can also be formed by a wet process such as spin coating, dipping, spray coating, ink-jetting, dispensing, screen printing, offset printing, doctor blade coating, slit coating, roll coating, curtain coating, or knife coating.

Thin films included in the display device can be processed by a photolithography technique, for example.

As light used for exposure in the photolithography technique, for example, light with an i-line (wavelength: 365 nm), light with a g-line (wavelength: 436 nm), light with an h-line (wavelength: 405 nm), or light in which the i-line, the g-line, and the h-line are mixed can be used. Alternatively, ultraviolet rays, KrF laser light, ArF laser light, or the like can be used. Exposure may be performed by liquid immersion exposure technique. As the light for exposure, extreme ultraviolet (EUV) light or X-rays may also be used. Furthermore, instead of the light used for exposure, an electron beam can be used.

For etching of thin films, a dry etching method, a wet etching method, a sandblast method, or the like can be used.

First, as illustrated in FIG. 3A, the insulating layer 171 is formed over a substrate (not illustrated). Next, the conductive layer 172 and a conductive layer 179 are formed over the insulating layer 171, and the insulating layer 173 is formed over the insulating layer 171 so as to cover the conductive layer 172 and the conductive layer 179. Then, the insulating layer 174 is formed over the insulating layer 173, and the insulating layer 175 is formed over the insulating layer 174.

As the substrate, a substrate that has heat resistance high enough to withstand at least heat treatment performed later can be used. For example, it is possible to use a glass substrate; a quartz substrate; a sapphire substrate; a ceramic substrate; an organic resin substrate; or a semiconductor substrate such as a single crystal semiconductor substrate or a polycrystalline semiconductor substrate of silicon, silicon carbide, or the like, a compound semiconductor substrate of silicon germanium or the like, or an SOI substrate.

Next, as illustrated in FIG. 3A, openings reaching the conductive layer 172 are formed in the insulating layers 175, 174, and 173. Then, the plugs 176 are formed to fill the openings.

Next, as illustrated in FIG. 3A, a conductive film 151f to be the conductive layers 151R, 151G, 1511B, and 151C is formed over the plugs 176 and the insulating layer 175. A metal material can be used for the conductive film 151f, for example.

Then, a resist mask 191 is formed over the conductive film 151f as illustrated in FIG. 3A. The resist mask 191 can be formed by application of a photosensitive material (photoresist), light exposure, and development.

Subsequently, as illustrated in FIG. 3B, the conductive film 151f in a region not overlapping with the resist mask 191 is removed, for example. In this manner, the conductive layer 151 is formed.

Next, the resist mask 191 is removed as illustrated in FIG. 3C. The resist mask 191 can be removed by ashing using oxygen plasma, for example.

Then, as illustrated in FIG. 3D, an insulating film 156f to be an insulating layer 156R, an insulating layer 156G, an insulating layer 156B, and an insulating layer 156C is formed over the conductive layer 151R, the conductive layer 151G, the conductive layer 151B, the conductive layer 151C, and the insulating layer 175.

As the insulating film 156f, an inorganic insulating film such as an oxide insulating film, a nitride insulating film, an oxynitride insulating film, or a nitride oxide insulating film, e.g., silicon oxynitride, can be used.

Subsequently, as illustrated in FIG. 3E, the insulating film 156f is processed to form the insulating layers 156R, 156G, 156B, and 156C.

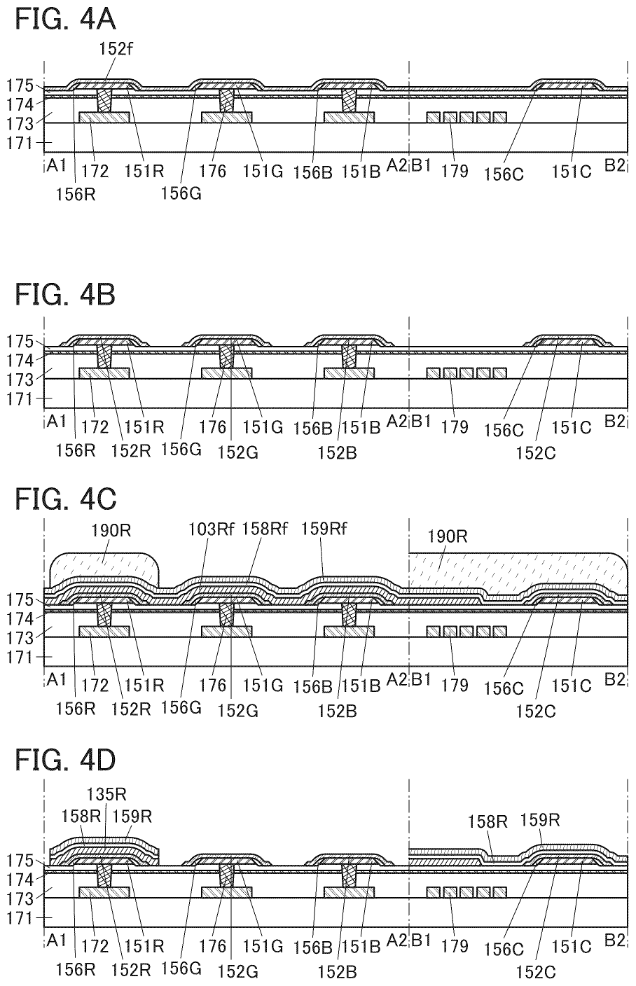

Next, as illustrated in FIG. 4A, a conductive film 152f is formed over the conductive layers 151R, 151G, 151B, and 151C and the insulating layers 156R, 156G, 156B, 156C, and 175.

A conductive oxide can be used for the conductive film 152f, for example. The conductive film 152f may have a stacked-layer structure.

Then, as illustrated in FIG. 4B, the conductive film 152f is processed, so that the conductive layers 152R, 152G, 152B, and 152C are formed.

Next, as illustrated in FIG. 4C, an organic compound film 103Rf is formed over the conductive layers 152R, 152G, and 152B and the insulating layer 175. As illustrated in FIG. 4C, the organic compound film 103Rf is not formed over the conductive layer 152C.

Then, as illustrated in FIG. 4C, a sacrificial film 158Rf and a mask film 159Rf are formed.

Providing the sacrificial film 158Rf over the organic compound film 103Rf can reduce damage to the organic compound film 103Rf in the manufacturing process of the display device, resulting in an increase in the reliability of the light-emitting device.

As the sacrificial film 158Rf, a film that is highly resistant to the process conditions for the organic compound film 103Rf, specifically, a film having high etching selectivity with respect to the organic compound film 103Rf is used. For the mask film 159Rf, a film having high etching selectivity with respect to the sacrificial film 158Rf is used.

The sacrificial film 158Rf and the mask film 159Rf are formed at a temperature lower than the upper temperature limit of the organic compound film 103Rf. The typical substrate temperatures in formation of the sacrificial film 158Rf and the mask film 159Rf are each higher than or equal to 100° C. and lower than or equal to 200° C., preferably higher than or equal to 100° C. and lower than or equal to 150° C., and further preferably higher than or equal to 100° C. and lower than or equal to 120° C.

The sacrificial film 158Rf and the mask film 159Rf are preferably films that can be removed by a wet etching method or a dry etching method.

Note that the sacrificial film 158Rf that is formed on and in contact with the organic compound film 103Rf is preferably formed by a formation method that is less likely to damage the organic compound film 103Rf than a formation method of the mask film 159Rf. For example, the sacrificial film 158Rf is preferably formed by an ALD method or a vacuum evaporation method rather than a sputtering method.

As each of the sacrificial film 158Rf and the mask film 159Rf, one or more of a metal film, an alloy film, a metal oxide film, a semiconductor film, an organic insulating film, and an inorganic insulating film, for example, can be used.

For each of the sacrificial film 158Rf and the mask film 159Rf, a metal material such as gold, silver, platinum, magnesium, nickel, tungsten, chromium, molybdenum, iron, cobalt, copper, palladium, titanium, aluminum, yttrium, zirconium, or tantalum or an alloy material containing any of the metal materials can be used, for example. It is particularly preferable to use a low-melting-point material such as aluminum or silver. It is preferable to use a metal material that can block ultraviolet rays for one or both of the sacrificial film 158Rf and the mask film 159Rf, in which case the organic compound film 103Rf can be inhibited from being irradiated with ultraviolet rays in patterning light exposure, and deterioration of the organic compound film 103Rf can be inhibited.

The sacrificial film 158Rf and the mask film 159Rf can each be formed using a metal oxide such as In—Ga—Zn oxide, indium oxide, In—Zn oxide, In—Sn oxide, indium titanium oxide (In—Ti oxide), indium tin zinc oxide (In—Sn—Zn oxide), indium titanium zinc oxide (In—Ti—Zn oxide), indium gallium tin zinc oxide (In—Ga—Sn—Zn oxide), or indium tin oxide containing silicon.

In the above metal oxide, in place of gallium, an element M (M is one or more of aluminum, silicon, boron, yttrium, copper, vanadium, beryllium, titanium, iron, nickel, germanium, zirconium, molybdenum, lanthanum, cerium, neodymium, hafnium, tantalum, tungsten, and magnesium) may be used.

The sacrificial film 158Rf and the mask film 159Rf are preferably formed using a semiconductor material such as silicon or germanium for excellent compatibility with a semiconductor manufacturing process. Alternatively, a compound containing the above semiconductor material can be used.

As each of the sacrificial film 158Rf and the mask film 159Rf, any of a variety of inorganic insulating films can be used. In particular, an oxide insulating film is preferable because its adhesion to the organic compound film 103Rf is higher than that of a nitride insulating film.

Subsequently, a resist mask 190R is formed as illustrated in FIG. 4C. The resist mask 190R can be formed by application of a photosensitive material (photoresist), light exposure, and development.

The resist mask 190R is provided at a position overlapping with the conductive layer 152R. The resist mask 190R is preferably provided also at a position overlapping with the conductive layer 152C. This can inhibit the conductive layer 152C from being damaged during the process of manufacturing the display device.

Next, as illustrated in FIG. 4D, part of the mask film 159Rf is removed using the resist mask 190R, so that a mask layer 159R is formed. The mask layer 159R remains over the conductive layers 152R and 152C. After that, the resist mask 190R is removed. Then, part of the sacrificial film 158Rf is removed using the mask layer 159R as a mask (also referred to as a hard mask), so that the sacrificial layer 158R is formed.

The use of a wet etching method can reduce damage to the organic compound film 103Rf in processing of the sacrificial film 158Rf and the mask film 159Rf, as compared to the case of using a dry etching method. In the case of using a wet etching method, it is preferable to use a developer, an alkaline aqueous solution such as a tetramethylammonium hydroxide (TMAH) aqueous solution, or an acid aqueous solution such as/of dilute hydrofluoric acid, oxalic acid, phosphoric acid, acetic acid, nitric acid, or a chemical solution containing a mixed solution of any of these acids, for example.

In the case of using a dry etching method to process the sacrificial film 158Rf, deterioration of the organic compound film 103Rf can be inhibited by not using a gas containing oxygen as the etching gas.

The resist mask 190R can be removed by a method similar to that for the resist mask 191.

Next, as illustrated in FIG. 4D, the organic compound film 103Rf is processed to form the first layer 135R. For example, part of the organic compound film 103Rf is removed using the mask layer 159R and the sacrificial layer 158R as a hard mask, whereby the first layer 135R is formed.

Accordingly, as illustrated in FIG. 4D, the stacked-layer structure of the first layer 135R, the sacrificial layer 158R, and the mask layer 159R remains over the conductive layer 152R. The conductive layers 152G and 152B are exposed.

The organic compound film 103Rf is preferably processed by anisotropic etching. Anisotropic dry etching is particularly preferable. Alternatively, wet etching may be used.

In the case of using a dry etching method, deterioration of the organic compound film 103Rf can be inhibited by not using a gas containing oxygen as the etching gas.

A gas containing oxygen may be used as the etching gas. When the etching gas contains oxygen, the etching rate can be increased. Therefore, the etching can be performed under a low-power condition while an adequately high etching rate is maintained. Accordingly, damage to the organic compound film 103Rf can be reduced. Furthermore, a defect such as attachment of a reaction product generated during the etching can be inhibited.

In the case of using a dry etching method, it is preferable to use a gas containing at least one of H₂, CF₄, C₄F₈, SF₆, CHF₃, Cl₂, H₂O, BCl₃, and a Group 18 element such as He or Ar as the etching gas, for example. Alternatively, a gas containing oxygen and at least one of the above is preferably used as the etching gas. Alternatively, an oxygen gas may be used as the etching gas.

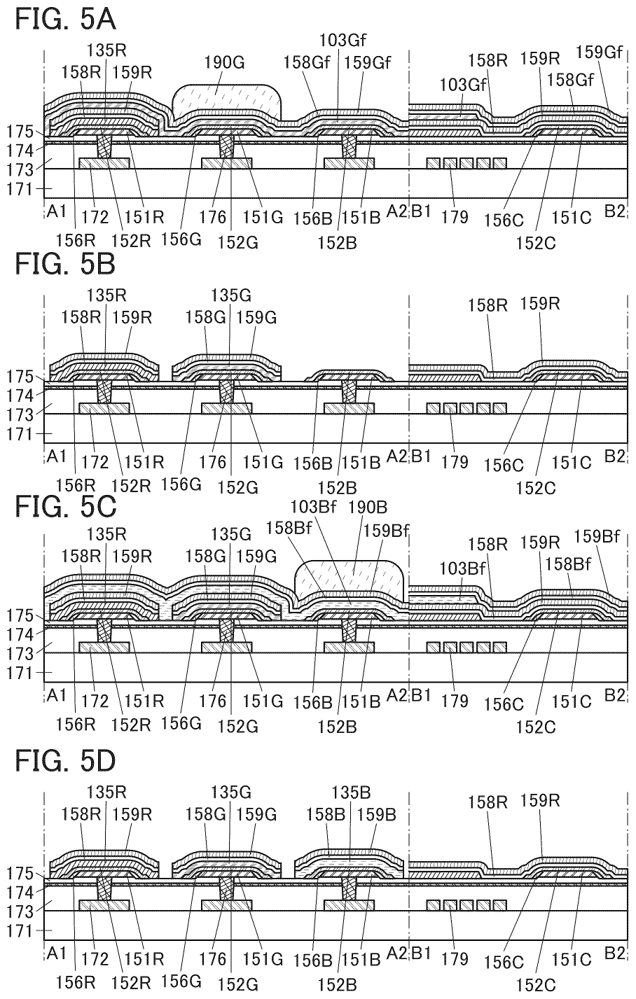

Then, as illustrated in FIG. 5A, an organic compound film 103Gf to be the first layer 135G is formed.

The organic compound film 103Gf can be formed by a method similar to that for forming the organic compound film 103Rf. The organic compound film 103Gf can have a structure similar to that of the organic compound film 103Rf.

Subsequently, as illustrated in FIG. 5A, a sacrificial film 158Gf and a mask film 159Gf are formed in this order. After that, a resist mask 190G is formed. The materials and the formation methods of the sacrificial film 158Gf and the mask film 159Gf are similar to those for the sacrificial film 158Rf and the mask film 159Rf. The material and the formation method of the resist mask 190G are similar to those for the resist mask 190R.

The resist mask 190G is provided at a position overlapping with the conductive layer 152G.

Subsequently, as illustrated in FIG. 5B, part of the mask film 159Gf is removed using the resist mask 190G, so that a mask layer 159G is formed. The mask layer 159G remains over the conductive layer 152G. After that, the resist mask 190G is removed. Then, part of the sacrificial film 158Gf is removed using the mask layer 159G as a mask, so that the sacrificial layer 158G is formed. Next, the organic compound film 103Gf is processed to form the first layer 135G.

Then, an organic compound film 103Bf is formed as illustrated in FIG. 5C.

The organic compound film 103Bf can be formed by a method similar to that for forming the organic compound film 103Rf. The organic compound film 103Bf can have a structure similar to that of the organic compound film 103Rf.

Subsequently, a sacrificial film 158Bf and a mask film 159Bf are formed in this order as illustrated in FIG. 5C. After that, a resist mask 190B is formed. The materials and the formation methods of the sacrificial film 158Bf and the mask film 159Bf are similar to those for the sacrificial film 158Rf and the mask film 159Rf. The material and the formation method of the resist mask 190B are similar to those for the resist mask 190R.

The resist mask 190B is provided at a position overlapping with the conductive layer 152B.

Subsequently, as illustrated in FIG. 5D, part of the mask film 159Bf is removed using the resist mask 190B, so that a mask layer 159B is formed. The mask layer 159B remains over the conductive layer 152B. After that, the resist mask 190B is removed. Then, part of the sacrificial film 158Bf is removed using the mask layer 159B as a mask, so that the sacrificial layer 158B is formed. Next, the organic compound film 103Bf is processed to form the first layer 135B. For example, part of the organic compound film 103Bf is removed using the mask layer 159B and the sacrificial layer 158B as a hard mask, whereby the first layer 135B is formed.

Accordingly, the stacked-layer structure of the first layer 135B, the sacrificial layer 158B, and the mask layer 159B remains over the conductive layer 152B as illustrated in FIG. 5D. The mask layers 159R and 159G are exposed.

Note that the side surfaces of the first layers 135R, 135G, and 135B are preferably perpendicular or substantially perpendicular to their formation surfaces. For example, the angle between the formation surfaces and these side surfaces is preferably greater than or equal to 60° and less than or equal to 90°.

The distance between two adjacent layers among the first layers 135R, 135G, and 135B, which are formed by a photolithography technique as described above, can be reduced to less than or equal to 8 μm, less than or equal to 5 μm, less than or equal to 3 μm, less than or equal to 2 Pm, or less than or equal to 1 μm. Here, the distance can be specified, for example, by the distance between opposite end portions of two adjacent layers among the first layers 135R, 135G, and 135B. Reducing the distance between the island-shaped organic compound layers makes it possible to provide a display device having high resolution and a high aperture ratio. In addition, the distance between the first electrodes of adjacent light-emitting devices can also be shortened to be, for example, less than or equal to 10 μm, less than or equal to 8 jam, less than or equal to 5 pam, less than or equal to 3 μm, or less than or equal to 2 μm. Note that the distance between the first electrodes of adjacent light-emitting devices is preferably greater than or equal to 2 μm and less than or equal to 5 μm.

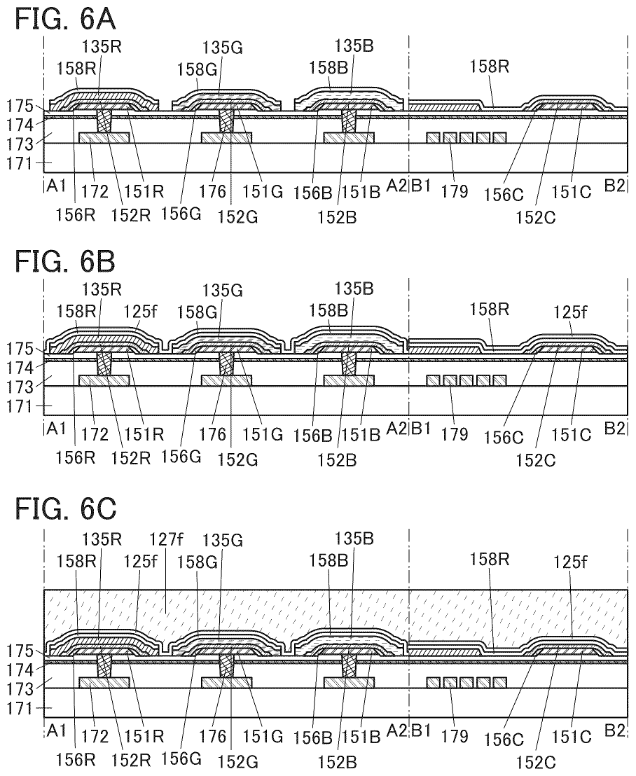

Next, the mask layers 159R, 159G, and 159B are preferably removed as illustrated in FIG. 6A.

The step of removing the mask layers can be performed by a method similar to that for the step of processing the mask films. Specifically, by using a wet etching method, damage caused to the first layer 135 at the time of removing the mask layers can be reduced as compared to the case of using a dry etching method.

The mask layers may be removed by being dissolved in a polar solvent such as water or an alcohol. Examples of an alcohol include ethyl alcohol, methyl alcohol, isopropyl alcohol (IPA), and glycerin.

After the mask layers are removed, drying treatment may be performed in order to remove water adsorbed on surfaces. For example, heat treatment in an inert gas atmosphere or a reduced-pressure atmosphere can be performed. The heat treatment can be performed at a substrate temperature higher than or equal to 50° C. and lower than or equal to 200° C., preferably higher than or equal to 60° C. and lower than or equal to 150° C., and further preferably higher than or equal to 70° C. and lower than or equal to 120° C. The heat treatment is preferably performed in a reduced-pressure atmosphere, in which case drying at a lower temperature is possible.

Next, an inorganic insulating film 125f is formed as illustrated in FIG. 6B.

Then, as illustrated in FIG. 6C, an insulating film 127f to be the insulating layer 127 is formed over the inorganic insulating film 125f.

The substrate temperature at the time of forming the inorganic insulating film 125f and the insulating film 127f is preferably higher than or equal to 60° C., higher than or equal to 80° C., higher than or equal to 100° C., or higher than or equal to 120° C. and lower than or equal to 200° C., lower than or equal to 180° C., lower than or equal to 160° C., lower than or equal to 150° C., or lower than or equal to 140° C.

As the inorganic insulating film 125f, an insulating film having a thickness of 3 nm or more, 5 nm or more, or 10 nm or more and 200 nm or less, 150 nm or less, 100 nm or less, or 50 nm or less is preferably formed at a substrate temperature in the above-described range.

The inorganic insulating film 125f is preferably formed by an ALD method, for example. An ALD method is preferably used, in which case deposition damage is reduced and a film with good coverage can be formed. As the inorganic insulating film 125f, an aluminum oxide film is preferably formed by an ALD method, for example.

The insulating film 127f is preferably formed by the aforementioned wet process. For example, the insulating film 127f is preferably formed by spin coating using a photosensitive material, and specifically preferably formed using a photosensitive resin composition containing an acrylic resin.

Then, part of the insulating film 127f is exposed to visible light or ultraviolet rays. The insulating layer 127 is formed in regions that are sandwiched between any two of the conductive layers 152R, 152G, and 152B and around the conductive layer 152C.

The width of the insulating layer 127 formed later can be controlled in accordance with the exposed region of the insulating film 127f. In this embodiment, processing is performed such that the insulating layer 127 includes a portion overlapping with the top surface of the conductive layer 151.

Light used for the exposure preferably includes the i-line (wavelength: 365 nm). Furthermore, light used for the exposure may include at least one of the g-line (wavelength: 436 nm) and the h-line (wavelength: 405 nm).

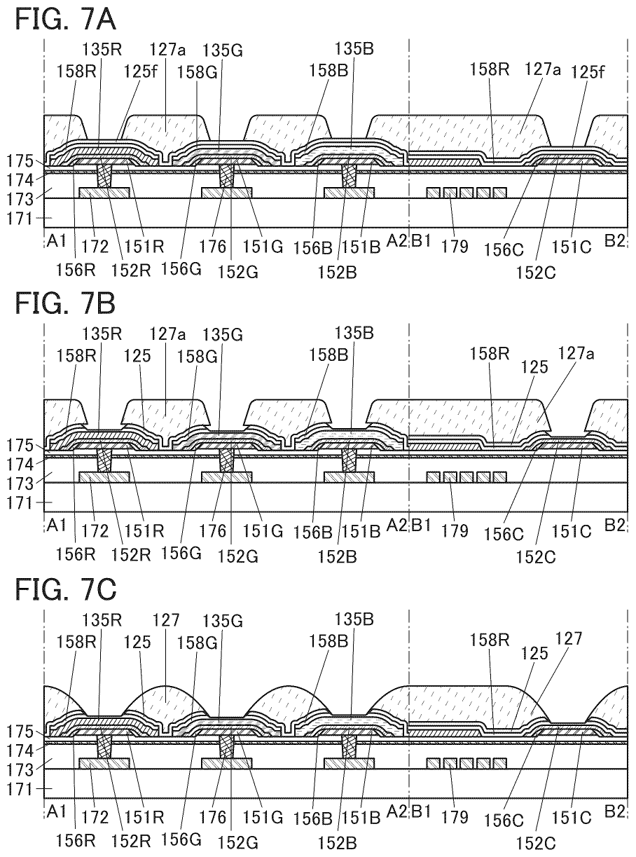

Next, the region of the insulating film 127f exposed to light is removed by development as illustrated in FIG. 7A, so that an insulating layer 127a is formed.

Next, as illustrated in FIG. 7B, etching treatment is performed using the insulating layer 127a as a mask to remove part of the inorganic insulating film 125f and reduce the thickness of part of the sacrificial layers 158R, 158G, and 158B. Thus, the inorganic insulating layer 125 is formed under the insulating layer 127a. Moreover, the surfaces of the thin portions in the sacrificial layers 158R, 158G, and 158B are exposed. Note that the etching treatment using the insulating layer 127a as a mask may be hereinafter referred to as first etching treatment.

The first etching treatment can be performed by dry etching or wet etching. Note that the inorganic insulating film 125f is preferably formed using a material similar to that of the sacrificial layers 158R, 158G, and 158B, in which case the first etching treatment can be performed concurrently.

In the case of performing dry etching, a chlorine-based gas is preferably used. As the chlorine-based gas, one of Cl₂, BCl₃, SiCl₄, CCl₄, and the like or a mixture of two or more of them can be used. Moreover, one of an oxygen gas, a hydrogen gas, a helium gas, an argon gas, and the like or a mixture of two or more of them can be added as appropriate to the chlorine-based gas. By the dry etching, the thin regions of the sacrificial layers 158R, 158G, and 158B can be formed with favorable in-plane uniformity.

As a dry etching apparatus, a dry etching apparatus including a high-density plasma source can be used. As the dry etching apparatus including a high-density plasma source, an inductively coupled plasma (ICP) etching apparatus can be used, for example. Alternatively, a capacitively coupled plasma (CCP) etching apparatus including parallel plate electrodes can be used.

The first etching treatment is preferably performed by wet etching. The use of a wet etching method can reduce damage to the first layers 135R, 135G, and 135B, as compared to the case of using a dry etching method. Wet etching can be performed using an alkaline solution, for example. For instance, TMAH, which is an alkaline solution, can be used for the wet etching of an aluminum oxide film. Alternatively, an acid solution containing fluoride can also be used. In this case, puddle wet etching can be performed. Note that the inorganic insulating film 125f is preferably formed using a material similar to that of the sacrificial layers 158R, 158G, and 158B, in which case the above etching treatment can be performed concurrently.

The sacrificial layers 158R, 158G, and 158B are not removed completely by the first etching treatment, and the etching treatment is stopped when the thickness of the sacrificial layers 158R, 158G, and 158B is reduced. The corresponding sacrificial layers 158R, 158G, and 158B remain over the first layers 135R, 135G, and 135B in this manner, whereby the first layers 135R, 135G, and 135B can be prevented from being damaged by treatment in a later step.

Next, light exposure is preferably performed on the entire substrate so that the insulating layer 127a is irradiated with visible light or ultraviolet rays. The energy density for the light exposure is preferably greater than 0 mJ/cm² and less than or equal to 800 mJ/cm², further preferably greater than 0 mJ/cm² and less than or equal to 500 mJ/cm². Performing such light exposure after the development can sometimes increase the degree of transparency of the insulating layer 127a. In addition, it is sometimes possible to lower the substrate temperature required for subsequent heat treatment for changing the shape of the insulating layer 127a into a tapered shape.

Here, when a barrier insulating layer against oxygen (e.g., an aluminum oxide film) exists as each of the sacrificial layers 158R, 158G, and 158B, diffusion of oxygen into the first layers 135R, 135G, and 135B can be suppressed.

Then, heat treatment (also referred to as post-baking) is performed. The heat treatment can change the insulating layer 127a into the insulating layer 127 having a tapered side surface (FIG. 7C). The heat treatment is conducted at a temperature lower than the upper temperature limit of the organic compound layer. The heat treatment can be performed at a substrate temperature higher than or equal to 50° C. and lower than or equal to 200° C., preferably higher than or equal to 60° C. and lower than or equal to 150° C., and further preferably higher than or equal to 70° C. and lower than or equal to 130° C. The heating atmosphere may be an air atmosphere or an inert gas atmosphere. Moreover, the heating atmosphere may be an atmospheric-pressure atmosphere or a reduced-pressure atmosphere. Accordingly, adhesion between the insulating layer 127 and the inorganic insulating layer 125 can be improved, and corrosion resistance of the insulating layer 127 can be increased.

When the sacrificial layers 158R, 158G, and 158B are not completely removed by the first etching treatment and the thinned sacrificial layers 158R, 158G, and 158B are left, the first layers 135R, 135G, and 135B can be prevented from being damaged and deteriorating in the heat treatment. This increases the reliability of the light-emitting device.

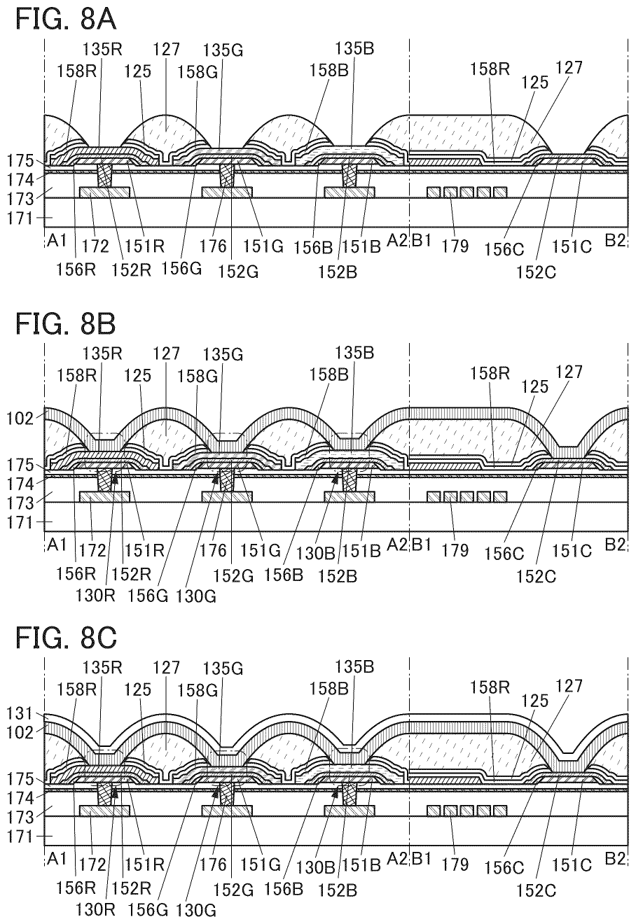

Next, as illustrated in FIG. 8A, etching treatment is performed using the insulating layer 127 as a mask to remove part of the sacrificial layers 158R, 158G, and 158B. Thus, openings are formed in the sacrificial layers 158R, 158G, and 158B, and the top surfaces of the first layers 135R, 135G, and 135B and the conductive layer 152C are exposed. Note that this etching treatment may be hereinafter referred to as second etching treatment.

An end portion of the inorganic insulating layer 125 is covered with the insulating layer 127. FIG. 8A illustrates an example in which part of an end portion of the sacrificial layer 158G (specifically, a tapered portion formed by the first etching treatment) is covered with the insulating layer 127 and a tapered portion formed by the second etching treatment is exposed.

The second etching treatment is performed by wet etching. The use of a wet etching method can reduce damage to the first layers 135R, 135G, and 135B, as compared to the case of using a dry etching method. Wet etching can be performed using an alkaline solution or an acidic solution, for example. An aqueous solution is preferably used in order that the first layer 135 is not dissolved.

Next, as illustrated in FIG. 8B, the second electrode 102 is formed over the first layers 135R, 135G, and 135B, the conductive layer 152C, and the insulating layer 127. The second electrode 102 can be formed by a sputtering method, a vacuum evaporation method, or the like. In this case, a stacked layer structure of the first layer 135 and the common layer 136 may be employed as illustrated in FIGS. 2A and 2B, and the second electrode 102 may be formed thereover.

Next, as illustrated in FIG. 8C, the protective layer 131 is formed over the second electrode 102. The protective layer 131 can be formed by a vacuum evaporation method, a sputtering method, a CVD method, an ALD method, or the like.

Then, the substrate 120 is bonded to the protective layer 131 with the resin layer 122, so that the display device can be manufactured. In the method for manufacturing the display device of one embodiment of the present invention, the insulating layer 156 is formed to include a region overlapping with the side surface of the conductive layer 151 and the conductive layer 152 is formed to cover the conductive layer 151 and the insulating layer 156 as described above. This can increase the yield of the display device and inhibit generation of defects.

As described above, in the method for manufacturing the display device in one embodiment of the present invention, the island-shaped first layers 135R, 135G, and 135B are formed not by using a fine metal mask but by processing a film formed on the entire surface; thus, the island-shaped layers can be formed to have a uniform thickness. Consequently, a high-resolution display device or a display device with a high aperture ratio can be obtained. Furthermore, even when the resolution or the aperture ratio is high and the distance between the subpixels is extremely short, the first layers 135R, 135G, and 135B can be inhibited from being in contact with each other in the adjacent subpixels. As a result, generation of leakage current between the subpixels can be inhibited. This can prevent crosstalk, so that a display device with extremely high contrast can be obtained. Moreover, even a display device that includes tandem light-emitting devices formed by a photolithography technique can have favorable characteristics.

### Embodiment 4

In this embodiment, a display device of one embodiment of the present invention will be described.

The display device in this embodiment can be a high-resolution display device. Thus, the display device in this embodiment can be used for display portions of information terminals (wearable devices) such as watch-type and bracelet-type information terminals and display portions of wearable devices capable of being worn on a head, such as a VR device like a head mounted display (HMD) and a glasses-type AR device.

The display device in this embodiment can be a high-definition display device or a large-sized display device. Accordingly, the display device in this embodiment can be used for display portions of a digital camera, a digital video camera, a digital photo frame, a mobile phone, a portable game console, a portable information terminal, and an audio reproducing device, in addition to display portions of electronic devices with a relatively large screen, such as a television device, desktop and notebook personal computers, a monitor of a computer and the like, digital signage, and a large game machine such as a pachinko machine.

### [Display Module]

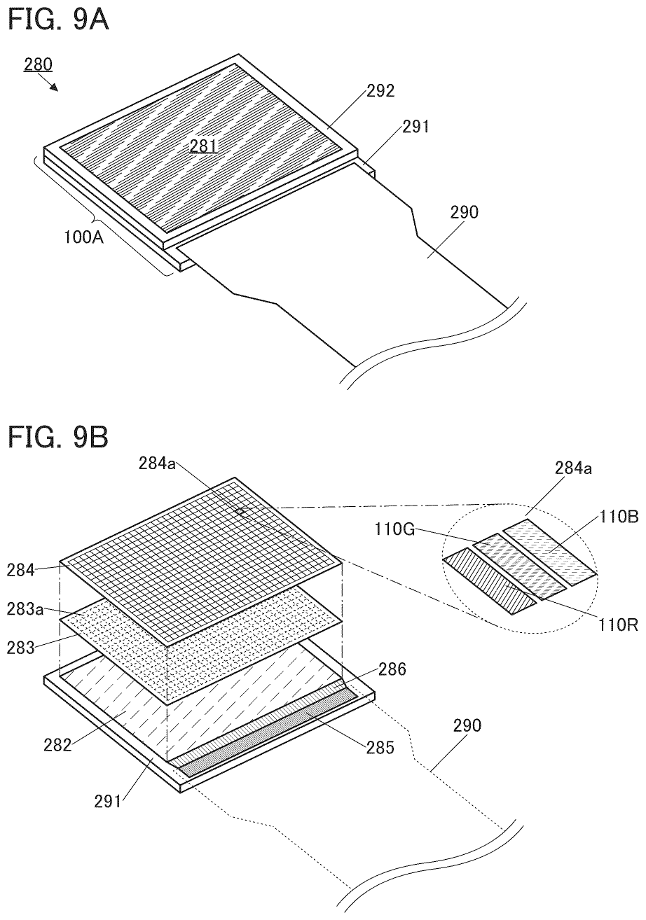

FIG. 9A is a perspective view of a display module 280. The display module 280 includes a display device 100A and an FPC 290. Note that the display device included in the display module 280 is not limited to the display device 100A and may be any of display devices 100B to 100E described later.

The display module 280 includes a substrate 291 and a substrate 292. The display module 280 includes a display portion 281. The display portion 281 is a region of the display module 280 where an image is displayed, and is a region where light emitted from pixels provided in a pixel portion 284 described later can be seen.

FIG. 9B is a perspective view schematically illustrating the structure on the substrate 291 side. Over the substrate 291, a circuit portion 282, a pixel circuit portion 283 over the circuit portion 282, and the pixel portion 284 over the pixel circuit portion 283 are stacked. In addition, a terminal portion 285 for connection to the FPC 290 is included in a portion over the substrate 291 that does not overlap with the pixel portion 284. The terminal portion 285 and the circuit portion 282 are electrically connected to each other through a wiring portion 286 formed of a plurality of wirings.

The pixel portion 284 includes a plurality of pixels 284a arranged periodically. An enlarged view of one pixel 284a is illustrated on the right side in FIG. 9B. The pixels 284a can employ any of the structures described in the above embodiments. FIG. 9B illustrates an example where the pixel 284a has a structure similar to that of the pixel 178 illustrated in FIGS. 2A and 2B.

The pixel circuit portion 283 includes a plurality of pixel circuits 283a arranged periodically.

One pixel circuit 283a is a circuit that controls driving of a plurality of devices included in one pixel 284a.

The circuit portion 282 includes a circuit for driving the pixel circuits 283a in the pixel circuit portion 283. For example, the circuit portion 282 preferably includes one or both of a gate line driver circuit and a source line driver circuit. The circuit portion 282 may also include at least one of an arithmetic circuit, a memory circuit, a power supply circuit, and the like.

The FPC 290 functions as a wiring for supplying a video signal, a power supply potential, or the like to the circuit portion 282 from the outside. An IC may be mounted on the FPC 290.

The display module 280 can have a structure in which one or both of the pixel circuit portion 283 and the circuit portion 282 are stacked below the pixel portion 284; hence, the aperture ratio (effective display area ratio) of the display portion 281 can be significantly high.

Such a display module 280 has extremely high resolution, and thus can be suitably used for a VR device such as an HMD or a glasses-type AR device. For example, even in the case of a structure in which the display portion of the display module 280 is seen through a lens, pixels of the extremely-high-resolution display portion 281 included in the display module 280 are prevented from being recognized when the display portion is enlarged by the lens, so that display providing a high sense of immersion can be performed. Without being limited thereto, the display module 280 can be suitably used for electronic devices including a relatively small display portion.

### [Display Device 100A]

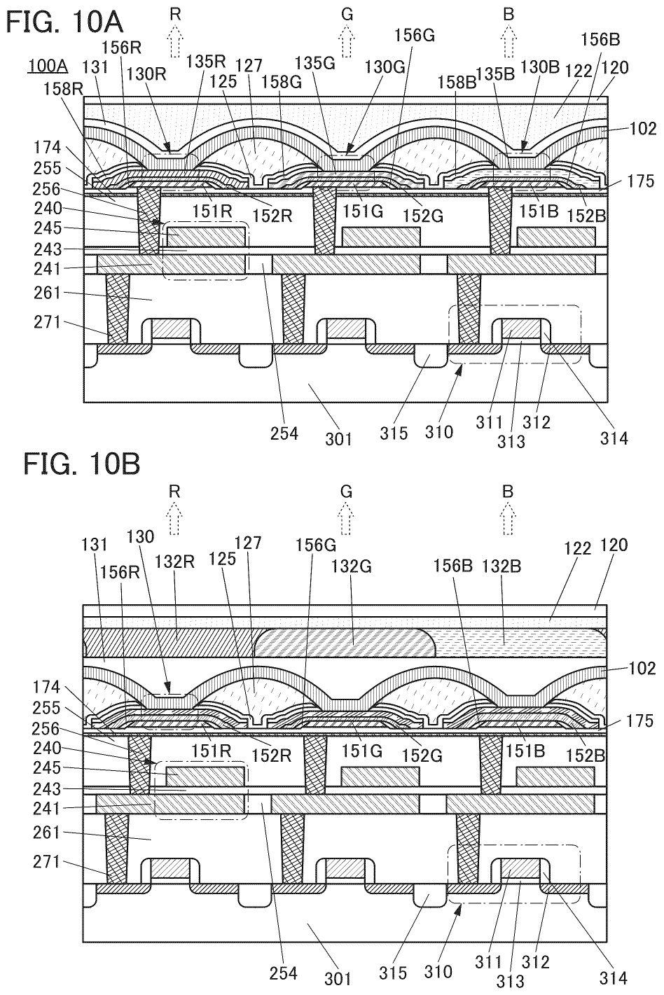

The display device 100A illustrated in FIG. 10A includes a substrate 301, the light-emitting devices 130R, 130G, and 130B, a capacitor 240, and a transistor 310.

The substrate 301 corresponds to the substrate 291 in FIGS. 9A and 9B. The transistor 310 includes a channel formation region in the substrate 301. As the substrate 301, a semiconductor substrate such as a single crystal silicon substrate can be used, for example. The transistor 310 includes part of the substrate 301, a conductive layer 311, a low-resistance region 312, an insulating layer 313, and an insulating layer 314. The conductive layer 311 functions as a gate electrode. The insulating layer 313 is positioned between the substrate 301 and the conductive layer 311 and functions as a gate insulating layer. The low-resistance region 312 is a region where the substrate 301 is doped with an impurity, and functions as a source or a drain. The insulating layer 314 is provided to cover the side surface of the conductive layer 311.

An element isolation layer 315 is provided between two adjacent transistors 310 so as to be embedded in the substrate 301.

An insulating layer 261 is provided to cover the transistor 310, and the capacitor 240 is provided over the insulating layer 261.

The capacitor 240 includes a conductive layer 241, a conductive layer 245, and an insulating layer 243 between the conductive layers 241 and 245. The conductive layer 241 functions as one electrode of the capacitor 240, the conductive layer 245 functions as the other electrode of the capacitor 240, and the insulating layer 243 functions as a dielectric of the capacitor 240.

The conductive layer 241 is provided over the insulating layer 261 and is embedded in an insulating layer 254. The conductive layer 241 is electrically connected to one of the source and the drain of the transistor 310 through a plug 271 embedded in the insulating layer 261. The insulating layer 243 is provided to cover the conductive layer 241. The conductive layer 245 is provided in a region overlapping with the conductive layer 241 with the insulating layer 243 therebetween.

An insulating layer 255 is provided to cover the capacitor 240. The insulating layer 174 is provided over the insulating layer 255. The insulating layer 175 is provided over the insulating layer 174. The light-emitting devices 130R, 130G, and 130B are provided over the insulating layer 175. An insulator is provided in regions between adjacent light-emitting devices.

The insulating layer 156R is provided to include a region overlapping with the side surface of the conductive layer 151R. The insulating layer 156G is provided to include a region overlapping with the side surface of the conductive layer 151G. The insulating layer 156B is provided to include a region overlapping with the side surface of the conductive layer 151B. The conductive layer 152R is provided to cover the conductive layer 151R and the insulating layer 156R. The conductive layer 152G is provided to cover the conductive layer 151G and the insulating layer 156G. The conductive layer 152B is provided to cover the conductive layer 151B and the insulating layer 156B. The sacrificial layer 158R is positioned over the first layer 135R. The sacrificial layer 158G is positioned over the first layer 135G. The sacrificial layer 158B is positioned over the first layer 135B.

Each of the conductive layers 151R, 151G, and 151B is electrically connected to one of the source and the drain of the corresponding transistor 310 through a plug 256 embedded in the insulating layers 243, 255, 174, and 175, the conductive layer 241 embedded in the insulating layer 254, and the plug 271 embedded in the insulating layer 261. Any of a variety of conductive materials can be used for the plugs.

The protective layer 131 is provided over the light-emitting devices 130R, 130G, and 130B. The substrate 120 is bonded to the protective layer 131 with the resin layer 122. Embodiment 3 can be referred to for the details of the light-emitting device 130 and the components thereover up to the substrate 120. The substrate 120 corresponds to the substrate 292 in FIG. 9A.

FIG. 10B illustrates a variation example of the display device 100A illustrated in FIG. 10A. The display device illustrated in FIG. 10B includes the coloring layers 132R, 132G, and 132B, and each of the light-emitting devices 130 includes a region overlapping with one of the coloring layers 132R, 132G, and 132B. In the display device illustrated in FIG. 10B, the light-emitting device 130 can emit white light, for example. The coloring layer 132R, the coloring layer 132G, and the coloring layer 132B can transmit red light, green light, and blue light, respectively, for example.

### [Display Device 100B]

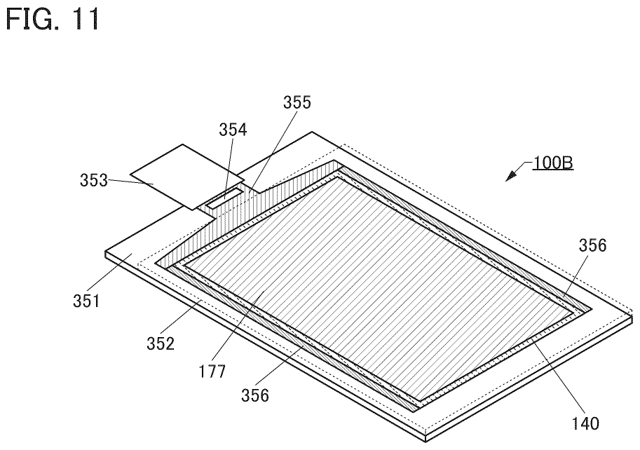

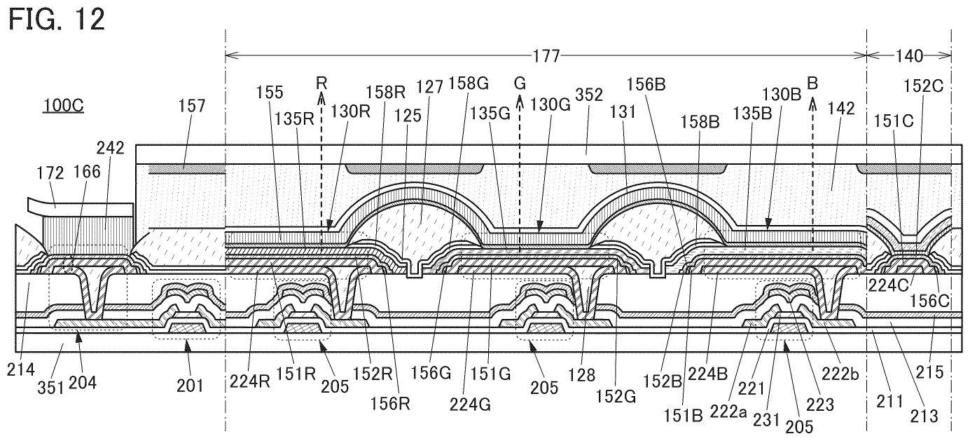

FIG. 11 is a perspective view of the display device 100B, and FIG. 12 is a cross-sectional view of the display device 100C.

In the display device 100B, a substrate 352 and a substrate 351 are bonded to each other. In FIG. 11, the substrate 352 is denoted by a dashed line.

The display device 100B includes the pixel portion 177, the connection portion 140, a circuit 356, a wiring 355, and the like. FIG. 11 illustrates an example in which an IC 354 and an FPC 353 are mounted on the display device 100B. Thus, the structure illustrated in FIG. 11 can be regarded as a display module including the display device 100B, the integrated circuit (IC), and the FPC. Here, a display device in which a substrate is equipped with a connector such as an FPC or mounted with an IC is referred to as a display module.

The connection portion 140 is provided outside the pixel portion 177. The number of connection portions 140 may be one or more. In the connection portion 140, a common electrode of a light-emitting device is electrically connected to a conductive layer, so that a potential can be supplied to the common electrode.

As the circuit 356, a scan line driver circuit can be used, for example.

The wiring 355 has a function of supplying a signal and power to the pixel portion 177 and the circuit 356. The signal and power are input to the wiring 355 from the outside through the FPC 353 or from the IC 354.

FIG. 11 illustrates an example in which the IC 354 is provided over the substrate 351 by a chip on glass (COG) method, a chip on film (COF) method, or the like. An IC including a scan line driver circuit, a signal line driver circuit, or the like can be used as the IC 354, for example. Note that the display device 100B and the display module are not necessarily provided with an IC. Alternatively, the IC may be mounted on the FPC by a COF method, for example.

FIG. 12 illustrates an example of cross sections of part of a region including the FPC 353, part of the circuit 356, part of the pixel portion 177, part of the connection portion 140, and part of a region including an end portion of the display device 100C.

### [Display Device 100C]

The display device 100C illustrated in FIG. 12 includes a transistor 201, a transistor 205, the light-emitting device 130R that emits red light, the light-emitting device 130G that emits green light, the light-emitting device 130B that emits blue light, and the like between the substrate 351 and the substrate 352.

Embodiment 1 can be referred to for the details of the light-emitting devices 130R, 130G, and 130B.

The light-emitting device 130R includes a conductive layer 224R, the conductive layer 151R over the conductive layer 224R, and the conductive layer 152R over the conductive layer 151R. The light-emitting device 130G includes a conductive layer 224G, the conductive layer 151G over the conductive layer 224G, and the conductive layer 152G over the conductive layer 151G. The light-emitting device 130B includes a conductive layer 224B, the conductive layer 151B over the conductive layer 224B, and the conductive layer 152B over the conductive layer 151B.

The conductive layer 224R is connected to a conductive layer 222b included in the transistor 205 through an opening provided in an insulating layer 214. An end portion of the conductive layer 151R is positioned outward from an end portion of the conductive layer 224R. The insulating layer 156R is provided to include a region that is in contact with the side surface of the conductive layer 151R, and the conductive layer 152R is provided to cover the conductive layer 151R and the insulating layer 156R.

The conductive layers 224G, 151G, and 152G and the insulating layer 156G in the light-emitting device 130G are not described in detail because they are respectively similar to the conductive layers 224R, 151R, and 152R and the insulating layer 156R in the light-emitting device 130R; the same applies to the conductive layers 224B, 151B, and 152B and the insulating layer 156B in the light-emitting device 130B.

The conductive layers 224R, 224G, and 224B each have a depression portion covering the opening provided in the insulating layer 214. A layer 128 is embedded in the depression portion.

The layer 128 has a function of filling the depression portions of the conductive layers 224R, 224G, and 224B to obtain planarity. Over the conductive layers 224R, 224G, and 224B and the layer 128, the conductive layers 151R, 151G, and 151B that are respectively electrically connected to the conductive layers 224R, 224G, and 224B are provided. Thus, the regions overlapping with the depression portions of the conductive layers 224R, 224G, and 224B can also be used as light-emitting regions, whereby the aperture ratio of the pixel can be increased.

The layer 128 may be an insulating layer or a conductive layer. Any of a variety of inorganic insulating materials, organic insulating materials, and conductive materials can be used for the layer 128 as appropriate. Specifically, the layer 128 is preferably formed using an insulating material and is particularly preferably formed using an organic insulating material. The layer 128 can be formed using an organic insulating material usable for the insulating layer 127, for example.

The protective layer 131 is provided over the light-emitting devices 130R, 130G, and 130B. The protective layer 131 and the substrate 352 are bonded to each other with an adhesive layer 142. The substrate 352 is provided with a light-blocking layer 157. A solid sealing structure, a hollow sealing structure, or the like can be employed to seal the light-emitting device 130. In FIG. 12, a solid sealing structure is employed, in which a space between the substrate 352 and the substrate 351 is filled with the adhesive layer 142. Alternatively, the space may be filled with an inert gas (e.g., nitrogen or argon), i.e., a hollow sealing structure may be employed. In that case, the adhesive layer 142 may be provided so as not to overlap with the light-emitting device. Alternatively, the space may be filled with a resin other than the frame-like adhesive layer 142.

FIG. 12 illustrates an example in which the connection portion 140 includes a conductive layer 224C obtained by processing the same conductive film as the conductive layers 224R, 224G, and 224B; the conductive layer 151C obtained by processing the same conductive film as the conductive layers 151R, 151G, and 151B; and the conductive layer 152C obtained by processing the same conductive film as the conductive layers 152R, 152G, and 152B. In the example illustrated in FIG. 12, the insulating layer 156C is provided to include a region overlapping with the side surface of the conductive layer 151C.

The display device 100C has a top-emission structure. Light from the light-emitting device is emitted toward the substrate 352. For the substrate 352, a material having a high visible-light-transmitting property is preferably used. In the case where the light-emitting device emits infrared or near-infrared light, a material having a high transmitting property with respect to infrared or near-infrared light is preferably used. The first electrode (pixel electrode) contains a material that reflects visible light, and the second electrode 155 (counter electrode) contains a material that transmits visible light.

An insulating layer 211, an insulating layer 213, an insulating layer 215, and the insulating layer 214 are provided in this order over the substrate 351. Part of the insulating layer 211 functions as a gate insulating layer of each transistor. Part of the insulating layer 213 functions as a gate insulating layer of each transistor. The insulating layer 215 is provided to cover the transistors. The insulating layer 214 is provided to cover the transistors and has a function of a planarization layer. Note that the number of gate insulating layers and the number of insulating layers covering the transistors are not limited and may each be one or more.

An inorganic insulating film is preferably used as each of the insulating layers 211, 213, and 215.

An organic insulating layer is suitable for the insulating layer 214 functioning as a planarization layer.

Each of the transistors 201 and 205 includes a conductive layer 221 functioning as a gate, the insulating layer 211 functioning as the gate insulating layer, a conductive layer 222a and the conductive layer 222b functioning as a source and a drain, a semiconductor layer 231, the insulating layer 213 functioning as the gate insulating layer, and a conductive layer 223 functioning as a gate.

A connection portion 204 is provided in a region of the substrate 351 that does not overlap with the substrate 352. In the connection portion 204, the wiring 355 is electrically connected to the FPC 353 through a conductive layer 166 and a connection layer 242. As an example, the conductive layer 166 has a stacked-layer structure of a conductive film obtained by processing the same conductive film as the conductive layers 224R, 224G, and 224B; a conductive film obtained by processing the same conductive film as the conductive layers 151R, 151G, and 151B; and a conductive film obtained by processing the same conductive film as the conductive layers 152R, 152G, and 152B. On the top surface of the connection portion 204, the conductive layer 166 is exposed. Thus, the connection portion 204 and the FPC 353 can be electrically connected to each other through the connection layer 242.

A light-blocking layer 157 is preferably provided on the surface of the substrate 352 on the substrate 351 side. The light-blocking layer 157 can be provided over a region between adjacent light-emitting devices, in the connection portion 140, in the circuit 356, and the like. A variety of optical members can be arranged on the outer surface of the substrate 352.

A material that can be used for the substrate 120 can be used for each of the substrates 351 and 352.

A material that can be used for the resin layer 122 can be used for the adhesive layer 142.

As the connection layer 242, an anisotropic conductive film (ACF), an anisotropic conductive paste (ACP), or the like can be used.

### [Display Device 100D]

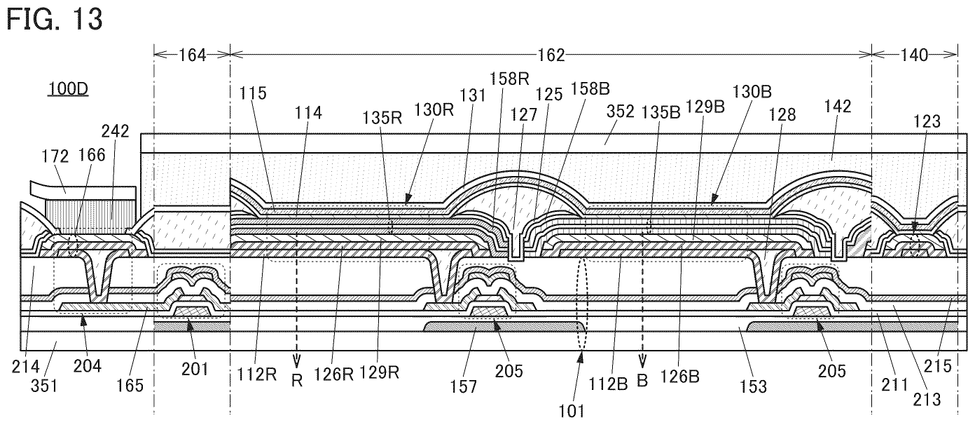

The display device 100D in FIG. 13 differs from the display device 100C in FIG. 12 mainly in having a bottom-emission structure.

Light from the light-emitting device is emitted toward the substrate 351. For the substrate 351, a material having a high visible-light-transmitting property is preferably used. By contrast, there is no limitation on the light-transmitting property of a material used for the substrate 352.

A light-blocking layer 157 is preferably formed between the substrate 351 and the transistor 201 and between the substrate 351 and the transistor 205. FIG. 13 illustrates an example in which the light-blocking layer 157 is provided over the substrate 351, an insulating layer 153 is provided over the light-blocking layer 157, and the transistors 201 and 205 and the like are provided over the insulating layer 153.

The light-emitting device 130R includes a conductive layer 112R, a conductive layer 126R over the conductive layer 112R, and a conductive layer 129R over the conductive layer 126R.

The light-emitting device 130B includes a conductive layer 112B, a conductive layer 126B over the conductive layer 112B, and a conductive layer 129B over the conductive layer 126B.

A material having a high visible-light-transmitting property is used for each of the conductive layers 112R, 112B, 126R, 126B, 129R, and 129B. A material that reflects visible light is preferably used for the second electrode.

Although not illustrated in FIG. 13, the light-emitting device 130G is also provided.

Although FIG. 13 and the like illustrate an example in which the top surface of the layer 128 includes a flat portion, the shape of the layer 128 is not particularly limited.

### [Display Device 100E]

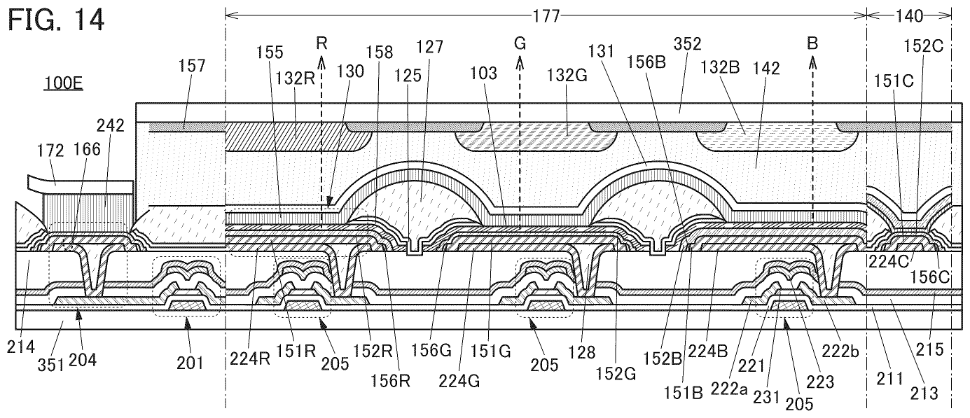

The display device 100E illustrated in FIG. 14 is a variation example of the display device 100C illustrated in FIG. 12 and differs from the display device 100C mainly in including the coloring layers 132R, 132G, and 132B.

In the display device 100E, the light-emitting device 130 includes a region overlapping with one of the coloring layers 132R, 132G, and 132B. The coloring layers 132R, 132G, and 132B can be provided on a surface of the substrate 352 on the substrate 351 side. End portions of the coloring layers 132R, 132G, and 132B can overlap with the light-blocking layer 157.

In the display device 100E, the light-emitting device 130 can emit white light, for example. The coloring layer 132R, the coloring layer 132G, and the coloring layer 132B can transmit red light, green light, and blue light, respectively, for example. Note that in the display device 100E, the coloring layers 132R, 132G, and 132B may be provided between the protective layer 131 and the adhesive layer 142.

Although FIGS. 12 and 14 and the like illustrate an example in which the top surface of the layer 128 includes a flat portion, the shape of the layer 128 is not particularly limited.

This embodiment can be combined as appropriate with the other embodiments or the examples. In this specification, in the case where a plurality of structure examples are shown in one embodiment, the structure examples can be combined as appropriate.

### Embodiment 5

In this embodiment, electronic devices of embodiments of the present invention will be described.

Electronic devices of this embodiment include the display device of one embodiment of the present invention in their display portions. The display device of one embodiment of the present invention has high display performance and can be easily increased in resolution and definition. Thus, the display device of one embodiment of the present invention can be used for display portions of a variety of electronic devices.

Examples of the electronic devices include a digital camera, a digital video camera, a digital photo frame, a mobile phone, a portable game console, a portable information terminal, and an audio reproducing device, in addition to electronic devices with a relatively large screen, such as a television device, desktop and notebook personal computers, a monitor of a computer and the like, digital signage, and a large game machine such as a pachinko machine.

In particular, the display device of one embodiment of the present invention can have high resolution, and thus can be favorably used for an electronic device having a relatively small display portion. Examples of such an electronic device include watch-type and bracelet-type information terminal devices (wearable devices) and wearable devices worn on the head, such as a VR device like a head-mounted display, a glasses-type AR device, and an MR device.

The electronic device in this embodiment may include a sensor (a sensor having a function of measuring force, displacement, position, speed, acceleration, angular velocity, rotational frequency, distance, light, liquid, magnetism, temperature, a chemical substance, sound, time, hardness, electric field, current, voltage, electric power, radiation, flow rate, humidity, gradient, oscillation, odor, or infrared rays).

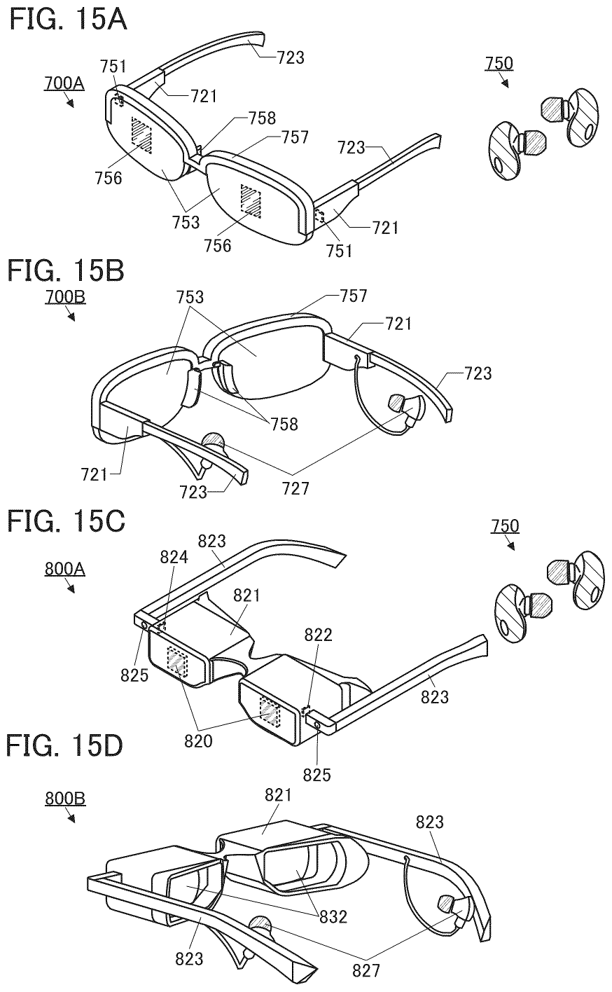

Examples of head-mounted wearable devices are described with reference to FIGS. 15A to 15D.

An electronic device 700A illustrated in FIG. 15A and an electronic device 700B illustrated in FIG. 15B each include a pair of display panels 751, a pair of housings 721, a communication portion (not illustrated), a pair of wearing portions 723, a control portion (not illustrated), an image capturing portion (not illustrated), a pair of optical members 753, a frame 757, and a pair of nose pads 758.

The display device of one embodiment of the present invention can be used for the display panels 751. Thus, the electronic devices can be highly reliable.

The electronic devices 700A and 700B can each project images displayed on the display panels 751 onto display regions 756 of the optical members 753. Since the optical members 753 have a light-transmitting property, the user can see images displayed on the display regions, which are superimposed on transmission images seen through the optical members 753.

In the electronic devices 700A and 700B, a camera capable of capturing images of the front side may be provided as the image capturing portion. Furthermore, when the electronic devices 700A and 700B are provided with an acceleration sensor such as a gyroscope sensor, the orientation of the user's head can be sensed and an image corresponding to the orientation can be displayed on the display regions 756.

The communication portion includes a wireless communication device, and a video signal, for example, can be supplied by the wireless communication device. Instead of or in addition to the wireless communication device, a connector that can be connected to a cable for supplying a video signal and a power supply potential may be provided.

The electronic devices 700A and 700B are provided with a battery, so that they can be charged wirelessly and/or by wire.

A touch sensor module may be provided in the housing 721.

Various touch sensors can be applied to the touch sensor module. For example, any of touch sensors of the following types can be used: a capacitive type, a resistive type, an infrared type, an electromagnetic induction type, a surface acoustic wave type, and an optical type. In particular, a capacitive sensor or an optical sensor is preferably used for the touch sensor module.

An electronic device 800A illustrated in FIG. 15C and an electronic device 800B illustrated in FIG. 15D each include a pair of display portions 820, a housing 821, a communication portion 822, a pair of wearing portions 823, a control portion 824, a pair of image capturing portions 825, and a pair of lenses 832.

The display device of one embodiment of the present invention can be used in the display portions 820. Thus, the electronic devices can be highly reliable.

The display portions 820 are positioned inside the housing 821 so as to be seen through the lenses 832. When the pair of display portions 820 display different images, three-dimensional display using parallax can be performed.

The electronic devices 800A and 800B preferably include a mechanism for adjusting the lateral positions of the lenses 832 and the display portions 820 so that the lenses 832 and the display portions 820 are positioned optimally in accordance with the positions of the user's eyes.

The electronic device 800A or the electronic device 800B can be mounted on the user's head with the wearing portions 823.

The image capturing portion 825 has a function of obtaining information on the external environment. Data obtained by the image capturing portion 825 can be output to the display portion 820. An image sensor can be used for the image capturing portion 825. Moreover, a plurality of cameras may be provided so as to cover a plurality of fields of view, such as a telescope field of view and a wide field of view.

The electronic device 800A may include a vibration mechanism that functions as bone-conduction earphones.

The electronic devices 800A and 800B may each include an input terminal. To the input terminal, a cable for supplying a video signal from a video output device or the like, power for charging a battery provided in the electronic device, and the like can be connected.

The electronic device of one embodiment of the present invention may have a function of performing wireless communication with earphones 750.

The electronic device may include an earphone portion. The electronic device 700B in FIG. 15B includes earphone portions 727. Part of a wiring that connects the earphone portion 727 and the control portion may be positioned inside the housing 721 or the wearing portion 723.

Similarly, the electronic device 800B in FIG. 15D includes earphone portions 827. For example, the earphone portion 827 can be connected to the control portion 824 by wire.

As described above, both the glasses-type device (e.g., the electronic devices 700A and 700B) and the goggles-type device (e.g., the electronic devices 800A and 800B) are preferable as the electronic device of one embodiment of the present invention.

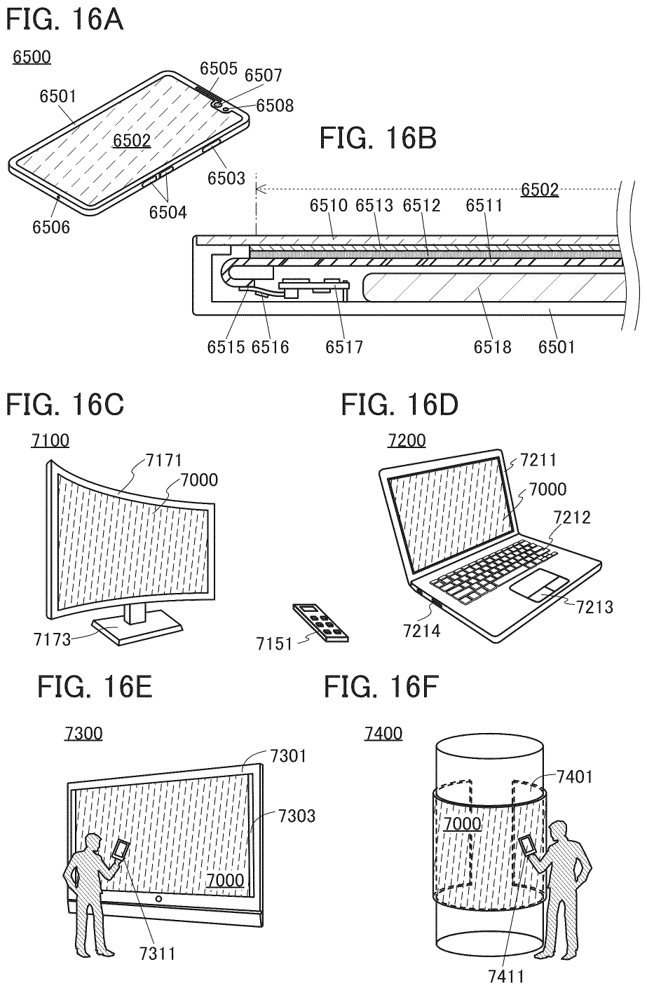

An electronic device 6500 in FIG. 16A is a portable information terminal that can be used as a smartphone.

The electronic device 6500 includes a housing 6501, a display portion 6502, a power button 6503, buttons 6504, a speaker 6505, a microphone 6506, a camera 6507, a light source 6508, and the like. The display portion 6502 has a touch panel function.

The display device of one embodiment of the present invention can be used in the display portion 6502. Thus, the electronic device can be highly reliable.

FIG. 16B is a schematic cross-sectional view including an end portion of the housing 6501 on the microphone 6506 side.

A protection member 6510 having a light-transmitting property is provided on the display surface side of the housing 6501. A display panel 6511, an optical member 6512, a touch sensor panel 6513, a printed circuit board 6517, a battery 6518, and the like are provided in a space surrounded by the housing 6501 and the protection member 6510.

The display panel 6511, the optical member 6512, and the touch sensor panel 6513 are fixed to the protection member 6510 with an adhesive layer (not illustrated).

Part of the display panel 6511 is folded back in a region outside the display portion 6502, and an FPC 6515 is connected to the part that is folded back. An IC 6516 is mounted on the FPC 6515. The FPC 6515 is connected to a terminal provided on the printed circuit board 6517.

The display device of one embodiment of the present invention can be used in the display panel 6511. Thus, the electronic device can be extremely lightweight. Since the display panel 6511 is extremely thin, the battery 6518 with high capacity can be mounted without an increase in the thickness of the electronic device. Moreover, part of the display panel 6511 is folded back so that a connection portion with the FPC 6515 is provided on the back side of the pixel portion, whereby the electronic device can have a narrow bezel.

FIG. 16C illustrates an example of a television device. In a television device 7100, a display portion 7000 is incorporated in a housing 7171. Here, the housing 7171 is supported by a stand 7173.

The display device of one embodiment of the present invention can be used in the display portion 7000. Thus, a highly reliable electronic device is obtained.

Operation of the television device 7100 illustrated in FIG. 16C can be performed with an operation switch provided in the housing 7171 and a separate remote controller 7151.

FIG. 16D illustrates an example of a notebook personal computer. A notebook personal computer 7200 includes a housing 7211, a keyboard 7212, a pointing device 7213, an external connection port 7214, and the like. The display portion 7000 is incorporated in the housing 7211.

The display device of one embodiment of the present invention can be used in the display portion 7000. Thus, a highly reliable electronic device is obtained.

FIGS. 16E and 16F illustrate examples of digital signage.

Digital signage 7300 illustrated in FIG. 16E includes a housing 7301, the display portion 7000, a speaker 7303, and the like. The digital signage 7300 can also include an LED lamp, operation keys (including a power switch or an operation switch), a connection terminal, a variety of sensors, a microphone, and the like.

FIG. 16F illustrates digital signage 7400 attached to a cylindrical pillar 7401. The digital signage 7400 includes the display portion 7000 provided along a curved surface of the pillar 7401.

In FIGS. 16E and 16F, the display device of one embodiment of the present invention can be used in the display portion 7000. Thus, the electronic devices can be highly reliable.

A larger area of the display portion 7000 can increase the amount of information that can be provided at a time. The display portion 7000 having a larger area attracts more attention, so that the effectiveness of the advertisement can be increased, for example.

As illustrated in FIGS. 16E and 16F, it is preferable that the digital signage 7300 or the digital signage 7400 can work with an information terminal 7311 or an information terminal 7411, such as a smartphone that a user has, through wireless communication.

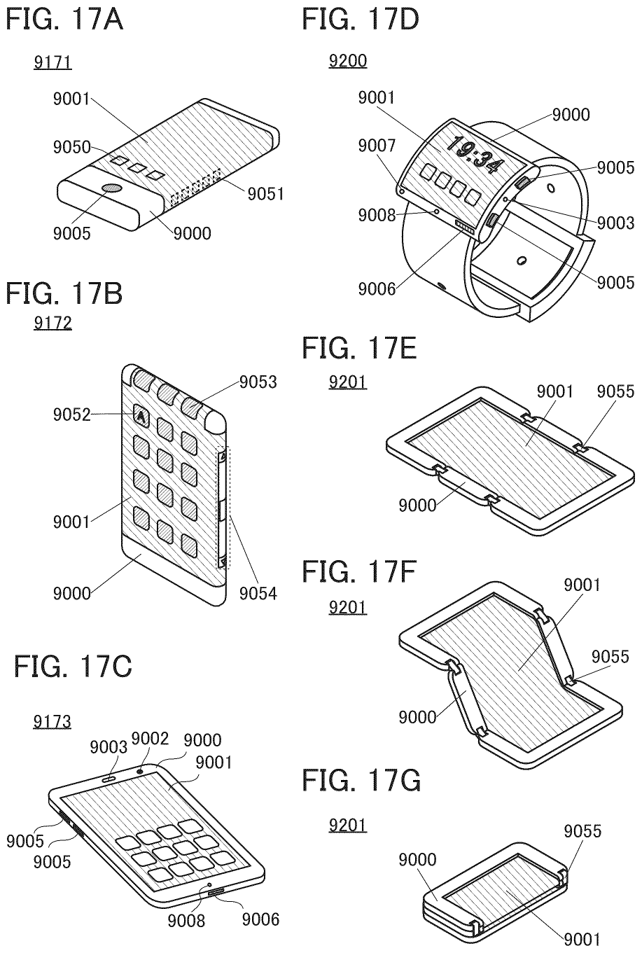

Electronic devices illustrated in FIGS. 17A to 17G include a housing 9000, a display portion 9001, a speaker 9003, an operation key 9005 (including a power switch or an operation switch), a connection terminal 9006, a sensor 9007 (a sensor having a function of measuring force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays), a microphone 9008, and the like.

The electronic devices illustrated in FIGS. 17A to 17G have a variety of functions. For example, the electronic devices can have a function of displaying a variety of information (e.g., a still image, a moving image, and a text image) on the display portion, a touch panel function, a function of displaying a calendar, date, time, and the like, a function of controlling processing with the use of a variety of software (programs), a wireless communication function, and a function of reading out and processing a program or data stored in a recording medium.

The electronic devices in FIGS. 17A to 17G are described in detail below.

FIG. 17A is a perspective view of a portable information terminal 9171. The portable information terminal 9171 can be used as a smartphone, for example. The portable information terminal 9171 may include the speaker 9003, the connection terminal 9006, the sensor 9007, or the like. The portable information terminal 9171 can display text and image information on its plurality of surfaces. FIG. 17A illustrates an example in which three icons 9050 are displayed. Furthermore, information 9051 indicated by dashed rectangles can be displayed on another surface of the display portion 9001. Examples of the information 9051 include notification of reception of an e-mail, an SNS message, an incoming call, or the like, the title and sender of an e-mail, an SNS message, or the like, the date, the time, remaining battery, and the radio field intensity. Alternatively, the icon 9050 or the like may be displayed at the position where the information 9051 is displayed.

FIG. 17B is a perspective view of a portable information terminal 9172. The portable information terminal 9172 has a function of displaying information on three or more surfaces of the display portion 9001. Here, information 9052, information 9053, and information 9054 are displayed on the respective surfaces. For example, the user of the portable information terminal 9172 can check the information 9053 displayed such that it can be seen from above the portable information terminal 9172, with the portable information terminal 9172 put in a breast pocket of his/her clothes.

FIG. 17C is a perspective view of a tablet terminal 9173. The tablet terminal 9173 is capable of executing a variety of applications such as mobile phone calls, e-mailing, viewing and editing texts, music reproduction, Internet communication, and a computer game, for example. The tablet terminal 9173 includes the display portion 9001, the camera 9002, the microphone 9008, and the speaker 9003 on the front surface of the housing 9000; the operation keys 9005 as buttons for operation on the left side surface of the housing 9000; and the connection terminal 9006 on the bottom surface of the housing 9000.

FIG. 17D is a perspective view of a watch-type portable information terminal 9200. The portable information terminal 9200 can be used as a Smartwatch (registered trademark), for example. The display surface of the display portion 9001 is curved, and an image can be displayed on the curved display surface. Furthermore, for example, mutual communication between the portable information terminal 9200 and a headset capable of wireless communication can be performed, and thus hands-free calling is possible. With the connection terminal 9006, the portable information terminal 9200 can perform mutual data transmission with another information terminal and charging. Note that the charging operation may be performed by wireless power feeding.

FIGS. 17E to 17G are perspective views of a foldable portable information terminal 9201. FIG. 17E is a perspective view showing the portable information terminal 9201 that is opened. FIG. 17G is a perspective view showing the portable information terminal 9201 that is folded. FIG. 17F is a perspective view showing the portable information terminal 9201 that is shifted from one of the states in FIGS. 17E and 17G to the other. The portable information terminal 9201 is highly portable when folded. When the portable information terminal 9201 is opened, a seamless large display region is highly browsable. The display portion 9001 of the portable information terminal 9201 is supported by three housings 9000 joined together by hinges 9055. The display portion 9001 can be folded with a radius of curvature of greater than or equal to 0.1 mm and less than or equal to 150 mm, for example.

This embodiment can be combined as appropriate with the other embodiments or the examples. In this specification, in the case where a plurality of structure examples are shown in one embodiment, the structure examples can be combined as appropriate.

### Example 1

### Synthesis Example 1

In this synthesis example, a method for synthesizing N-[4-(2,2′-binaphthyl-6-yl)phenyl]-N-(9,9-dimethyl-9H-fluoren-4-yl)dibenzofuran-2-amine (abbreviation: Fr(2)F(4)A(βN2)B), which is represented by Structural Formula (162) in Embodiment, will be described. The structural formula of Fr(2)F(4)A(βN2)B is shown below.

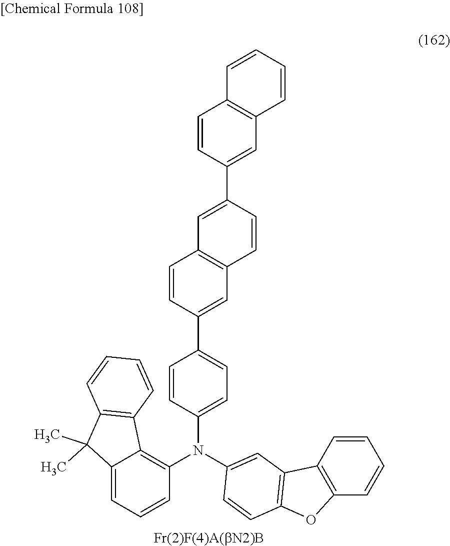 [SMILES: CC1(C)c2ccccc2-c2c(N(c3ccc(-c4ccc5cc(-c6ccc7ccccc7c6)ccc5c4)cc3)c3ccc4oc5ccccc5c4c3)cccc21]

### Step 1: Synthesis of N-(chlorophenyl-4-yl)dibenzofuran-2-amine

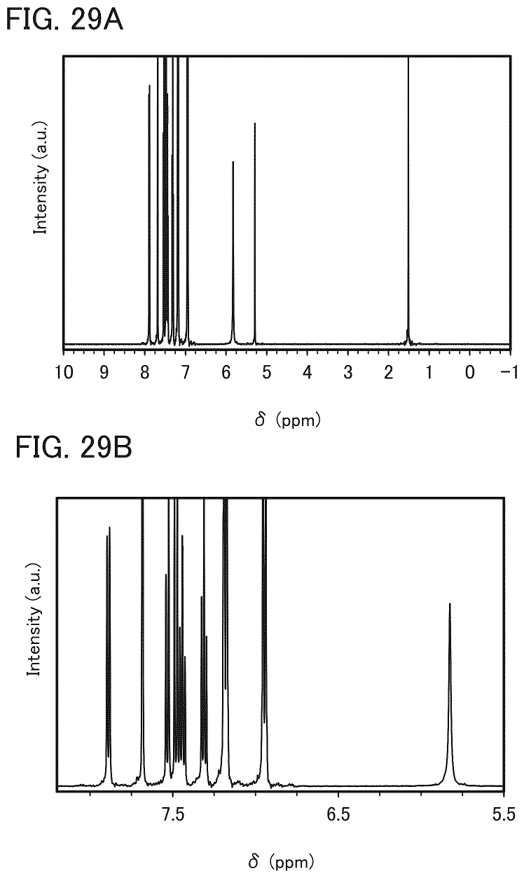

Into a 500-mL three-neck flask were added 4.2 g (4.0 mmol) of 4-chloroaniline and 10 g (40 mmol) of 2-bromodibenzofuran. After the air in the flask was replaced with nitrogen, 7.8 g (81 mmol) of sodium tert-butoxide and 202 mL of toluene were added thereto. This mixture was degassed by being stirred under reduced pressure. To this mixture, 2.4 ml. (0.81 mmol) of tris(tert-butyl)phosphine (10 wt % hexane solution) was added, the temperature was raised to 60° C., and the mixture was stirred. After the stirring, 0.23 g (0.40 mmol) of bis(dibenzylideneacetone)palladium(0) was added, the temperature was raised to 120° C., and stirring was performed for an hour. After the stirring, the reacted solution was suction-filtered through alumina, Celite (Catalog No. 537-02305 produced by FUJIFILM Wako Pure Chemical Corporation), and Florisil (Catalog No. 066-05265 produced by FUJIFILM Wako Pure Chemical Corporation). The obtained filtrate was concentrated to give a solid, and the solid was purified by silica gel column chromatography (hexane:toluene=9:1), whereby 5.4 g of a pale yellow solid containing a target substance was obtained. Hexane was added to the ethyl acetate solution of the obtained solid, and the precipitated solid was collected by suction filtration, whereby 5.2 g of a target white solid was obtained in a yield of 44%. Synthesis Scheme (s1-1) of Step 1 is shown below. FIGS. 29A and 29B show ¹H NMR measurement results of the white solid. Note that FIG. 29B is a chart where the range from 5.5 ppm to 7.8 ppm in FIG. 29A is enlarged. The results show that N-(chlorophenyl-4-yl)dibenzofuran-2-amine was obtained.

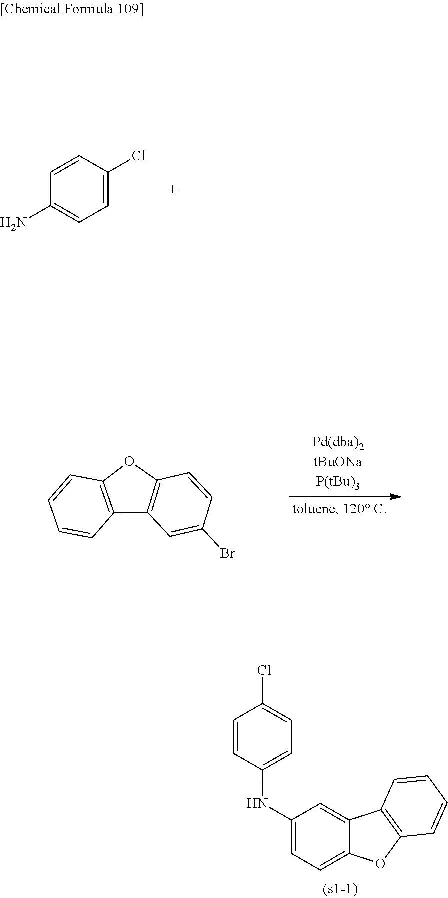 [SMILES: Nc1ccc(Cl)cc1 ; Brc1ccc2oc3ccccc3c2c1 ; Clc1ccc(Nc2ccc3oc4ccccc4c3c2)cc1]

### Step 2: Synthesis of N-[4-(2,2′-binaphthyl-6-yl)phenyl]-N-(9,9-dimethyl-9H-fluoren-4-yl)dibenzofuran-2-amine

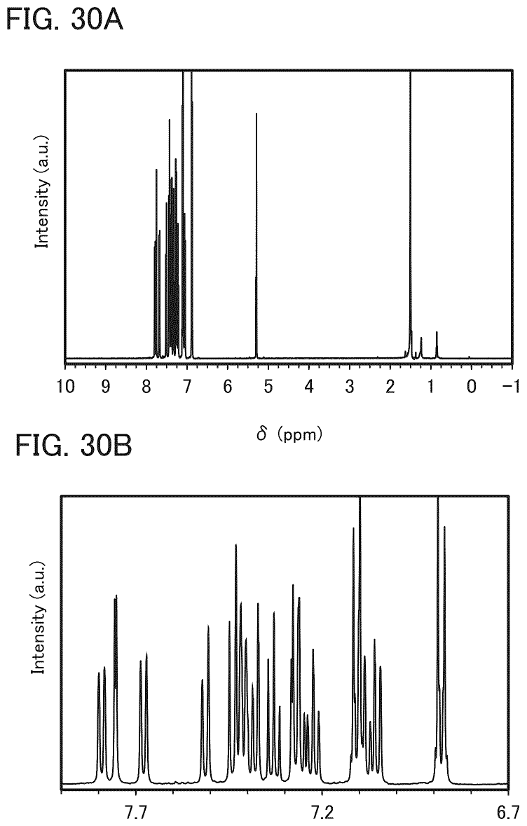

Into a 300-mL, three-neck flask were added 5.0 g (17 mmol) of N-(chlorophenyl-4-yl)dibenzofuran-2-amine synthesized in Step 1 and 4.7 g (17 mmol) of 4-bromo-9,9′-dimethylfluorene. After the air in the flask was replaced with nitrogen, 3.3 g (34 mmol) of sodium tert-butoxide and 85 mL of toluene were added thereto. This mixture was degassed by being stirred under reduced pressure. To this mixture, 1.0 ml, (0.34 mmol) of tris(tert-butyl)phosphine (10 wt % hexane solution) was added, the temperature was raised to 60° C., and the mixture was stirred. After the stirring, 98 mg (0.17 mmol) of bis(dibenzylideneacetone)palladium(0) was added, the temperature was raised to 120° C., and stirring was performed for an hour. After the stirring, the reacted solution was suction-filtered through alumina, Celite (Catalog No. 537-02305 produced by FUJIFILM Wako Pure Chemical Corporation), and Florisil (Catalog No. 066-05265 produced by FUJIFILM Wako Pure Chemical Corporation). The obtained filtrate was concentrated to give a solid, the solid was purified by silica gel column chromatography (hexane), whereby 4.5 g of a target white solid was obtained in a yield of 65%. Synthesis Scheme (s1-2) of Step 2 is shown below. FIGS. 30A and 30B show ¹H NMR measurement results of the white solid. FIG. 30B is a chart where the range from 6.7 ppm to 7.9 ppm in FIG. 30A is enlarged. The results show that N-[4-(2,2′-binaphthyl-6-yl)phenyl]-N-(9,9-dimethyl-9H-fluoren-4-yl)dibenzofuran-2-amine was obtained.

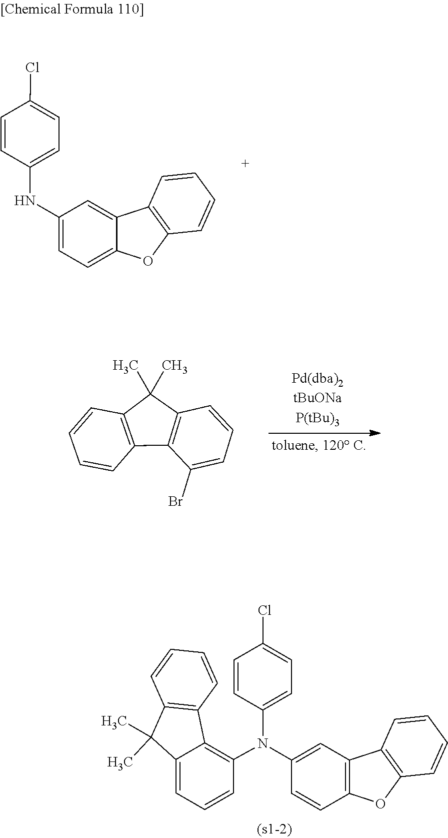 [SMILES: Clc1ccc(Nc2ccc3oc4ccccc4c3c2)cc1 ; CC1(C)c2ccccc2-c2c(Br)cccc21 ; CC1(C)c2ccccc2-c2c(N(c3ccc(Cl)cc3)c3ccc4oc5ccccc5c4c3)cccc21]

### Step 3: Synthesis of Fr(2)F(4)A(βN2)B

Into a 200-mL three-neck flask were put 4.5 g (9.2 mmol) of N-[4-(2,2′-binaphthyl-6-yl)phenyl]-N-(9,9-dimethyl-91-fluoren-4-yl)dibenzofuran-2-amine, 3.5 g (9.2 mmol) of 2-[2,2′-binaphthalen-6-yl]-4,4,5,5-tetramethyl-1,3,2-dioxaborolane, 110 mg (0.37 mmol) of tri(o-tolyl)phosphine, 7.8 g (37 mmol) of tripotassium phosphate, 73 mL of toluene, 18 mL of ethanol, and 18 mL of pure water. This mixture was degassed by being stirred under reduced pressure and then heated to 60° C.; 41 mg of palladium(II) acetate was added thereto; the temperature was raised to 90° C.; and the mixture was stirred for 24 hours. After the stirring, the mixture was cooled to 60° C., 41 mg of palladium(II) acetate, 130 mg of di(1-adamantyl)-n-butylphosphine, and 5.0 g of potassium carbonate were added, and the mixture was stirred at 90° C. for a week. After the stirring, the mixture was cooled to room temperature, the reacted solution was concentrated, pure water and toluene were added thereto, so that solution separation was performed. The organic layer was dehydrated with magnesium sulfate and suction filtration was performed. The filtrate was concentrated to give a solid, and the solid was purified by silica gel column chromatography (hexane:toluene=9:1), whereby 1.8 g of a target pale yellow solid was obtained in a yield of 30%. Synthesis Scheme (s1-3) of Step 3 is shown below.

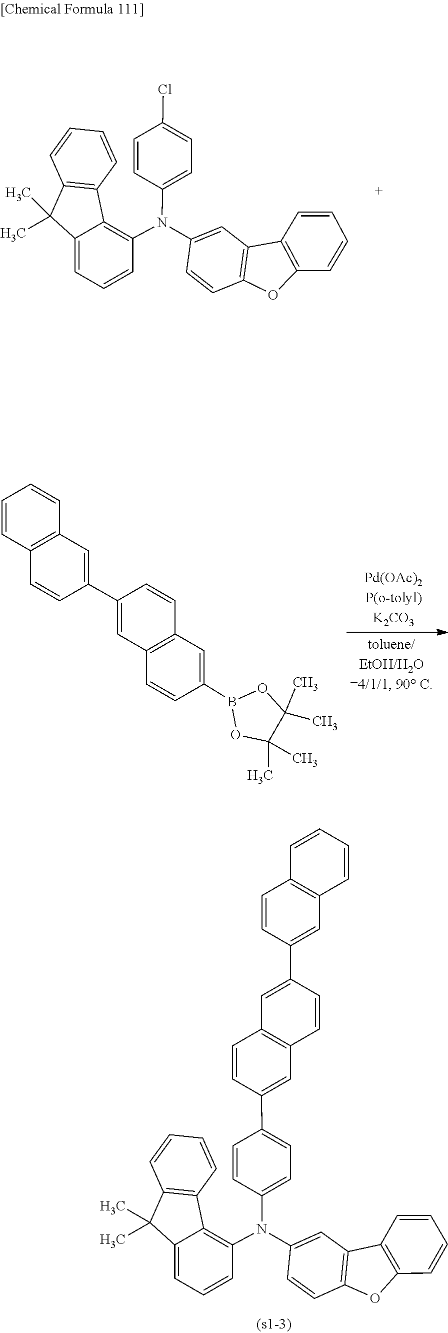

By a train sublimation method, 1.4 g of the obtained solid was purified. In the purification by sublimation, the solid was heated at 310° C. for 45 hours under a pressure of 2.3 Pa with an argon flow rate of 10 mL/min. After the purification by sublimation, 910 mg of a target solid was obtained at a collection rate of 65%.

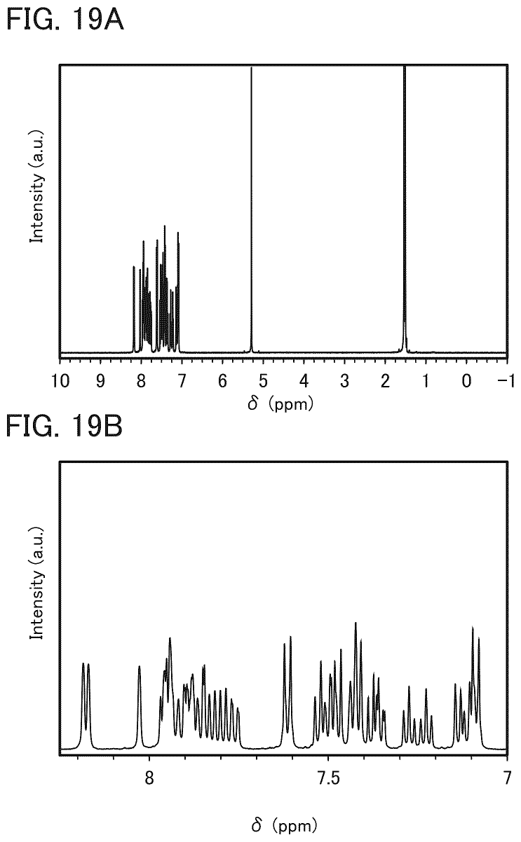

The ¹H NMR measurement results of the obtained solid is shown below and in FIGS. 19A and 19B. Note that FIG. 19B is a chart where the range from 7.00 ppm to 8.25 ppm in FIG. 19A is enlarged. The results show that Fr(2)F(4)A(βN2)B was obtained in this synthesis example.

¹H NMR (dichloromethane-d₂, 500 MHz, 20° C.): δ=8.18 (d, J=7.5 Hz, 2H), δ=8.03 (s, 1H), δ=7.97-7.75 (m, 11H), δ=7.61 (d, J=8.5 Hz, 2H), δ=7.54-7.34 (m, 9H), δ=7.28 (t, J=7.5 Hz, 1H), δ=7.23 (t, J=7.5 Hz, 1H), δ=7.14 (d, J=7.5 Hz, 11H), δ=7.11 (d, J=7.5 Hz, 1H), δ=7.09 (d, J=8.5 Hz, 2H), δ=1.54 (s, 6H).

### <Measurement of Physical Properties>

Next, the ultraviolet-visible absorption spectrum (hereinafter, simply referred to as “absorption spectrum”) and the photolumrinescence (PL) spectrum of a toluene solution of Fr(2)F(4)A(βN2)B and a thin film of Fr(2)F(4)A(βN2)B were measured.

The absorption spectrum was measured with an ultraviolet-visible spectrophotometer (V-770DS, produced by JASCO Corporation). The PL spectrum was measured with a fluorescence spectrophotometer (FP-8600DS, JASCO Corporation).

To calculate the absorption spectrum of Fr(2)F(4)A(βN2) in a toluene solution, the absorption spectrum of toluene put in a quartz cell was measured and then subtracted from the absorption spectrum of the toluene solution of Fr(2)F(4)A(βN2)B put in a quartz cell.

For obtaining the absorption spectrum and the PL spectrum of the thin film, a measurement sample was measured. The measurement sample was fabricated in such a manner that FrFA(βN2)B was deposited over a quartz substrate by a vacuum evaporation method and sealed using a quartz substrate as a counter substrate. Note that the PL spectrum was obtained by measurement of the measurement sample sealed, and the absorption spectrum was obtained by measurement of the sample from which the sealing was removed and the counter substrate was detached. The absorption spectrum was obtained by subtraction of the absorption spectrum of the quartz substrate from the absorption spectrum of the FrFA(βN2)B film formed over the quartz substrate.

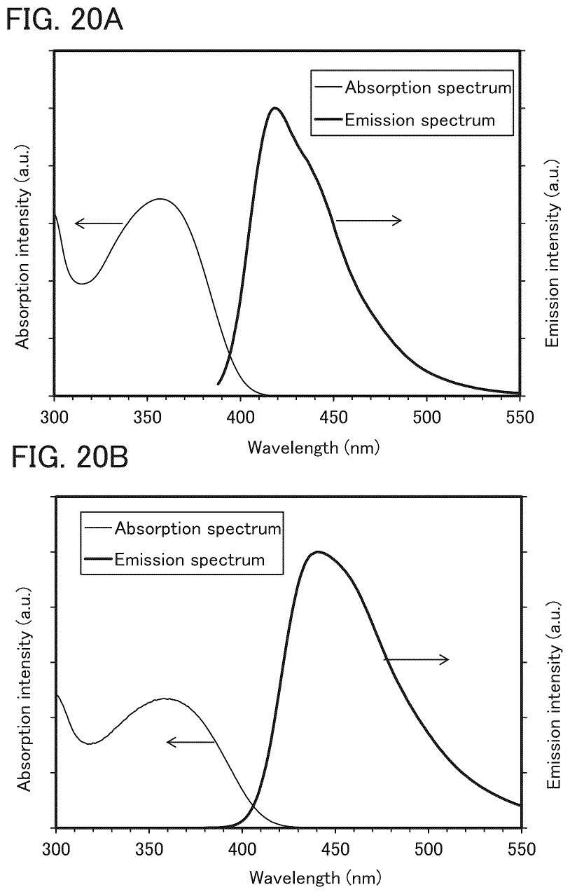

FIG. 20A shows the measurement results of the toluene solution and FIG. 20B shows the measurement results of the thin film. The measurement results show that Fr(2)F(4)A(βN2)B in the toluene solution has an absorption spectrum peak (absorption peak) at around 360 nm; the thin film of Fr(2)F(4)A(βN2)B has an absorption peak at around 361 nm; and in both the toluene solution and the thin film, an absorption band is not observed on the longer wavelength side than 430 nm. This indicates that in the case where Fr(2)F(4)A(βN2)B is used in a light-emitting device, a reduction in emission efficiency caused by absorption does not occur at a light wavelength used in a display and thus Fr(2)F(4)A(βN2)B can be suitably used. The toluene solution of Fr(2)F(4)A(βN2)B exhibited an emission wavelength peak at around 418 nm (excitation wavelength: 359 nm), and the thin film of Fr(2)F(4)A(βN2)B exhibited an emission spectrum peak (emission peak) at around 441 nm (excitation wavelength: 369 nm). The HOMO level and the LUMO level of Fr(2)F(4)A(βN2)B were calculated by cyclic voltammetry (CV) measurement. The calculation method is described below.

An electrochemical analyzer (ALS model 600A or 600C, produced by BAS Inc.) was used as a measurement apparatus. To prepare a solution for the CV measurement, dehydrated dimethylformamide (DMF; produced by Sigma-Aldrich Inc., 99.8%, catalog No. 22705-6) was used as a solvent, and tetra-n-butylammonium perchlorate (abbreviation: n-Bu₄NCIO₄; produced by Tokyo Chemical Industry Co., Ltd., catalog No. T0836) as a supporting electrolyte was dissolved at a concentration of 100 mmol/L. Furthermore, the object to be measured was also dissolved at a concentration of 2 mmol/L.

A platinum electrode (PTE platinum electrode, produced by BAS Inc.) was used as a working electrode, another platinum electrode (Pt counter electrode for VC-3 (5 cm), produced by BAS Inc.) was used as an auxiliary electrode, and an Ag/Ag⁺ electrode (RE7 reference electrode for nonaqueous solvent, produced by BAS Inc.) was used as a reference electrode. Note that the measurement was performed at room temperature (20° C. to 25° C.). The scan speed in the CV measurement was fixed to 0.1 V/sec, and an oxidation potential Ea [V] and a reduction potential Ec [V] with respect to the reference electrode were measured. The potential Ea is an intermediate potential of an oxidation-reduction wave, and the potential Ec is an intermediate potential of a reduction-oxidation wave. Here, since the potential energy of the reference electrode used in this example with respect to the vacuum level is known to be −4.94 [eV], the HOMO level and the LUMO level can be calculated by the following formulae: HOMO level [eV]=−4.94−Ea and LUMO level [eV]=−4.94−Ec.

The CV measurement was repeated 100 times, and the oxidation—reduction wave in the 100th cycle was compared with the oxidation-reduction wave in the first cycle to examine the electrical stability of the compound.

As a result, in the measurement of an oxidation potential Ea [V] of Fr(2)F(4)A(βN2)B, the HOMO level was found to be −5.51 eV. In contrast, the LUMO level was −2.45 eV in the measurement of the reduction potential Ec [V]. When the oxidation-reduction wave was repeatedly measured, in the Ea measurement, the peak intensity of the oxidation-reduction wave after the 100th cycle was maintained to be 92% of that of the oxidation-reduction wave at the first cycle, and in the Ec measurement, the peak intensity of the oxidation-reduction wave after the 100th cycle was maintained to be 99% of that of the oxidation-reduction wave at the first cycle; thus, resistance of Fr(2)F(4)A(βN2) to repetitive oxidation and repetitive reduction was found to be extremely high.

Differential scanning calorimetry (DSC) measurement of Fr(2)F(4)A(βN2)B was performed with DSC8500 produced by PerkinElmer, Inc. The DSC measurement was performed in the following manner: the temperature was raised from −10° C. to 320° C. at a temperature rising rate of 40° C./min and held for three minutes, and then the temperature was decreased to −10° C. at a temperature decreasing rate of 40° C./min and held for three minutes. This operation was performed twice in succession. From the DSC measurement results in the second cycle, the glass transition point (Tg) of Fr(2)F(4)A(βN2)B was 133° C., and the crystallization temperature and the melting point were not observed. This indicates that Fr(2)F(4)A(βN2)B is a substance having extremely high heat resistance and the film of Fr(2)F(4)A(βN2)B can maintain a thermally stable quality.

The thermogravimetry-differential thermal analysis (TG-DTA) of Fr(2)F(4)A(βN2)B was performed. For the measurement, a high-sensitivity differential type differential thermogravimeter (STA 2500 Regulus produced by NETZSCH Japan K.K.) was used. The measurement was performed under an atmospheric pressure at a temperature rising rate of 10° C./min under a nitrogen stream (flow rate: 200 mL/min). In the thermogravimetry-differential thermal analysis, the temperature (decomposition temperature) at which the weight obtained by thermogravimetry was reduced by 5% of the weight at the beginning of the measurement was found to be 472° C. or higher, which shows that Fr(2)F(4)A(βN2)B is a substance having high heat resistance.

### Example 2

### Synthesis Example 2

In this synthesis example, a method for synthesizing N-[4-(2,2′-binaphthyl-6-yl)phenyl]-N-(9,9-dimethyl-9H-fluoren-2-yl)dibenzofuran-4-amine (abbreviation: FrFA(βN2)B), which is represented by Structural Formula (152) in Embodiment, will be described. The structural formula of FrFA(βN2)B is shown below.

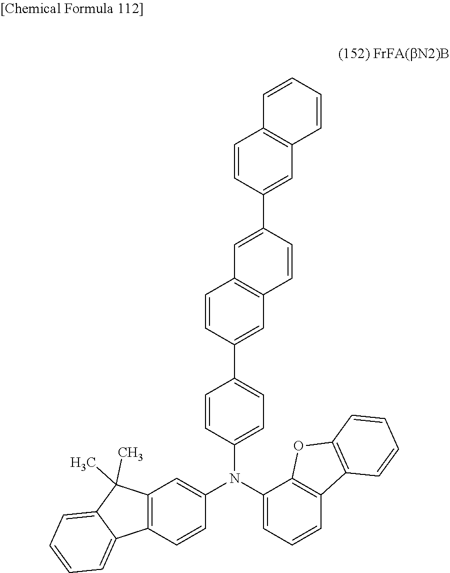 [SMILES: CC1(C)c2ccccc2-c2ccc(N(c3ccc(-c4ccc5cc(-c6ccc7ccccc7c6)ccc5c4)cc3)c3cccc4c3oc3ccccc34)cc21]

### Step 1: Synthesis of N-[4-(2,2′-binaphthyl-6-yl)phenyl]-9,9-dimethyl-9H-fluoren-2-amine

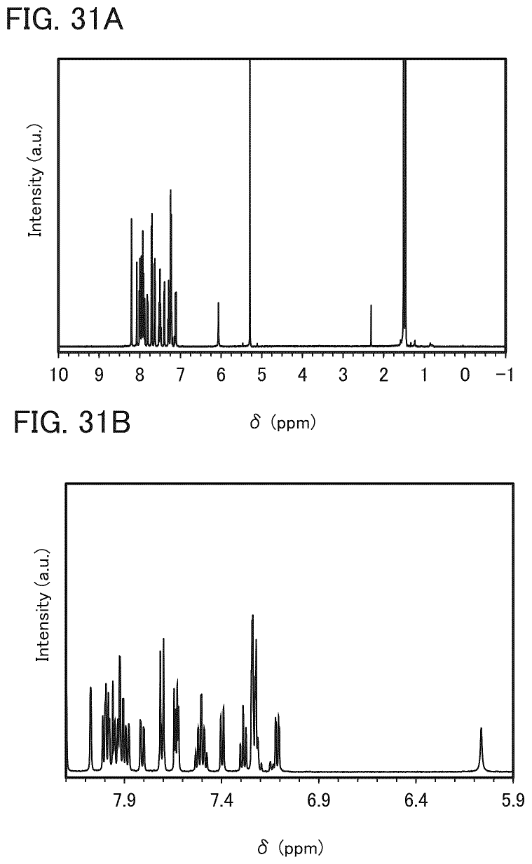

Into a 200-mL three-neck flask were put 4.8 g (13.1 mmol) of N-(4-bromophenyl)-9,9-dimethyl-91H-fluoren-2-anine, 5.0 g (13.1 mmol) of 2-[(2,2′-binaphthalen)-6-yl]-4,4,5,5-tetramethyl-1,3,2-dioxaborolane, 160 mg of tris(o-tolyl)phosphine, 5.5 g of potassium carbonate, 40 mL of toluene, 10 mL of ethanol, and 10 mL of pure water. The mixture was degassed by being stirred under reduced pressure, the temperature was raised to 60° C., and stirring was performed for 10 minutes. After the stirring, palladium(II) acetate was added and stirring was performed at 90° C. for an hour. After the stirring, the reacted solution was cooled to room temperature, suction filtration was performed, and washing with ethanol was performed, whereby 6.0 g of a gray-brown solid was obtained. The gray-brown solid was dissolved in dichloromethane and gravity filtration was performed, whereby 5.1 g of a yellow solid was obtained. The yellow solid was dissolved in hot toluene, the solution was cooled down to room temperature, and recrystallization treatment was performed, whereby 4.4 g of a target pale yellow solid was obtained in a yield of 63%. Synthesis Scheme (s2-1) of Step 1 is shown below. FIGS. 31A and 31B show ¹H NMR measurement results of the pale yellow solid. Note that FIG. 31B is a chart where the range from 5.9 ppm to 8.2 ppm in FIG. 31A is enlarged. The results show that N-[4-(2,2′-binaphthyl-6-yl)phenyl]-9,9-dimethyl-9H-fluoren-2-amine was obtained.

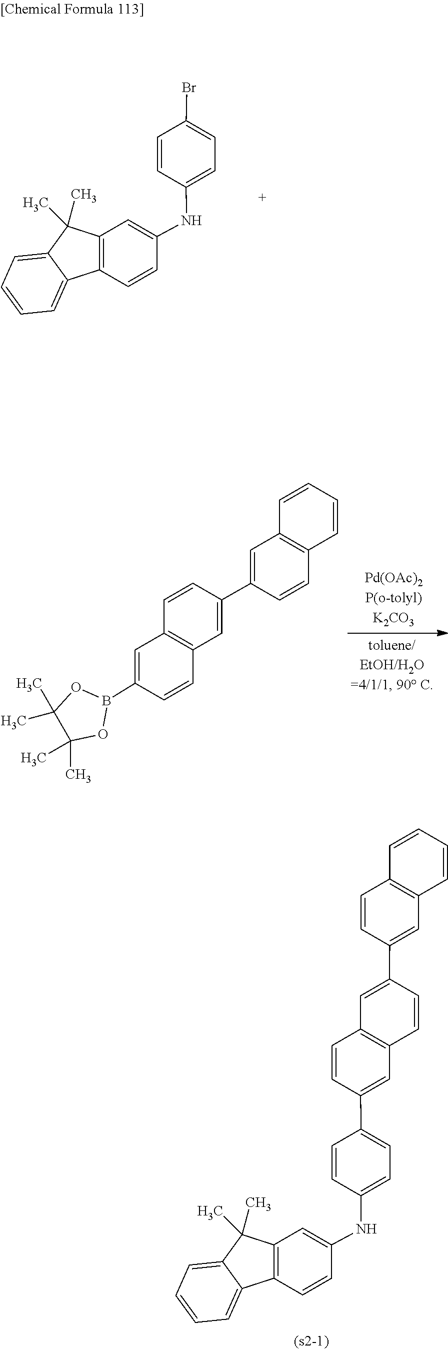 [SMILES: CC1(C)c2ccccc2-c2ccc(Nc3ccc(Br)cc3)cc21 ; CC1(C)OB(c2ccc3cc(-c4ccc5ccccc5c4)ccc3c2)OC1(C)C ; CC1(C)c2ccccc2-c2ccc(Nc3ccc(-c4ccc5cc(-c6ccc7ccccc7c6)ccc5c4)cc3)cc21]

### Step 2: Synthesis of FrFA(βN2)B

Into a 50-mL three-neck flask were put 1.0 g (1.8 mmol) of N-[4-(2,2′-binaphthyl-6-yl)phenyl]-9,9-dimethyl-9H-fluoren-2-amine and 500 mg (1.8 mmol) of 4-bromodibenzofuran. After the air in the flask was replaced with nitrogen, 360 mg (3.7 mmol) of sodium tert-butoxide and 18 mL of toluene were added thereto. This mixture was degassed by being stirred under reduced pressure. To this mixture, 37 μL (37 μmol) of tris(tert-butyl)phosphine (10 wt % hexane solution) was dripped, the temperature was raised to 60° C., stirring was performed, 11 mg (18 μmol) of bis(dibenzylideneacetone)palladium(0) was added, and stirring was performed at 120° C. for an hour. After the stirring, the reacted solution was suction-filtered through alumina, Celite (Catalog No. 537-02305 produced by FUJIFILM Wako Pure Chemical Corporation), and Florisil (Catalog No. 066-05265 produced by FUJIFILM Wako Pure Chemical Corporation). The obtained filtrate was concentrated to give a solid, the solid was purified by silica gel column chromatography (hexane:toluene=7:1), whereby 675 mg of a target white solid was obtained in a yield of 52%. Synthesis Scheme (s2-2) of Step 2 is shown below.

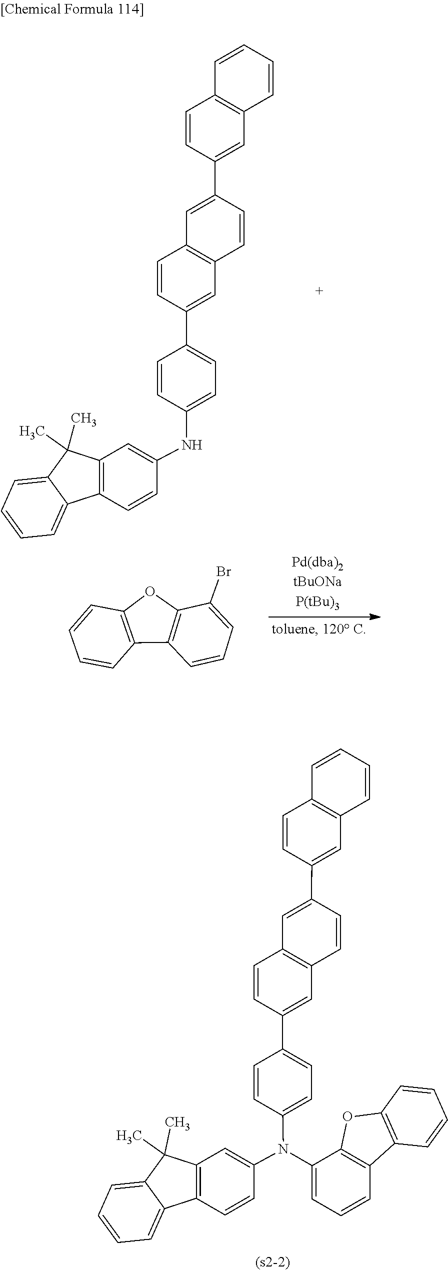 [SMILES: CC1(C)c2ccccc2-c2ccc(Nc3ccc(-c4ccc5cc(-c6ccc7ccccc7c6)ccc5c4)cc3)cc21 ; Brc1cccc2c1oc1ccccc12 ; CC1(C)c2ccccc2-c2ccc(N(c3ccc(-c4ccc5cc(-c6ccc7ccccc7c6)ccc5c4)cc3)c3cccc4c3oc3ccccc34)cc21]

By a train sublimation method, 0.67 g of the obtained solid was purified. In the purification by sublimation, the solid was heated at 295° C. for 20 hours under a pressure of 2.9 Pa with an argon flow rate of 8.0 mL/min. After the purification by sublimation, 0.33 g of a target solid was obtained at a collection rate of 49%.

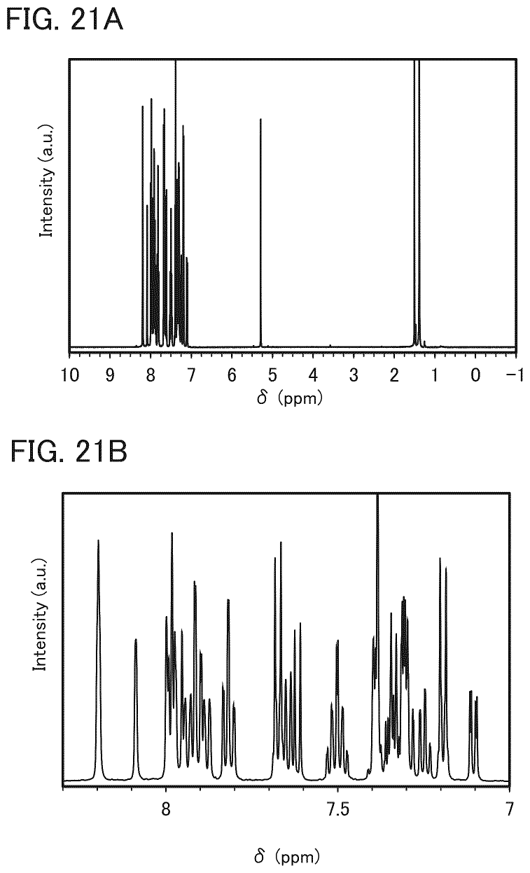

The ¹H NMR measurement results of the obtained solid is shown below and in FIGS. 21A and 21B. Note that FIG. 21B is a chart where the range from 7.00 ppm to 8.30 ppm in FIG. 21A is enlarged. The results show that FrFA(βN2)B was obtained in this synthesis example.

¹H NMR (dichloromethane-d₂, 500 MHz, 20° C.): δ=8.20 (s, 2H), δ=8.09 (s, 1H), δ=8.00-7.87 (m, 8H), δ=7.82 (dt, J=8.0 Hz, 2.0 Hz, 2H), δ=7.67 (d, J=8.5 Hz, 2H), δ=7.64 (d, J=7.5 Hz, 1H), δ=7.62 (d, J=8.0 Hz, 1H), δ=7.53-7.47 (m, 2H), δ=7.41-7.23 (m, 9H), δ=7.19 (d, J=8.5 Hz, 2H), δ=7.11 (dd, J=8.0 Hz, 2.0 Hz, 1H), δ=1.54 (s, 6H).

### <Measurement of Physical Properties>

Next, the absorption spectra and the PL spectra of a toluene solution of FrFA(βN2)B and a thin film of FrFA(βN2)B were measured. The measurement was performed by a method similar to that in Example 1.

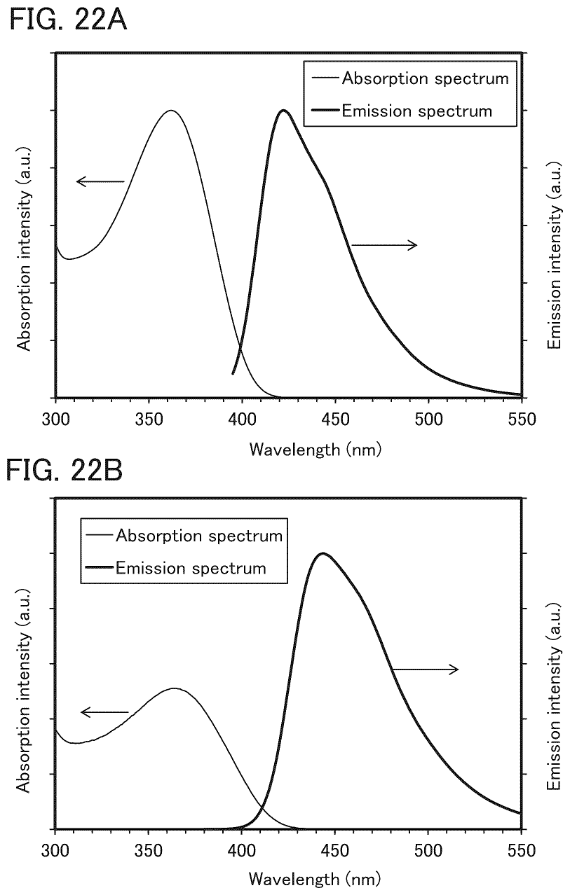

FIG. 22A shows the measurement results of the toluene solution and FIG. 22B shows the measurement results of the thin film. The measurement results show that FrFA(βN2)B in the toluene solution has an absorption peak at around 362 un, the thin film of FrFA(βN2)B has an absorption peak at around 365 nm, and an absorption band is not observed on the wavelength side longer than 440 nm. This indicates that in the case where FrFA(βN2)B is used in a light-emitting device, a reduction in emission efficiency caused by absorption does not occur at a light wavelength used in a display and thus FrFA(βN2)B can be suitably used. The toluene solution of FrFA(βN2)B exhibited an emission wavelength peak at around 422 nm (excitation wavelength: 366 nm), and the thin film of FrFA(βN2)B exhibited an emission peak at around 444 nm (excitation wavelength: 365 nm).

The HOMO level and the LUMO level of FrFA(βN2) were calculated by cyclic voltammetry (CV) measurement. A calculation method is similar to that described in Example 1. As a result, in the measurement of an oxidation potential Ea [V] of FrFA(βN2), the HOMO level was found to be −5.47 eV. In contrast, the LUMO level was −2.47 eV in the measurement of the reduction potential Ec [V]. When the oxidation-reduction wave was repeatedly measured, in the Ea measurement, the peak intensity of the oxidation-reduction wave after the 100th cycle was maintained to be 96% of that of the oxidation-reduction wave at the first cycle, and in the Ec measurement, the peak intensity of the oxidation-reduction wave after the 100th cycle was maintained to be 98% of that of the oxidation-reduction wave at the first cycle; thus, resistance of FrFA(βN2) to repetitive oxidation and repetitive reduction was found to be extremely high.

DSC measurement of FrFA(βN2) was performed. The measurement was performed by a method similar to that in Example 1. From the DSC measurement results in the second cycle, the glass transition point of FrFA(βN2) was 128° C., and the crystallization temperature and the melting point were not observed. This indicates that FrFA(βN2) is a substance having extremely high heat resistance and the film of FrFA(βN2) can maintain a thermally stable quality.

TG-DTA of FrFA(βN2) was performed. The measurement was performed by a method similar to that in Example 1. As a result, it was found that the decomposition temperature of FrFA(βN2) is higher than or equal to 460° C., which indicates that FrFA(βN2) is a substance having high heat resistance.

### Example 3

In this example, a light-emitting device of one embodiment of the present invention will be described in detail. Structural formulae of typical organic compounds used in this example are shown below.

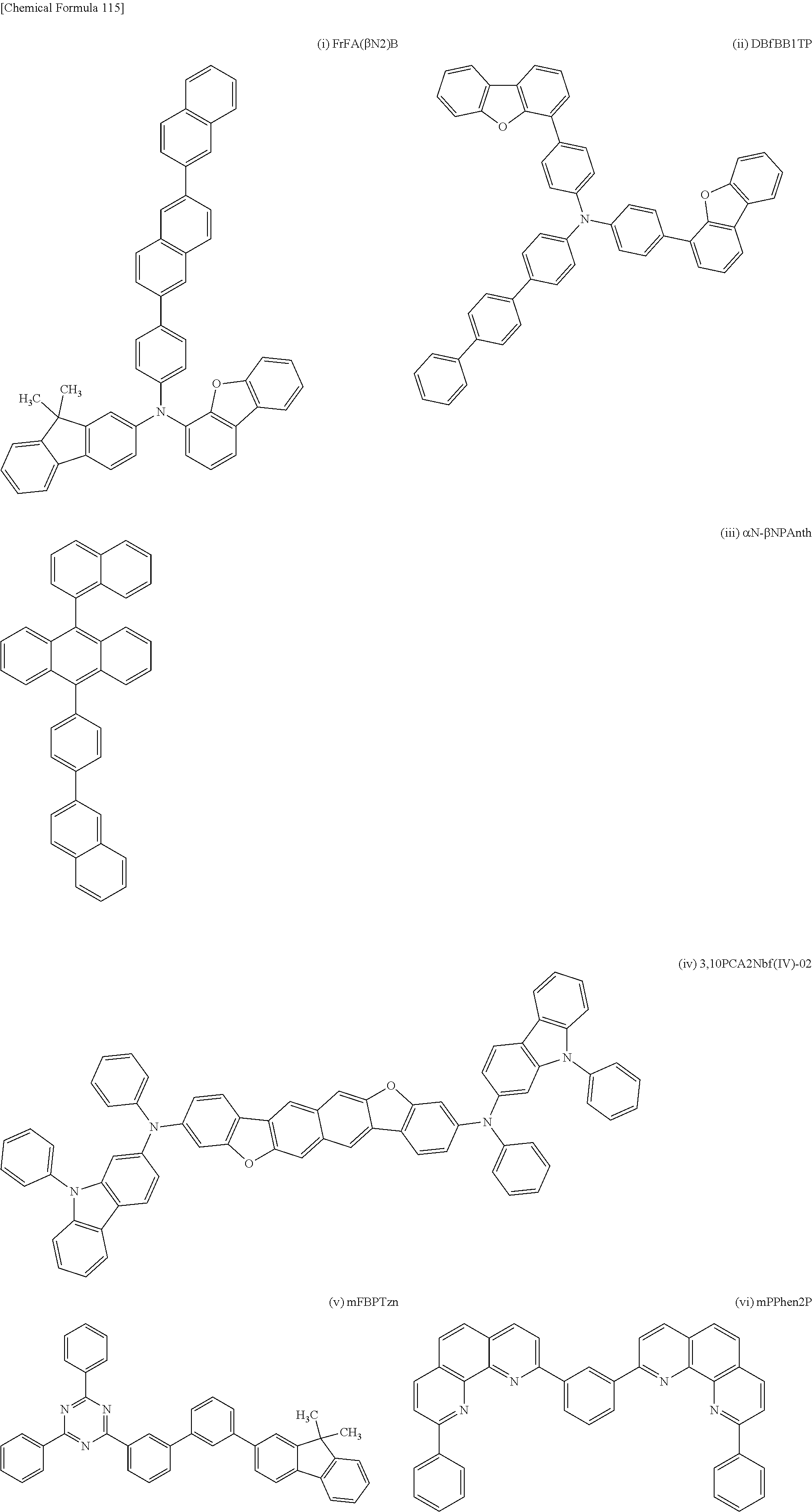

### (Method for Fabricating Light-Emitting Device)

First, indium tin oxide containing silicon oxide (ITSO) was deposited over a glass substrate to a thickness of 110 nm by a sputtering method to form the first electrode 101. Note that the electrode area was 2 mm×2 mm.

Next, in pretreatment for forming the light-emitting device over the substrate, a surface of the substrate was washed with water and baked at 200° C. for an hour, and then, UV ozone treatment was performed for 370 seconds.

After that, the substrate was transferred into a vacuum evaporation apparatus where the pressure had been reduced to approximately 1×10⁻⁴ Pa, and was subjected to vacuum baking at 170° C. for 30 minutes in a heating chamber of the vacuum evaporation apparatus, and then the substrate was cooled down for approximately 30 minutes.

Next, the substrate provided with the first electrode 101 was fixed to a substrate holder provided in the vacuum evaporation apparatus such that the surface on which the first electrode 101 was formed faced downward. Then, N-[4-(2,2′-binaphthyl-6-yl)phenyl]-N,N-(9,9-dimethyl-9H-fluoren-2-yl)dibenzofuran-4-amine (abbreviation: FrFA(βN2)B) represented by Structural Formula (i) and a fluorine-containing electron acceptor material with a molecular weight of 672 (OCPD-003) were deposited on the first electrode 101 to a thickness of 10 nm by co-evaporation using resistance heating such that the weight ratio of FrFA(βN2)B to OCPD-003 was 1:0.1, whereby the hole-injection layer 111 was formed.

Subsequently, over the hole-injection layer 111, FrFA(βN2)B was deposited to a thickness of 90 nm by evaporation to form a first hole-transport layer, and then N,N-bis[4-(dibenzofuran-4-yl)phenyl]-4-amino-p-terphenyl (abbreviation: DBfBBITP) represented by Structural Formula (ii) was deposited to a thickness of 10 nm to form a second hole-transport layer, whereby the hole-transport layer 112 was formed. Note that the second hole-transport layer also functions as an electron-blocking layer.

Then, 9-(1-naphthyl)-10-[4-(2-naphthyl)phenyl]anthracene (abbreviation: αN-bNPAnth) represented by Structural Formula (iii) and 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02) represented by Structural Formula (iv) were deposited on the hole-transport layer 112 to a thickness of 25 nm by co-evaporation such that the weight ratio of aN-bNPAnth to 3,10PCA2Nbf(IV)-02 was 1:0.015, whereby the light-emitting layer 113 was formed.

After that, 2-[3′-(9,9-dimethyl-9H-fluoren-2-yl)biphenyl-3-yl]-4,6-diphenyl-1,3,5-triazine (abbreviation: mFBIPTzn) represented by Structural Formula (v) was deposited on the light-emitting layer 113 to a thickness of 10 nm to form a first electron-transport layer, and then 2,2′-(1,3-phenylene)bis(9-phenyl-1,10-phenanthroline) (abbreviation: mPPhen2P) represented by Structural Formula (vi) was deposited to a thickness of 15 nm to form a second electron-transport layer, whereby the electron-transport layer 114 was formed.

Lithium fluoride was deposited over the electron-transport layer 114 to a thickness of 1 nm, whereby the electron-injection layer 115 was formed.

Lastly, aluminum was deposited by evaporation to a thickness of 200 nm to form the second electrode 102, whereby the light-emitting device of one embodiment of the present invention was fabricated.

The device structure of the light-emitting device is listed in the following table.

TABLE 1

| | Film | |
|---|---|---|
| | thickness | |
| | (nm) | Structure |
| Second electrode | 200 | Al |
| Electron-injection | 1 | LiF |
| layer | | |
| Electron-transport | 15 | mPPhen2P |
| layer | 10 | mFBPTzn |
| Light-emitting layer | 25 | αN-βNP Anth:3,10PCA2Nmf(IV)-02 |
| | | (1:0.015) |
| Hole-transport layer | 10 | DBfBB1TP |
| | 90 | FrFA(βN2)B |
| Hole-injection layer | 10 | FrFA(βN2)B:OCPD-003 |
| | | (1:0.1) |
| First electrode | 110 | ITSO |

The light-emitting device was sealed using a glass substrate in a glove box containing a nitrogen atmosphere so as not to be exposed to the air (a sealing material was applied to surround the device and UV treatment and heat treatment at 80° C. for an hour were performed at the time of sealing). Then, the initial characteristics of the light-emitting device were measured.

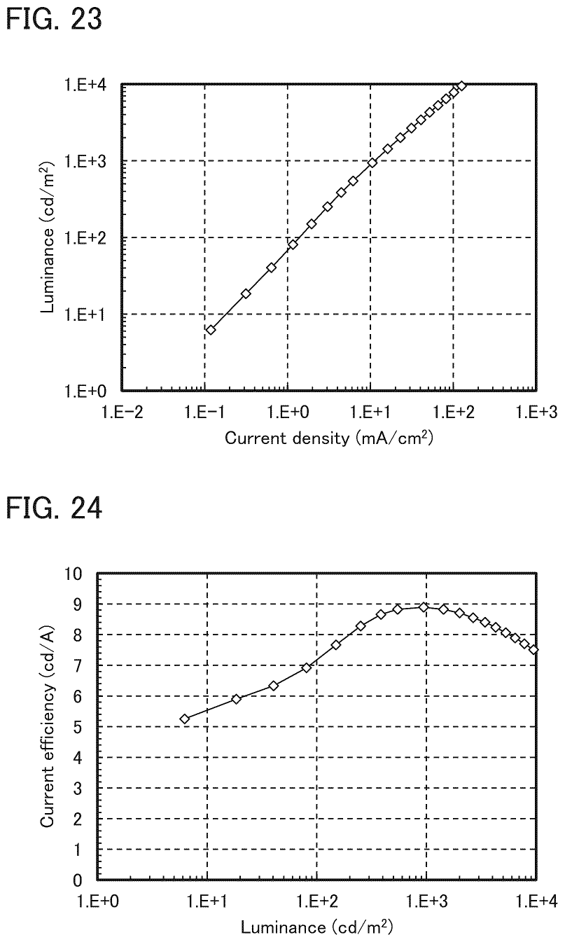

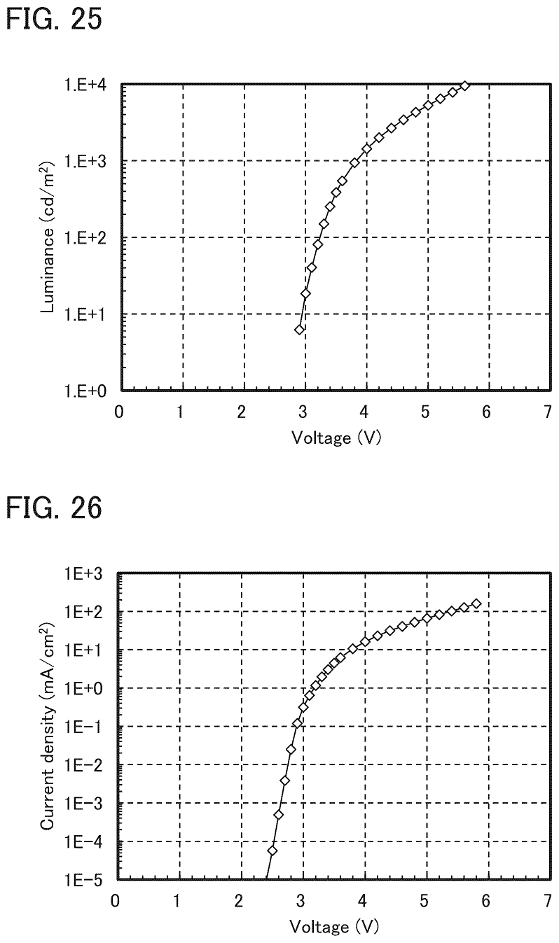

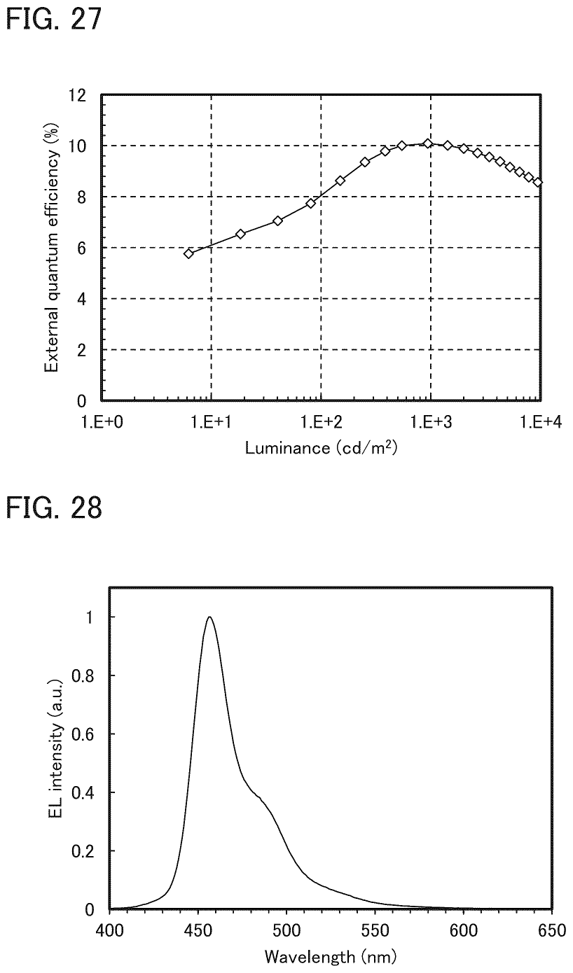

FIG. 23 shows the luminance-current density characteristics of the light-emitting device. FIG. 24 shows the current efficiency-luminance characteristics thereof. FIG. 25 shows the luminance-voltage characteristics thereof. FIG. 26 shows the current density-voltage characteristics thereof. FIG. 27 shows the external quantum efficiency-luminance characteristics thereof. FIG. 28 shows the electroluminescence spectrum thereof. Table 2 shows the main characteristics of the light-emitting device at a luminance of approximately 1000 cd/M². Note that the luminance, CIE chromaticity, and emission spectrum were measured at normal temperature with a spectroradiometer (SR-ULIR produced by TOPCON TECHNOHOUSE CORPORATION). The external quantum efficiency was calculated from the measured luminance and emission spectrum, on the assumption that the light-emitting device had Lambertian light-distribution characteristics.

TABLE 2

| | | Current | | Current | External |
|---|---|---|---|---|---|
| Voltage | Current | density | Chromaticity | efficiency | quantum |
| (V) | (mA) | (mA/cm²) | (x, y) | (cd/A) | efficiency (%) |
| 3.8 | 0.42 | 10.6 | (0.14, 0.10) | 8.9 | 10.1 |

FIG. 23 to FIG. 28 and Table 2 show that the light-emitting device of one embodiment of the present invention has favorable characteristics.

### Example 4

### Synthesis Example 3

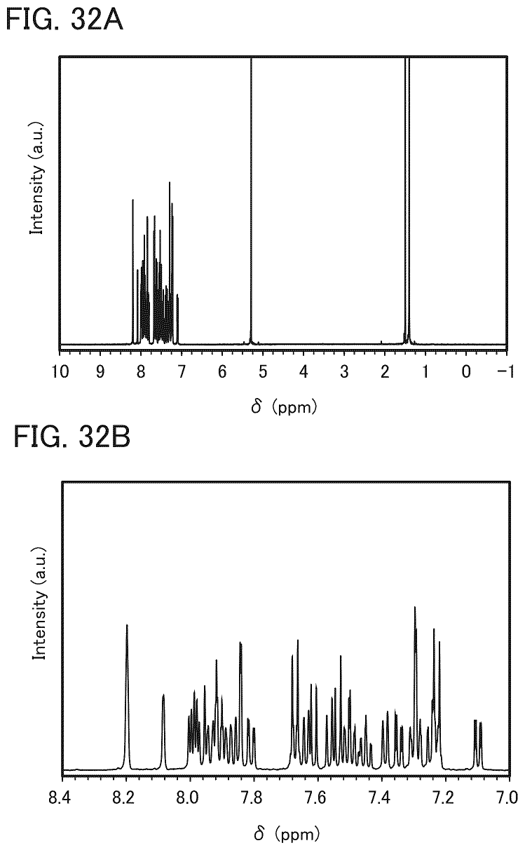

In this synthesis example, N-[4-(2,2′-binaphthyl-6-yl)phenyl]-N-(9,9′-dimethyl-9H-fluoren-2-yl)dibenzofuran-2-amine (abbreviation: Fr(2)FA(βN2)B) represented by Structural Formula (153) in Embodiment 1 was synthesized. Fr(2)FA(βN2)B was synthesized by a method similar to that of FrFA(βN2)B synthesized in Example 2. The structural formula of Fr(2)FA(βN2)B, the synthesis scheme (s3), and ¹H NMR values are shown below and the chart is shown in FIGS. 32A and 32B. Note that FIG. 32B is a chart where the range from 7.00 ppm to 8.40 ppm in FIG. 32A is enlarged. The results show that Fr(2)FA(βN2)B was obtained.

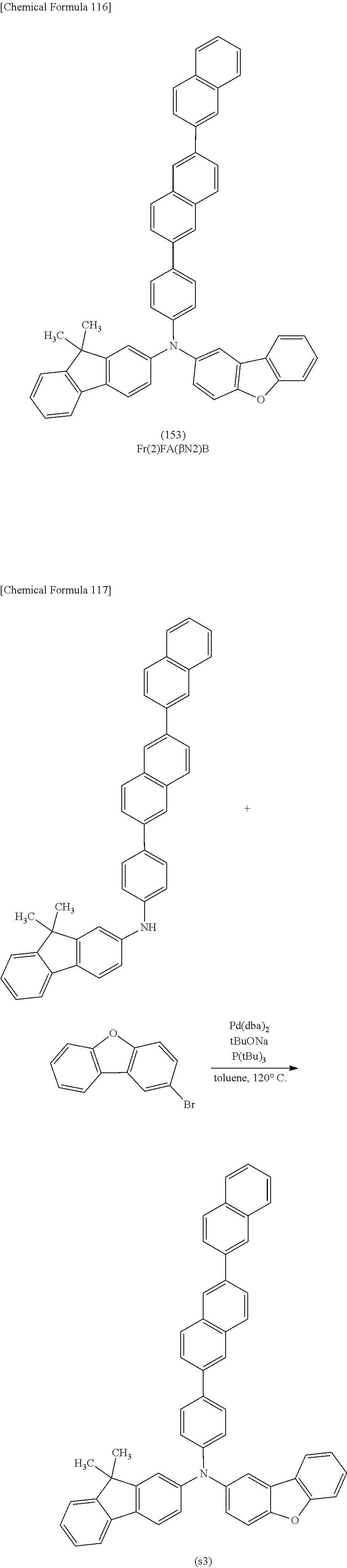 [SMILES: CC1(C)c2ccccc2-c2ccc(N(c3ccc(-c4ccc5cc(-c6ccc7ccccc7c6)ccc5c4)cc3)c3ccc4oc5ccccc5c4c3)cc21 ; CC1(C)c2ccccc2-c2ccc(Nc3ccc(-c4ccc5cc(-c6ccc7ccccc7c6)ccc5c4)cc3)cc21 ; Brc1ccc2oc3ccccc3c2c1 ; CC1(C)c2ccccc2-c2ccc(N(c3ccc(-c4ccc5cc(-c6ccc7ccccc7c6)ccc5c4)cc3)c3ccc4oc5ccccc5c4c3)cc21]

¹H NMR (dichloromethane-d₂, 500 MHz, 20° C.): δ=8.20 (s, 2H), δ=8.08 (s, 1H), δ=8.01-7.84 (m, 9H), δ=7.81 (dd, J=8.5 Hz, J=2.0 Hz, 1H), δ=7.67 (d, J=8.5 Hz, 2H), δ=7.65-7.61 (m, 2H), δ=7.57-7.44 (m, 5H), δ=7.39 (d, J=7.5 Hz, 1H), δ=7.35 (dd, J=10 Hz, J=2.5 Hz, 1H), δ=7.31-7.22 (m, 6H), δ=7.10 (dd, J=8.0 Hz, J=2.5 Hz, 1H), δ=1.40 (s, 6H).

The measurement results of the physical properties of Fr(2)FA(βN2)B are shown in the table below. Note that the measurement was performed by a method similar to that in Example 1. As a result, an absorption band of Fr(2)FA(βN2)B is not observed on the wavelength side longer than 440 nm, and it is considered that a reduction in emission efficiency of the light-emitting device due to absorption does not occur in the visible light wavelength region and thus Fr(2)FA(βN2)B can be suitably used. In addition, Fr(2)FA(βN2)B was found to be a substance having high heat resistance.

TABLE 3

| Absorption | | Emission | | | | | Decom- |
|---|---|---|---|---|---|---|---|
| peak (nm) | | peak (nm) | | | | | position |
| Thin | | Thin | | HOMO | LUMO | Tg | temperature |
| film | Solution | film | Solution | (eV) | (eV) | (° C.) | (° C.) |
| 380 | 356 | 453 | 418 | −5.37 | −2.46 | 125.0 | 497.0 |

### Example 5

### Synthesis Example 4

In this synthesis example, a method for synthesizing N-[4-(2,2′-binaphthyl-6-yl)phenyl]-N-(9,9-dimethyl-9H-fluoren-4-yl)dibenzofuran-4-amine (abbreviation: FrF(4)A(βN2)B), which is represented by Structural Formula (157) in Embodiment 1, was synthesized. The structural formula of FrF(4)A(βN2)B is shown below.

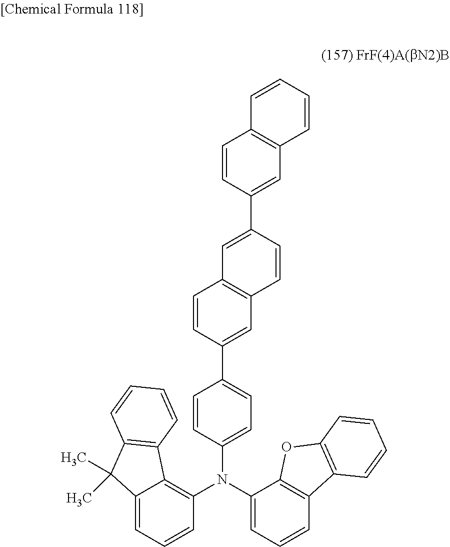 [SMILES: CC1(C)c2ccccc2-c2c(N(c3ccc(-c4ccc5cc(-c6ccc7ccccc7c6)ccc5c4)cc3)c3cccc4c3oc3ccccc34)cccc21]

### Step 1: Synthesis of N-(chlorophenyl-4-yl)-N-(9,9-dimethyl-9H-fluoren-4-yl)dibenzofuran-4-amine

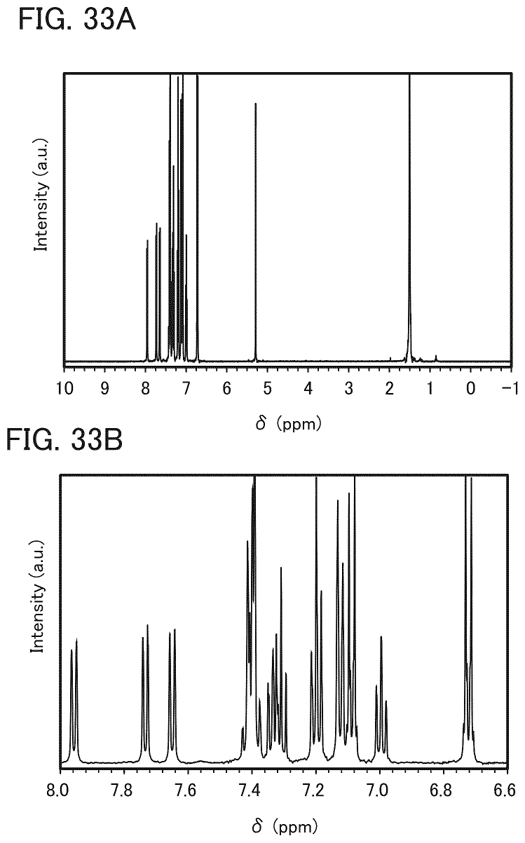

This synthesis step was performed in a manner similar to that of Step 2 in Example 1. Synthesis Scheme (s4-1) of Step 1 is shown below. ¹H NMR of the obtained white solid was measured. The values are shown below and the chart is shown in FIGS. 33A and 33B. Note that FIG. 33B is a chart where the range from 6.60 ppm to 8.00 ppm in FIG. 33A is enlarged. This indicates that N-(chlorophenyl-4-yl)-N-(9,9-dimethyl-9H-fluoren-4-yl)dibenzofuran-4-amine was obtained in this step.

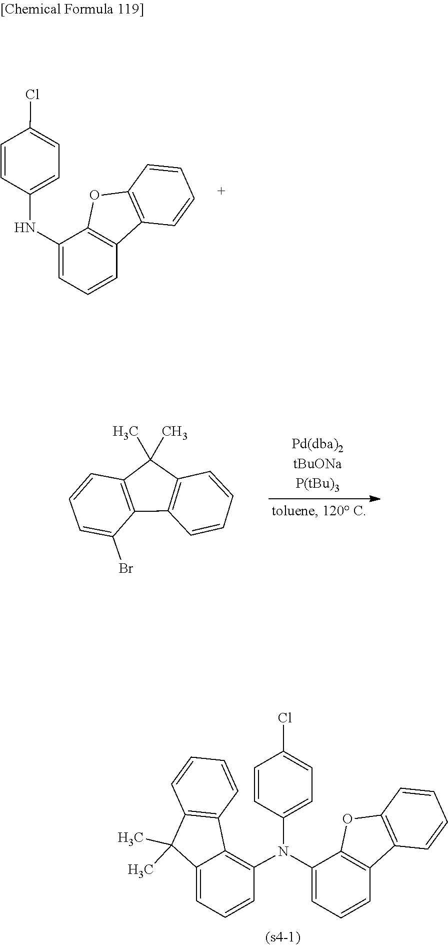 [SMILES: Clc1ccc(Nc2cccc3c2oc2ccccc23)cc1 ; CC1(C)c2ccccc2-c2c(Br)cccc21 ; CC1(C)c2ccccc2-c2c(N(c3ccc(Cl)cc3)c3cccc4c3oc3ccccc34)cccc21]

¹H NMR (dichloromethane-d₂, 500 MHz, 20° C.): δ=7.96 (d, J=8.0 Hz, 1H), δ=7.73 (d, J=8.0 Hz, 1H), δ=7.65 (d, J=7.5 Hz, 1H), δ=7.43-7.29 (m, 6H), δ=7.20 (t, J=7.5 Hz, 2H), δ=7.12 (d, J=8.0 Hz, 2H), δ=7.09 (d, J=8.5 Hz, 2H), δ=7.00 (t, J=7.5 Hz, 1H), δ=6.72 (d, J=9.5 Hz, 2H), δ=1.51 (s, 6H).

### Step 2: Synthesis of FrF(4)A(βN2)B

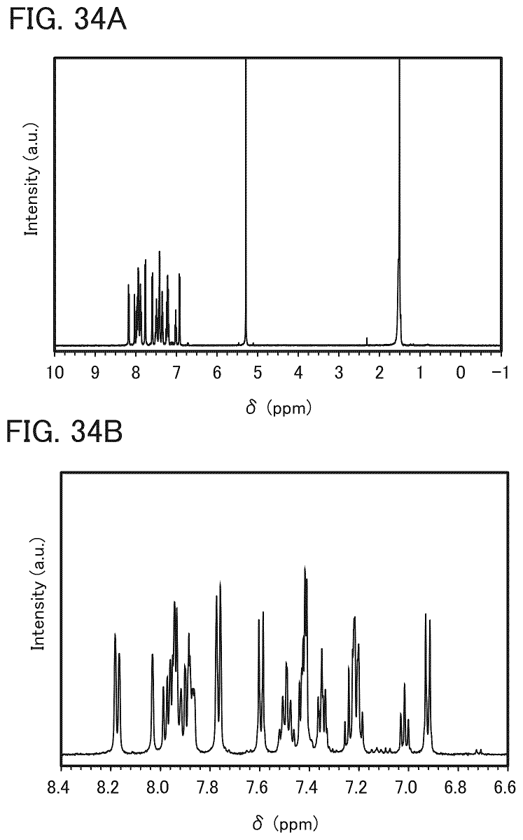

Into a 200-mL three-neck flask were put 2.0 g (4.1 mmol) of N-(chlorophenyl-4-yl)-N-(9,9-dimethyl-9H-fluoren-4-yl)dibenzofuran-4-amine obtained in Step 1, 1.6 g (4.1 mmol) of 2-[2,2′-binaphthalen-6-yl]-4,4,5,5-tetramethyl-1,3,2-dioxaborolane, 1.4 g (12 mmol) of potassium tert-butoxide, 41 mL of toluene, 10 mL of ethanol, and 10 mL of pure water. This mixture was degassed by being stirred under reduced pressure and then heated to 60° C.; 56 mg of [1,3-bis(2,6-diisopropylphenyl)imidazol-2-ylidene](3-chloropyridyl)palladium(II) dichloride (abbreviation: PEPPSI™—IPr) was added thereto; and the temperature was raised to 90° C. and the mixture was stirred for an hour. After the stirring, the mixture was cooled to room temperature, the reacted mixture was concentrated, and suction filtration was performed using hot toluene. The obtained filtrate was concentrated, the obtained yellow solid was dissolved in toluene, and then ethanol was added to cause recrystallization, so that 2.1 g of a target pale yellow solid was obtained in a yield of 74%. Synthesis Scheme (s4-2) of Step 2 is shown below. ¹H NMR of the obtained pale yellow solid was measured. The values are shown below and the chart is shown in FIGS. 34A and 34B. Note that FIG. 34B is a chart where the range from 6.60 ppm to 8.40 ppm in FIG. 34A is enlarged. This indicates that FrF(4)A(βN2)B was obtained in this step.

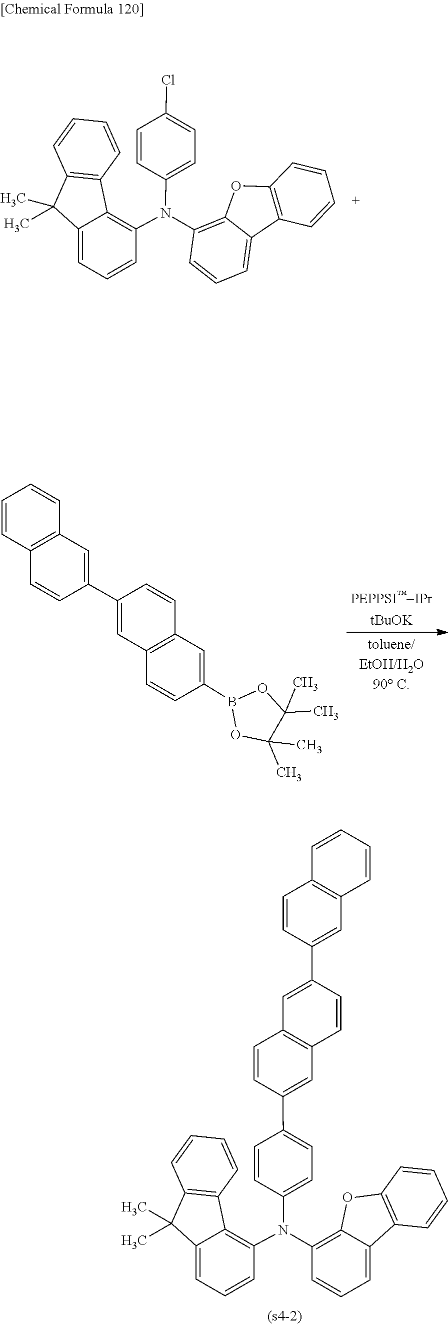

¹H NMR (dichloromethane-d₂, 500 MHz, 20° C.): δ=8.17 (d, J=9.0 Hz, 2H), δ=8.03 (s, 1H), δ=7.99-7.86 (m, 8H), δ=7.77 (d, J=7.5 Hz, 3H), δ=7.59 (d, J=8.5 Hz, 2H), δ=7.52-7.46 (m, 2H), δ=7.44-7.39 (m, 4H), δ=7.37-7.32 (m, 2H), δ=7.26-7.19 (m, 4H), δ=7.02 (td, J=7.5 Hz, 1.0 Hz, 1H), δ=6.92 (d, J=9 Hz, 2H), δ=1.51 (s, 6H).

The measurement results of the physical properties of FrF(4)A(βN2)B are shown in the table below. Note that the measurement was performed by a method similar to that in Example 1. As a result, an absorption band of FrF(4)A(βN2)B is not observed on the wavelength side longer than 440 nm, and it is considered that a reduction in emission efficiency of the light-emitting device due to absorption does not occur in the visible light wavelength region and thus FrF(4)A(βN2)B can be suitably used. In addition, FrF(4)A(βN2)B was found to be a substance having high heat resistance.

TABLE 4

| Absorption | | Emission | | | | | Decom- |
|---|---|---|---|---|---|---|---|
| peak (nm) | | peak (nm) | | | | | position |
| Thin | | Thin | | HOMO | LUMO | Tg | temperature |
| film | Solution | film | Solution | (eV) | (eV) | (° C.) | (° C.) |
| — | 352 | — | 409 | −5.60 | −2.44 | 133.7 | 474.5 |

### Example 6

### Synthesis Example 5

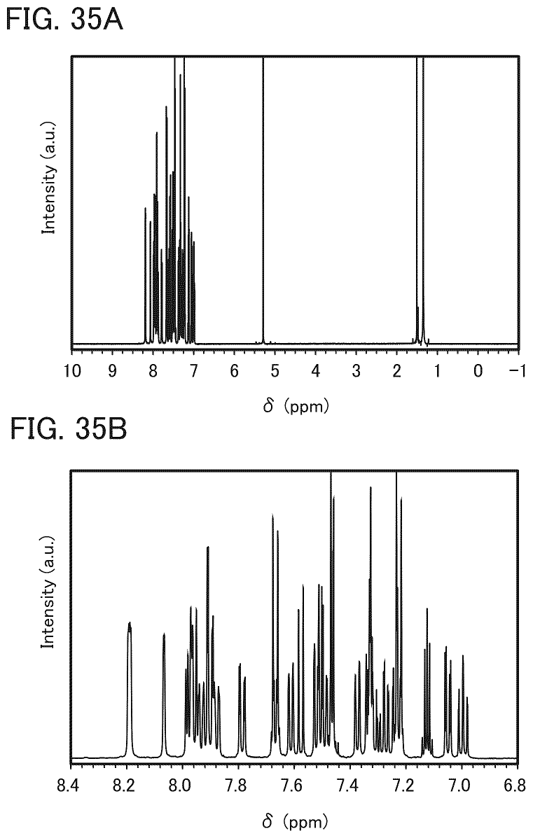

In this synthesis example, N1-[4-(2,2′-binaphthyl-6-yl)phenyl]-N-(9,9-dimethyl-9H-fluoren-2-yl)dibenzofuran-1-amine (abbreviation: Fr(1)FA(βN2)B) represented by Structural Formula (154) in Embodiment 1 was synthesized. Fr(1)FA(βN2)B was synthesized by a method similar to that of FrFA(βN2)B synthesized in Example 2. The structural formula of Fr(1)FA(βN2)B, the synthesis scheme (s5), and ¹H NMR values are shown below and the ¹H NMR chart is shown in FIGS. 35A and 35B. Note that FIG. 35B is a chart where the range from 6.80 ppm to 8.40 ppm in FIG. 35A is enlarged. The results show that Fr(1)FA(βN2)B was obtained in this synthesis example.

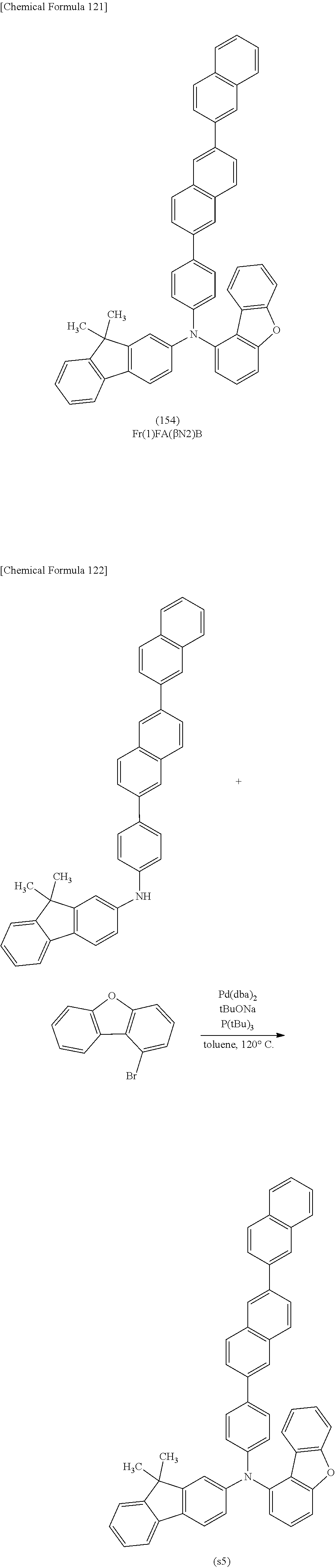 [SMILES: CC1(C)c2ccccc2-c2ccc(N(c3ccc(-c4ccc5cc(-c6ccc7ccccc7c6)ccc5c4)cc3)c3cccc4oc5ccccc5c34)cc21 ; CC1(C)c2ccccc2-c2ccc(Nc3ccc(-c4ccc5cc(-c6ccc7ccccc7c6)ccc5c4)cc3)cc21 ; Brc1cccc2oc3ccccc3c12 ; CC1(C)c2ccccc2-c2ccc(N(c3ccc(-c4ccc5cc(-c6ccc7ccccc7c6)ccc5c4)cc3)c3cccc4oc5ccccc5c34)cc21]

¹H NMR (dichloromethane-d₂, 500 MHz, 20° C.): δ=8.19 (s, 2H), =8.07 (d, J=1.5 Hz, 1H), δ=7.99-7.87 (m, 7H), δ=7.79 (dd, J=8.5 Hz, 2.0 Hz, 1H), =7.67 (dt, J=9.0 Hz, 5.0 Hz, 2H), δ=7.61 (d, J=7.5 Hz, 1H), δ=7.58 (d, J=8.5 Hz, 1H), δ=7.53-7.44 (m, 5H), =7.37 (d, J=7.0 Hz, 1H), δ=7.34-7.21 (m, 7H), δ=7.14-7.11 (m, 1H), δ=7.05 (dd, J=8.0 Hz, 2.0 Hz, 1H), δ=7.00 (t, J=2.5 Hz, 1H), δ=1.35 (s, 6H).

The measurement results of the physical properties of Fr(1)FA(βN2)B are shown in the table below. Note that the measurement was performed by a method similar to that in Example 1. As a result, an absorption band of Fr(l)FA(βN2)B is not observed on the wavelength side longer than 440 nm, and it is considered that a reduction in emission efficiency of the light-emitting device due to absorption does not occur in the visible light wavelength region and thus Fr(1)FA(βN2)B can be suitably used. In addition, Fr(2)FA(βN2)B was found to be a substance having high heat resistance.

TABLE 5

| Absorption | | Emission | | | | | Decom- |
|---|---|---|---|---|---|---|---|
| peak (nm) | | peak (nm) | | | | | position |
| Thin | | Thin | | HOMO | LUMO | Tg | temperature |
| film | Solution | film | Solution | (eV) | (eV) | (° C.) | (° C.) |
| — | 366 | — | 432 | −5.46 | −2.46 | 129.4 | 487.6 |

### Example 7

### Synthesis Example 6

In this synthesis example, a method for synthesizing N-[4-(2,2′-binaphthyl-6-yl)phenyl]-N-(9,9′-diphenyl-9H-fluoren-2-yl)dibenzofuran-2-amine (abbreviation: Fr(2)FLP(2)A(βN2)B), which is represented by Structural Formula (123) in Embodiment 1, was synthesized. The structural formula of Fr(2)FLP(2)A(βN2)B is shown below.

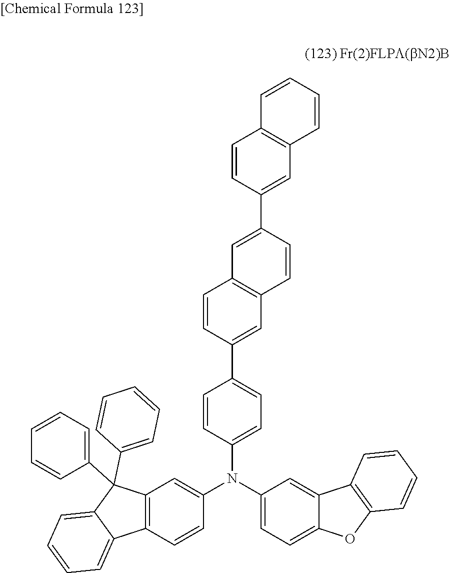 [SMILES: c1ccc(C2(c3ccccc3)c3ccccc3-c3ccc(N(c4ccc(-c5ccc6cc(-c7ccc8ccccc8c7)ccc6c5)cc4)c4ccc5oc6ccccc6c5c4)cc32)cc1]

### Step 1: Synthesis of N-(chlorophenyl-4-yl)-N-(9,9′-diphenyl-9H-fluoren-2-yl)dibenzofuran-2-amine

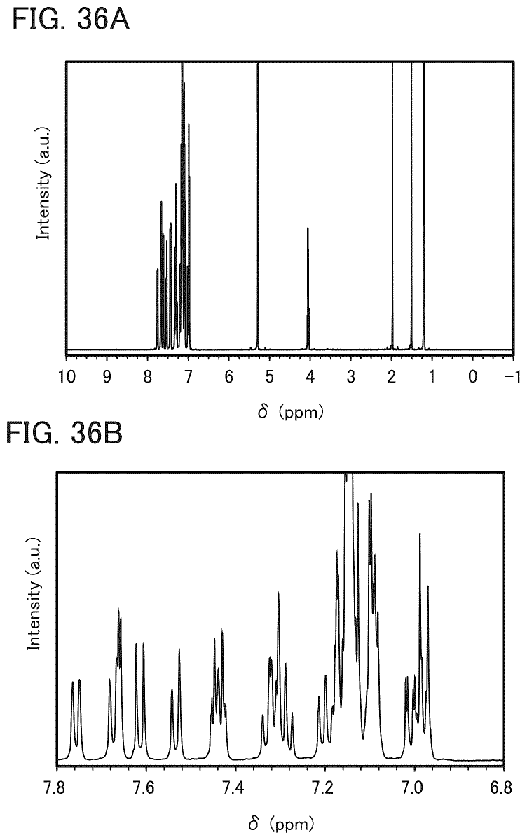

This synthesis step was performed in a manner similar to that of Step 2 in Example 1. Synthesis Scheme (s6-1) of Step 1 is shown below. ¹H NMR of the obtained white solid was measured. The values are shown below and the chart is shown in FIGS. 36A and 36B. Note that FIG. 36B is a chart where the range from 6.80 ppm to 7.80 ppm in FIG. 36A is enlarged. This indicates that N-(chlorophenyl-4-yl)-N-(9,9-diphenyl-9H-fluoren-2-yl)dibenzofuran-2-amine was obtained in this step.

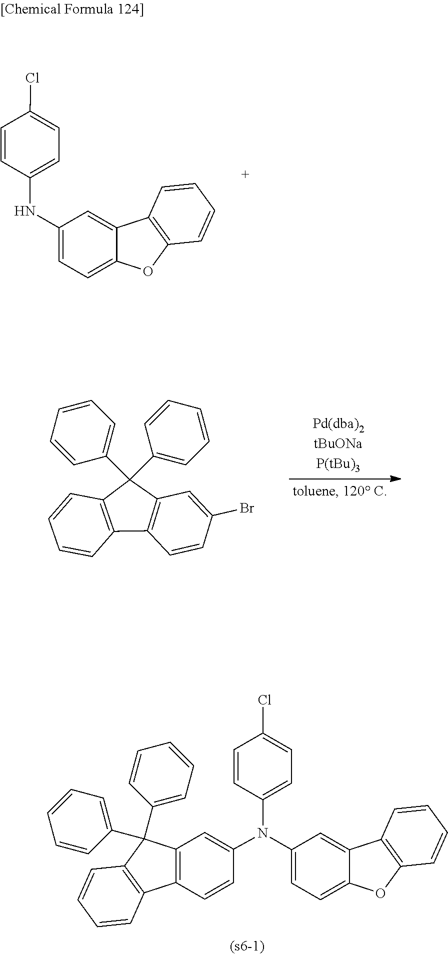 [SMILES: Clc1ccc(Nc2ccc3oc4ccccc4c3c2)cc1 ; Brc1ccc2c(c1)C(c1ccccc1)(c1ccccc1)c1ccccc1-2 ; Clc1ccc(N(c2ccc3c(c2)C(c2ccccc2)(c2ccccc2)c2ccccc2-3)c2ccc3oc4ccccc4c3c2)cc1]

¹H NMR (dichloropethane-d₂, 500 MHz, 20° C.): δ=7.76 (d, J=7.5 Hz, 1H), δ=7.67 (d, J=8.5 Hz, 1H), δ=7.66 (d, J=2.5 Hz, 1H), δ=7.62 (d, J=8.0 Hz, 1H), δ=7.54 (d, J=8.0 Hz, 1H), δ=7.45-7.42 (m, 2H), δ=7.34-7.27 (m, 3H), δ=7.20 (d, J=7.5 Hz, 1H), δ=7.18-7.14 (m, 9H), δ=7.13 (d, J=2.0 Hz, 1H), δ=7.10-7.08 (m, 4H), δ=7.01 (dd, J=8.5 Hz, 2.0 Hz, 1H), δ=7.00-6.97 (m, 2H).

### Step 2: Synthesis of Fr(2)FLP(2)A(βN2)B

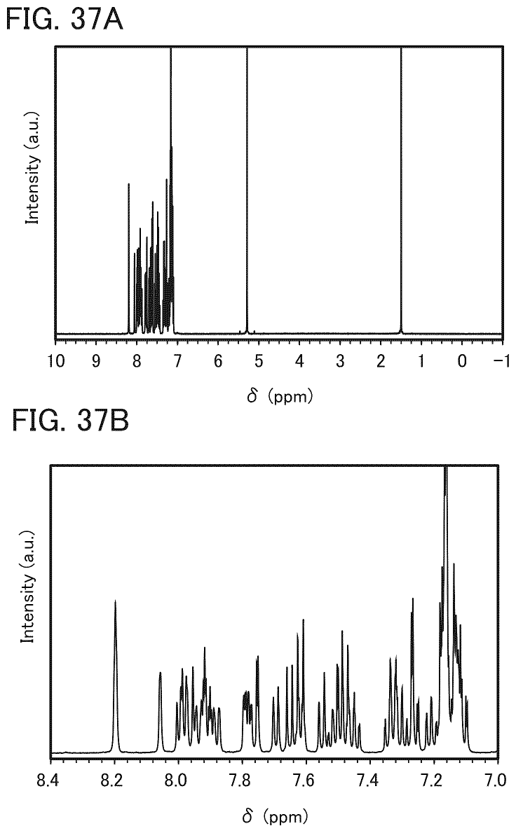

This synthesis step was performed in a manner similar to that of Step 2 in Synthesis Example 4. Synthesis Scheme (s6-2) of Step 2 is shown below. ¹H NMR of the obtained solid was measured. The values are shown below and the chart is shown in FIGS. 37A and 37B. Note that FIG. 37B is a chart where the range from 7.00 ppm to 8.40 ppm in FIG. 37A is enlarged. This indicates that Fr(2)FLP(2)A(βN2)B was obtained in this synthesis example.

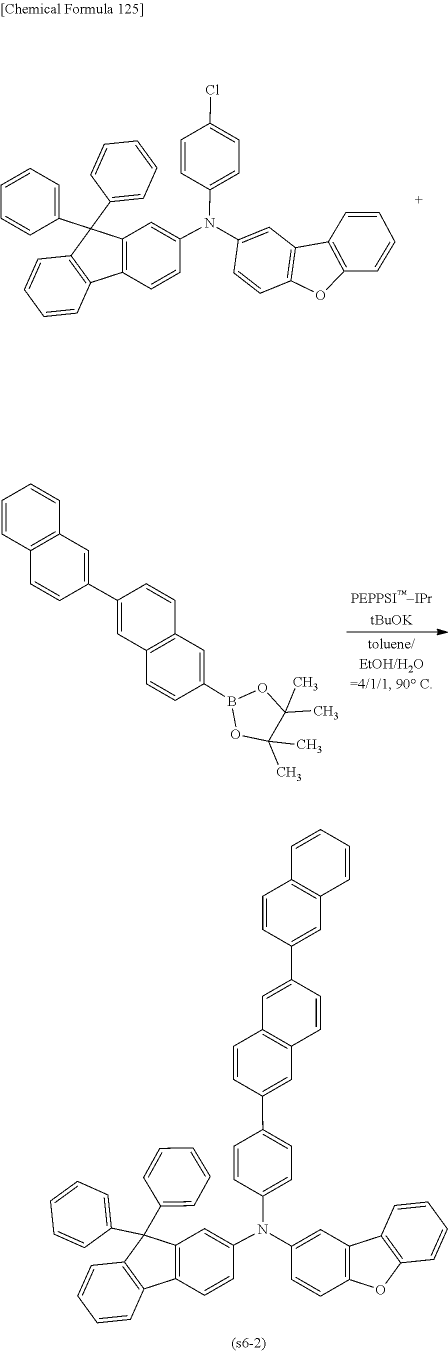 [SMILES: Clc1ccc(N(c2ccc3c(c2)C(c2ccccc2)(c2ccccc2)c2ccccc2-3)c2ccc3oc4ccccc4c3c2)cc1 ; CC1(C)OB(c2ccc3cc(-c4ccc5ccccc5c4)ccc3c2)OC1(C)C ; c1ccc(C2(c3ccccc3)c3ccccc3-c3ccc(N(c4ccc(-c5ccc6cc(-c7ccc8ccccc8c7)ccc6c5)cc4)c4ccc5oc6ccccc6c5c4)cc32)cc1]

¹H NMR (dichloromethane-d₂, 500 MHz, 20° C.): δ=8.20 (s, 2H), δ=8.06 (s, 1H), δ=8.01-7.87 (m, 7H), δ=7.80-7.77 (m, 2H), δ=7.76 (d, J=2.5 Hz, 2H), δ=7.70 (d, J=7.5 Hz, 1H), δ=7.65 (d, J=8.0 Hz, 1H), δ=7.62 (d, J=7.0 Hz, 2H), δ=7.55 (d, J=8.0 Hz, 1H), δ=7.53-7.43 (m, 4H), δ=7.36-7.25 (m, 5H), δ=7.22 (d, J=7.0 Hz, 1H), δ=7.19-7.10 (m, 12H).

The measurement results of the physical properties of Fr(2)FLP(2)A(βN2)B are shown in the table below. Note that the measurement was performed by a method similar to that in Example 1. As a result, an absorption band of Fr(2)FLP(2)A(βN2)B is not observed on the wavelength side longer than 440 nm, and it is considered that a reduction in emission efficiency of the light-emitting device due to absorption does not occur in the visible light wavelength region and thus Fr(2)FLP(2)A(βN2)B can be suitably used. In addition, Fr(2)FLP(2)A(βN2)B was found to be a substance having high heat resistance.

TABLE 6

| Absorption | | Emission | | | | | Decom- |
|---|---|---|---|---|---|---|---|
| peak (nm) | | peak (nm) | | | | | position |
| Thin | | Thin | | HOMO | LUMO | Tg | temperature |
| film | Solution | film | Solution | (eV) | (eV) | (° C.) | (° C.) |
| 381 | 375 | 450 | 432 | −5.41 | −2.47 | 141.7 | 499.4 |

### Example 8

### Synthesis Example 7

In this synthesis example, N-[4-(2,2′-binaphthyl-6-yl)phenyl]-N-(9,9′-diphenyl-9H-fluoren-2-yl)dibenzofuran-4-amine (abbreviation: FrFLP(2)A(βN2)B), which is represented by Structural Formula (148) in Embodiment 1, was synthesized. The structural formula of FrFLP(2)A(βN2) is shown below.

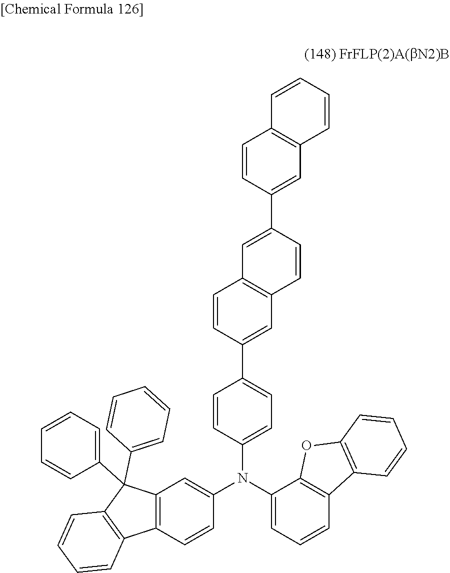 [SMILES: c1ccc(C2(c3ccccc3)c3ccccc3-c3ccc(N(c4ccc(-c5ccc6cc(-c7ccc8ccccc8c7)ccc6c5)cc4)c4cccc5c4oc4ccccc45)cc32)cc1]

### Step 1: Synthesis of N-(chlorophenyl-4-yl)-N-(9,9-diphenyl-9H-fluoren-2-yl)dibenzofuran-4-amine

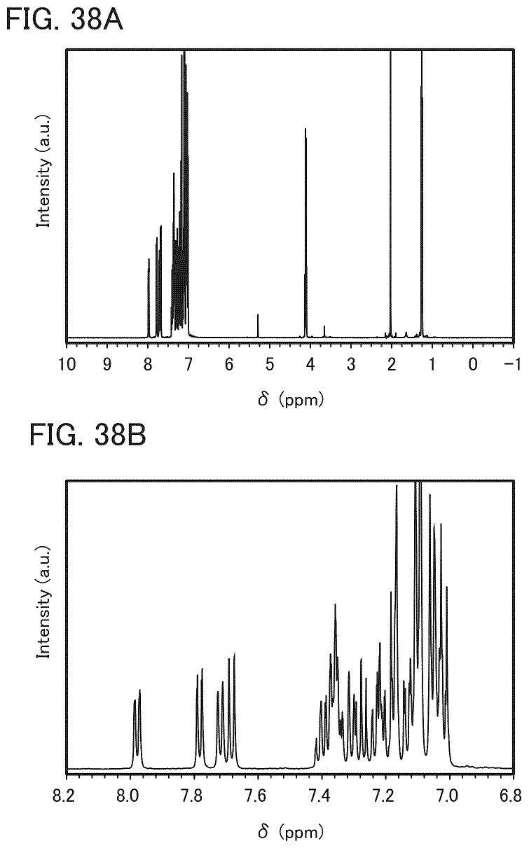

This synthesis step was performed in a manner similar to that of Step 2 in Example 1. Synthesis Scheme (s7-1) of Step 1 is shown below. ¹H NMR of the obtained white solid was measured. The values are shown below and the chart is shown in FIGS. 38A and 38B. Note that FIG. 38B is a chart where the range from 6.80 ppm to 8.20 ppm in FIG. 38A is enlarged. This indicates that N-(chlorophenyl-4-yl)-N-(9,9-diphenyl-9H-fluoren-2-yl)dibenzofuran-4-amine was obtained in this step.

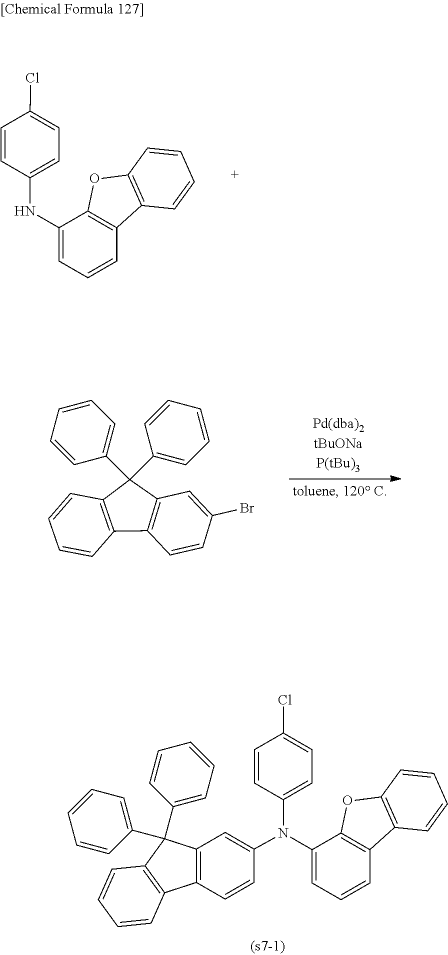 [SMILES: Clc1ccc(Nc2cccc3c2oc2ccccc23)cc1 ; Brc1ccc2c(c1)C(c1ccccc1)(c1ccccc1)c1ccccc1-2 ; Clc1ccc(N(c2ccc3c(c2)C(c2ccccc2)(c2ccccc2)c2ccccc2-3)c2cccc3c2oc2ccccc23)cc1]

¹H NMR (dichloromethane-d₂, 500 MHz, 20° C.): δ=7.98 (d, J=7.5 Hz, 1H), δ=7.78 (dd, J=7.5 Hz, 1.0 Hz, 1H), δ=7.72 (d, J=7.5 Hz, 1H), δ=7.68 (d, J=8.5 Hz, 1H), δ=7.42-7.34 (m, 4H), δ=7.32-7.17 (m, 7H), δ=7.14-7.09 (m, 7H), δ=7.06-7.01 (m, 6H).

### Step 2: Synthesis of FrFLP(2)A(βN2)B

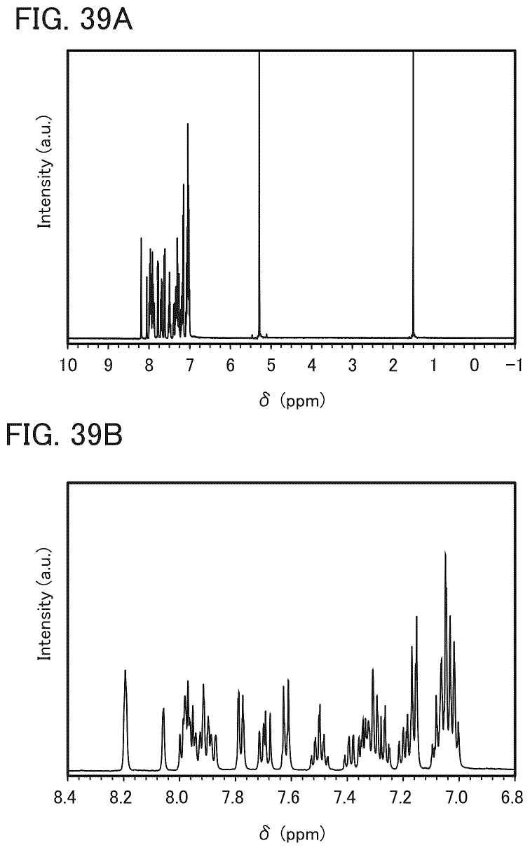

This synthesis step was performed in a manner similar to that of Step 2 in Synthesis Example 4. Synthesis Scheme (s7-2) of Step 2 is shown below. ¹H NMR of the obtained solid was measured. The values are shown below and the chart is shown in FIGS. 39A and 39B. Note that FIG. 39B is a chart where the range from 6.80 ppm to 8.40 ppm in FIG. 39A is enlarged. This indicates that FrFLP(2)A(βN2)B was obtained in this synthesis example.

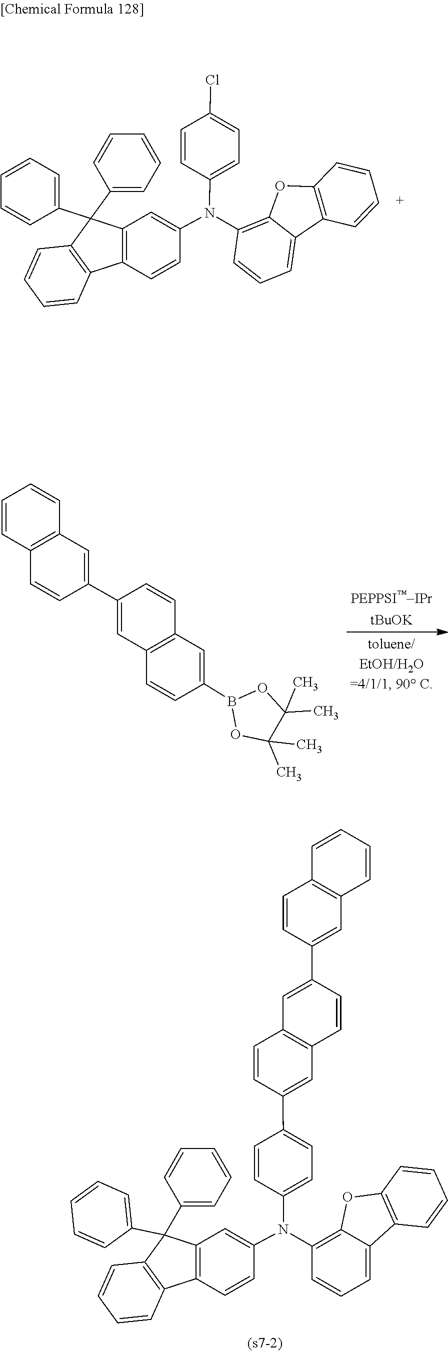 [SMILES: Clc1ccc(N(c2ccc3c(c2)C(c2ccccc2)(c2ccccc2)c2ccccc2-3)c2cccc3c2oc2ccccc23)cc1 ; CC1(C)OB(c2ccc3cc(-c4ccc5ccccc5c4)ccc3c2)OC1(C)C ; c1ccc(C2(c3ccccc3)c3ccccc3-c3ccc(N(c4ccc(-c5ccc6cc(-c7ccc8ccccc8c7)ccc6c5)cc4)c4cccc5c4oc4ccccc45)cc32)cc1]

¹H NMR (dichloromnethane-d₂, 500 MHz, 20° C.): δ=8.20 (s, 2H), δ=8.06 (s, 1H), δ=8.00-7.87 (m, 8H), δ=7.78 (d, J=7.0 Hz, 2H), δ=7.71 (d, J=7.5 Hz, 1H), δ=7.69 (d, J=8.0 Hz, 1H), δ=7.62 (d, J=9.0 Hz, 2H), δ=7.53-7.47 (m, 2H), δ=7.41-7.25 (m, 7H), δ=7.22-7.15 (m, 5H), δ=7.10-7.00 (m, 10H).

The measurement results of the physical properties of FrFLP(2)A(βN2)B are shown in the table below. Note that the measurement was performed by a method similar to that in Example 1. As a result, an absorption band of FrFLP(2)A(βN2)B is not observed on the wavelength side longer than 440 nm, and it is considered that a reduction in emission efficiency of the light-emitting device due to absorption does not occur in the visible light wavelength region and thus FrFLP(2)A(βN2)B can be suitably used. In addition, FrFLP(2)A(βN2)B was found to be a substance having high heat resistance.

TABLE 7

| Absorption | | Emission | | | | | Decom- |
|---|---|---|---|---|---|---|---|
| peak (nm) | | peak (nm) | | | | | position |
| Thin | | Thin | | HOMO | LUMO | Tg | temperature |
| film | Solution | film | Solution | (eV) | (eV) | (° C.) | (° C.) |
| 368 | 366 | 460 | 421 | −5.51 | −2.47 | 146.2 | 497.9 |

### Example 9

### Synthesis Example 8

In this synthesis example, N-[4-(2,2′-binaphthyl-6-yl)phenyl]-N-[9,9′-spirobi[9H-fluoren]-2-yl]dibenzofuran-4-amine (abbreviation: FrSFA(βN2)B), which is represented by Structural Formula (100) in Embodiment 1, was synthesized. The structural formula of FrSFA(βN2)B is shown below.

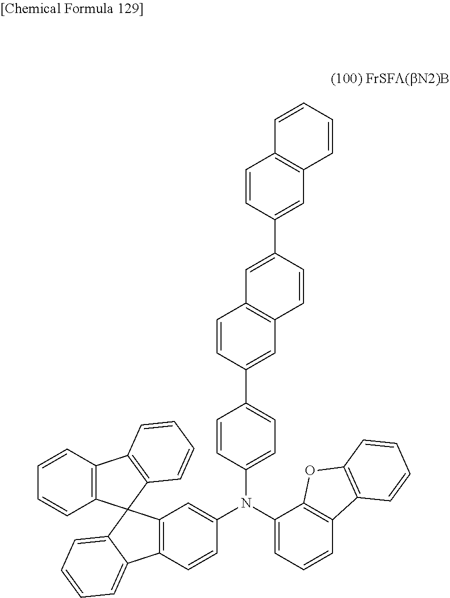 [SMILES: c1ccc2c(c1)-c1ccccc1C21c2ccccc2-c2ccc(N(c3ccc(-c4ccc5cc(-c6ccc7ccccc7c6)ccc5c4)cc3)c3cccc4c3oc3ccccc34)cc21]

### Step 1: Synthesis of FrSFA(βN2)B

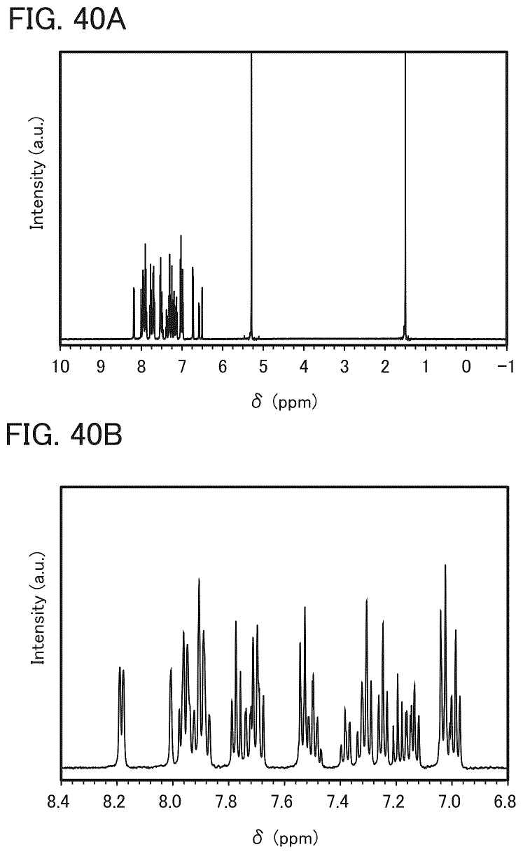

This synthesis step was performed in a manner similar to that of Step 2 in Synthesis Example 4. Synthesis Scheme (s8) of Step 1 is shown below. ¹H NMR of the obtained solid was measured. The values are shown below and the chart is shown in FIGS. 40A and 40B. Note that FIG. 40B is a chart where the range from 6.80 ppm to 8.40 ppm in FIG. 40A is enlarged. This indicates that FrSFA(βN2)B was obtained in this synthesis example.

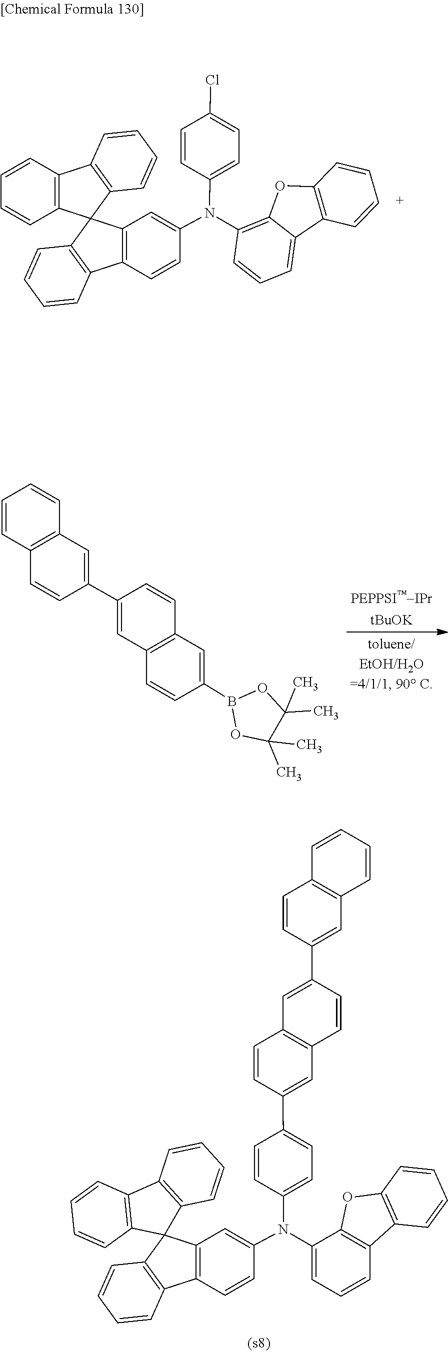 [SMILES: Clc1ccc(N(c2ccc3c(c2)C2(c4ccccc4-c4ccccc42)c2ccccc2-3)c2cccc3c2oc2ccccc23)cc1 ; CC1(C)OB(c2ccc3cc(-c4ccc5ccccc5c4)ccc3c2)OC1(C)C ; c1ccc2c(c1)-c1ccccc1C21c2ccccc2-c2ccc(N(c3ccc(-c4ccc5cc(-c6ccc7ccccc7c6)ccc5c4)cc3)c3cccc4c3oc3ccccc34)cc21]

¹H NMR (dichloromethane-d₂, 500 MHz, 20° C.): δ=8.18 (d, 6.5 Hz, 2H), δ=8.01 (s, 1H), δ=7.98-7.87 (m, 8H), δ=7.77 (t, J=8.0 Hz, 2H), δ=7.74-7.68 (m, 4H), δ=7.54-7.47 (m, 4H), δ=7.38 (t, J=7.0 Hz, 1H), δ=7.33 (d, J=9.5 Hz, 1H), δ=7.31 (t, J=8.5 Hz, 2H), δ=7.25 (t, J=7.5 Hz, 2H), δ=7.19 (t, J=7.5 Hz, 1H), δ=7.18 (dd, J=8.5 Hz, 2.0 Hz, 1H), δ=7.13 (d, J=7.5 Hz, 1H), δ=7.04-6.97 (m, 5H), δ=6.73 (d, J=8.5 Hz, 2H), δ=6.58 (d, J=7.5 Hz, 1H), δ=6.51 (d, J=2.0 Hz, 1H).

The measurement results of the physical properties of FrSFA(βN2)B are shown in the table below. Note that the measurement was performed by a method similar to that in Example 1. As a result, an absorption band of FrSFA(βN2)B is not observed on the wavelength side longer than 440 nm, and it is considered that a reduction in emission efficiency of the light-emitting device due to absorption does not occur in the visible light wavelength region and thus FrSFA(βN2)B can be suitably used. In addition, FrSFA(βN2)B was found to be a substance having high heat resistance.

TABLE 8

| Absorption | | Emission | | | | | Decom- |
|---|---|---|---|---|---|---|---|
| peak (nm) | | peak (nm) | | | | | position |
| Thin | | Thin | | HOMO | LUMO | Tg | temperature |
| film | Solution | film | Solution | (eV) | (eV) | (° C.) | (° C.) |
| 367 | 364 | 463 | 421 | −5.50 | −2.47 | 152.5 | 499.5 |

This application is based on Japanese Patent Application Serial No. 2023-170482 filed with Japan Patent Office on Sep. 29, 2023, the entire contents of which are hereby incorporated by reference.

What is claimed is:

1. An organic compound represented by General Formula (G1): 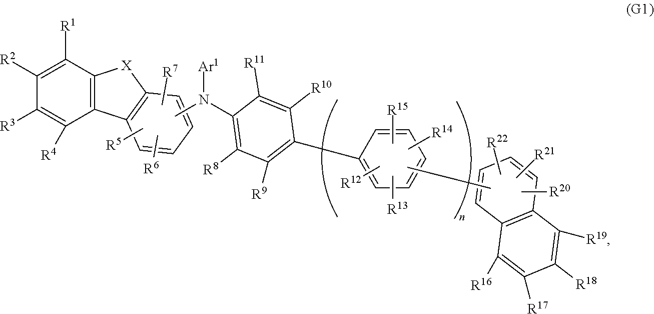
wherein X represents a sulfur atom or an oxygen atom,
wherein each of R¹ to R²² independently represents any one of hydrogen, halogen, a nitrile group, a substituted or unsubstituted alkenyl group, a substituted or unsubstituted vinyl group, a substituted or unsubstituted alkynyl group, a substituted or unsubstituted straight-chain alkyl group having 1 to 6 carbon atoms, a substituted or unsubstituted cycloalkyl group having 3 to 10 carbon atoms, a substituted or unsubstituted alkoxy group having 1 to 6 carbon atoms, a substituted or unsubstituted alkylsilyl group having 3 to 12 carbon atoms, a substituted or unsubstituted aryl group having 6 to 30 carbon atoms, and a substituted or unsubstituted heteroaryl group having 2 to 30 carbon atoms,
wherein at least one of R¹⁶ to R²² represents a substituted or unsubstituted naphthyl group,
wherein n represents an integer of 0 to 4, and
wherein Ar¹ is represented by General Formula (g1-1) or General Formula (g1-2): 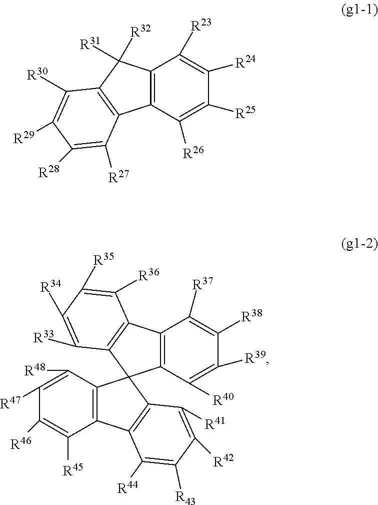
wherein any one of R²³ to R³⁰ or any one of R³³ to R⁴⁸ is bonded to nitrogen of amine, and
wherein each of the others, R³¹, and R³² independently represents any one of hydrogen, halogen, a nitrile group, a substituted or unsubstituted alkenyl group, a substituted or unsubstituted vinyl group, a substituted or unsubstituted alkynyl group, a substituted or unsubstituted straight-chain alkyl group having 1 to 6 carbon atoms, a substituted or unsubstituted cycloalkyl group having 3 to 10 carbon atoms, a substituted or unsubstituted alkoxy group having I to 6 carbon atoms, a substituted or unsubstituted alkylsilyl group having 3 to 12 carbon atoms, a substituted or unsubstituted aryl group having 6 to 30 carbon atoms, and a substituted or unsubstituted heteroaryl group having 2 to 30 carbon atoms.

2. The organic compound according to claim 1, wherein n is 0 or 1.

3. The organic compound according to claim 1, wherein when n is greater than or equal to 2, R¹²s are the same as each other, R¹³s are the same as each other, R¹⁴s are the same as each other, and R¹⁵s are the same as each other.

4. The organic compound according to claim 1,
wherein the organic compound is represented by General Formula (G2): 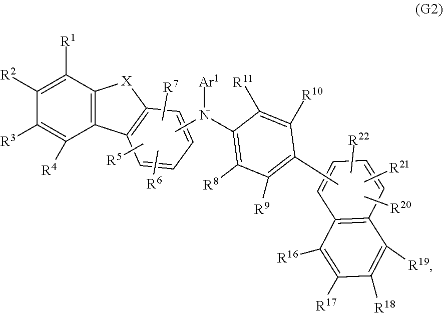
and
wherein at least one of R¹⁶ to R¹⁹ represents a substituted or unsubstituted naphthyl group.

5. The organic compound according to claim 1,
wherein the organic compound is represented by General Formula (G3): 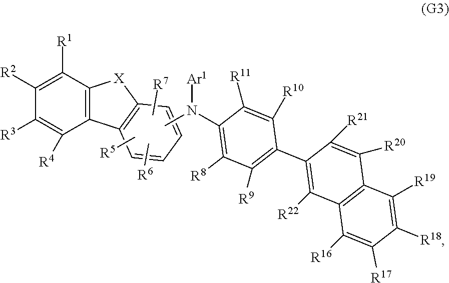
and
wherein at least one of R¹⁶ to R²² represents a substituted or unsubstituted naphthyl group.

6. The organic compound according to claim 1,
wherein any one of R¹⁶ to R²² is represented by General Formula (g2): 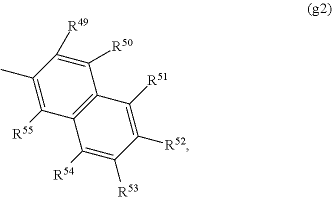
wherein each of R⁴⁹ to R⁵⁵ independently represents any one of hydrogen, halogen, a nitrile group, a substituted or unsubstituted alkenyl group, a substituted or unsubstituted vinyl group, a substituted or unsubstituted alkynyl group, a substituted or unsubstituted straight-chain alkyl group having 1 to 6 carbon atoms, a substituted or unsubstituted cycloalkyl group having 3 to 10 carbon atoms, a substituted or unsubstituted alkoxy group having 1 to 6 carbon atoms, a substituted or unsubstituted alkylsilyl group having 3 to 12 carbon atoms, a substituted or unsubstituted aryl group having 6 to 30 carbon atoms, and a substituted or unsubstituted heteroaryl group having 2 to 30 carbon atoms.

7. An organic semiconductor device comprising the organic compound according to claim 1.

8. A light-emitting device comprising the organic compound according to claim 1.

9. An electronic device comprising the organic semiconductor device according to claim 7.

10. An organic compound represented by General Formula (G4): 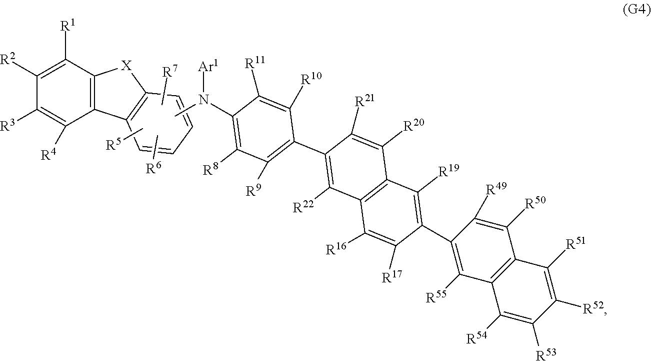
wherein X represents a sulfur atom or an oxygen atom,
wherein each of R¹ to R¹¹, R¹⁶, R¹⁷, R¹⁹ to R²², and R⁴⁹ to R⁵⁵ independently represents any one of hydrogen, halogen, a nitrile group, a substituted or unsubstituted alkenyl group, a substituted or unsubstituted vinyl group, a substituted or unsubstituted alkynyl group, a substituted or unsubstituted straight-chain alkyl group having 1 to 6 carbon atoms, a substituted or unsubstituted cycloalkyl group having 3 to 10 carbon atoms, a substituted or unsubstituted alkoxy group having 1 to 6 carbon atoms, a substituted or unsubstituted alkylsilyl group having 3 to 12 carbon atoms, a substituted or unsubstituted aryl group having 6 to 30 carbon atoms, and a substituted or unsubstituted heteroaryl group having 2 to 30 carbon atoms, and
wherein Ar¹ is represented by General Formula (g1-1) or General Formula (g1-2): 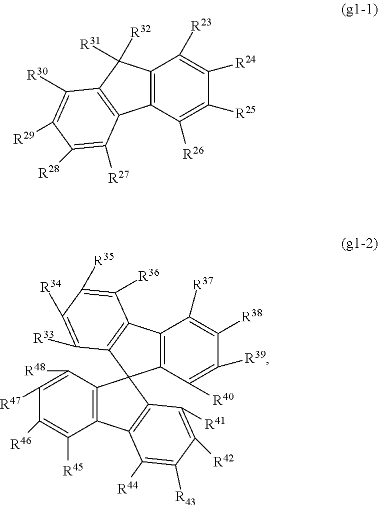
wherein any one of R²³ to R³⁰ or any one of R³³ to R⁴⁸ is bonded to nitrogen of amine, and
wherein each of the others, R³¹, and R³² independently represents any one of hydrogen, halogen, a nitrile group, a substituted or unsubstituted alkenyl group, a substituted or unsubstituted vinyl group, a substituted or unsubstituted alkynyl group, a substituted or unsubstituted straight-chain alkyl group having 1 to 6 carbon atoms, a substituted or unsubstituted cycloalkyl group having 3 to 10 carbon atoms, a substituted or unsubstituted alkoxy group having 1 to 6 carbon atoms, a substituted or unsubstituted alkylsilyl group having 3 to 12 carbon atoms, a substituted or unsubstituted aryl group having 6 to 30 carbon atoms, and a substituted or unsubstituted heteroaryl group having 2 to 30 carbon atoms.

11. The organic compound according to claim 10,
wherein the organic compound is represented by General Formula (G5):

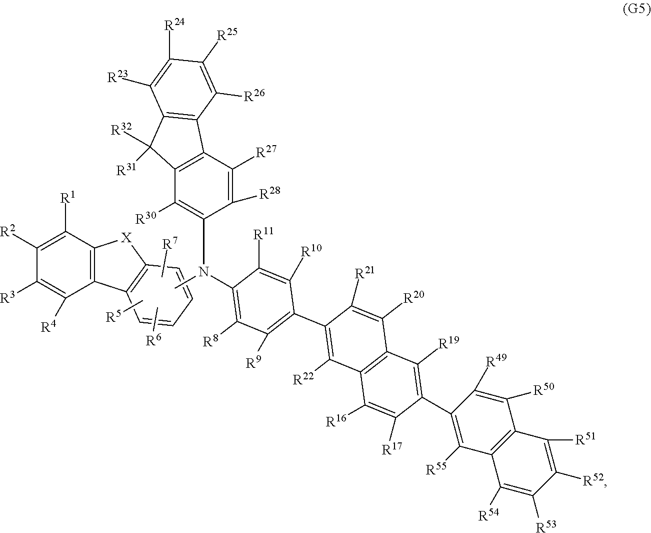

12. The organic compound according to claim 10,
wherein the organic compound is represented by General Formula (G6):

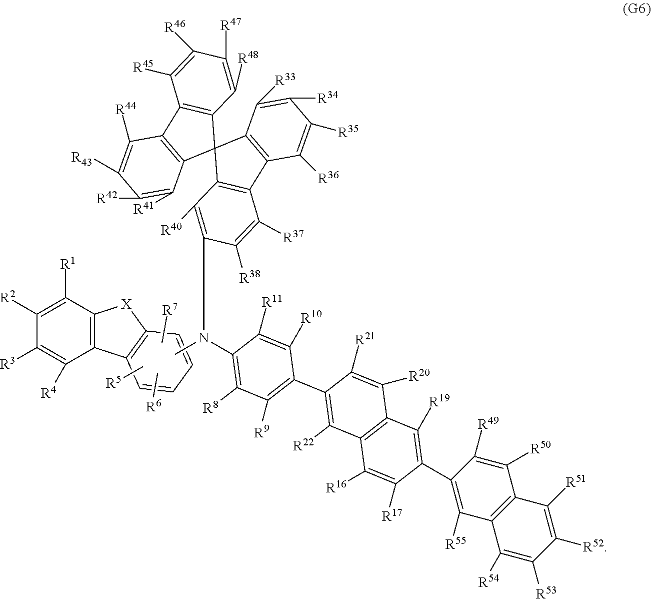

13. An organic semiconductor device comprising the organic compound according to claim 10.

14. A light-emitting device comprising the organic compound according to claim 10.

15. An electronic device comprising the organic semiconductor device according to claim 13.
